(19) United States
(12) Patent Application Publication
(10) Pub. No.: US 2010/0009969 A1
(43) Pub. Date: Jan. 14, 2010

(54) Fused Oxazoles & Thiazoles As Histamine H3- Receptor Ligands

(75) Inventors: Frédéric Denonne, Brussels (BE); Sylvain Celanire, Feigeres (FR); Laurent Provins, Soignies (BE); Sabine Defays, Tubize (BE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB PHARMA, S.A., Bruxelles (BE)

(21) Appl. No.: 12/375,288

(22) PCT Filed: Jul. 17, 2007
(86) PCT No.: PCT/EP07/06338

§ 371 Date: Sep. 2, 2009

(30) Foreign Application Priority Data
Jul. 27, 2006 (EP) 06015639.5

(51) Int. Cl. A61K 31/55; A61K 31/5377; C07D 513/04; C07D 415/00

(52) U.S. Cl. 514/215; 514/233.8; 540/578; 544/133

(57) ABSTRACT

The present invention relates to compounds comprising fused oxazole or thiazole derivatives of formula (I), processes for preparing them, pharmaceutical compositions comprising said compounds and their uses as H₃-receptor ligands, wherein A is a cyclic amine which is linked to the propylene group via an amino nitrogen; B is selected from the group consisting of heteroaryl, 5-8-membered heterocycloalkyl, 5-8-membered cycloalkyl; X is either N or CH; Y is either O or S.

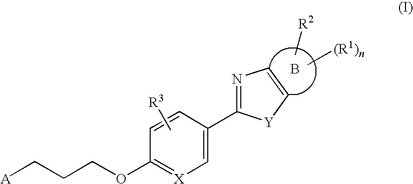

The present invention relates to compounds comprising a fused oxazole or thiazole moiety, processes for preparing them, pharmaceutical compositions comprising said compounds and their uses as pharmaceuticals.

The histamine H₃ receptor has been known for several years and identified pharmacologically in 1983 by Arrang, J. M. et al. (Nature 1983, 302, 832). Since the cloning of the human histamine H₃ receptor in 1999, histamine H₃ receptors have been successively cloned by sequence homology from a variety of species, including rat, guinea pig, mouse and monkey.

Histamine H₃-receptor agonists, antagonists and inverse agonists have shown potential therapeutic applications as described in the literature, for example by Stark, H. (Exp. Opin. Ther. Patents 2003, 13, 851) and by Leurs R. et al. (Nature Reviews Drug Discovery 2005, 4, 107-120).

The histamine H₃ receptor is predominantly expressed in the mammalian central nervous system but can also be found in the autonomic nervous system. Evidence has been shown that the histamine H₃ receptor displays high constitutive activity, which activity occurs in the absence of endogenous histamine or of a H₃-receptor agonist. Thus, a histamine H₃-receptor antagonist and/or inverse agonist could inhibit this activity.

The general pharmacology of histamine H₃ receptor, including H₃-receptor subtypes, has been reviewed by Hancock, A. A (Life Sci. 2003, 73, 3043). The histamine H₃ receptor is not only considered as a presynaptic autoreceptor on histaminergic neurons, but also as a heteroreceptor on non-histaminergic neurons (Barnes, W. et al., Eur. J. Pharmacol. 2001, 431, 215). Indeed, the histamine H₃ receptor has been shown to regulate the release of histamine but also of other important neurotransmitters, including acetylcholine, dopamine, serotonin, norepinephrin and γ-aminobutyric acid (GABA).

Thus, the histamine H₃ receptor is of current interest for the development of new therapeutics and the literature suggests that novel histamine H₃-receptor antagonists or inverse agonists may be useful for the treatment and prevention of diseases or pathological conditions of the central nervous system including Mild Cognitive Impairment (MCI), Alzheimer's disease, learning and memory disorders, cognitive disorders, attention deficit disorder (ADD), attention-deficit hyperactivity disorder (ADHD), Parkinson's disease, schizophrenia, dementia, depression, epilepsy, seizures or convulsions, sleep/wake disorders, narcolepsy, and/or obesity.

H₃-receptor ligands alone or in combination with an acetylcholinesterase inhibitor may also be useful in the treatment of cholinergic-deficit disorders, Mild Cognitive Impairment and Alzheimer's disease as reported by Morisset, S. et al. in Eur. J. Pharmacol. 1996, 315, R1-R2.

H₃-receptor ligands, alone or in combination with a histamine H₁-receptor antagonist may be useful for the treatment of upper airway allergic disorders, as reported by McLeod, R. et al. in J. Pharmacol. Exp. Ther. 2003, 305, 1037.

As described in international patent application WO02/072093, H₃-receptor ligands alone or in combination with a muscarinic receptor ligand and particularly with a muscarinic M₂-receptor antagonist, may be useful for the treatment of cognitive disorders, Alzheimer's disease, attention-deficit hyperactivity disorder.

As described in international patent application WO2005/056056, H₃-receptor ligands alone or in combination with a serotonin reuptake inhibitor may be useful for the treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitor.

H₃-receptor ligands may also be useful in the treatment of sleep/wake and arousal/vigilance disorders such as hypersomnia, and narcolepsy according to Passani, M. B. et al. in Trends Pharmacol. Sci. 2004, 25(12), 618-25.

In general, H₃-receptor ligands, and particularly H₃-receptor antagonists or inverse agonists may be useful in the treatment of all type of cognitive-related disorders as reviewed by Hancock, A. A and Fox, G. B. in Expert Opin. Invest. Drugs 2004, 13, 1237. In particular, histamine H₃-receptor antagonists or inverse agonists may be useful in the treatment of cognitive dysfunctions in diseases such as mild cognitive impairment, dementia, Alzheimer's disease, Parkinson's disease, Down's syndrome as well as in the treatment of attention-deficit hyperactivity disorder (ADHD) as non-psychostimulant agents (see for example Witkin, J. M. et al., Pharmacol. Ther. 2004, 103(1), 1-20).

H₃-receptor antagonists or inverse agonists may also be useful in the treatment of psychotic disorders such as schizophrenia, migraine, eating disorders such as obesity, inflammation, pain, anxiety, stress, depression and cardiovascular disorders, in particular acute myocardial infarction.

There is therefore a need to manufacture new compounds which can potentially act as H₃-receptor ligands.

Early literature reports (e.g. Ali, S. M. et al. in J. Med. Chem. 1999, 42, 903 and Drugs Fut. 1996, 21, 507) describe that an imidazole function is essential for high affinity histamine H₃-receptor ligands; this is confirmed, for example, by U.S. Pat. No. 6,506,756B2, U.S. Pat. No. 6,518,287B2, U.S. Pat. No. 6,528,522B2 and U.S. Pat. No. 6,762,186B2 which relate to substituted imidazole compounds that have H₃-receptor antagonist or dual histamine H₁-receptor and H₃-receptor antagonist activity.

International patent application WO 02/12214 describes non-imidazole aryloxyalkylamines useful for the treatment of conditions and disorders mediated by the histamine receptor. U.S. Pat. No. 6,436,939B2 relates to H₃-receptor antagonists comprising a benzoxazole or benzothiazole moiety.

International patent application WO 03/097047 relates to compounds comprising an oxazole moiety for use as melanin concentrating hormone antagonist in the treatment of obesity and diabetes.

International patent application WO 98/27061 describes compounds comprising an oxazole moiety which may be used, for example, for the treatment of hypertension, angina pectoris and diabetic complications.

It has now surprisingly been found that fused oxazole and fused thiazole derivatives may act as H₃-receptor ligands and therefore may demonstrate therapeutic properties for one or more pathologies that we have described above.

### SUMMARY OF THE INVENTION

Thus, the present invention relates to compounds of formula (I), geometrical isomers, enantiomers, diastereoisomers, pharmaceutically acceptable salts and all possible mixtures thereof,

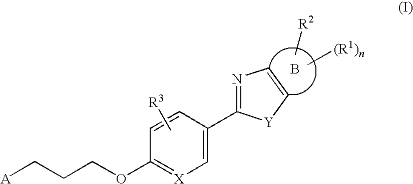

In formula (I)

A is a substituted or unsubstituted cyclic amine which is linked to the propylene group via an amino nitrogen.

B is selected from the group consisting of heteroaryl, 5-8-membered heterocycloalkyl, 5-8-membered cycloalkyl. In a specific embodiment B is selected from the group consisting of 5-7-membered heterocycloalkyl, 5-7-membered cycloalkyl. In a further specific embodiment, B may be a tetrahydropyridyl.

X is either N or CH. In a specific embodiment X is CH.

Y is either O or S. In a specific embodiment Y is S.

R¹ is selected from the group comprising or consisting of sulfonyl, amino, substituted or unsubstituted C₁-C₆-alkyl, substituted or unsubstituted C₂-C₆-alkenyl, substituted or unsubstituted C₂-C₆-alkynyl, substituted or unsubstituted aryl, substituted or unsubstituted heteroaryl, substituted or unsubstituted C₃-C₈-cycloalkyl, substituted or unsubstituted 3-8-membered heterocycloalkyl, acyl, substituted or unsubstituted C₁-C₆-alkyl aryl, substituted or unsubstituted C₁-C₆-alkyl heteroaryl, substituted or unsubstituted C₂-C₆-alkenyl aryl, substituted or unsubstituted C₂-C₆-alkenyl heteroaryl, substituted or unsubstituted C₂-C₆-alkynyl aryl, substituted or unsubstituted C₂-C₆-alkynyl heteroaryl, substituted or unsubstituted C₁-C₆-alkyl cycloalkyl, substituted or unsubstituted C₁-C₆-alkyl heterocycloalkyl, substituted or unsubstituted C₂-C₆-alkenyl cycloalkyl, substituted or unsubstituted C₂-C₆-alkenyl heterocycloalkyl, substituted or unsubstituted C₂-C₆-alkynyl cycloalkyl, substituted or unsubstituted C₂-C₆-alkynyl heterocycloalkyl, alkoxycarbonyl, aminocarbonyl, substituted or unsubstituted C₁-C₆-alkyl carboxy, substituted or unsubstituted C₁-C₆-alkyl acyl, substituted or unsubstituted aryl acyl, substituted or unsubstituted heteroaryl acyl, substituted or unsubstituted C₃-C₈-(hetero)cycloalkyl acyl, substituted or unsubstituted C₁-C₆-alkyl acyloxy, substituted or unsubstituted C₁-C₆-alkyl alkoxy, substituted or unsubstituted C₁-C₆-alkyl alkoxycarbonyl, substituted or unsubstituted C₁-C₆-alkyl aminocarbonyl, substituted or unsubstituted C₁-C₆-alkyl acylamino, acylamino, acylaminocarbonyl, ureido, substituted or unsubstituted C₁-C₆-alkyl ureido, substituted or unsubstituted C₁-C₆-alkyl carbamate, substituted or unsubstituted C₁-C₆-alkyl amino, substituted or unsubstituted C₁-C₆-alkyl sulfonyloxy, substituted or unsubstituted C₁-C₆-alkyl sulfonyl, substituted or unsubstituted C₁-C₆-alkyl sulfinyl, substituted or unsubstituted C₁-C₆-alkyl sulfanyl, substituted or unsubstituted C₁-C₆-alkyl sulfonylamino, substituted or unsubstituted C₁-C₆-alkyl aminosulfonyl, hydroxy, halogen, cyano, carboxy, oxo, thioxo.

n is equal to 0, 1, 2 or 3. In a specific embodiment, n is either 0 or 1.

R² is selected from the group comprising or consisting of hydrogen, sulfonyl, amino, substituted or unsubstituted C₁-C₆-alkyl, substituted or unsubstituted C₂-C₆-alkenyl, substituted or unsubstituted C₂-C₆-alkynyl, substituted or unsubstituted aryl, substituted or unsubstituted heteroaryl, substituted or unsubstituted C₃-C₈-cycloalkyl, substituted or unsubstituted 3-8-membered heterocycloalkyl, acyl, substituted or unsubstituted C₁-C₆-alkyl aryl, substituted or unsubstituted C₁-C₆-alkyl heteroaryl, substituted or unsubstituted C₂-C₆-alkenyl aryl, substituted or unsubstituted C₂-C₆-alkenyl heteroaryl, substituted or unsubstituted C₂-C₆-alkynyl aryl, substituted or unsubstituted C₂-C₆-alkynyl heteroaryl, substituted or unsubstituted C₁-C₆-alkyl cycloalkyl, substituted or unsubstituted C₁-C₆-alkyl heterocycloalkyl, substituted or unsubstituted C₂-C₆-alkenyl cycloalkyl, substituted or unsubstituted C₂-C₆-alkenyl heterocycloalkyl, substituted or unsubstituted C₂-C₆-alkynyl cycloalkyl, substituted or unsubstituted C₂-C₆-alkynyl heterocycloalkyl, alkoxycarbonyl, aminocarbonyl, substituted or unsubstituted C₁-C₆-alkyl carboxy, substituted or unsubstituted C₁-C₆-alkyl acyl, substituted or unsubstituted aryl acyl, substituted or unsubstituted heteroaryl acyl, substituted or unsubstituted C₃-C₈-(hetero)cycloalkyl acyl, substituted or unsubstituted C₁-C₆-alkyl acyloxy, substituted or unsubstituted C₁-C₆-alkyl alkoxy, substituted or unsubstituted C₁-C₆-alkyl alkoxycarbonyl, substituted or unsubstituted C₁-C₆-alkyl aminocarbonyl, substituted or unsubstituted C₁-C₆-alkyl acylamino, acylamino, acylaminocarbonyl, ureido, substituted or unsubstituted C₁-C₆-alkyl ureido, substituted or unsubstituted C₁-C₆-alkyl carbamate, substituted or unsubstituted C₁-C₆-alkyl amino, substituted or unsubstituted C₁-C₆-alkyl sulfonyloxy, substituted or unsubstituted C₁-C₆-alkyl sulfonyl, substituted or unsubstituted C₁-C₆-alkyl sulfinyl, substituted or unsubstituted C₁-C₆-alkyl sulfanyl, substituted or unsubstituted C₁-C₆-alkyl sulfonylamino, substituted or unsubstituted C₁-C₆-alkyl aminosulfonyl, hydroxy, halogen, cyano, carboxy, oxo, thioxo.

R³ is hydrogen or C₁-C₆-alkyl or halogen or C₁-C₆-alkoxy. In a specific embodiment R³ is hydrogen.

### DETAILED DESCRIPTION OF THE INVENTION

The following definitions apply for the chemical moieties used herein, unless otherwise specified.

“C₁-C₆-alkyl” refers to alkyl groups having 1 to 6 carbon atoms. This term is exemplified by groups such as methyl, ethyl, n-propyl, isopropyl, n-butyl, isobutyl, tert-butyl, n-pentyl, n-hexyl, trifluoromethyl, trifluoropropyl and the like.

“Aryl” refers to an unsaturated aromatic carbocyclic group of from 6 to 14 carbon atoms having a single ring (e.g., phenyl) or multiple condensed rings (e.g., naphthyl). Preferred aryl include phenyl, 4-fluorophenyl, 4-methylphenyl, naphthyl, phenantrenyl and the like.

“C₁-C₆-alkyl aryl” refers to C₁-C₆-alkyl groups having an aryl substituent, including benzyl, phenethyl and the like.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

“C₁-C₆-alkyl heteroaryl” refers to C₁-C₆-alkyl groups having a heteroaryl substituent, including (2-furyl)methyl, (2-thienyl)methyl, (2-thienyl)ethyl, 2-(1H-indol-3-yl)ethyl and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (allyl, —CH₂CH═CH₂) and the like.

“C₂-C₆-alkenyl aryl” refers to C₂-C₆-alkenyl groups having an aryl substituent, including 2-phenylvinyl and the like.

“C₂-C₆-alkenyl heteroaryl” refers to C₂-C₆-alkenyl groups having a heteroaryl substituent, including 2-(3-pyridinyl)vinyl and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

“C₂-C₆-alkynyl aryl” refers to C₂-C₆-alkynyl groups having an aryl substituent, including phenylethynyl and the like.

“C₂-C₆-alkynyl heteroaryl” refers to C₂-C₆-alkynyl groups having a heteroaryl substituent, including 2-thienylethynyl and the like.

“C₃-C₈-cycloalkyl” refers to a saturated or partially unsaturated carbocyclic group of from 3 to 8 carbon atoms having a single ring (e.g., cyclohexyl or cyclohexenyl) or multiple condensed rings (e.g., norbornyl). Preferred cycloalkyl include cyclobutyl, cyclobutenyl, cyclopentyl, cyclopentenyl, cyclohexyl, cyclohexenyl, cycloheptyl, cycloheptenyl, norbornyl and the like.

“C₁-C₆-alkyl cycloalkyl” refers to C₁-C₆-alkyl groups having a cycloalkyl substituent, including cyclohexylmethyl, cyclopentylpropyl, and the like.

“Heterocycloalkyl” refers to a C₃-C₈-cycloalkyl group according to the definition above, in which 1 to 3 carbon atoms are replaced by hetero atoms chosen from the group consisting of O, S, NR, R being defined as hydrogen or C₁-C₆ alkyl. Preferred heterocycloalkyl include pyrrolidine, piperidine, piperazine, 1-methylpiperazine, morpholine, tetrahydropyridyl, dihydro-1H-pyrrolyl, tetrahydro-1H-azepinyl and the like.

“C₁-C₆-alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

“Carboxy” refers to the group —C(O)OH.

“C₁-C₆-alkyl carboxy” refers to C₁-C₆-alkyl groups having a carboxy substituent, including 2-carboxyethyl and the like.

“Acyl” refers to the group —C(O)R where R includes H, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Particular examples of acyl groups include acetyl, trifluoroacetyl, benzoyl, cyclohexylcarbonyl, thien-2-ylcarbonyl, 2,2-dimethylpropanoyl, butyryl, cyclopropylcarbonyl, isonicotinoyl, methoxyacetyl, 3,3,3-trifluoropropanoyl.

“C₁-C₆-alkyl acyl” refers to C₁-C₆-alkyl groups having an acyl substituent, including 2-acetylethyl and the like.

“Aryl acyl” refers to aryl groups having an acyl substituent, including 2-acetylphenyl and the like.

“Heteroaryl acyl” refers to hetereoaryl groups having an acyl substituent, including 2-acetylpyridyl and the like.

“C₃-C₈-(hetero)cycloalkyl acyl” refers to 3 to 8 membered cycloalkyl or heterocycloalkyl groups having an acyl substituent.

“Acyloxy” refers to the group —OC(O)R where R includes H, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acyloxy” refers to C₁-C₆-alkyl groups having an acyloxy substituent, including 2-(acetyloxy)ethyl and the like.

“Alkoxy” refers to the group —O—R where R includes “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl alkoxy” refers to C₁-C₆-alkyl groups having an alkoxy substituent, including 2-ethoxyethyl and the like.

“Alkoxycarbonyl” refers to the group —C(O)OR where R includes “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred alkoxycarbonyl groups include ethoxycarbonyl and tert-butoxycarbonyl.

“C₁-C₆-alkyl alkoxycarbonyl” refers to C₁-C₆-alkyl groups having an alkoxycarbonyl substituent, including 2-(benzyloxycarbonyl)ethyl and the like.

“Aminocarbonyl” refers to the group —C(O)NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocyclo-alkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred aminocarbonyl groups include (ethylamino)carbonyl, (benzylamino)carbonyl, anilinocarbonyl, {[2-(2-thienyl)ethyl]amino}carbonyl, aminocarbonyl, (isopropylamino)carbonyl, (cyclohexylamino)carbonyl, {[1-(trifluoroacetyl)piperidin-4-yl]amino}carbonyl, [(2-ethoxy-2-oxoethyl)amino]carbonyl, [(2,4-difluorophenyl)amino]carbonyl, (butylamino)carbonyl, piperidin-1-ylcarbonyl.

“C₁-C₆-alkyl aminocarbonyl” refers to C₁-C₆-alkyl groups having an aminocarbonyl substituent, including 2-(dimethylaminocarbonyl)ethyl and the like.

“Acylamino” refers to the group —NRC(O)R′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, including acetylamino and the like.

“C₁-C₆-alkyl acylamino” refers to C₁-C₆-alkyl groups having an acylamino substituent, including 2-(propionylamino)ethyl and the like.

“Acylaminocarbonyl” refers to the group —C(O)NRC(O)R′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Ureido” refers to the group —NRC(O)NR′R″ where each R, R′, R″ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R′ and R″, together with the nitrogen atom to which they are attached, can optionally form a 3-8-membered heterocycloalkyl ring. Preferred ureido groups include [(2-methylpyrrolidin-1-yl)carbonyl]amino.

“C₁-C₆-alkyl ureido” refers to C₁-C₆-alkyl groups having an ureido substituent, including 2-(N′-methylureido)ethyl and the like.

“Carbamate” refers to the group —NRC(O)OR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred carbamate groups include [(benzyloxy)carbonyl]amino.

“Amino” refers to the group —NRR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R and R′, together with the nitrogen atom to which they are attached, can optionally form a 3-8-membered hetero-cycloalkyl ring. Preferred amino groups include amino, methylamino, dimethylamino, piperidin-1-yl, pyrrolidin-1-yl, piperazin-1-yl and the like.

“C₁-C₆-alkyl amino” refers to C₁-C₆-alkyl groups having an amino substituent, including 2-(1-pyrrolidinyl)ethyl and the like.

“Halogen” refers to fluoro, chloro, bromo and iodo atoms.

“Sulfonyloxy” refers to a group —OSO₂—R wherein R is selected from H, “C₁-C₆-alkyl”, “C₁-C₆-alkyl” substituted with halogens, e.g., an —OSO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyloxy” refers to C₁-C₆-alkyl groups having a sulfonyloxy substituent, including 2-(methylsulfonyloxy)ethyl and the like.

“Sulfonyl” refers to group “—SO₂—R” wherein R is selected from H, “aryl”, “heteroaryl”, “C₁-C₆-alkyl”, “C₁-C₆-alkyl” substituted with halogens, e.g., an —SO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred sulfonyl groups include methylsulfonyl and (4-methylphenyl)sulfonyl.

“C₁-C₆-alkyl sulfonyl” refers to C₁-C₆-alkyl groups having a sulfonyl substituent, including 2-(methylsulfonyl)ethyl and the like.

“Sulfinyl” refers to a group “—S(O)—R” wherein R is selected from H, “C₁-C₆-alkyl”, “C₁-C₆-alkyl” substituted with halogens, e.g., an —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfinyl” refers to C₁-C₆-alkyl groups having a sulfinyl substituent, including 2-(methylsulfinyl)ethyl and the like.

“Sulfanyl” refers to groups —S—R where R includes H, “C₁-C₆-alkyl”, “C₁-C₆-alkyl” optionally substituted with halogens, e.g a —S—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

Preferred sulfanyl groups include methylsulfanyl, ethylsulfanyl, and the like.

“C₁-C₆-alkyl sulfanyl” refers to C₁-C₆-alkyl groups having a sulfanyl substituent, including 2-(ethylsulfanyl)ethyl and the like.

“Sulfonylamino” refers to a group —NRSO₂—R′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonylamino” refers to C₁-C₆-alkyl groups having a sulfonylamino substituent, including 2-(ethylsulfonylamino)ethyl and the like.

“Aminosulfonyl” refers to a group —SO₂—NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl aminosulfonyl” refers to C₁-C₆-alkyl groups having an aminosulfonyl substituent, including 2-(cyclohexylaminosulfonyl)ethyl and the like.

“Hydroxy” refers to the group —OH.

“C₁-C₆-alkyl hydroxy” refers to C₁-C₆-alkyl groups having a hydroxy substituent, including 2-hydroxyethyl, 2,3-dihydroxypropyl, 3-hydroxybutyl, and the like.

“C₃-C₈-cycloalkyl hydroxy” refers to C₃-C₈-cycloalkyl groups having a hydroxy substituent, including 3-hydroxycyclobutyl, and the like.

“Oxo” refers to ═O.

“Thioxo” refers to ═S.

“Substituted or unsubstituted”: Unless otherwise constrained by the definition of the individual substituent, the above set out groups, like “alkyl”, “alkenyl”, “alkynyl”, “aryl” and “heteroaryl” etc. groups can optionally be substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “acyl”, “acyloxy”, “acylamino”, “aminocarbonyl”, “alkoxycarbonyl”, “ureido”, “carbamate”, “aryl”, “heteroaryl”, “sulfinyl”, “sulfonyl”, “alkoxy”, “sulfanyl”, “halogen”, “carboxy”, trihalomethyl, cyano, hydroxy, mercapto, nitro, oxo, thioxo, silyl and the like. Alternatively, said substitution could also comprise situations where neighbouring substituents have undergone ring closure, notably when vicinal functional substituents are involved, thus forming, e.g., lactams, lactons, cyclic anhydrides, but also acetals, thioacetals, aminals formed by ring closure for instance in an effort to obtain a protective group.

In a specific embodiment the present invention comprises compounds of formula (I) wherein A is substituted by a moiety selected from a substituted or unsubstituted C₁-C₆-alkyl, substituted or unsubstituted C₂-C₆-alkenyl, substituted or unsubstituted C₂-C₆-alkynyl, substituted or unsubstituted aryl, substituted or unsubstituted heteroaryl, substituted or unsubstituted saturated or unsaturated 3-8-membered cycloalkyl (e.g. a cyclopropyl), substituted or unsubstituted 3-8-membered heterocycloalkyl or an amino. In a specific embodiment the present invention comprises compounds of formula (I) wherein A is a pyrrolidinyl, an azepanyl, a piperazinyl or a piperidinyl group, optionally substituted by substituted or unsubstituted C₁-C₆-alkyl, substituted or unsubstituted C₂-C₆-alkenyl, substituted or unsubstituted C₂-C₆-alkynyl, substituted or unsubstituted aryl, substituted or unsubstituted heteroaryl, substituted or unsubstituted saturated or unsaturated 3-8-membered cycloalkyl, substituted or unsubstituted saturated or unsaturated 3-8-membered heterocycloalkyl, or an amino. A specific substituent for A is a C₁-C₆-alkyl, e.g. a methyl, or an amino, e.g. a dimethylamino.

Examples of A are: piperidin-1-yl, 2-methylpyrrolidin-1-yl and enantiomers, e.g. (2R)-2-methylpyrrolidin-1-yl and (2S)-2-methylpyrrolidin-1-yl, azepan-1-yl, (2-pyrrolidin-1-yl-methyl)pyrrolidin-1-yl, 4-isopropylpiperazin-1-yl, 2-methylpiperidin-1-yl, 3-(dimethylamino)pyrrolidin-1-yl and enantiomers, e.g. (3R)-3-(dimethylamino)pyrrolidin-1-yl and (3S)-3-(dimethylamino)pyrrolidin-1-yl, 3,5-dimethylpiperidin-1-yl, 2-(hydroxymethyl)pyrrolidin-1-yl and enantiomers, e.g. (2R)-2-(hydroxymethyl)pyrrolidin-1-yl and (2S)-2-(hydroxymethyl)pyrrolidin-1-yl.

In a further specific embodiment B is a 5, 6 or 7-membered cycloalkyl or heterocycloalkyl, such as a tetrahydropyridyl, a dihydro-1H-pyrrolyl, a tetrahydro-1H-azepinyl, a cyclohexenyl or a cyclopentenyl.

In a further specific embodiment B is a 5, 6 or 7-membered cycloalkyl or heterocycloalkyl, and forms together with the oxazole or the thiazole ring fused heterocycles including 4,5,6,7-tetrahydro[1,3]thiazolopyridine, 4,5,6,7-tetrahydro[1,3]oxazolopyridine, 5,6-dihydro-4H-pyrrolo[1,3]thiazole, 5,6,7,8-tetrahydro-4H-[1,3]thiazoloazepine, 4,5,6,7-tetrahydro-1,3-benzothiazole, 5,6-dihydro-4H-cyclopenta[d][1,3]thiazole. Examples of such heterocycles are 4,5,6,7-tetrahydro[1,3]thiazolo[5,4-c]pyridine, 4,5,6,7-tetrahydro[1,3]thiazolo[4,5-b]pyridine, 4,5,6,7-tetrahydro[1,3]thiazolo[4,5-c]pyridine, 4,5,6,7-tetrahydro[1,3]oxazolo-[4,5-c]pyridine, 5,6-dihydro-4H-pyrrolo[3,4-d][1,3]thiazole, 5,6,7,8-tetrahydro-4H-[1,3]thiazolo[4,5-d]azepine, 5,6,7,8-tetrahydro-4H-[1,3]thiazolo[5,4-c]azepine, 5,6,7,8-tetrahydro-4H-[1,3]thiazolo[5,4-b]azepine, 4,5,6,7-tetrahydro-1,3-benzothiazole, 5,6-dihydro-4H-cyclopenta[d][1,3]thiazole.

In a specific embodiment the present invention comprises compounds of formula (I) wherein R¹ is selected from the group comprising or consisting of substituted or unsubstituted C₁-C₆-alkyl, substituted or unsubstituted aryl, substituted or unsubstituted heteroaryl, substituted or unsubstituted C₃-C₈-cycloalkyl, substituted or unsubstituted 3-8-membered heterocycloalkyl, acyl, substituted or unsubstituted C₁-C₆-alkyl cycloalkyl, substituted or unsubstituted C₁-C₆-alkyl heterocycloalkyl, alkoxycarbonyl, aminocarbonyl, substituted or unsubstituted C₁-C₆-alkyl alkoxycarbonyl, substituted or unsubstituted C₁-C₆-alkyl aminocarbonyl, hydroxy, halogen, cyano, carboxy, oxo, thioxo; and n is 0 or 1, in particular 1. In a further embodiment n is 0.

In a further specific embodiment R¹ is selected from the group comprising or consisting of substituted or unsubstituted C₁-C₆-alkyl, hydroxy, oxo; and n is 0 or 1, in particular 1. In a further embodiment n is 0.

In a specific embodiment the present invention comprises compounds of formula (I) wherein R² is selected from the group consisting of hydrogen, sulfonyl, amino, substituted or unsubstituted C₁-C₆-alkyl, substituted or unsubstituted C₂-C₆-alkenyl, substituted or unsubstituted C₂-C₆-alkynyl, substituted or unsubstituted aryl, substituted or unsubstituted heteroaryl, substituted or unsubstituted C₃-C₈-cycloalkyl, substituted or unsubstituted 3-8-membered heterocycloalkyl, acyl, substituted or unsubstituted C₁-C₆-alkyl aryl, substituted or unsubstituted C₁-C₆-alkyl heteroaryl, substituted or unsubstituted C₂-C₆-alkenyl aryl, substituted or unsubstituted C₂-C₆-alkenyl heteroaryl, substituted or unsubstituted C₂-C₆-alkynyl aryl, substituted or unsubstituted C₂-C₆-alkynyl heteroaryl, substituted or unsubstituted C₁-C₆-alkyl cycloalkyl, substituted or unsubstituted C₁-C₆-alkyl heterocycloalkyl, substituted or unsubstituted C₂-C₆-alkenyl cycloalkyl, substituted or unsubstituted C₂-C₆-alkenyl heterocycloalkyl, substituted or unsubstituted C₂-C₆-alkynyl cycloalkyl, substituted or unsubstituted C₂-C₆-alkynyl heterocycloalkyl, alkoxycarbonyl, aminocarbonyl, substituted or unsubstituted C₁-C₆-alkyl carboxy, substituted or unsubstituted C₁-C₆-alkyl acyl, substituted or unsubstituted aryl acyl, substituted or unsubstituted heteroaryl acyl, substituted or unsubstituted C₃-C₈-(hetero)cycloalkyl acyl, substituted or unsubstituted C₁-C₆-alkyl acyloxy, substituted or unsubstituted C₁-C₆-alkyl alkoxy, substituted or unsubstituted C₁-C₆-alkyl alkoxycarbonyl, substituted or unsubstituted C₁-C₆-alkyl aminocarbonyl, substituted or unsubstituted C₁-C₆-alkyl acylamino, acylamino, ureido, substituted or unsubstituted C₁-C₆-alkyl ureido, substituted or unsubstituted C₁-C₆-alkyl carbamate, substituted or unsubstituted C₁-C₆-alkyl amino, hydroxy, oxo, thioxo.

In a further specific embodiment the present invention comprises compounds of formula (I) wherein R² is selected from the group consisting of hydrogen, sulfonyl, substituted or unsubstituted C₁-C₆-alkyl, substituted or unsubstituted C₂-C₆-alkenyl, substituted or unsubstituted C₂-C₆-alkynyl, substituted or unsubstituted aryl, substituted or unsubstituted heteroaryl, substituted or unsubstituted C₃-C₈-cycloalkyl, substituted or unsubstituted 3-8-membered heterocycloalkyl, an acyl moiety, substituted or unsubstituted C₁-C₆-alkyl cycloalkyl, substituted or unsubstituted C₁-C₆-alkyl heterocycloalkyl, alkoxycarbonyl, aminocarbonyl, acylamino, ureido, substituted or unsubstituted C₁-C₆-alkyl ureido, substituted or unsubstituted C₁-C₆-alkyl carbamate, amino, substituted or unsubstituted C₁-C₆-alkyl amino, hydroxy, oxo.

In a more specific embodiment the present invention comprises compounds of formula (I) wherein R² is selected from the group consisting of hydrogen, sulfonyl, substituted or unsubstituted C₁-C₆-alkyl, substituted or unsubstituted aryl, substituted or unsubstituted C₃-C₈-cycloalkyl, substituted or unsubstituted 3-8-membered heterocycloalkyl, acyl, substituted or unsubstituted C₁-C₆-alkyl cycloalkyl, alkoxycarbonyl, aminocarbonyl, acylamino, ureido, substituted or unsubstituted C₁-C₆-alkyl carbamate, amino, hydroxy, oxo.

Examples of R² are: hydrogen, methyl, ethyl, acetyl, cyclohexylmethyl, cyclopentyl, trifluoroacetyl, 4-fluorophenyl, benzoyl, cyclohexylcarbonyl, thien-2-ylcarbonyl, 2,2-dimethylpropanoyl, butyryl, tert-butoxycarbonyl, (ethylamino)carbonyl, cyclopropylcarbonyl, isonicotinoyl, methoxyacetyl, methylsulfonyl, (benzylamino)carbonyl, anilinocarbonyl, {[2-(2-thienyl)ethyl]amino}carbonyl, aminocarbonyl, (isopropylamino)carbonyl, (cyclohexyl-amino)carbonyl, {[1-(trifluoroacetyl)piperidin-4-yl]amino}carbonyl, [(2-ethoxy-2-oxoethyl)-amino]carbonyl, [(2,4-difluorophenyl)amino]carbonyl, 3,3,3-trifluoropropanoyl, (benzoyl-amino)carbonyl, 4-methylphenylsulfonyl, (butylamino)carbonyl, hydroxy, piperidin-1-yl, acetylamino, ethoxycarbonyl, [(benzyloxy)carbonyl]amino, [(2-methylpyrrolidin-1-yl)carbonyl]amino, piperidin-1-ylcarbonyl, [(benzyloxy)carbonyl]amino, amino, oxo, morpholin-4-ylsulfonyl, pyrrolidin-1-carbonyl, morpholin-4-carbonyl, (diethylamino)-carbonyl, (4-methylpiperazin-1-yl)carbonyl, (4,4-difluoropiperidin-1-yl)carbonyl, 5,5,5-trifluoropentanoyl, 3-hydroxybutyl, 3-hydroxycyclobutyl, 3-methoxy-3-oxopropanoyl, 2-hydroxyethyl, hydroxyacetyl, 3-oxocyclobut-1-en-1-yl, 3-fluorocyclobutyl, 3-amino-3-oxopropanoyl, 3-methoxy-3-oxopropanoyl, carboxyacetyl, pyrrolidin-1-ylsulfonyl, 2-amino-2-oxoethyl, 2-oxopropyl, 2,3-dihydroxypropyl, 2-tert-butoxy-2-oxoethyl, carboxymethyl, 1,4-dioxa-8-azaspiro[4.5]dec-8-ylcarbonyl, (4-methylpiperazin-1-yl)carbonyl, thiomorpholin-4-ylcarbonyl, (4-isopropylpiperazin-1-yl)carbonyl, octahydroisoquinolin-2(1H)-ylcarbonyl, [(cyclopropylmethyl)amino]carbonyl, [(cyclopropylmethyl)(propyl)amino]carbonyl.

In a specific embodiment R² is selected from hydrogen, methyl, ethyl, acetyl, cyclopentyl, trifluoroacetyl, benzoyl, cyclohexylcarbonyl, thien-2-ylcarbonyl, 2,2-dimethylpropanoyl, butyryl, tert-butoxycarbonyl, (ethylamino)carbonyl, cyclopropylcarbonyl, isonicotinoyl, methoxyacetyl, methylsulfonyl, (benzylamino)carbonyl, anilinocarbonyl, {[2-(2-thienyl)ethyl]amino}carbonyl, aminocarbonyl, (cyclohexylamino)carbonyl, {[1-(trifluoroacetyl)piperidin-4-yl]amino}carbonyl, [(2,4-difluorophenyl)amino]carbonyl, 3,3,3-trifluoropropanoyl, 4-methylphenylsulfonyl, (butylamino)carbonyl, piperidin-1-yl, acetylamino, [(benzyloxy)carbonyl]amino, amino, oxo, morpholin-4-ylsulfonyl, pyrrolidin-1-carbonyl, morpholin-4-carbonyl, (4-methylpiperazin-1-yl)carbonyl, (4,4-difluoropiperidin-1-yl)carbonyl, 5,5,5-trifluoropentanoyl, 3-hydroxybutyl, 3-hydroxycyclobutyl, 2-hydroxyethyl, hydroxyacetyl, 3-oxocyclobut-1-en-1-yl, 3-amino-3-oxopropanoyl, 3-methoxy-3-oxopropanoyl, carboxyacetyl, 2-amino-2-oxoethyl, 2-oxopropyl, 2,3-dihydroxypropyl, 2-tert-butoxy-2-oxoethyl, carboxymethyl.

In a specific embodiment the present invention comprises compounds of formula (I) wherein either of R¹ or R² is an acyl or an aminocarbonyl.

A specific series of compounds are those of the formula (IA):

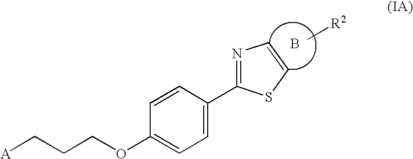

wherein A is either a pyrrolidinyl or a piperidinyl and B and R² are as above defined, in particular those wherein A is a pyrrolidin-1-yl which may be substituted by a C₁-C₆-alkyl or an amino, e.g. a methyl or dimethylamino, B is a tetrahydropyridyl or a tetrahydro-1H-azepinyl, and R² is linked to the tetrahydropyridyl or the tetrahydro-1H-azepinyl nitrogen and is selected from hydrogen, C₁-C₆-alkyl, C₃-C₈-cycloalkyl, sulfonyl, acyl, C₁-C₆-alkyl acyl, alkoxycarbonyl, C₁-C₆-alkyl alkoxycarbonyl, aminocarbonyl, C₁-C₆-alkyl aminocarbonyl, C₁-C₆-alkyl carboxy, C₁-C₆-alkyl hydroxy or C₃-C₈-cycloalkyl hydroxy.

Another particular embodiment of the invention concerns compounds of the formula (IA) wherein A is a pyrrolidin-1-yl which may be substituted by a C₁-C₆-alkyl, e.g. a methyl, B is a cyclopentenyl or a cyclohexenyl, and R² is a 3-8 membered heterocycloalkyl, acylamino, carbamate, amino, aminocarbonyl.

Another specific series of compounds are those of the formula (IB):

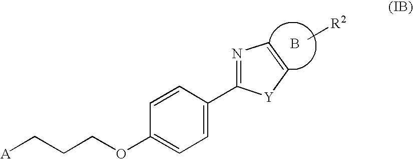

wherein R² is C₃-C₈-cycloalkyl hydroxy, sulfonyl, acyl, aminocarbonyl or C₁-C₆-alkyl aminocarbonyl, A, B and Y are as above defined, in particular those wherein A is a pyrrolidin-1-yl which may be substituted by a C₁-C₆-alkyl or an amino, e.g. a methyl or dimethylamino.

Specific compounds of formula (I) according to the invention are:
2-[4-(3-piperidin-1-ylpropoxy)phenyl]-4,5,6,7-tetrahydro[1,3]thiazolo[5,4-c]pyridine;
2-{4-[3-(2-methylpyrrolidin-1-yl)propoxy]phenyl}-4,5,6,7-tetrahydro[1,3]thiazolo[5,4-c]pyridine;
5-ethyl-2-{4-[3-(2-methylpyrrolidin-1-yl)propoxy]phenyl}-4,5,6,7-tetrahydro[1,3]thiazolo[5,4-c]pyridine;
5-acetyl-2-{4-[3-(2-methylpyrrolidin-1-yl)propoxy]phenyl}-4,5,6,7-tetrahydro[1,3]thiazolo-[5,4-c]pyridine;
5-acetyl-2-(4-{3-[(2R)-2-methylpyrrolidin-1-yl]propoxy}phenyl)-4,5,6,7-tetrahydro[1,3-]thiazolo[5,4-c]pyridine;
5-acetyl-2-(4-{3-[(2S)-2-methylpyrrolidin-1-yl]propoxy}phenyl)-4,5,6,7-tetrahydro-[1,3]thiazolo[5,4-c]pyridine;
5-(cyclohexylmethyl)-2-{4-[3-(2-methylpyrrolidin-1-yl)propoxy]phenyl}-4,5,6,7-tetrahydro[1,3]thiazolo[5,4-c]pyridine;
5-cyclopentyl-2-{4-[3-(2-methylpyrrolidin-1-yl)propoxy]phenyl}-4,5,6,7-tetrahydro[1,3-]thiazolo[5,4-c]pyridine;
2-{4-[3-(2-methylpyrrolidin-1-yl)propoxy]phenyl}-5-(trifluoroacetyl)-4,5,6,7-tetrahydro[1,3]thiazolo[5,4-c]pyridine;
5-(4-fluorophenyl)-2-{4-[3-(2-methylpyrrolidin-1-yl)propoxy]phenyl}-4,5,6,7-tetrahydro[1,3]thiazolo[5,4-c]pyridine;
5-benzoyl-2-{4-[3-(2-methylpyrrolidin-1-yl)propoxy]phenyl}-4,5,6,7-tetrahydro[1,3]-thiazolo[5,4-c]pyridine;
5-(cyclohexylcarbonyl)-2-{4-[3-(2-methylpyrrolidin-1-yl)propoxy]phenyl}-4,5,6,7-tetrahydro[1,3]thiazolo[5,4-c]pyridine;
2-{4-[3-(2-methylpyrrolidin-1-yl)propoxy]phenyl}-5-(thien-2-ylcarbonyl)-4,5,6,7-tetrahydro[1,3]thiazolo[5,4-c]pyridine;
5-(2,2-dimethylpropanoyl)-2-{4-[3-(2-methylpyrrolidin-1-yl)propoxy]phenyl}-4,5,6,7-tetrahydro[1,3]thiazolo[5,4-c]pyridine;
5-butyryl-2-{4-[3-(2-methylpyrrolidin-1-yl)propoxy]phenyl}-4,5,6,7-tetrahydro[1,3]thiazolo-[5,4-c]pyridine;
tert-butyl 2-{4-[3-(2-methylpyrrolidin-1-yl)propoxy]phenyl}-6,7-dihydro[1,3]thiazolo[5,4-c]pyridine-5(4H)-carboxylate;
N-ethyl-2-{4-[3-(2-methylpyrrolidin-1-yl)propoxy]phenyl}-6,7-dihydro[1,3]thiazolo[5,4-c]pyridine-5(4H)-carboxamide;
N-ethyl-2-(4-{3-[(2R)-2-methylpyrrolidin-1-yl]propoxy}phenyl)-6,7-dihydro[1,3]thiazolo[5,4-c]pyridine-5(4H)-carboxamide;
N-ethyl-2-(4-{3-[(2S)-2-methylpyrrolidin-1-yl]propoxy}phenyl)-6,7-dihydro[1,3]thiazolo[5,4-c]pyridine-5(4H)-carboxamide;
5-(cyclopropylcarbonyl)-2-{4-[3-(2-methylpyrrolidin-1-yl)propoxy]phenyl}-4,5,6,7-tetrahydro[1,3]thiazolo[5,4-c]pyridine;
5-isonicotinoyl-2-{4-[3-(2-methylpyrrolidin-1-yl)propoxy]phenyl}-4,5,6,7-tetrahydro[1,3]-thiazolo[5,4-c]pyridine;
5-(methoxyacetyl)-2-(4-{3-[(2R)-2-methylpyrrolidin-1-yl]propoxy}phenyl)-4,5,6,7-tetrahydro[1,3]thiazolo[5,4-c]pyridine;
5-(methoxyacetyl)-2-{4-[3-(2-methylpyrrolidin-1-yl)propoxy]phenyl}-4,5,6,7-tetrahydro-[1,3]thiazolo[5,4-c]pyridine;
5-(methoxyacetyl)-2-(4-{3-[(2S)-2-methylpyrrolidin-1-yl]propoxy}phenyl)-4,5,6,7-tetrahydro[1,3]thiazolo[5,4-c]pyridine;
N-ethyl-2-(4-{3-[2-(4-{3-[(2S)-2-methylpyrrolidin-1-yl]propoxy}phenyl)-6,7-dihydro[1,3]thiazolo[5,4-c]pyridin-5(4H)-yl]propoxy}phenyl)-6,7-dihydro[1,3]thiazolo[5,4-c]pyridine-5(4H)-carboxamide;
2-{4-[3-(2-methylpyrrolidin-1-yl)propoxy]phenyl}-5-(methylsulfonyl)-4,5,6,7-tetrahydro[1,3]thiazolo[5,4-c]pyridine;
N-benzyl-2-{4-[3-(2-methylpyrrolidin-1-yl)propoxy]phenyl}-6,7-dihydro[1,3]thiazolo[5,4-c]pyridine-5(4H)-carboxamide;
2-{4-[3-(2-methylpyrrolidin-1-yl)propoxy]phenyl}-N-phenyl-6,7-dihydro[1,3]thiazolo[5,4-c]pyridine-5(4H)-carboxamide;
2-{4-[3-(2-methylpyrrolidin-1-yl)propoxy]phenyl}-N-(2-thien-2-ylethyl)-6,7-dihydro[1,3]-thiazolo[5,4-c]pyridine-5(4H)-carboxamide;
2-{4-[3-(2-methylpyrrolidin-1-yl)propoxy]phenyl}-6,7-dihydro[1,3]thiazolo[5,4-c]pyridine-5(4H)-carboxamide;
N-isopropyl-2-{4-[3-(2-methylpyrrolidin-1-yl)propoxy]phenyl}-6,7-dihydro[1,3]thiazolo[5,4-c]pyridine-5(4H)-carboxamide;
N-cyclohexyl-2-{4-[3-(2-methylpyrrolidin-1-yl)propoxy]phenyl}-6,7-dihydro[1,3]thiazolo[5,4-c]pyridine-5(4H)-carboxamide;
2-{4-[3-(2-methylpyrrolidin-1-yl)propoxy]phenyl}-N-[1-(trifluoroacetyl)piperidin-4-yl]-6,7-dihydro[1,3]thiazolo[5,4-c]pyridine-5(4H)-carboxamide;
ethyl ({[2-{4-[3-(2-methylpyrrolidin-1-yl)propoxy]phenyl}-6,7-dihydro[1,3]thiazolo[5,4-c]pyridin-5(4H)-yl]carbonyl}amino)acetate;
N-(2,4-difluorophenyl)-2-{4-[3-(2-methylpyrrolidin-1-yl)propoxy]phenyl}-6,7-dihydro[1,3]thiazolo[5,4-c]pyridine-5(4H)-carboxamide;
2-{4-[3-(2-methylpyrrolidin-1-yl)propoxy]phenyl}-5-(3,3,3-trifluoropropanoyl)-4,5,6,7-tetrahydro[1,3]thiazolo[5,4-c]pyridine;
N-benzoyl-2-{4-[3-(2-methylpyrrolidin-1-yl)propoxy]phenyl}-6,7-dihydro[1,3]thiazolo[5,4-c]pyridine-5(4H)-carboxamide
5-[(4-methylphenyl)sulfonyl]-2-{4-[3-(2-methylpyrrolidin-1-yl)propoxy]phenyl}-4,5,6,7-tetrahydro[1,3]thiazolo[5,4-c]pyridine;
N-butyl-2-{4-[3-(2-methylpyrrolidin-1-yl)propoxy]phenyl}-6,7-dihydro[1,3]thiazolo[5,4-c]pyridine-5(4H)-carboxamide;
5-acetyl-2-(4-{3-[2-(pyrrolidin-1-ylmethyl)pyrrolidin-1-yl]propoxy}phenyl)-4,5,6,7-tetrahydro[1,3]thiazolo[5,4-c]pyridine;
5-acetyl-2-[4-(3-azepan-1-ylpropoxy)phenyl]-4,5,6,7-tetrahydro[1,3]thiazolo[5,4-c]pyridine;
5-acetyl-2-{4-[3-(4-isopropylpiperazin-1-yl)propoxy]phenyl}-4,5,6,7-tetrahydro[1,3]thiazolo[5,4-c]pyridine;
5-acetyl-2-{4-[3-(2-methylpiperidin-1-yl)propoxy]phenyl}-4,5,6,7-tetrahydro[1,3]thiazolo[5,4-c]pyridine;
1-{3-[4-(5-acetyl-4,5,6,7-tetrahydro[1,3]thiazolo[5,4-c]pyridin-2-yl)phenoxy]propyl}-N,N-dimethylpyrrolidin-3-amine;
5-acetyl-2-{4-[3-(3,5-dimethylpiperidin-1-yl)propoxy]phenyl}-4,5,6,7-tetrahydro[1,3]-thiazolo[5,4-c]pyridine;
5-[(4-methylphenyl)sulfonyl]-2-{4-[3-(2-methylpyrrolidin-1-yl)propoxy]phenyl}-5,6-dihydro-4H-pyrrolo[3,4-d][1,3]thiazole;
2-{4-[3-(2-methylpyrrolidin-1-yl)propoxy]phenyl}-5,6,7,8-tetrahydro-4H-[1,3]thiazolo[4,5-d]azepine;
2-{4-[3-(2-methylpyrrolidin-1-yl)propoxy]phenyl}-4,5,6,7-tetrahydro-1,3-benzothiazol-4-ol;
2-{4-[3-(2-methylpyrrolidin-1-yl)propoxy]phenyl}-4-piperidin-1-yl-4,5,6,7-tetrahydro-1,3-benzothiazole;
2-{4-[3-(2-methylpyrrolidin-1-yl)propoxy]phenyl}-7-piperidin-1-yl-4,5,6,7-tetrahydro-1,3-benzothiazole;
N-(2-{4-[3-(2-methylpyrrolidin-1-yl)propoxy]phenyl}-4,5,6,7-tetrahydro-1,3-benzothiazol-6-yl)acetamide;
ethyl 2-{4-[3-(2-methylpyrrolidin-1-yl)propoxy]phenyl}-4,5,6,7-tetrahydro-1,3-benzothiazole-4-carboxylate;
benzyl 2-{4-[3-(2-methylpyrrolidin-1-yl)propoxy]phenyl}-4,5,6,7-tetrahydro-1,3-benzothiazol-4-ylcarbamate;
N-(2-{4-[3-(2-methylpyrrolidin-1-yl)propoxy]phenyl}-4,5,6,7-tetrahydro-1,3-benzothiazol-4-yl)acetamide;
2-methyl-N-(2-{4-[3-(2-methylpyrrolidin-1-yl)propoxy]phenyl}-4,5,6,7-tetrahydro-1,3-benzothiazol-4-yl)pyrrolidine-1-carboxamide;
N-(2-{4-[3-(2-methylpyrrolidin-1-yl)propoxy]phenyl}-4,5,6,7-tetrahydro-1,3-benzothiazol-5-yl)acetamide;
ethyl 2-{4-[3-(2-methylpyrrolidin-1-yl)propoxy]phenyl}-5,6-dihydro-4H-cyclopenta[d][1,3]-thiazole-4-carboxylate;
2-{4-[3-(2-methylpyrrolidin-1-yl)propoxy]phenyl}-4-(piperidin-1-ylcarbonyl)-5,6-dihydro-4H-cyclopenta[d][1,3]thiazole;
benzyl 2-{4-[3-(2-methylpyrrolidin-1-yl)propoxy]phenyl}-5,6-dihydro-4H-cyclopenta[d]-[1,3]thiazol-4-ylcarbamate;
ethyl 4-methyl-2-{4-[3-(2-methylpyrrolidin-1-yl)propoxy]phenyl}-5,6-dihydro-4H-cyclopenta-[d][1,3]thiazole-4-carboxylate;
(2-{4-[3-(2-methyl-pyrrolidin-1-yl)-propoxy]-phenyl}-5,6-dihydro-4H-cyclopentathiazol-4-ylamine;
N-(2-{4-[3-(2-methylpyrrolidin-1-yl)propoxy]phenyl}-5,6-dihydro-4H-cyclopenta[d][1,3]-thiazol-4-yl)acetamide;
2-{4-[3-(2-methylpyrrolidin-1-yl)propoxy]phenyl}-4-(methylsulfonyl)-4,5,6,7-tetrahydro-[1,3]thiazolo[4,5-b]pyridine;
4-(methoxyacetyl)-2-{4-[3-(2-methylpyrrolidin-1-yl)propoxy]phenyl}-4,5,6,7-tetrahydro-[1,3]thiazolo[4,5-b]pyridine;
4-acetyl-2-{4-[3-(2-methylpyrrolidin-1-yl)propoxy]phenyl}-4,5,6,7-tetrahydro[1,3]-thiazolo[4,5-b]pyridine;
N-ethyl-2-{4-[3-(2-methylpyrrolidin-1-yl)propoxy]phenyl}-6,7-dihydro[1,3]thiazolo[4,5-b]pyridine-4(5H)-carboxamide;
4-isonicotinoyl-2-{4-[3-(2-methylpyrrolidin-1-yl)propoxy]phenyl}-4,5,6,7-tetrahydro-[1,3]thiazolo[4,5-b]pyridine;
2-{4-[3-(2-methylpyrrolidin-1-yl)propoxy]phenyl}-4-(morpholin-4-ylsulfonyl)-4,5,6,7-tetrahydro[1,3]thiazolo[4,5-b]pyridine;
2-{4-[3-(2-methylpyrrolidin-1-yl)propoxy]phenyl}-4,5,6,7-tetrahydro[1,3]thiazolo[4,5-b]pyridine;
2-{4-[3-(2-methylpyrrolidin-1-yl)propoxy]phenyl}-4-(morpholin-4-ylcarbonyl)-4,5,6,7-tetrahydro[1,3]thiazolo[4,5-b]pyridine;
2-{4-[3-(2-methylpyrrolidin-1-yl)propoxy]phenyl}-6,7-dihydro[1,3]thiazolo[4,5-b]pyridin-5(4H)-one;
5-methyl-2-{4-[3-(2-methylpyrrolidin-1-yl)propoxy]phenyl}-4,5,6,7-tetrahydro[1,3]-thiazolo[4,5-c]pyridine;
5-acetyl-2-(4-{3-[(2R)-2-methylpyrrolidin-1-yl]propoxy}phenyl)-4,5,6,7-tetrahydro[1,3]-thiazolo[4,5-c]pyridine;
N-ethyl-2-(4-{3-[(2R)-2-methylpyrrolidin-1-yl]propoxy}phenyl)-6,7-dihydro[1,3]thiazolo[4,5-c]pyridine-5(4H)-carboxamide;
5-(methoxyacetyl)-2-(4-{3-[(2R)-2-methylpyrrolidin-1-yl]propoxy}phenyl)-4,5,6,7-tetrahydro[1,3]thiazolo[4,5-c]pyridine;
5-methyl-2-(4-{3-[(2R)-2-methylpyrrolidin-1-yl]propoxy}phenyl)-4,5,6,7-tetrahydro[1,3]-oxazolo[4,5-c]pyridine;
2-{4-[3-(2-methylpyrrolidin-1-yl)propoxy]phenyl}-5-(pyrrolidin-1-ylcarbonyl)-4,5,6,7-tetrahydro[1,3]thiazolo[5,4-c]pyridine;
2-{4-[3-(2-methylpyrrolidin-1-yl)propoxy]phenyl}-5-(morpholin-4-ylcarbonyl)-4,5,6,7-tetrahydro[1,3]thiazolo[5,4-c]pyridine;
N,N-diethyl-2-{4-[3-(2-methylpyrrolidin-1-yl)propoxy]phenyl}-6,7-dihydro[1,3]thiazolo[5,4-c]pyridine-5(4H)-carboxamide;
5-[(4-methylpiperazin-1-yl)carbonyl]-2-{4-[3-(2-methylpyrrolidin-1-yl)propoxy]phenyl}-4,5,6,7-tetrahydro[1,3]thiazolo[5,4-c]pyridine;
5-[(4,4-difluoropiperidin-1-yl)carbonyl]-2-{4-[3-(2-methylpyrrolidin-1-yl)propoxy]phenyl}-4,5,6,7-tetrahydro[1,3]thiazolo[5,4-c]pyridine;
2-{4-[3-(2-methylpyrrolidin-1-yl)propoxy]phenyl}-5-(5,5,5-trifluoropentanoyl)-4,5,6,7-tetrahydro[1,3]thiazolo[5,4-c]pyridine;
4-[2-{4-[3-(2-methylpyrrolidin-1-yl)propoxy]phenyl}-6,7-dihydro[1,3]thiazolo[5,4-c]pyridin-5(4H)-yl]butan-2-ol;
(3R)-1-{3-[4-(5-acetyl-4,5,6,7-tetrahydro[1,3]thiazolo[5,4-c]pyridin-2-yl)phenoxy]propyl}-N,N-dimethylpyrrolidin-3-amine;
cis-3-[2-{4-[3-(2-methylpyrrolidin-1-yl)propoxy]phenyl}-6,7-dihydro[1,3]thiazolo[5,4-c]pyridin-5(4H)-yl]cyclobutanol;
(3S)-1-{3-[4-(5-acetyl-4,5,6,7-tetrahydro[1,3]thiazolo[5,4-c]pyridin-2-yl)phenoxy]propyl}-N,N-dimethylpyrrolidin-3-amine;
methyl 3-[2-{4-[3-(2-methylpyrrolidin-1-yl)propoxy]phenyl}-6,7-dihydro[1,3]thiazolo[5,4-c]pyridin-5(4H)-yl]-3-oxopropanoate;
2-[2-{4-[3-(2-methylpyrrolidin-1-yl)propoxy]phenyl}-6,7-dihydro[1,3]thiazolo[5,4-c]pyridin-5(4H)-yl]ethanol;
3-[2-{4-[3-(2-methylpyrrolidin-1-yl)propoxy]phenyl}-6,7-dihydro[1,3]thiazolo[5,4-c]pyridin-5(4H)-yl]-3-oxopropanoic acid;
3-[2-(4-{3-[(2R)-2-methylpyrrolidin-1-yl]propoxy}phenyl)-6,7-dihydro[1,3]thiazolo[5,4-c]pyridin-5(4H)-yl]cyclobut-2-en-1-one;
5-(trans-3-fluorocyclobutyl)-2-{4-[3-(2-methylpyrrolidin-1-yl)propoxy]phenyl}-4,5,6,7-tetrahydro[1,3]thiazolo[5,4-c]pyridine;
2-(4-{3-[(2R)-2-methylpyrrolidin-1-yl]propoxy}phenyl)-5-(morpholin-4-ylsulfonyl)-4,5,6,7-tetrahydro[1,3]thiazolo[5,4-c]pyridine;
3-[2-(4-{3-[(2R)-2-methylpyrrolidin-1-yl]propoxy}phenyl)-6,7-dihydro[1,3]thiazolo[5,4-c]pyridin-5(4H)-yl]-3-oxopropanamide;
methyl [2-(4-{3-[(2R)-2-methylpyrrolidin-1-yl]propoxy}phenyl)-6,7-dihydro[1,3]thiazolo[5,4-c]pyridin-5(4H)-yl]acetate;
2-[2-(4-{3-[(2R)-2-methylpyrrolidin-1-yl]propoxy}phenyl)-6,7-dihydro[1,3]thiazolo[5,4-c]pyridin-5(4H)-yl]-2-oxoethanol;
2-(4-{3-[(2R)-2-methylpyrrolidin-1-yl]propoxy}phenyl)-5-(pyrrolidin-1-ylsulfonyl)-4,5,6,7-tetrahydro[1,3]thiazolo[5,4-c]pyridine;
2-[2-(4-{3-[(2R)-2-methylpyrrolidin-1-yl]propoxy}phenyl)-6,7-dihydro[1,3]thiazolo[5,4-c]pyridin-5(4H)-yl]acetamide;
1-[2-(4-{3-[(2R)-2-methylpyrrolidin-1-yl]propoxy}phenyl)-6,7-dihydro[1,3]thiazolo[5,4-c]pyridin-5(4H)-yl]acetone;
3-[2-(4-{3-[(2R)-2-methylpyrrolidin-1-yl]propoxy}phenyl)-6,7-dihydro[1,3]thiazolo[5,4-c]pyridin-5(4H)-yl]propane-1,2-diol;
((2S)-1-{3-[4-(5-acetyl-4,5,6,7-tetrahydro[1,3]thiazolo[5,4-c]pyridin-2-yl)phenoxy]-propyl}pyrrolidin-2-yl)methanol;
tert-butyl [2-(4-{3-[(2R)-2-methylpyrrolidin-1-yl]propoxy}phenyl)-6,7-dihydro[1,3]thiazolo-[5,4-c]pyridin-5(4H)-yl]acetate;
[2-(4-{3-[(2R)-2-methylpyrrolidin-1-yl]propoxy}phenyl)-6,7-dihydro[1,3]thiazolo[5,4-c]pyridin-5(4H)-yl]acetic acid;
6-acetyl-2-{4-[3-(2-methylpyrrolidin-1-yl)propoxy]phenyl}-5,6,7,8-tetrahydro-4H-[1,3]thiazolo[4,5-d]azepine;
2-{4-[3-(2-methylpyrrolidin-1-yl)propoxy]phenyl}-6-(methylsulfonyl)-5,6,7,8-tetrahydro-4H-[1,3]thiazolo[4,5-d]azepine;
N-ethyl-2-{4-[3-(2-methylpyrrolidin-1-yl)propoxy]phenyl}-4,5,7,8-tetrahydro-6H-[1,3]thiazolo[4,5-d]azepine-6-carboxamide;
2-{4-[3-(2-methylpyrrolidin-1-yl)propoxy]phenyl}-6-(morpholin-4-ylcarbonyl)-5,6,7,8-tetrahydro-4H-[1,3]thiazolo[4,5-d]azepine;
2-(2-{4-[3-(2-methylpyrrolidin-1-yl)propoxy]phenyl}-4,5,7,8-tetrahydro-6H-[1,3]thiazolo[4,5-d]azepin-6-yl)acetamide;
2-{4-[3-(2-methylpyrrolidin-1-yl)propoxy]phenyl}-6-(morpholin-4-ylsulfonyl)-5,6,7,8-tetrahydro-4H-[1,3]thiazolo[4,5-d]azepine;
6-isonicotinoyl-2-{4-[3-(2-methylpyrrolidin-1-yl)propoxy]phenyl}-5,6,7,8-tetrahydro-4H-[1,3]thiazolo[4,5-d]azepine;
(2-{4-[3-(2-methylpyrrolidin-1-yl)propoxy]phenyl}-4,5,7,8-tetrahydro-6H-[1,3]thiazolo[4,5-d]azepin-6-yl)acetic acid;
6-(1,4-dioxa-8-azaspiro[4.5]dec-8-ylcarbonyl)-2-{4-[3-(2-methylpyrrolidin-1-yl)propoxy]-phenyl}-5,6,7,8-tetrahydro-4H-[1,3]thiazolo[4,5-d]azepine;
6-[(4,4-difluoropiperidin-1-yl)carbonyl]-2-{4-[3-(2-methylpyrrolidin-1-yl)propoxy]phenyl}-5,6,7,8-tetrahydro-4H-[1,3]thiazolo[4,5-d]azepine;
7,7-dimethyl-2-{4-[3-(2-methylpyrrolidin-1-yl)propoxy]phenyl}-5,6,7,8-tetrahydro-4H-[1,3]thiazolo[5,4-c]azepin-4-one;
5,7,7-trimethyl-2-{4-[3-(2-methylpyrrolidin-1-yl)propoxy]phenyl}-5,6,7,8-tetrahydro-4H-[1,3]thiazolo[5,4-c]azepin-4-one;
4-acetyl-2-(4-{3-[(2R)-2-methylpyrrolidin-1-yl]propoxy}phenyl)-5,6,7,8-tetrahydro-4H-[1,3]thiazolo[5,4-b]azepine;
2-{4-[3-(2-methylpyrrolidin-1-yl)propoxy]phenyl}-5-(morpholin-4-ylcarbonyl)-5,6-dihydro-4H-cyclopenta[d][1,3]thiazole;
2-{4-[3-(2-methylpyrrolidin-1-yl)propoxy]phenyl}-5,6-dihydro-4H-cyclopenta[d][1,3]thiazol-5-amine;
N-(2-{4-[3-(2-methylpyrrolidin-1-yl)propoxy]phenyl}-5,6-dihydro-4H-cyclopenta[d][1,3]-thiazol-5-yl)acetamide;
5-[(4,4-difluoropiperidin-1-yl)carbonyl]-2-(4-{3-[(2R)-2-methylpyrrolidin-1-yl]propoxy}-phenyl)-5,6-dihydro-4H-cyclopenta[d][1,3]thiazole;
N-ethyl-2-(4-{3-[(2R)-2-methylpyrrolidin-1-yl]propoxy}phenyl)-5,6-dihydro-4H-cyclopenta-[d][1,3]thiazole-5-carboxamide;
2-(4-{3-[(2R)-2-methylpyrrolidin-1-yl]propoxy}phenyl)-5-(pyrrolidin-1-ylcarbonyl)-5,6-dihydro-4H-cyclopenta[d][1,3]thiazole;
5-[(4-methylpiperazin-1-yl)carbonyl]-2-(4-{3-[(2R)-2-methylpyrrolidin-1-yl]propoxy}phenyl)-5,6-dihydro-4H-cyclopenta[d][1,3]thiazole;
5-[(4,4-difluoropiperidin-1-yl)carbonyl]-2-{4-[3-(4-isopropylpiperazin-1-yl)propoxy]phenyl}-5,6-dihydro-4H-cyclopenta[d][1,3]thiazole;
2-{4-[3-(4-isopropylpiperazin-1-yl)propoxy]phenyl}-5-(thiomorpholin-4-ylcarbonyl)-5,6-dihydro-4H-cyclopenta[d][1,3]thiazole;
2-{4-[3-(4-isopropylpiperazin-1-yl)propoxy]phenyl}-5-(morpholin-4-ylcarbonyl)-5,6-dihydro-4H-cyclopenta[d][1,3]thiazole;
5-[(4-isopropylpiperazin-1-yl)carbonyl]-2-{4-[3-(4-isopropylpiperazin-1-yl)propoxy]phenyl}-5,6-dihydro-4H-cyclopenta[d][1,3]thiazole;
2-[(2-{4-[3-(4-isopropylpiperazin-1-yl)propoxy]phenyl}-5,6-dihydro-4H-cyclopenta[d]-[1,3]thiazol-5-yl)carbonyl]decahydroisoquinoline;
N-(cyclopropylmethyl)-2-{4-[3-(4-isopropylpiperazin-1-yl)propoxy]phenyl}-5,6-dihydro-4H-cyclopenta[d][1,3]thiazole-5-carboxamide;
N-(cyclopropylmethyl)-2-{4-[3-(4-isopropylpiperazin-1-yl)propoxy]phenyl}-N-propyl-5,6-dihydro-4H-cyclopenta[d][1,3]thiazole-5-carboxamide;
2-{4-[3-(4-isopropylpiperazin-1-yl)propoxy]phenyl}-5-(pyrrolidin-1-ylcarbonyl)-5,6-dihydro-4H-cyclopenta[d][1,3]thiazole;
5-acetyl-2-{4-[3-(2-methylpyrrolidin-1-yl)propoxy]phenyl}-5,6-dihydro-4H-pyrrolo[3,4-d][1,3]thiazole; and
N-ethyl-2-{4-[3-(2-methylpyrrolidin-1-yl)propoxy]phenyl}-4,6-dihydro-5H-pyrrolo[3,4-d][1,3]thiazole-5-carboxamide.

The “pharmaceutically acceptable salts” according to the invention include all therapeutically active, non-toxic acid salt forms which the compounds of formula (I) are able to form.

The acid addition salt form of a compound of formula (I) that occurs in its free form as a base can be obtained by treating the free base with an appropriate acid such as an inorganic acid, for example, a hydrohalic such as hydrochloric or hydrobromic, sulfuric, nitric, phosphoric and the like; or an organic acid, such as, for example, acetic, trifluoroacetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclamic, salicylic, p-aminosalicylic, pamoic, and the like.

Conversely said salt forms can be converted into the free forms by treatment with an appropriate base.

Preferred salt forms are maleate, tartrate, fumarate, chlorhydrate, and trifluoroacetate.

Compounds of the formula (I) and their salts can be in the form of a solvate, which is included within the scope of the present invention. Such solvates include for example hydrates, alcoholates and the like.

Many of the compounds of formula (I) and some of their intermediates have at least one stereogenic center in their structure. This stereogenic center may be present in a R or a S configuration, said R and S notation is used in correspondence with the rules described in Pure Appl. Chem., 45 (1976) 11-30.

The invention also relates to all stereoisomeric forms such as enantiomeric and diastereoisomeric forms of the compounds of formula (I) or mixtures thereof (including all possible mixtures of stereoisomers).

With respect to the present invention reference to a compound or compounds is intended to encompass that compound in each of its possible isomeric forms and mixtures thereof, unless the particular isomeric form is referred to specifically.

Compounds according to the present invention may exist in different polymorphic forms. Although not explicitly indicated in the above formula, such forms are included within the scope of the present invention.

The invention also includes within its scope prodrug forms of the compounds of formula (I) and its various sub-scopes and sub-groups.

The term “prodrug” as used herein includes compound forms which are rapidly transformed in vivo to the parent compound according to the invention, for example, by hydrolysis in blood. Prodrugs are compounds bearing groups which are removed by biotransformation prior to exhibiting their pharmacological action. Such groups include moieties which are readily cleaved in vivo from the compound bearing it, which compound after cleavage remains or becomes pharmacologically active. Metabolically cleavable groups form a class of groups well known to practitioners of the art. They include, but are not limited to such groups as alkanoyl (i.e. acetyl, propionyl, butyryl, and the like), unsubstituted and substituted carbocyclic aroyl (such as benzoyl, substituted benzoyl and 1- and 2-naphthoyl), alkoxycarbonyl (such as ethoxycarbonyl), trialkysilyl (such as trimethyl- and triethylsilyl), monoesters formed with dicarboxylic acids (such as succinyl), phosphate, sulfate, sulfonate, sulfonyl, sulfinyl and the like. The compounds bearing the metabolically cleavable groups have the advantage that they may exhibit improved bioavailability as a result of enhanced solubility and/or rate of absorption conferred upon the parent compound by virtue of the presence of the metabolically cleavable group. T. Higuchi and V. Stella, “Pro-drugs as Novel Delivery System”, Vol. 14 of the A.C.S. Symposium Series; “Bioreversible Carriers in Drug Design”, ed. Edward B. Roche, American Pharmaceutical Association and Pergamon Press, 1987.

It has now been found that compounds of formula (I) according to the present invention and their pharmaceutically acceptable salts are useful in a variety of medical disorders.

For example, the compounds according to the invention are useful for the treatment and prevention of diseases or pathological conditions of the central nervous system including mild-cognitive impairment, Alzheimer's disease, learning and memory disorders, cognitive disorders, attention deficit disorder, attention-deficit hyperactivity disorder, Parkinson's disease, schizophrenia, dementia, depression, epilepsy, seizures, convulsions, sleep/wake disorders, narcolepsy, and/or obesity.

Furthermore, compounds according to the invention alone or in combination with an antiepileptic drug (AED) may be useful in the treatment of epilepsy, seizure or convulsions. It is known from literature that the combination of H₃-receptor ligands with an AED may produce additive synergistic effects on efficacy with reduced side-effects such as decreased vigilance, sedation or cognitive problems.

Furthermore, compounds of general formula (I) alone or in combination with a histamine H₁-receptor antagonist may also be used for the treatment of upper airway allergic disorders.

In a particular embodiment of the present invention, compounds of general formula (I), alone or in combination with muscarinic receptor ligands and particularly with a muscarinic M₂-receptor antagonist, may be useful for the treatment of cognitive disorders, Alzheimer's disease, and attention-deficit hyperactivity disorder.

Particularly, compounds of general formula (I) displaying NO-donor properties, alone or in combination with a nitric oxide (NO) releasing agent may be useful in the treatment of cognitive dysfunctions.

In another particular embodiment, compounds of general formula (I), alone or in combination with a serotonin reuptake inhibitor, may be useful in the treatment of depression, anxiety disorders and other affective disorders.

Compounds of general formula (I) may also be used in the treatment of sleep/wake and arousal/vigilance disorders such as hypersomnia, and narcolepsy.

Usually, compounds of general formula (I) may be used in the treatment of all types of cognitive-related disorders.

Preferably, compounds of general formula (I) may be used for the treatment of cognitive dysfunctions in diseases such as mild cognitive impairment, dementia, Alzheimer's disease, Parkinson's disease, Down's syndrome as well as for the treatment of attention-deficit hyperactivity disorder.

In another preferred embodiment, compounds of general formula (I) may also be used for the treatment of psychotic disorders, such as schizophrenia; or for the treatment of eating disorders, such as obesity; or for the treatment of inflammation and pain; or for the treatment of anxiety, stress and depression; or for the treatment of cardiovascular disorders, for example, myocardial infarction.

In a further aspect, compounds of formula (I) according to the present invention may be used as a medicament.

In a particular embodiment, the present invention concerns the use of a compound of formula (I) or a pharmaceutically acceptable salt thereof or of a pharmaceutical composition comprising an effective amount of said compound for the manufacture of a medicament for the treatment and prevention of mild-cognitive impairment, Alzheimer's disease, learning and memory disorders, attention-deficit hyperactivity disorder, Parkinson's disease, schizophrenia, dementia, depression, epilepsy, seizures, convulsions, sleep/wake disorders, cognitive dysfunctions, narcolepsy, hypersomnia, obesity, upper airway allergic disorders, Down's syndrome, anxiety, stress, cardiovascular disorders, inflammation and pain.

Preferably, the present invention concerns the use of a compound of formula (I) or a pharmaceutically acceptable salt thereof or a pharmaceutical composition comprising an effective amount of said compound for the manufacture of a medicament for the treatment of cognitive dysfunctions in diseases such as mild cognitive impairment, dementia, Alzheimer's disease, Parkinson's disease, Down's syndrome as well as for the treatment of attention-deficit hyperactivity disorder.

The methods of the invention comprise administration to a mammal (preferably human) suffering from above mentioned conditions or disorders, of a compound according to the invention in an amount sufficient to alleviate or prevent the disorder or condition.

The compound is conveniently administered in any suitable unit dosage form, including but not limited to one containing 3 to 3000 mg of active ingredient per unit dosage form.

The term “treatment” as used herein includes curative treatment and prophylactic treatment.

By “curative” is meant efficacy in treating a current symptomatic episode of a disorder or condition.

By “prophylactic” is meant prevention of the occurrence or recurrence of a disorder or condition.

The expression “cognitive disorders” as used herein refers to disturbances of cognition, which encompasses perception, learning and reasoning or in other terms the physiological (mental/neuronal) process of selectively acquiring, storing, and recalling information.

The expression “attention-deficit hyperactivity disorder” (ADHD) as used herein refers to a problem with inattentiveness, over-activity, impulsivity, or a combination of these. For these problems to be diagnosed as ADHD, they must be out of the normal range for the child's age and development. The term “attention-deficit disorder” (ADD) is also commonly used for the same disorder.

The expression “Alzheimer's disease” (AD) as used herein refers to a progressive, neurodegenerative disease characterized in the brain by abnormal clumps (amyloid plaques) and tangled bundles of fibers (neurofibrillary tangles) composed of misplaced proteins. Age is the most important risk factor for AD; the number of people with the disease doubles every 5 years beyond age 65. Three genes have been discovered that cause early onset (familial) AD. Other genetic mutations that cause excessive accumulation of amyloid protein are associated with age-related (sporadic) AD. Symptoms of AD include memory loss, language deterioration, impaired ability to mentally manipulate visual information, poor judgment, confusion, restlessness, and mood swings. Eventually AD destroys cognition, personality, and the ability to function. The early symptoms of AD, which include forgetfulness and loss of concentration, are often missed because they resemble natural signs of aging.

The expression “Parkinson's disease” (PD) as used herein refers to a group of conditions called motor system disorders, which are the result of the loss of dopamine-producing brain cells. The four primary symptoms of PD are tremor, or trembling in hands, arms, legs, jaw, and face; rigidity, or stiffness of the limbs and trunk; bradykinesia, or slowness of movement; and postural instability, or impaired balance and coordination. As these symptoms become more pronounced, patients may have difficulty walking, talking, or completing other simple tasks. PD usually affects people over the age of 50. Early symptoms of PD are subtle and occur gradually. In some people the disease progresses more quickly than in others. As the disease progresses, the shaking, or tremor, which affects the majority of PD patients may begin to interfere with daily activities. Other symptoms may include depression and other emotional changes; difficulty in swallowing, chewing, and speaking; urinary problems or constipation; skin problems; and sleep disruptions.

The expression “Down's syndrome” as used herein refers to a chromosome abnormality, usually due to an extra copy of the 21st chromosome. This syndrome, usually but not always, results in mental retardation and other conditions. The term “mental retardation” refers to a below-average general intellectual function with associated deficits in adaptive behavior that occurs before age 18.

The term “mild-cognitive impairment” as used herein refers to a transitional stage of cognitive impairment between normal aging and early Alzheimer's disease. It refers particularly to a clinical state of individuals who are memory impaired but are otherwise functioning well and do not meet clinical criteria for dementia.

The term “obesity” as used herein refers to a body mass index (BMI) which is greater than 30 kg/m².

The term “dementia” as used herein refers to a group of symptoms involving progressive impairment of brain function. American Geriatrics Society refers to dementia as a condition of declining mental abilities, especially memory. The person will have problems doing things he or she used to be able to do, like keep the check book, drive a car safely, or plan a meal. He or she will often have problems finding the right words and may become confused when given too many things to do at once. The person with dementia may also change in personality, becoming aggressive, paranoid, or depressed.

The term “schizophrenia” as used herein refers to a group of psychotic disorders characterized by disturbances in thought, perception, attention, affect, behavior, and communication that last longer than 6 months. It is a disease that makes it difficult for a person to tell the difference between real and unreal experiences, to think logically, to have normal emotional responses to others, and to behave normally in social situations.

The term “anxiety” as used herein refers to a feeling of apprehension or fear. Anxiety is often accompanied by physical symptoms, including twitching or trembling, muscle tension, headaches, sweating, dry mouth, difficulty swallowing and/or abdominal pain.

The term “narcolepsy” as used herein refers to a sleep disorder associated with uncontrollable sleepiness and frequent daytime sleeping.

The term “depression” as used herein refers to a disturbance of mood and is characterized by a loss of interest or pleasure in normal everyday activities. People who are depressed may feel “down in the dumps” for weeks, months, or even years at a time. Some of the following symptoms may be symptoms of depression: persistent sad, anxious, or “empty” mood; feelings of hopelessness, pessimism; feelings of guilt, worthlessness, helplessness; loss of interest or pleasure in hobbies and activities that were once enjoyed, including sex; decreased energy, fatigue, being “slowed down”; difficulty concentrating, remembering, making decisions; insomnia, early-morning awakening, or oversleeping; appetite and/or weight loss or overeating and weight gain; thoughts of death or suicide; suicide attempts; restlessness, irritability; persistent physical symptoms that do not respond to treatment, such as headaches, digestive disorders, and chronic pain.

The term “epilepsy” as used herein refers a brain disorder in which clusters of nerve cells, or neurons, in the brain sometimes signal abnormally. In epilepsy, the normal pattern of neuronal activity becomes disturbed, causing strange sensations, emotions, and behavior or sometimes convulsions, muscle spasms, and loss of consciousness. Epilepsy is a disorder with many possible causes. Anything that disturbs the normal pattern of neuron activity—from illness to brain damage to abnormal brain development—can lead to seizures. Epilepsy may develop because of an abnormality in brain wiring, an imbalance of nerve signaling chemicals called neurotransmitters, or some combination of these factors. Having a seizure does not necessarily mean that a person has epilepsy. Only when a person has had two or more seizures is he or she considered to have epilepsy.

The term “seizure” as used herein refers to a transient alteration of behaviour due to the disordered, synchronous, and rhythmic firing of populations of brain neurones.

The term “migraine” as used herein means a disorder characterised by recurrent attacks of headache that vary widely in intensity, frequency, and duration. The pain of a migraine headache is often described as an intense pulsing or throbbing pain in one area of the head. It is often accompanied by extreme sensitivity to light and sound, nausea, and vomiting. Some individuals can predict the onset of a migraine because it is preceded by an “aura,” visual disturbances that appear as flashing lights, zig-zag lines or a temporary loss of vision. People with migraine tend to have recurring attacks triggered by a lack of food or sleep, exposure to light, or hormonal irregularities (only in women). Anxiety, stress, or relaxation after stress can also be triggers. For many years, scientists believed that migraines were linked to the dilation and constriction of blood vessels in the head.

Investigators now believe that migraine is caused by inherited abnormalities in genes that control the activities of certain cell populations in the brain. The International Headache Society (IHS, 1988) classifies migraine with aura (classical migraine) and migraine without aura (common migraine) as the major types of migraine.

Activity in any of the above-mentioned indications can of course be determined by carrying out suitable clinical trials in a manner known to a person skilled in the relevant art for the particular indication and/or in the design of clinical trials in general.

For treating diseases, compounds of formula (I) or their pharmaceutically acceptable salts may be employed at an effective daily dosage and administered in the form of a pharmaceutical composition.

Therefore, another embodiment of the present invention concerns a pharmaceutical composition comprising an effective amount of a compound of formula (I) or a pharmaceutically acceptable salt thereof in combination with a pharmaceutically acceptable diluent or carrier.

To prepare a pharmaceutical composition according to the invention, one or more of the compounds of formula (I) or a pharmaceutically acceptable salt thereof is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, parenteral or intranasal.

Pharmaceutical compositions comprising compounds according to the invention can, for example, be administered orally, parenterally, i.e., intravenously, intramuscularly or subcutaneously, intrathecally, by inhalation or intranasally.

Pharmaceutical compositions suitable for oral administration can be solids or liquids and can, for example, be in the form of tablets, pills, dragees, gelatin capsules, solutions, syrups, chewing-gums and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus the quantity of compound of formula (I) in compositions for oral administration is at least 0.5% by weight and can be up to 80% by weight with respect to the total weight of the composition.

For the preferred oral compositions, the daily dosage is in the range 3 to 3000 milligrams (mg) of compounds of formula (I).

In compositions for parenteral administration, the quantity of compound of formula (I) present is at least 0.5% by weight and can be up to 33% by weight with respect to the total weight of the composition. For the preferred parenteral compositions, the dosage unit is in the range 3 mg to 3000 mg of compounds of formula (I).

The daily dose can fall within a wide range of dosage units of compound of formula (I) and is generally in the range 3 to 3000 mg. However, it should be understood that the specific doses can be adapted to particular cases depending on the individual requirements, at the physician's discretion.

A. According to one embodiment, compounds of general formula (I) may be prepared by reaction of a compound of formula (II) with a cyclic amine (AH), according to the equation:

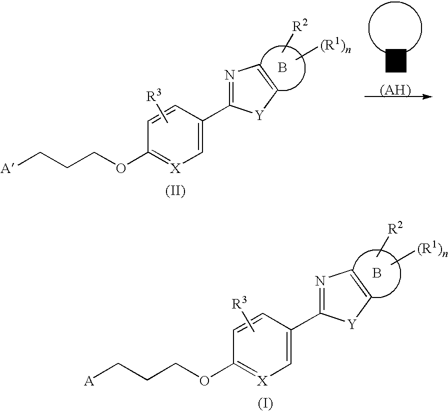

wherein A′ is a leaving group such as a halogen atom or a sulfonate group, A, B, X, Y, R¹, R², n and R³ having the same definitions as described above for compounds of formula (I).

This reaction may be carried out in the presence of a base, such as triethylamine, in a solvent such as acetonitrile, and in the presence of an additive, such as sodium iodide, or according to any other conventional methods known to the man skilled in the art.

The term “leaving group”, as used herein, has the same meaning by the person skilled in the art as defined in “Advanced Organic Chemistry: reactions, mechanisms and structure —Third Edition by Jerry March, John Wiley and Sons Ed.; 1985 page 179”. Examples of leaving group are fluorine, chlorine, bromine and methylsulfonate. A suitable leaving group is e.g. a chlorine atom.

(A.1) Compounds of formula (II) may be prepared according to one of the following methods.

(A.1.1) Compounds of formula (II) may be prepared by reaction of a compound of formula (III) with a compound of formula (IV) according to the equation:

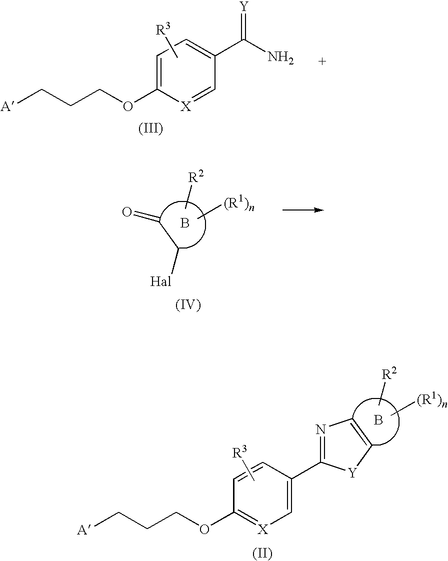

wherein A′, A, B, X, Y, R¹, R², n and R³ have the same definitions as described above and Hal is a leaving group, preferably a bromine atom.

This reaction may be carried out in the presence of a solvent, such as iso-propanol or dimethylformamide, at a temperature ranging from 50° C. to 130° C., or according to the method described by Ashton, W. T. et al. in Bioorg. Med. Chem. Lett. 2005, 15, 2253, or according to any other conventional methods known to the man skilled in the art. Preferably, Y is S.

In a particular embodiment, this method may be used for the synthesis of compounds of formula (II), hereafter referenced as compounds (IIa), wherein Y is S, B is a 5-8-membered heterocycloalkyl group containing a nitrogen atom and R² is linked to the nitrogen atom, A′, X, R¹, R², n and R³ having the same definitions as described above for compounds of formula (II). Preferably, n is equal to 0.

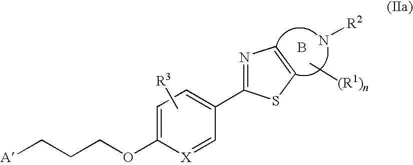

In another particular embodiment, the same method may be used for the synthesis of compounds of formula (II), hereafter referenced as compounds (IIb), wherein Y is S, B is a 5-8-membered cycloalkyl or heterocycloalkyl group, R² is an oxo group, A′, X, R¹, n and R³ having the same definitions as described above for compounds of formula (II). Preferably, n is equal to 0.

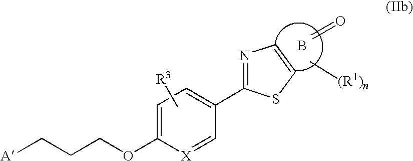

In another particular embodiment, the same method may be used for the synthesis of compound of formula (II), hereafter referenced as compounds (IIc), wherein Y is S, B is a 5-8-membered cycloalkyl group, R² is alkoxycarbonyl or acylamino, A′, X, R¹, n and R³ having the same definitions as described above for compounds of formula (II). Preferably, n is equal to 0.

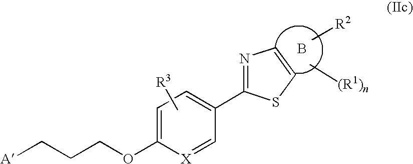

In a another particular embodiment, the same method may be used for the synthesis of compounds of formula (II), hereafter referenced as compounds (IIe), wherein Y is S, B is a 5-8-membered heterocycloalkyl group containing a nitrogen atom and R² is linked to this nitrogen atom, one of the R¹ is an oxo group to form with the nitrogen atom a lactam moiety, A′, X, R¹, R², n and R³ having the same definitions as described above for compounds of formula (II). Preferably, n is equal to 1.

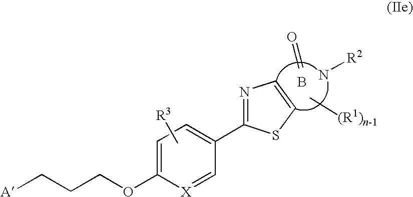

In some cases, an intermediate of formula (V) is formed, according to equation:

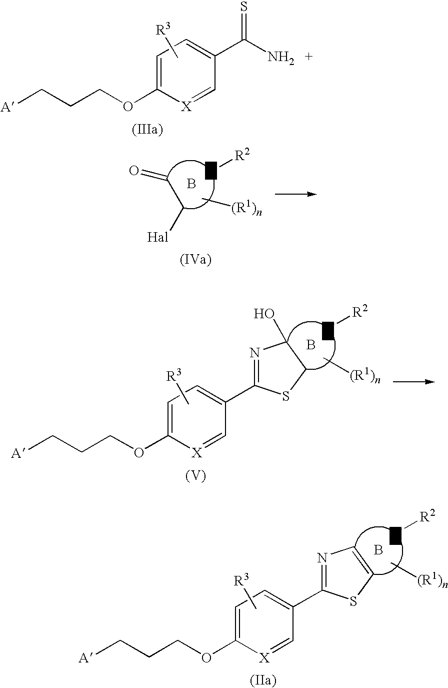

wherein A′, B, X, R¹, n and R³ have the same definitions as described above for compounds of formula (IIa) and Hal is a halogen atom. Preferably, R¹ is hydrogen. Intermediate (V) may be in turn transformed to compound (IIa) wherein R² is sulfonyl using an activating agent, such as mesyl chloride (MsCl), in dichloromethane as solvent according to the method described by Kim, W.-J. et al. in Heterocycles (1995), 41, 1389; or according to any other conventional methods known to the man skilled in the art.

(A.1.2) Compounds of formula (II) wherein Y is S and R² is OH may be prepared by reduction of a compound of formula (IIb) using a reducing agent, such as sodium borohydride or any other reagents and reaction conditions that the man skilled in the art will deem appropriate, and according to conventional methods known to him.

(A.1.3) Some compounds of formula (II) wherein R² is carboxy may be prepared by hydrolysis of the corresponding compound of formula (II) wherein R² is alkoxycarbonyl, according to conventional methods known to the man skilled in the art.

(A.1.4) Some compounds of formula (II) wherein R² is aminocarbonyl may be prepared by reaction of the corresponding compound of formula (II) wherein R² is carboxy with an amine, according to conventional methods known to the man skilled in the art.

For example, this reaction may be carried out using a coupling agent such as hydroxybenzotriazole, an activating agent, such as EDCI (1-(3-dimethylaminopropyl)-3-ethylcarbodiimide), in a solvent such as dichloromethane, or using any other reagents and reaction conditions that the man skilled in the art will deem appropriate, and according to conventional methods known to him.

(A.1.5) Compounds of formula (II) wherein R² is carbamate may be prepared from the corresponding compounds of formula (II) wherein R² is carboxy according to the method so-called “Curtius rearrangement” or to any other conventional methods known to the man skilled in the art.

For example, this reaction may be carried out using diphenylphosphorylazide in the presence of a base, such as triethylamine, and an alcohol, such as benzyl alcohol, at a temperature ranging from 0° C. to 100° C., as described by Gomez-Sanchez, E. and Marco-Contelles, J. in Tetrahedron (2005), 61, 1207; or using any other reagents and reaction conditions that the man skilled in the art will deem appropriate, and according to conventional methods known to him.

(A.1.6) Compounds of formula (IIc) wherein R² is alkoxycarbonyl, R¹ is C₁-C₆-alkyl and n is 1, R² and R¹ being linked to the same carbon atom of the B ring, may be prepared by alkylation of the corresponding compound of formula (IIc) wherein n is 0. This reaction may be carried out using a base, such as lithium diisopropylamide, in a solvent such as tetrahydrofuran, at a temperature ranging from −78° C. to 0° C., or at any other reaction conditions that the man skilled in the art will deem appropriate, and according to conventional methods known to him.

(A.1.7) In a particular embodiment, compounds of formula (IId) wherein B is pyridyl, hereafter referenced as compounds of formula (IId), may be prepared from compounds of formula (VI) by an intramolecular cyclisation reaction, according to conventional methods known to the man skilled in the art, according to the equation:

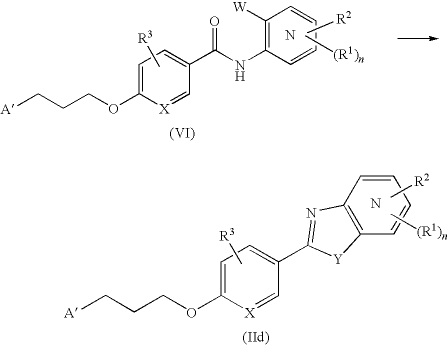

In a particular embodiment, compounds of formula (IId) wherein Y is oxygen may be prepared from compounds of formula (VI) wherein W is hydroxy, according to the method described by Heuser, S. et al. Tetrahedron Lett (2005), 46, 9001, or according to any other conventional methods known to the man skilled in the art.

In another particular embodiment, compounds of formula (IId) wherein Y is sulfur may be prepared by reaction of a compound of formula (VI) wherein W is a halogen atom, preferably chlorine atom, with a sulfur-releasing agent, such as 2,4-bis(4-methoxyphenyl)-1,3-dithia-2,4-diphosphetane-2,4-disulfide, in a solvent such as chloroform, at a temperature ranging from 50° C. to 130° C., or using any other reagents or reaction conditions that the man skilled in the art will deem appropriate, and according to conventional methods known to him.

(A.1.8) Some compounds of formula (IIa) may be prepared from compound of formula (If) according to the equation:

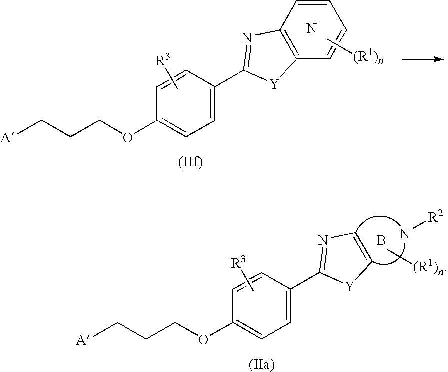

In a particular embodiment, compounds of formula (IIa) wherein R² is hydrogen are obtained from compounds of formula (If) in a single step reaction, using a reducing agent, for example hydrogen, in the presence of a catalyst, such as platinium oxide, in a polar and/or protic solvent, such as methanol. Alternatively, this step may be performed using acetyl chloride in an aprotic solvent, such as tetrahydrofuran, at a temperature ranging from −70° C. to 70° C., preferably at −40° C., followed by the use of a reducing agent, such as sodium borohydride; or according to any other method known to the person skilled in the art.

In another particular embodiment, compounds of formula (If) may be converted to compounds of formula (IIa) wherein R² is C₁-C₆-alkyl, preferably a methyl, or an C₁-C₆-alkyl aryl group, preferably a benzyl, in a two step sequence, via an alkyl or a benzyl pyridinium intermediate. This reaction may be carried out using either an alkyl halide, such as methyl iodide, methyl triflate or an alkyl halide, such as benzyl bromide. The corresponding alkyl or benzyl pyridinium intermediate prepared may be reduced using sodium borohydride or any other related reducing agent, in a protic solvent, such as methanol, at a temperature ranging from 0° C. to 60° C., preferably at room temperature. Alternatively, these steps may be performed according to any other conventional method known to the man skilled in the art.

(A.1.9) Compound of formula (IIa) wherein R² is acetyl may be prepared from the corresponding compound of formula (IIc), wherein R² is (4-methylphenyl)sulfonyl. This reaction may be carried out using bromide in acetic acid, at a temperature ranging from 0° C. to 100° C., preferably at room temperature, or according to any conventional method known to the man skilled in the art.

(A.1.10) Compound of formula (IIa) wherein R² is an aminosulfonyl may be prepared from the corresponding compound of formula (IIa), wherein R² is hydrogen. For example, this reaction may be carried out using an aminosulfonyl chloride in the presence of a base such as triethylamine, in a solvent such as dichloromethane and at a temperature ranging from 0° C. to 100° C., preferably at room temperature. Alternatively, this reaction may be performed according to the method described by Beaudoin et al. in J. Org. Chem., 2003 (68) 115-119, or any modification of this present route.

(A.1.11) Compound of formula (IIe) wherein R² is C₁-C₆-alkyl, preferably a methyl, may be prepared by alkylation of the corresponding compound of formula (IIe) wherein R² is hydrogen. For example, this reaction may be carried out using an alkyl halide, preferably methyl iodide, in the presence of a base such as sodium hydride, in a solvent such as dimethylformamide and at a temperature ranging from 0° C. to 100° C., preferably at room temperature, or according to any other conventional method known to the man skilled in the art.

(A.1.12) Compounds of formula (IIa) wherein R² is an acyl group may be prepared from the corresponding compounds of formula (IIa), wherein R² is hydrogen, using an acyl halide, in the presence of a base, such as triethylamine, in a solvent such as dichloromethane, according to any conventional method known to the man skilled in the art.

(A.1.13) Compounds of formula (IIa) wherein R² is hydrogen may react with an isocyanate to provide corresponding compounds of formula (IIa) wherein R² is ureido, according to conventional methods known to the man skilled in the art. For example, this reaction may be carried out in a solvent such as dichloromethane or in any other reaction conditions that the man skilled in the art will deem appropriate, and according to conventional methods known to him.

(A.1.14) Alternatively, compounds of general formula (II) may be prepared by reaction of a compound of formula (XVIII) with a dihalopropane, preferably 1-bromo-3-chloropropane, according to the equation:

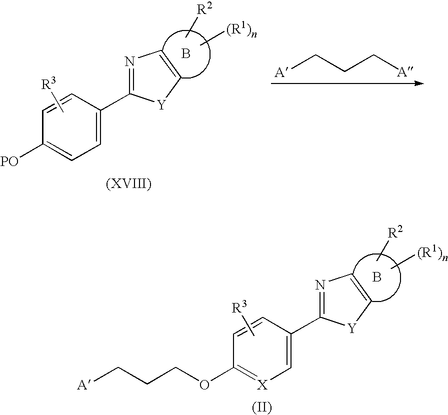

wherein X is CH, P is a hydrogen, A and A′ are halogen atoms, preferably, A′ is chloride and A is bromine or iodide, Y, B, R¹, R², R³, n having the same definition as described above for compound of formula (I).

This reaction may be carried out in the presence of a base, for example potassium carbonate, in a solvent, for example acetone or acetonitrile, at a temperature ranging from 0° C. to 100° C., according to the method described by Walsh et al. (J. Med. Chem. 1989, 32, 105). An additive, such as potassium iodide, may be used. Alternatively, this reaction may be carried out using alternative experimental conditions that the man skilled in the art will deem appropriate, and according to conventional methods known to him.

(=A.1.11)(A.2) Compounds of formula (III) may be commercially available or prepared according to one of the following methods.

Compounds of formula (III) wherein Y is an oxygen atom may be obtained by the reaction of a para-hydroxybenzamide derivatives and a di-halopropane, preferably 1-chloro-3-bromopropane, according to methods described by Walsh et al. in J. Med. Chem. (1989), 32, 105, or according to any other conventional methods known to the man skilled in the art. Usually, para-hydroxybenzamide derivatives may be commercially available or may be prepared according to conventional methods known to the man skilled in the art.

Compounds of formula (III) wherein Y is a sulfur atom may be prepared from the corresponding compounds of formula (III) wherein Y is an oxygen atom, according to conventional methods known to the man skilled in the art. For example, this reaction, may be achieved by reacting said compounds (III) with 2,4-bis(4-methoxyphenyl)-1,3-dithia-2,4-diphosphetane-2,4-disulfide in a solvent, such as chloroform, at a temperature ranging from 50° C. to 130° C.

(A.3) Compounds of formula (IV) may be commercially available or prepared according to one of the following methods.

(A.3.1) Compounds of formula (IV) wherein Hal is a halogen atom, R¹, R² and n being as defined in the specifications for compounds of general formula (I), may be prepared by reaction of a compound of formula (VII) with a halogen-releasing agent according to the equation:

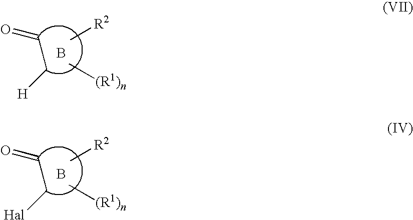

This reaction may be carried out using bromine (Br₂) or polymer-supported pyridinium tribromide, in a solvent such as dichloromethane or chloroform, at a temperature ranging from 0° C. to 25° C., according to the following methods described by Marinko, P. et al. (Eur. J. Med. Chem. (2004), 39, 257) or Habermann, J. et al. (J. Chem. Soc., Perkin Trans. 1 (1999) 2425), or using any other reagents or reaction conditions that the man skilled in the art will deem appropriate, and according to conventional methods known to him. Preferably, Hal is a bromine atom.

(A.3.2) Alternatively, some compounds of formula (IV) may be prepared in two steps according to the equation:

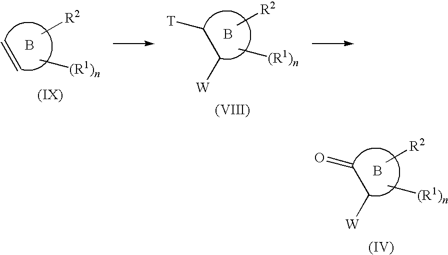

wherein W represents a halogen atom, preferably a bromine atom, T is hydroxy, B is a 5-8-membered heterocycloalkyl or a 5-8-membered cycloalkyl, R¹, R² and n having the same definitions as described above.

In this case, compounds of formula (IV) may be prepared by reaction of a “halohydrine” of formula (VIII) with an oxidizing agent, such as Dess-Martin periodinane reagent or pyridinium chlorochromate, or according to any conventional methods known to the man skilled in the art.

Compounds of formula (VII) wherein T is hydroxy and W is halogen may be commercially available or may be prepared by the reaction of a 5-8-membered cycloalkene or a 5-8-membered heterocycloalkene of formula (IX) with a halogen-releasing agent, such as N-bromosuccinimide, in the presence of water, according to the method described by Kim, W.-J. et al. in Heterocycles (1995), 41, 1389; or according to any other conventional methods known to the man skilled in the art.

Compounds of formula (IX) may be commercially available or may be prepared according to any other conventional methods known to the man skilled in the art. For example, compounds (IX) may be prepared by intramolecular metathesis reaction of a di-alkene according to the method described by Yao, Q. et al. in Angew. Chem. Int. Ed. (2000), 39, 3896.

(A.4) Compounds of formula (VI) may be prepared by reaction of compounds of formula (X) with compounds of formula (XI) according to conventional methods known to the man skilled in the art, according to the equation:

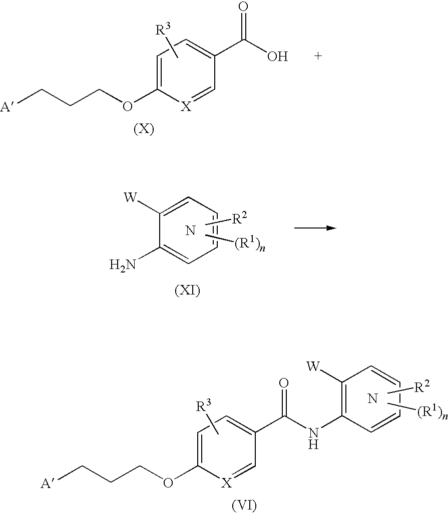

For example, this reaction may be carried out using an activated agent, such as oxalyl chloride, in a solvent such as dimethylformamide or dichloromethane, at a temperature ranging from 0° C. to 50° C., or using in any other reagents or reaction conditions that the man skilled in the art will deem appropriate, and according to conventional methods known to him.

Compounds of formula (X) are commercially available, or may be prepared by reaction of the corresponding unsubstituted or substituted para-hydroxybenzoic acid with a 1,3-di-halogenoalkane, preferably 1-chloro-3-bromopropane, followed by hydrolysis, according to methods described previously in A.2 or to any other conventional method known to the man skilled in the art.

Compounds of formula (XI) are commercially available, or may be prepared according to methods described previously or to any other conventional methods known to the man skilled in the art.

(A.5) Compounds of formula (XVIII) may be prepared according to the following methods.

(A.5.1) Compounds of formula (XVIII), wherein P is a hydrogen may be obtained from corresponding compounds of formula (XVIII) wherein P is a protecting group. Examples of protecting group may be a benzyl group, a trialkylsilyl group, a tert-butoxy group, an acetyl group, an alkyl group or any other phenol-related protecting groups known by the man skilled in the art will deem appropriate. Such protecting group may be removed using any methodologies and experimental conditions that the man skilled in the art will deem appropriate, and according to conventional methods known to him.

In a specific embodiment, when P is a benzyl group, compounds of formula (XVIII), wherein P is H, may be obtained using hydrogen and a catalyst, such as palladium on charcoal, in a solvent, preferably a protic solvent such as methanol. The reaction may be conducted at a temperature ranging from room temperature to 60° C., either at atmospheric pressure or under high pressure.

(A.5.2) Compounds of formula (XVIII), wherein P is a protecting group as described above, may be prepared from compounds of formula (XX) according to the equation:

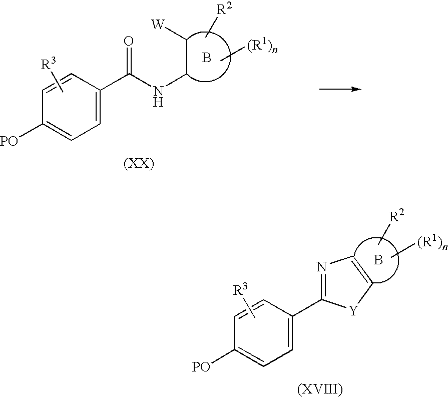

wherein B, W is hydroxy, halogen or oxo, B, R¹, R², R³ and n having the same definitions as described above for compound of formula (I).

In a specific embodiment, compounds of formula (XVIII), wherein P is benzyl, Y is a sulfur atom, B is a 5-8-membered heterocycloalkyl containing a nitrogen atom to which R² is linked, may be prepared from compound of formula (XX), wherein B is a 5-8-membered heterocycloalkyl containing a nitrogen atom to which R² is linked and W is an oxo group. This reaction may be carried out using a sulfur-releasing agent, such as 2,4-bis(4-methoxyphenyl)-1,3-dithia-2,4-diphosphetane-2,4-disulfide, in an inert solvent such as chloroform, at a temperature ranging from 50° C. to 130° C., or using any other reagents or reaction conditions that the man skilled in the art will deem appropriate, and according to conventional methods known to him.

In another specific embodiment, compounds of formula (XVIII), wherein P is benzyl, Y is an oxygen atom, B is pyridyl, R² is hydrogen, R¹, R³ and n having the same definitions as described above for compound of formula (I), may be prepared from compounds of formula (XX), wherein B is pyridyl, R² is hydrogen and W is hydroxy. This reaction may be carried out according to the procedure described in (A.1.7) or according to any other conventional methods known to the man skilled in the art.

Compounds of formula (XX) may be prepared according to the following methods.

(i) Compounds of formula (XX), wherein P is a protecting group as described above, W is halogen, hydroxy or oxo, B is a 5-8-membered cycloalkyl or a 5-8-membered heterocycloalkyl may be prepared by the reaction of a compound of formula (XXI) with a compound of formula (VIII), according to the equation:

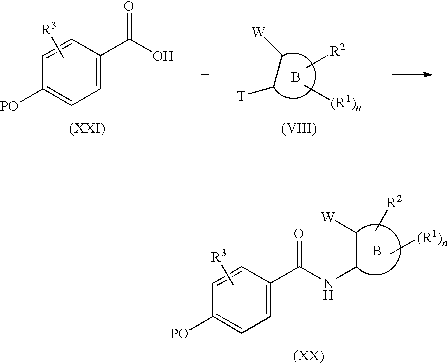

wherein T is NH₂, W, B, R¹, R², R³ and n having the same definition as described above.

In a specific embodiment, compounds of formula (XX), wherein B is a 5-8-membered heterocycloalkyl containing a nitrogen atom to which R² is linked, R² having the same definition as described above, and W is an oxo group, may be prepared by the reaction of a compound of formula (XXI) wherein P is a protecting group, preferably a benzyl, with a compound of formula (VIII), wherein B is a 5-8 membered heterocycloalkyl containing a nitrogen atom to which R² is linked, T is NH₂ and W is an oxo group. This reaction may be carried out using the procedure described in (A.4) or according to any other conventional methods known to the man skilled in the art.

(ii) Compounds of formula (XX) wherein B is pyridyl, R² is hydrogen and W is hydroxy may be prepared by reaction of a compound of formula (XXI) with a compound of formula (IX), according to the equation

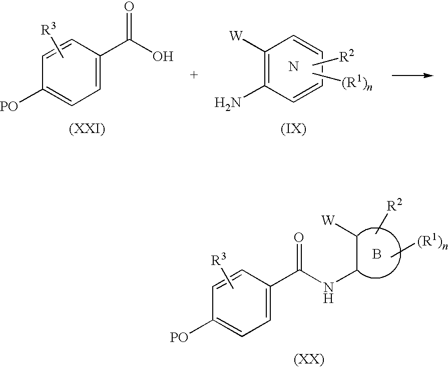

wherein P is a protecting group, preferably a benzyl, and W is hydroxy.

This reaction may be carried out using the procedure described in (A.4) or according to any other conventional methods known to the man skilled in the art.

Compounds of formula (XXI) may be commercially available or may be prepared according to any conventional methods known to the man skilled in the art.

(A.5.3) Compounds of formula (XVIII), wherein P is a protecting group as described above and B is a 6-membered heterocycloalkyl containing a nitrogen atom to which R² is linked may be prepared from compounds of formula (XVIII) wherein B is pyridyl and R² is hydrogen. This reaction may be performed according to the procedure described in (A.1.8) or according to any other conventional methods known to the man skilled in the art.

In a specific embodiment, P is benzyl, and Y is an oxygen atom.

B. According to another embodiment, some compounds of general formula (I) may be prepared by reaction of a compound of formula (XII) with a compound of formula (IV) according to the equation:

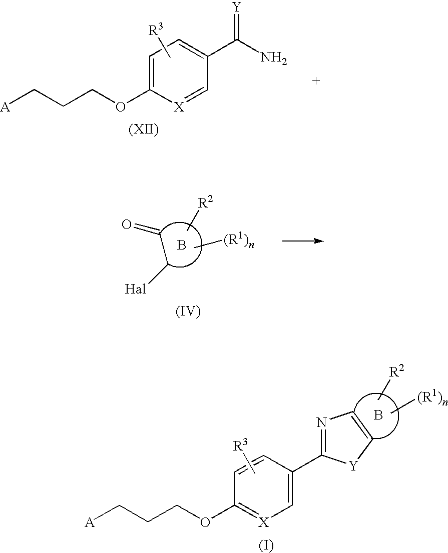

wherein A, B, X, Y, R¹, R², n and R³ are as defined for compounds of general formula (I) and Hal is a halogen atom, preferably a bromine atom.

This reaction may be carried out according to the method previously described in A.1.1, or according to any other conventional methods known to the man skilled in the art.

Compounds of formula (XII) wherein X is CH may be commercially available or may be obtained by reaction of a compound of formula (III) with a cyclic amine (AH) according to the method previously described in method A, or according to any other conventional methods known to the man skilled in the art.

Compounds of formula (XII) wherein X is N may be obtained by reaction of 2-halopyridyl-carboxamide derivatives and the like, preferably 2-chloro- or 2-bromopyridyl-5-carboxamide derivatives, with a substituted or unsubstituted aminoalcohol of formula HO—(CH₂)₃-A, according to conventional methods known to the man skilled in the art. For example, this reaction may be carried out in the presence of a base, such as potassium tert-butylate, cesium carbonate or sodium hydride, and a solvent, such as dimethylformamide or tetrahydrofuran, at a temperature ranging from 25° C. to 130° C. A palladium- or a copper-based catalyst, may be added according to the method described by Penning et al. in J. Med. Chem. (2000), 43, 721.

C. Alternatively, some compounds of general formula (I) may be prepared by the reaction of a compound of formula (XIII) with a compound of formula (XIV) according to the equation:

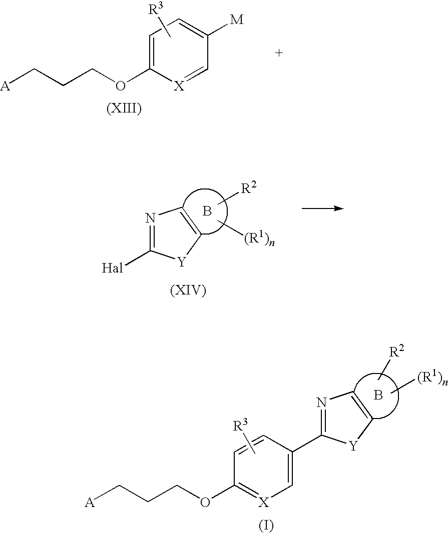

wherein M is either a boronic acid (M=B(OH)₂) or a boronate ester (M=B(OR)₂), a trialkyltin (M=Sn(R)₃), a zinc derivative (M=ZnHal) or a magnesium derivative (M=MgHal); and Hal is a halogen atom (Hal=Cl, Br, I).

This reaction may be carried out using conventional methods of cross-coupling known to the man skilled in the art. For examples, reactions may be carried out in the presence of a transition metal catalyst, such as palladium, nickel or copper, a ligand such as a trialkylphosphine, in the presence of a base and a solvent, at a temperature ranging from 25° C. to 130° C., or according to any other reaction conditions that the man skilled in the art will deem appropriate, and according to conventional methods known to him.

For example, when R² is an oxo group, compounds of formula I may be prepared by reaction of a compound of formula (XIII) wherein X is CH, M is B(OR)₂ and R³ is C₁-C₆-alkyl or C₃-C₈-cycloalkyl, with a compound of formula (XIV), wherein Hal is a chlorine atom and R² is oxo group, in the presence of a catalyst, such as tetrakis-triphenylphosphine palladium (Pd(PPh₃)₄), a base, such as potassium acetate, a solvent, like dimethylformamide, and at a temperature comprised between 25° C. and 130° C.

Compounds of formula (XIII) wherein M is B(OR)₂ may be obtained by reaction of a compound of formula (XV), wherein Hal is a halogen atom, with a suitable boron reagent, according to the equation:

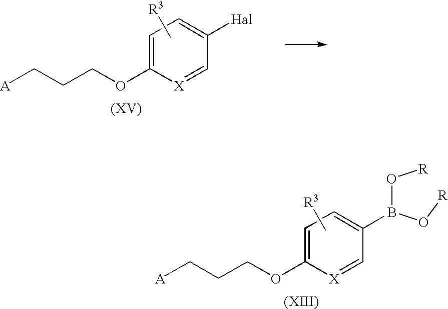

This reaction may be performed according to any conventional methods known to the man skilled in the art. For example, compounds (XV), wherein Hal is a bromine or iodine atom, may react with bis(pinacolato)diboron in the presence of a palladium-based catalyst, such as 1,1′-bis(diphenylphosphino)ferrocene palladium dichloride, in the presence of a base, such as potassium acetate, in a solvent such as dimethylformamide or DMSO, at a temperature ranging from 60° C. to 130° C., according to the method described by Ishiyama, T. et al. in J. Org. Chem. (1995), 60, 7508, or using any other reaction conditions that the man skilled in the art will deem appropriate, and according to conventional methods known to him.

Compound of formula (XV) wherein X is CH may be commercially available or may be prepared by reaction of a compound of formula (XVI) with a cyclic amine (AH) according to the equation:

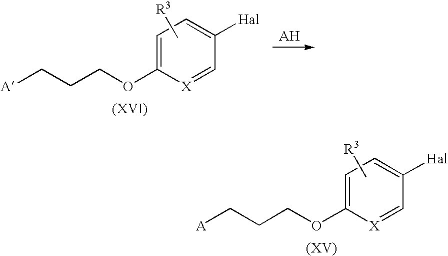

wherein A′ is a leaving group and Hal a halogen atom. This reaction may be performed according to any conventional methods known to the man skilled in the art.

Compounds of formula (XVI) wherein X is N may be commercially available or may be prepared by reaction of a 2-halopyridyl derivative, preferably 2-chloro- or 2-bromo-pyridyl derivative, with amino-alcohols of formula HO—(CH₂)₃-A according to conventional methods known to the man skilled in the art. For example, this reaction may be carried out in the presence of a base, such as potassium tert-butylate, cesium carbonate or sodium hydride, in a solvent, such as dimethylformamide or tetrahydrofuran, at a temperature ranging from 25° C. to 130° C. A palladium- or a copper-based catalyst may be added according to the method described by Penning et al. in J. Med. Chem. (2000), 43, 721. Compounds of formula (XIV) may be commercially available or may be prepared from compounds of formula (XVII) according to the equation:

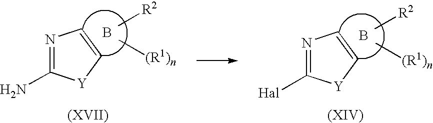

wherein B, Y, R¹, R² and n are defined above in the specification for compounds of general formula (I) and Hal is a halogen atom.

This reaction may be performed according to conventional methods known to the man skilled in the art. For example, it may be carried out in the presence of a halogen releasing agent such as copper chloride or copper bromide, and tert-butylnitrite (tBuONO), in a solvent such as acetonitrile or dimethylformamide, according to the method described by Haginoya, N. et al in Heterocycles (2004), 63, 1555; or according to other conditions that the man skilled in the art will deem appropriate.

Compounds of formula (XVII) may be commercially available or may be prepared by reaction of a thiourea with a previously described compound of formula (IV), according to the method described by Marinko, P. et al. in Eur. J. Med. Chem. (2004), 39, 257, or according to any other conventional methods known to the man skilled in the art.

D. In a specific embodiment, some compounds of general formula (I) wherein B is a 5-8-membered cycloalkyl and R² an ureido group of formula —NHC(O)A, hereafter referenced as compounds (Id), may be prepared by reaction of the corresponding compounds of formula (II) wherein R² is carbamate, with the amine (AH), according to the equation:

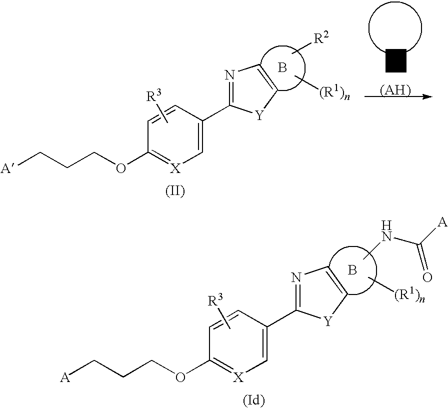

wherein A′ is a leaving group such as a halogen atom or a sulfonate group, A, B, X, Y, R¹, n and R³ having the same definitions as described above for compounds of formula (I). The reaction may be carried out according to the reaction conditions described in method A or according to any conventional methods known to the man skilled in the art.

E. According to another embodiment, some compounds of general formula (I) may be prepared by functional group transformations.

(E.1) Compounds of formula (I) wherein B is a 5-8-membered cycloalkyl and R² is carbamate may be transformed into the corresponding amine of formula (I) wherein R² is NH₂, according to conventional methods known to the man skilled in the art. For example, this reaction may be carried out using aqueous hydrochloric acid at a temperature ranging from 25° C. to 100° C., or according to any reaction conditions that the man skilled in the art will deem appropriate, and according to conventional methods known to him.

(E.2) Compounds of formula (I), wherein B is a 5-8-membered cycloalkyl and R² is acylamino may be prepared from corresponding compound of formula I wherein R² is NH₂, according to conventional methods known to the man skilled in the art. For example, this reaction may be carried out in the presence of acetyl chloride and a base, such as triethylamine, in a solvent such as dichloromethane, or using any reaction conditions that the man skilled in the art will deem appropriate, and according to conventional methods known to him.

(E.3) Compounds of formula (I) wherein B is a 5-8-membered cycloalkyl and R² an amino group may be prepared from the reaction of the corresponding compound of formula (I) wherein R² is hydroxy, according to conventional methods known to the man skilled in the art. For example, this reaction may be carried out in the presence of an activating agent, such as mesyl chloride, a base, such as triethylamine, a solvent such as dichloromethane, followed by the addition of an amine; or using any reaction conditions that the man skilled in the art will deem appropriate, and according to conventional methods known to him.

(E.4) Compounds of formula (I) wherein B is a 5-8 membered heterocycloalkyl containing a nitrogen atom and R² is hydrogen may react with an isocyanate to provide corresponding compounds of formula (I) wherein R² is ureido, according to conventional methods known to the man skilled in the art. For example, this reaction may be carried out in a solvent such as dichloromethane or in any other reaction conditions that the man skilled in the art will deem appropriate, and according to conventional methods known to him.

(E.5) Compounds of formula (I) wherein B is a 5-8 membered heterocycloalkyl containing a nitrogen atom and R² is hydrogen may react with an amino group and a di-activated carbonyl group, such as triphosgene, to provide corresponding compounds of formula (I) wherein R² is an acylamino group, according to the method described by Lemoucheux, L. et al. in J. Org. Chem. (2003), 68, 7285, or according to any conventional methods known to the man skilled in the art. For example, this reaction may be carried out in the presence of a base, in a solvent such as dichloromethane or in any other reaction conditions that the man skilled in the art will deem appropriate, and according to conventional methods known to him.

(E.6) Compounds of formula (I) wherein B is a 5-8 membered heterocycloalkyl containing a nitrogen atom and R² is hydrogen may react with an acyl chloride to provide the corresponding compounds of formula (I) wherein R² is an acyl group, according to conventional methods known to the man skilled in the art. For example, this reaction may be carried out in the presence of a base, in a solvent such as dichloromethane or in any other reaction conditions that the man skilled in the art will deem appropriate, and according to conventional methods known to him. Alternatively, this reaction may be carried out using a carboxylic acid, in the presence of an activated agent such as N-hydroxybenzotriazole, a coupling agent, such as EDCI, in a solvent such as dichloromethane, or using any other reagents and reaction conditions that the man skilled in the art will deem appropriate, and according to conventional methods known to him.

(E.7) Compounds of formula (I) wherein B is a 5-8 membered heterocycloalkyl containing a nitrogen atom and R² is hydrogen may react with a carbonyl derivative in the presence of a reducing agent to provide compounds of formula (I) wherein R² is an alkyl or a cycloalkyl group, according to conventional methods known to the man skilled in the art. For example, this reaction may be carried out in the presence of sodium cyanoborohydride in acidic media and a solvent such as methanol or in any other reaction conditions that the man skilled in the art will deem appropriate, and according to conventional methods known to him.

Alternatively, compounds of formula (I) wherein R² is a C₁-C₆-alkyl or a C₃-C₈-cycloalkyl may be prepared by reaction of a compound of formula (I) wherein R² is hydrogen with an alkyl halide or a cycloalkyl halide, in the presence of a base such as di-isopropylethyl amine, potassium carbonate, sodium hydride in an inert solvent such as dimethylsulfoxyde, dichloromethane, dimethylformamide or acetonitrile, at a temperature ranging from 0° C. to 100° C., preferably at room temperature.

(E.8) Compounds of formula (I) wherein B is a 5-8-membered heterocycloalkyl containing a nitrogen atom and R² is hydrogen may react with an aryl or heteroaryl halide group, in the presence of a transition metal catalyst, to provide the corresponding compounds of formula (I) wherein R² is an aryl or heteroaryl group, according to conventional methods known to the man skilled in the art. For example, this reaction may be carried out in the presence of bis(dibenzylideneacetone)palladium and a ligand, such as 2-(dicyclohexylphosphino)-biphenyl, a base such as sodium tert-butylate and a solvent such as toluene, or in any other reaction conditions that the man skilled in the art will deem appropriate, and according to conventional methods known to him.

(E.9) Some compounds of formula (Ia) may be obtained by reduction of corresponding compounds of formula (Ie) according to the equation

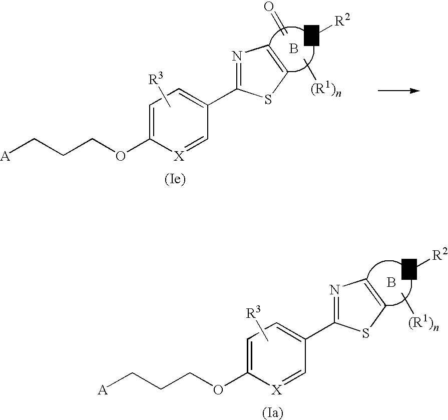

wherein A, X, R¹, R², n and R³ have the same definitions as described above for compounds of formula (I). For example, this reaction may be carried out by the use of a reducing agent such as borane derivatives (e.g., borane-dimethyl sulfide complex) in a solvent such as THF or ether and at a temperature ranging from 0° C. to 100° C., preferably at room temperature. Alternatively, this reaction may be carried out using other experimental conditions that the man skilled in the art will deem appropriate, and according to conventional methods known to him.

(E.10) Compounds of formula (Ia) wherein R² is hydrogen may react with amino sulfonyl chloride to provide the corresponding compounds of formula (I) wherein R² is an aminosulfonyl group, according to conventional methods known to the man skilled in the art. For example, this reaction may be carried out in the presence of a base such as triethylamine, in a solvent such as dichloromethane and at a temperature ranging from 0° C. to 100° C., preferably at room temperature. Alternatively, this reaction may be performed according to the method described by Beaudoin et al. in J. Org. Chem., 2003 (68) 115-119, or any modification of this present route.

(E.11) Compounds of formula (I) wherein R² contains a alkoxycarbonyl group may react with aqueous ammonia in a protic solvent such as methanol, at a temperature ranging from 25° C. to 100° C., to provide the corresponding compounds of formula (I) wherein R² contains an aminocarbonyl. Alternatively, this reaction may be carried out using other experimental conditions that the man skilled in the art will deem appropriate, and according to conventional methods known to him.

(E.12) Compounds of formula (I) wherein A is substituted by a benzyloxycarbonyl may react with a reducing agent to provide the corresponding compounds of formula (I) wherein A is substituted by hydroxymethyl. This reaction may be carried out using lithium borohydride or sodium borohydride, in a solvent such as tetrahydrofuran or methanol, at a temperature ranging from 25° C. to 100° C., preferably at room temperature. Alternatively, this reaction may be performed using other experimental conditions that the man skilled in the art will deem appropriate, and according to conventional methods known to him.

(E.13) Compounds of formula (I) wherein R² contains an alkoxycarbonyl group may react with trifluoroacetic acid in an inert solvent such as dichloromethane to provide the corresponding compounds of formula (I) wherein R² contains a carboxy group. This reaction may be carried out at a temperature ranging from 25° C. to 100° C., preferably at room temperature. Alternatively, this reaction may be performed using other experimental conditions that the man skilled in the art will deem appropriate, and according to conventional methods known to him.

(E.14) Compounds of formula (I) R² contains a alkoxycarbonyl may react with aqueous potassium hydroxide in an inert solvent such as tetrahydrofuran, at a temperature ranging from 0° C. to 100° C., preferably at room temperature, to provide the corresponding compounds of formula (I) wherein R² contains a potassium oxycarbonyl. Alternatively, this reaction may be carried out using other experimental conditions that the man skilled in the art will deem appropriate, and according to conventional methods known to him.

(E.15) Compounds of formula (Ia) wherein R² is hydrogen may react with an alkyldione or a cycloalkyldione to provide the corresponding compounds of formula (I) wherein R² is a hydroxyalkyl or a hydroxycycloalkyl group. For example, this reaction may be carried out in the presence of sodium cyanoborohydride in acidic media and a solvent such as methanol, or in any other reaction conditions that the man skilled in the art will deem appropriate, and according to conventional methods known to him.

(E.16) Compounds of formula (Ia) wherein R² is a hydroxyalkyl or a hydroxycycloalkyl may react with a halide releasing agent to provide the corresponding compounds of formula (I) wherein R² is a (di)halo alkyl or a (di)halo cycloalkyl group. For example, this reaction may be carried out using the mixture of triphenylphosphine/carbon tetrabromide or the dimethylaminosulfur trifluoride reagent in an inert solvent such as dichloromethane. Alternatively, this reaction may be carried out using other experimental conditions that the man skilled in the art will deem appropriate, and according to conventional methods known to him.

(E.17) Compounds of formula (Ia) wherein R² is hydrogen may react with cycloalkan-1,3-dione, to provide the corresponding compounds of formula (I) wherein R² is 3-oxocycloalkyl-1-en-1-yl. For example, this reaction may be carried out in an inert solvent such as dioxane at a temperature ranging from 20° C. to 100° C., preferably at room temperature. Alternatively, this reaction may be carried out using other experimental conditions that the man skilled in the art will deem appropriate, and according to conventional methods known to him.

In a particular embodiment, the present invention relates to synthetic intermediates of formula (II), geometrical isomers, enantiomers, diastereoisomers, pharmaceutically acceptable salts and all possible mixtures thereof,

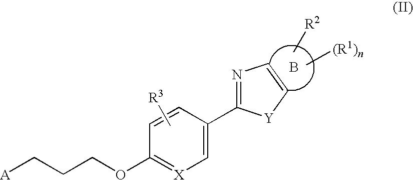

wherein
A′ is a leaving group; preferably A′ is chlorine;
B is selected from the group consisting of heteroaryl (e.g. pyridyl), 5-8-membered heterocycloalkyl, 5-8-membered cycloalkyl. In a specific embodiment B is selected from the group consisting of 5-7-membered heterocycloalkyl, 5-7-membered cycloalkyl;
X is either N or CH; in a specific embodiment X is CH;
Y is either O or S; in a specific embodiment Y is S;
R¹ is selected from the group comprising or consisting of sulfonyl, amino, substituted or unsubstituted C₁-C₆-alkyl, substituted or unsubstituted C₂-C₆-alkenyl, substituted or unsubstituted C₂-C₆-alkynyl, substituted or unsubstituted aryl, substituted or unsubstituted heteroaryl, substituted or unsubstituted C₃-C₈-cycloalkyl, substituted or unsubstituted 3-8-membered heterocycloalkyl, acyl, substituted or unsubstituted C₁-C₆-alkyl aryl, substituted or unsubstituted C₁-C₆-alkyl heteroaryl, substituted or unsubstituted C₂-C₆-alkenyl aryl, substituted or unsubstituted C₂-C₆-alkenyl heteroaryl, substituted or unsubstituted C₂-C₆-alkynyl aryl, substituted or unsubstituted C₂-C₆-alkynyl heteroaryl, substituted or unsubstituted C₁-C₆-alkyl cycloalkyl, substituted or unsubstituted C₁-C₆-alkyl heterocycloalkyl, substituted or unsubstituted C₂-C₆-alkenyl cycloalkyl, substituted or unsubstituted C₂-C₆-alkenyl heterocycloalkyl, substituted or unsubstituted C₂-C₆-alkynyl cycloalkyl, substituted or unsubstituted C₂-C₆-alkynyl heterocycloalkyl, alkoxycarbonyl, aminocarbonyl, substituted or unsubstituted C₁-C₆-alkyl carboxy, substituted or unsubstituted C₁-C₆-alkyl acyl, substituted or unsubstituted aryl acyl, substituted or unsubstituted heteroaryl acyl, substituted or unsubstituted C₃-C₈-(hetero)cycloalkyl acyl, substituted or unsubstituted C₁-C₆-alkyl acyloxy, substituted or unsubstituted C₁-C₆-alkyl alkoxy, substituted or unsubstituted C₁-C₆-alkyl alkoxycarbonyl, substituted or unsubstituted C₁-C₆-alkyl aminocarbonyl, substituted or unsubstituted C₁-C₆-alkyl acylamino, acylamino, acylaminocarbonyl, ureido, substituted or unsubstituted C₁-C₆-alkyl ureido, substituted or unsubstituted C₁-C₆-alkyl carbamate, substituted or unsubstituted C₁-C₆-alkyl amino, substituted or unsubstituted C₁-C₆-alkyl sulfonyloxy, substituted or unsubstituted C₁-C₆-alkyl sulfonyl, substituted or unsubstituted C₁-C₆-alkyl sulfinyl, substituted or unsubstituted C₁-C₆-alkyl sulfanyl, substituted or unsubstituted C₁-C₆-alkyl sulfonylamino, substituted or unsubstituted C₁-C₆-alkyl aminosulfonyl, hydroxy, halogen, cyano, carboxy, oxo, thioxo;
n is equal to 0, 1 or 2. In a specific embodiment, n is either 0 or 1;
R² is selected from the group comprising or consisting of hydrogen, sulfonyl, amino, substituted or unsubstituted C₁-C₆-alkyl, substituted or unsubstituted C₂-C₆-alkenyl, substituted or unsubstituted C₂-C₆-alkynyl, substituted or unsubstituted aryl, substituted or unsubstituted heteroaryl, substituted or unsubstituted C₃-C₈-cycloalkyl, substituted or unsubstituted 3-8-membered heterocycloalkyl, acyl, substituted or unsubstituted C₁-C₆-alkyl aryl, substituted or unsubstituted C₁-C₆-alkyl heteroaryl, substituted or unsubstituted C₂-C₆-alkenyl aryl, substituted or unsubstituted C₂-C₆-alkenyl heteroaryl, substituted or unsubstituted C₂-C₆-alkynyl aryl, substituted or unsubstituted C₂-C₆-alkynyl heteroaryl, substituted or unsubstituted C₁-C₆-alkyl cycloalkyl, substituted or unsubstituted C₁-C₆-alkyl heterocycloalkyl, substituted or unsubstituted C₂-C₆-alkenyl cycloalkyl, substituted or unsubstituted C₂-C₆-alkenyl heterocycloalkyl, substituted or unsubstituted C₂-C₆-alkynyl cycloalkyl, substituted or unsubstituted C₂-C₆-alkynyl heterocycloalkyl, alkoxycarbonyl, aminocarbonyl, substituted or unsubstituted C₁-C₆-alkyl carboxy, substituted or unsubstituted C₁-C₆-alkyl acyl, substituted or unsubstituted aryl acyl, substituted or unsubstituted heteroaryl acyl, substituted or unsubstituted C₃-C₈-(hetero)cycloalkyl acyl, substituted or unsubstituted C₁-C₆-alkyl acyloxy, substituted or unsubstituted C₁-C₆-alkyl alkoxy, substituted or unsubstituted C₁-C₆-alkyl alkoxycarbonyl, substituted or unsubstituted C₁-C₆-alkyl aminocarbonyl, substituted or unsubstituted C₁-C₆-alkyl acylamino, acylamino, acylaminocarbonyl, ureido, substituted or unsubstituted C₁-C₆-alkyl ureido, substituted or unsubstituted C₁-C₆-alkyl carbamate, substituted or unsubstituted C₁-C₆-alkyl amino, substituted or unsubstituted C₁-C₆-alkyl sulfonyloxy, substituted or unsubstituted C₁-C₆-alkyl sulfonyl, substituted or unsubstituted C₁-C₆-alkyl sulfinyl, substituted or unsubstituted C₁-C₆-alkyl sulfanyl, substituted or unsubstituted C₁-C₆-alkyl sulfonylamino, substituted or unsubstituted C₁-C₆-alkyl aminosulfonyl, hydroxy, halogen, cyano, carboxy, oxo, thioxo;
R³ is hydrogen or C₁-C₆-alkyl or halogen or C₁-C₆-alkoxy.

In a particular embodiment, the present invention relates to synthetic intermediates of formula (VI), geometrical isomers, enantiomers, diastereoisomers, pharmaceutically acceptable salts and all possible mixtures thereof,

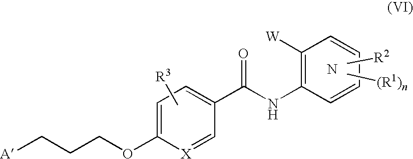

wherein
A′ is a leaving group; preferably A′ is chlorine;
W is hydroxy or a halogen atom;
X is either N or CH; in a specific embodiment X is CH;
R¹ is selected from the group comprising or consisting of sulfonyl, amino, substituted or unsubstituted C₁-C₆-alkyl, substituted or unsubstituted C₂-C₆-alkenyl, substituted or unsubstituted C₂-C₆-alkynyl, substituted or unsubstituted aryl, substituted or unsubstituted heteroaryl, substituted or unsubstituted C₃-C₈-cycloalkyl, substituted or unsubstituted 3-8-membered heterocycloalkyl, acyl, substituted or unsubstituted C₁-C₆-alkyl aryl, substituted or unsubstituted C₁-C₆-alkyl heteroaryl, substituted or unsubstituted C₂-C₆-alkenyl aryl, substituted or unsubstituted C₂-C₆-alkenyl heteroaryl, substituted or unsubstituted C₂-C₆-alkynyl aryl, substituted or unsubstituted C₂-C₆-alkynyl heteroaryl, substituted or unsubstituted C₁-C₆-alkyl cycloalkyl, substituted or unsubstituted C₁-C₆-alkyl heterocycloalkyl, substituted or unsubstituted C₂-C₆-alkenyl cycloalkyl, substituted or unsubstituted C₂-C₆-alkenyl heterocycloalkyl, substituted or unsubstituted C₂-C₆-alkynyl cycloalkyl, substituted or unsubstituted C₂-C₆-alkynyl heterocycloalkyl, alkoxycarbonyl, aminocarbonyl, substituted or unsubstituted C₁-C₆-alkyl carboxy, substituted or unsubstituted C₁-C₆-alkyl acyl, substituted or unsubstituted aryl acyl, substituted or unsubstituted heteroaryl acyl, substituted or unsubstituted C₃-C₈-(hetero)cycloalkyl acyl, substituted or unsubstituted C₁-C₆-alkyl acyloxy, substituted or unsubstituted C₁-C₆-alkyl alkoxy, substituted or unsubstituted C₁-C₆-alkyl alkoxycarbonyl, substituted or unsubstituted C₁-C₆-alkyl aminocarbonyl, substituted or unsubstituted C₁-C₆-alkyl acylamino, acylamino, acylaminocarbonyl, ureido, substituted or unsubstituted C₁-C₆-alkyl ureido, substituted or unsubstituted C₁-C₆-alkyl carbamate, substituted or unsubstituted C₁-C₆-alkyl amino, substituted or unsubstituted C₁-C₆-alkyl sulfonyloxy, substituted or unsubstituted C₁-C₆-alkyl sulfonyl, substituted or unsubstituted C₁-C₆-alkyl sulfinyl, substituted or unsubstituted C₁-C₆-alkyl sulfanyl, substituted or unsubstituted C₁-C₆-alkyl sulfonylamino, substituted or unsubstituted C₁-C₆-alkyl aminosulfonyl, hydroxy, halogen, cyano, carboxy, oxo, thioxo;
n is equal to 0, 1 or 2. In a specific embodiment, n is either 0 or 1;
R² is selected from the group comprising or consisting of hydrogen, sulfonyl, amino, substituted or unsubstituted C₁-C₆-alkyl, substituted or unsubstituted C₂-C₆-alkenyl, substituted or unsubstituted C₂-C₆-alkynyl, substituted or unsubstituted aryl, substituted or unsubstituted heteroaryl, substituted or unsubstituted C₃-C₈-cycloalkyl, substituted or unsubstituted 3-8-membered heterocycloalkyl, acyl, substituted or unsubstituted C₁-C₆-alkyl aryl, substituted or unsubstituted C₁-C₆-alkyl heteroaryl, substituted or unsubstituted C₂-C₆-alkenyl aryl, substituted or unsubstituted C₂-C₆-alkenyl heteroaryl, substituted or unsubstituted C₂-C₆-alkynyl aryl, substituted or unsubstituted C₂-C₆-alkynyl heteroaryl, substituted or unsubstituted C₁-C₆-alkyl cycloalkyl, substituted or unsubstituted C₁-C₆-alkyl heterocycloalkyl, substituted or unsubstituted C₂-C₆-alkenyl cycloalkyl, substituted or unsubstituted C₂-C₆-alkenyl heterocycloalkyl, substituted or unsubstituted C₂-C₆-alkynyl cycloalkyl, substituted or unsubstituted C₂-C₆-alkynyl heterocycloalkyl, alkoxycarbonyl, aminocarbonyl, substituted or unsubstituted C₁-C₆-alkyl carboxy, substituted or unsubstituted C₁-C₆-alkyl acyl, substituted or unsubstituted aryl acyl, substituted or unsubstituted heteroaryl acyl, substituted or unsubstituted C₃-C₈-(hetero)cycloalkyl acyl, substituted or unsubstituted C₁-C₆-alkyl acyloxy, substituted or unsubstituted C₁-C₆-alkyl alkoxy, substituted or unsubstituted C₁-C₆-alkyl alkoxycarbonyl, substituted or unsubstituted C₁-C₆-alkyl aminocarbonyl, substituted or unsubstituted C₁-C₆-alkyl acylamino, acylamino, acylaminocarbonyl, ureido, substituted or unsubstituted C₁-C₆-alkyl ureido, substituted or unsubstituted C₁-C₆-alkyl carbamate, substituted or unsubstituted C₁-C₆-alkyl amino, substituted or unsubstituted C₁-C₆-alkyl sulfonyloxy, substituted or unsubstituted C₁-C₆-alkyl sulfonyl, substituted or unsubstituted C₁-C₆-alkyl sulfinyl, substituted or unsubstituted C₁-C₆-alkyl sulfanyl, substituted or unsubstituted C₁-C₆-alkyl sulfonylamino, substituted or unsubstituted C₁-C₆-alkyl aminosulfonyl, hydroxy, halogen, cyano, carboxy, oxo, thioxo;
R³ is hydrogen or C₁-C₆-alkyl or halogen or C₁-C₆-alkoxy.

Specific synthetic intermediates are selected from the group consisting of:
2-{4-[(3-chloropropyl)oxy]phenyl}-4,5,6,7-tetrahydro[1,3]thiazolo[5,4-c]pyridine;
3-bromo-1-(trifluoroacetyl)piperidin-4-one;
2-[4-(3-chloropropoxy)phenyl]-5-(trifluoroacetyl)-4,5,6,7-tetrahydro[1,3]thiazolo[5,4-c]pyridine;
5-acetyl-2-[4-(3-chloropropoxy)phenyl]-4,5,6,7-tetrahydro[1,3]thiazolo[5,4-c]pyridine;
2-[4-(3-chloropropoxy)phenyl]-5-(methoxyacetyl)-4,5,6,7-tetrahydro[1,3]thiazolo[5,4-c]pyridine;
2-[4-(3-chloropropoxy)phenyl]-N-ethyl-6,7-dihydro[1,3]thiazolo[5,4-c]pyridine-5(4H)-carboxamide;
2-[4-(3-chloropropoxy)phenyl]-5-[(4-methylphenyl)sulfonyl]-4,5,6,6a-tetrahydro-3aH-pyrrolo[3,4-d][1,3]thiazol-3a-ol;
2-[4-(3-chloropropoxy)phenyl]-5-[(4-methylphenyl)sulfonyl]-5,6-dihydro-4H-pyrrolo[3,4-d][1,3]thiazole;
tert-butyl 4-bromo-5-oxoazepane-1-carboxylate;
tert-butyl 2-[4-(3-chloropropoxy)phenyl]-4,5,7,8-tetrahydro-6H-[1,3]thiazolo[4,5-d]azepine-6-carboxylate;
2-[4-(3-chloropropoxy)phenyl]-5,6,7,8-tetrahydro-4H-[1,3]thiazolo[4,5-d]azepine;
ethyl 2-[4-(3-chloropropoxy)phenyl]-5,6-dihydro-4H-cyclopenta[d][1,3]thiazole-4-carboxylate;
ethyl 2-[4-(3-chloropropoxy)phenyl]-4,5,6,7-tetrahydro-1,3-benzothiazole-4-carboxylate;
2-[4-(3-chloropropoxy)phenyl]-5,6-dihydro-4H-cyclopenta[d][1,3]thiazole-4-carboxylic acid;
benzyl {2-[4-(3-chloropropoxy)phenyl]-5,6-dihydro-4H-cyclopenta[d][1,3]thiazol-4-yl}carbamate;
2-[4-(3-chloropropoxy)phenyl]-4-(piperidin-1-ylcarbonyl)-5,6-dihydro-4H-cyclopenta[d][1,3]thiazole;
ethyl 2-[4-(3-chloropropoxy)phenyl]-4-methyl-5,6-dihydro-4H-cyclopenta[d][1,3]thiazole-4-carboxylate;
2-[4-(3-chloropropoxy)phenyl]-6,7-dihydro-1,3-benzothiazol-4(5H)-one;
2-[4-(3-chloropropoxy)phenyl]-4,5,6,7-tetrahydro-1,3-benzothiazol-4-ol;
2-methyl-1-{3-[4-(4,4,5,5-tetramethyl-1,3,2-dioxaborolan-2-yl)phenoxy]propyl}pyrrolidine;
2-{4-[3-(2-methylpyrrolidin-1-yl)propoxy]phenyl}-5,6-dihydro-1,3-benzothiazol-7(4H)-one;
2-{4-[3-(2-methylpyrrolidin-1-yl)propoxy]phenyl}-4,5,6,7-tetrahydro-1,3-benzothiazol-7-ol;
N-{2-[4-(3-chloropropoxy)phenyl]-4,5,6,7-tetrahydro-1,3-benzothiazol-6-yl}acetamide;
2-[4-(3-chloropropoxy)phenyl]-4,5,6,7-tetrahydro-1,3-benzothiazole-4-carboxylic acid;
benzyl {2-[4-(3-chloropropoxy)phenyl]-4,5,6,7-tetrahydro-1,3-benzothiazol-4-yl}carbamate;
2-{4-[3-(2-methylpyrrolidin-1-yl)propoxy]phenyl}-4,5,6,7-tetrahydro-1,3-benzothiazol-4-amine;
methyl 2-[4-(3-chloropropoxy)phenyl]-4,5,6,7-tetrahydro-1,3-benzothiazole-5-carboxylate, enantiomer 1;
2-[4-(3-chloropropoxy)phenyl]-4,5,6,7-tetrahydro-1,3-benzothiazole-5-carboxylic acid, enantiomer 1;
benzyl {2-[4-(3-chloropropoxy)phenyl]-4,5,6,7-tetrahydro-1,3-benzothiazol-5-yl}carbamate, enantiomer 1;
benzyl (2-{4-[3-(2-methylpyrrolidin-1-yl)propoxy]phenyl}-4,5,6,7-tetrahydro-1,3-benzothiazol-5-yl)carbamate;
2-{4-[3-(2-methylpyrrolidin-1-yl)propoxy]phenyl}-4,5,6,7-tetrahydro-1,3-benzothiazol-5-amine;
2-(trimethylsilyl)ethyl {2-[4-(3-chloropropoxy)phenyl]-4,5,6,7-tetrahydro-1,3-benzothiazol-4-yl}carbamate;
4-[3-(2-methylpyrrolidin-1-yl)propoxy]benzenecarbothioamide;
4-(3-chloropropoxy)-N-(4-chloropyridin-3-yl)benzamide;
2-[4-(3-chloropropoxy)phenyl][1,3]thiazolo[4,5-c]pyridine;
2-[4-(3-chloropropoxy)phenyl]-5-methyl[1,3]thiazolo[4,5-c]pyridin-5-ium;
2-[4-(3-chloropropoxy)phenyl]-5-methyl-4,5,6,7-tetrahydro[1,3]thiazolo[4,5-c]pyridine;
2-[4-(3-chloropropoxy)phenyl]-4,5,6,7-tetrahydro[1,3]thiazolo[4,5-c]pyridine;
5-acetyl-2-[4-(3-chloropropoxy)phenyl]-4,5,6,7-tetrahydro[1,3]thiazolo[4,5-c]pyridine;
2-(4-{3-[(2R)-2-methylpyrrolidin-1-yl]propoxy}phenyl)-4,5,6,7-tetrahydro[1,3]thiazolo[4,5-c]pyridine;
4-(benzyloxy)-N-(4-hydroxypyridin-3-yl)benzamide;
2-[4-(benzyloxy)phenyl][1,3]oxazolo[4,5-c]pyridine;
2-[4-(benzyloxy)phenyl]-5-methyl[1,3]oxazolo[4,5-c]pyridin-5-ium;
2-[4-(benzyloxy)phenyl]-5-methyl-4,5,6,7-tetrahydro[1,3]oxazolo[4,5-c]pyridine;
4-(5-methyl-4,5,6,7-tetrahydro[1,3]oxazolo[4,5-c]pyridin-2-yl)phenol;
2-[4-(3-chloropropoxy)phenyl]-5-methyl-4,5,6,7-tetrahydro[1,3]oxazolo[4,5-c]pyridine;
tert-butyl 2-[4-(3-chloropropoxy)phenyl]-7a-hydroxy-3a,6,7,7a-tetrahydro[1,3]thiazolo[5,4-c]pyridine-5(4H)-carboxylate;
tert-butyl 2-[4-(3-chloropropoxy)phenyl]-6,7-dihydro[1,3]thiazolo[5,4-c]pyridine-5(4H)-carboxylate;
tert-butyl 2-(4-{3-[(2R)-2-methylpyrrolidin-1-yl]propoxy}phenyl)-6,7-dihydro[1,3]thiazolo[5,4-c]pyridine-5(4H)-carboxylate;
2-(4-{3-[(2R)-2-methylpyrrolidin-1-yl]propoxy}phenyl)-4,5,6,7-tetrahydro[1,3]thiazolo[5,4-c]pyridine;
3-methyl-1-(morpholin-4-ylsulfonyl)-1H-imidazol-3-ium trifluoromethanesulfonate;
2-[4-(3-chloropropoxy)phenyl]-5-(morpholin-4-ylsulfonyl)-4,5,6,7-tetrahydro[1,3]thiazolo[5,4-c]pyridine;
methyl 3-[2-(4-{3-[(2R)-2-methylpyrrolidin-1-yl]propoxy}phenyl)-6,7-dihydro[1,3]thiazolo[5,4-c]pyridin-5(4H)-yl]-3-oxopropanoate;
benzyl (2S)-1-{3-[4-(5-acetyl-4,5,6,7-tetrahydro[1,3]thiazolo[5,4-c]pyridin-2-yl)phenoxy]propyl}pyrrolidine-2-carboxylate;
3-bromo-6,6-dimethylazepane-2,4-dione;
2-[4-(3-chloropropoxy)phenyl]-7,7-dimethyl-5,6,7,8-tetrahydro-4H-[1,3]thiazolo[5,4-c]azepin-4-one;
2-[4-(3-chloropropoxy)phenyl]-5,7,7-trimethyl-5,6,7,8-tetrahydro-4H-[1,3]thiazolo[5,4-c]azepin-4-one;
4-(benzyloxy)-N-(2-oxoazepan-3-yl)benzamide;
2-[4-(benzyloxy)phenyl]-5,6,7,8-tetrahydro-4H-[1,3]thiazolo[5,4-b]azepine hydrochloride;
4-acetyl-2-[4-(benzyloxy)phenyl]-5,6,7,8-tetrahydro-4H-[1,3]thiazolo[5,4-b]azepine;
4-(4-acetyl-5,6,7,8-tetrahydro-4H-[1,3]thiazolo[5,4-b]azepin-2-yl)phenol;
4-acetyl-2-[4-(3-chloropropoxy)phenyl]-5,6,7,8-tetrahydro-4H-[1,3]thiazolo[5,4-b]azepine;
methyl 3-bromo-4-hydroxycyclopentanecarboxylate;
methyl 3-bromo-4-oxocyclopentanecarboxylate;
methyl 2-[4-(3-chloropropoxy)phenyl]-5,6-dihydro-4H-cyclopenta[d][1,3]thiazole-5-carboxylate;
2-[4-(3-chloropropoxy)phenyl]-5,6-dihydro-4H-cyclopenta[d][1,3]thiazole-5-carboxylic acid;
2-[4-(3-chloropropoxy)phenyl]-5-(morpholin-4-ylcarbonyl)-5,6-dihydro-4H-cyclopenta[d][1,3]thiazole;
2-[4-(3-chloropropoxy)phenyl]-5-[(4,4-difluoropiperidin-1-yl)carbonyl]-5,6-dihydro-4H-cyclopenta[d][1,3]thiazole;
2-[4-(3-chloropropoxy)phenyl]-N-ethyl-5,6-dihydro-4H-cyclopenta[d][1,3]thiazole-5-carboxamide;
2-[4-(3-chloropropoxy)phenyl]-5-(pyrrolidin-1-ylcarbonyl)-5,6-dihydro-4H-cyclopenta[d][1,3]thiazole;
benzyl {2-[4-(3-chloropropoxy)phenyl]-5,6-dihydro-4H-cyclopenta[d][1,3]thiazol-5-yl}carbamate;
benzyl (2-{4-[3-(2-methylpyrrolidin-1-yl)propoxy]phenyl}-5,6-dihydro-4H-cyclopenta[d][1,3]thiazol-5-yl)carbamate; and
5-acetyl-2-[4-(3-chloropropoxy)phenyl]-5,6-dihydro-4H-pyrrolo[3,4-d][1,3]thiazole.

The following examples illustrate how the compounds covered by formula (I) may be synthesized. They are provided for illustrative purposes only and are not intended, nor should they be construed, as limiting the invention in any manner. Those skilled in the art will appreciate that routine variations and modifications of the following examples can be made without exceeding the spirit or scope of the invention.

Unless specified otherwise in the examples, characterization of the compounds is performed according to the following methods:

NMR spectra are recorded on a BRUKER AC 250 Fourier Transform NMR Spectrometer fitted with an Aspect 3000 computer and a 5 mm ¹H/¹³C dual probehead or BRUKER DRX 400 FT NMR fitted with a SG Indigo² computer and a 5 mm inverse geometry ¹H/¹³C/¹⁵N triple probehead. The compound is studied in dimethylsulfoxide-d₆ (DMSO-d₆) or chloroform-d (CDCl₃) solution at a probe temperature of 313 K or 300 K and at a concentration of 20 mg/ml. The instrument is locked respectively on the deuterium signal of dimethylsulfoxide-d₆ (DMSO-d₆) or chloroform-d (CDCl₃). Chemical shifts are given in ppm downfield from TMS taken as internal standard.

HPLC analyses are performed using one of the following systems:
an Agilent 1100 series HPLC system mounted with an INERTSIL ODS 3 C18, DP 5 μm, 250×4.6 mm column. The gradient runs from 100% solvent A (acetonitrile, water, phosphoric acid (5/95/0.001, v/v/v)) to 100% solvent B (acetonitrile, water, phosphoric acid (95/5/0.001, v/v/v)) in 6 min with a hold at 100% B of 4 min. The flow rate is set at 2.5 ml/min. The chromatography is carried out at 35° C.
a HP 1090 series HPLC system mounted with a HPLC Waters Symetry C18, 250×4.6 mm column. The gradient runs from 100% solvent A (methanol, water, phosphoric acid (15/85/0.001M, v/v/M)) to 100% solvent B (methanol, water, phosphoric acid (85/15/0.001 M, v/v/M)) in 10 min with a hold at 100% B of 10 min. The flow rate is set at 1 ml/min. The chromatography is carried out at 40° C.

Mass spectrometric measurements in LC/MS mode are performed as follows:

### HPLC Conditions

Analyses are performed using a WATERS Alliance HPLC system mounted with an INERTSIL ODS 3, DP 5 μm, 250×4.6 mm column.

The gradient runs from 100% solvent A (acetonitrile, water, trifluoroacetic acid (10/90/0.1, v/v/v)) to 100% solvent B (acetonitrile, water, trifluoroacetic acid (90/10/0.1, v/v/v)) in 7 min with a hold at 100% B of 4 min. The flow rate is set at 2.5 ml/min and a split of 1/25 is used just before API source.

### MS Conditions

Samples are dissolved in acetonitrile/water, 70/30, v/v at the concentration of about 250 μg/ml. API spectra (+ or −) are performed using a FINNIGAN LCQ ion trap mass spectrometer. APCI source operated at 450° C. and the capillary heater at 160° C. ESI source operated at 3.5 kV and the capillary heater at 210° C.

Mass spectrometric measurements in DIP/EI mode are performed as follows: samples are vaporized by heating the probe from 50° C. to 250° C. in 5 min. EI (Electron Impact) spectra are recorded using a FINNIGAN TSQ 700 tandem quadrupole mass spectrometer. The source temperature is set at 150° C.

Mass spectrometric measurements on a TSQ 700 tandem quadrupole mass spectrometer (Finnigan MAT) in GC/MS mode are performed with a gas chromatograph model 3400 (Varian) fitted with a split/splitless injector and a DB-5MS fused-silica column (15 m×0.25 mm I.D., 1 μm) from J&W Scientific. Helium (purity 99.999%) is used as carrier gas. The injector (CTC A200S autosampler) and the transfer line operate at 290 and 250° C., respectively. Sample (1 μl) is injected in splitless mode and the oven temperature is programmed as follows: 50° C. for 5 min., increasing to 280° C. (23° C./min) and holding for 10 min. The TSQ 700 spectrometer operates in electron impact (EI) or chemical ionization (Cl/CH₄) mode (mass range 33-800, scan time 1.00 sec). The source temperature is set at 150° C.

Specific rotation is recorded on a Perkin-Elmer 341 polarimeter. The angle of rotation is recorded at 25° C. on 1% solutions in methanol, at 589 nm. For some molecules, the solvent is dichloromethane or dimethylsulfoxide, due to solubility problems.

Melting points are determined on a Büch±535 or 545 Tottoli-type fusionometre, and are not corrected, or by the onset temperature on a Perkin Elmer DSC 7.

Preparative chromatographic separations are performed on silicagel 60 Merck, particle size 15-40 μm, reference 1.15111.9025, using Novasep axial compression columns (80 mm i.d.), flow rates between 70 and 150 ml/min. Amount of silicagel and solvent mixtures as described in individual procedures.

Preparative Chiral Chromatographic separations are performed on a DAICEL Chiralpak AD 20 μm, 100*500 mm column using an in-house build instrument with various mixtures of lower alcohols and C5 to C8 linear, branched or cyclic alkanes at ±350 ml/min. Solvent mixtures as described in individual procedures.

Experiments requiring microwave irradiation were performed either on a CEM Discover apparatus (CEM corporation) or on a Biotage Initiator (Biotage AB) microwave oven using the flasks and stirrers sold by these companies.

### EXAMPLES

### Example 1

### Synthesis of 2-{4-[3-(2-methylpyrrolidin-1-yl)propoxy]phenyl}-4,5,6,7-tetrahydro[1,3]thiazolo[5,4-c]pyridine 2 and 5-ethyl-2-{4-[3-(2-methylpyrrolidin-1-yl)propoxy]phenyl}-4,5,6,7-tetrahydro[1,3]thiazolo[5,4-c]pyridine 3

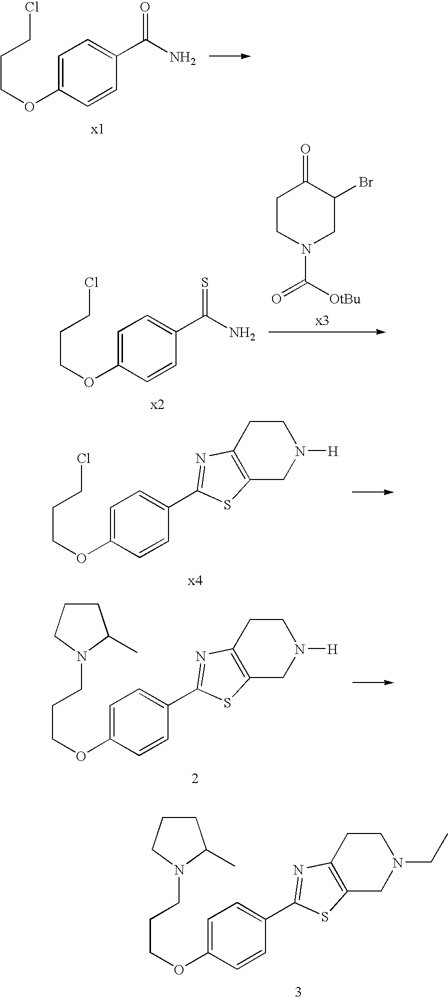

### 1.1 Synthesis of 4-(3-chloropropoxy)benzenecarbothioamide x2

4-(3-Chloropropoxy)benzamide x1 (1.0 g, 4.6 mmol, 1 eq) is dissolved in a 1:1 mixture of chloroform and toluene, and 2,4-bis-(4-methoxyphenyl)-1,3-dithia-2,4-diphosphetane-2,4-disulfide (1.5 g, 3.7 mmol, 0.8 eq) is added. The reaction is stirred overnight at 85° C. Water is then added and the phases are separated. The organic layer is dried over magnesium sulfate and concentrated under reduced pressure. The crude material is purified by chromatography over silicagel (dichloromethane/methanol/ammonia 98:1.8:0.2) to afford 0.91 g of 4-(3-chloropropoxy)benzenecarbothioamide x2 as a white solid.

Yield: 85%.

LC-MS (MH⁺): 230/232.

### 1.2 Synthesis of 2-{4-[(3-chloropropyl)oxy]phenyl}-4,5,6,7-tetrahydro[1,3]thiazolo[5,4-c]pyridine x4

A suspension of 4-(3-chloropropoxy)benzenecarbothioamide x2 (11.0 g, 47.88 mmol, 1 eq) and tert-butyl 3-bromo-4-oxopiperidine-1-carboxylate x3 (14.64 g, 52.67 mmol, 1.1 eq) in isopropanol (100 ml) is heated at 80° C. during 20 hours. After cooling and concentration, the mixture is taken up with dioxane (100 ml), a 1 M aqueous solution of hydrochloric acid (20 ml) is added and the mixture is stirred during 3 hours at room temperature. The dioxane is removed under vacuum and the resulting material is brought to pH 8 by a 1 M solution of sodium hydroxide. The product is extracted with a mixture of ethyl acetate and ethanol (90/10, 2×400 ml), the organic solution is dried over magnesium sulfate and concentrated in vacuum. The resulting solid is taken up with acetone (100 ml) and filtered to afford 6.4 g of 2-{4-[(3-chloropropyl)oxy]phenyl}-4,5,6,7-tetrahydro[1,3]thiazolo[5,4-c]pyridine x4 as a white solid.

Yield: 43%.

LC-MS (MH⁺): 309/311.

### 1.3 Synthesis of 2-{4-[3-(2-methylpyrrolidin-1-yl)propoxy]phenyl}-4,5,6,7-tetrahydro[1,3]thiazolo[5,4-c]pyridine 2

A mixture of 2-{4-[(3-chloropropyl)oxy]phenyl}-4,5,6,7-tetrahydro[1,3]thiazolo[5,4-c]pyridine x4 (7.0 g, 22.67 mmol, 1 eq), 2-methylpyrrolidine (4.25 g, 49.87 mmol, 2.2 eq) potassium carbonate (7.83 g, 56.67 mmol, 2.5 eq) and sodium iodide (0.34 g, 2.27 mmol, 0.1 eq) is stirred at 90° C. in acetonitrile (200 ml) for 60 hours. The mixture is poured in water (200 ml) and extracted with dichloromethane/methanol (90/10, 2×200 ml). After concentration, the crude material is purified by chromatography over silicagel (gradient: dichloromethane/methanol/ammonia 95:5:0.5 to 90:9:1) to provide 4.8 g of pure 2-{4-[3-(2-methylpyrrolidin-1-yl)propoxy]phenyl}-4,5,6,7-tetrahydro[1,3]thiazolo[5,4-c]pyridine 2.

Yield: 59%.

LC-MS (MH⁺): 358.

Compound 1 may be prepared according to the same method.

### 1.4 Synthesis of 5-ethyl-2-{4-[3-(2-methylpyrrolidin-1-yl)propoxy]phenyl}-4,5,6,7-tetrahydro[1,3]thiazolo[5,4-c]pyridine 3

A suspension of 2-{4-[3-(2-methylpyrrolidin-1-yl)propoxy]phenyl}-4,5,6,7-tetrahydro[1,3]thiazolo[5,4-c]pyridine 2 (0.14 g, 0.39 mmol, 1 eq) and molecular sieves (powder, 0.4 g in methanol/acetic acid 1% (10 ml)) is treated with acetaldehyde (0.49 ml, 0.43 mmol, 1.1 eq) and the slurry is stirred for 1 hour at room temperature. Sodium cyanoborohydride (0.03 g, 0.43 mmol, 1.1 eq) is added and the mixture is stirred overnight. Dichloromethane (50 ml) and water (50 ml) are added. The aqueous layer is brought to pH 10 with saturated sodium bicarbonate. The organic layer is dried over magnesium sulfate, concentrated under reduced pressure and purified over silicagel (dichloromethane/methanol/ammonia 96:4:0.4) to yield 17 mg of 5-ethyl-2-{4-[3-(2-methylpyrrolidin-1-yl)propoxy]phenyl}-4,5,6,7-tetrahydro[1,3]thiazolo[5,4-c]pyridine 3.

Yield: 11%.

LC-MS (MH⁺): 386.

Compounds 7 and 8 may be synthesized according to the same method.

### Example 2

### Synthesis of 2-{4-[3-(2-methylpyrrolidin-1-yl)propoxy]phenyl}-5-(trifluoroacetyl)-4,5,6,7-tetrahydro[1,3]thiazolo[5,4-c]pyridine 9

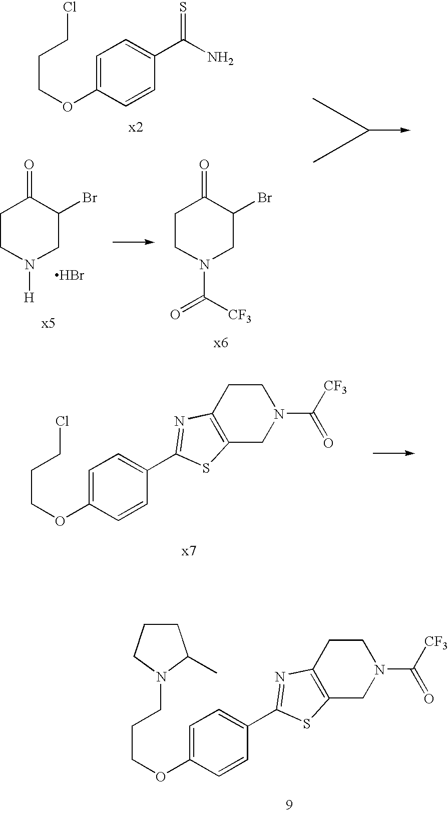

### 2.1 Synthesis of 3-bromo-1-(trifluoroacetyl)piperidin-4-one x6

A suspension of 3-bromopiperidin-4-one hydrobromide x5 (7.3 g, 36 mmol, 1 eq) in dichloromethane (200 ml) is treated dropwise with trifluoroacetic anhydride (25 ml, 170 mmol, 5 eq) and the resulting mixture is stirred for 2 hours at 20° C. and 1 hour at reflux. After cooling back to 20° C., water is added carefully. The organic layer is decanted, washed with a saturated solution of sodium bicarbonate and dried over magnesium sulfate. After concentration, 6.3 g of crude material is obtained. Purification by chromatography over silicagel (dichloromethane/hexane 80:20) affords 2.44 g of pure 3-bromo-1-(trifluoroacetyl)piperidin-4-one x6.

Yield: 24%.

GC-MS (M⁺.): 273/275.

### 2.2 Synthesis of 2-[4-(3-chloropropoxy)phenyl]-5-(trifluoroacetyl)-4,5,6,7-tetrahydro[1,3]thiazolo[5,4-c]pyridine x7

A suspension of 4-(3-chloropropoxy)benzenecarbothioamide x2 (2 g, 9 mmol, 1 eq) and 3-bromo-1-(trifluoroacetyl)piperidin-4-one x6 (2.7 g, 9.9 mmol, 1.1 eq) in isopropanol (20 ml) is heated at 80° C. during 20 hours. After cooling and concentration, the mixture is taken up with dioxane (20 ml), a 1 M aqueous solution of hydrochloric acid (10 ml) is added and the mixture is stirred during 3 hours at room temperature. The dioxane is removed under vacuum and the resulting material is brought to pH 8 by a 1M solution of sodium hydroxide. The product is extracted with a mixture of ethyl acetate and ethanol (90/10, 2×100 ml) and the organic solution is dried over magnesium sulfate and concentrated in vacuum. The resulting solid is taken up with acetone (100 ml) and filtered to afford 2-[4-(3-chloropropoxy)phenyl]-5-(trifluoroacetyl)-4,5,6,7-tetrahydro[1,3]thiazolo[5,4-c]pyridine x7 as a white solid.

Yield: 50%.

LC-MS (MH⁺): 405/407.

### 2.3 Synthesis of 2-{4-[3-(2-methylpyrrolidin-1-yl)propoxy]phenyl}-5-(trifluoroacetyl)-4,5,6,7-tetrahydro[1,3]thiazolo[5,4-c]pyridine 9

A mixture of 2-[4-(3-chloropropoxy)phenyl]-5-(trifluoroacetyl)-4,5,6,7-tetrahydro[1,3]thiazolo[5,4-c]pyridine x7 (1.7 g, 4.2 mmol, 1 eq), 2-methylpyrrolidine (0.43 g, 5.04 mmol, 1.2 eq), potassium carbonate (2.32 g, 16.8 mmol, 4 eq) and sodium iodide (0.06 g, 0.4 mmol, 0.1 eq) is stirred at 90° C. in acetonitrile (10 ml) for 24 hours. The mixture is concentrated and taken up with dichloromethane. The organic layer is washed with water, dried over magnesium sulfate and concentrated under reduced pressure. Purification over silicagel (dichloromethane/methanol/ammonia 96:4:0.4) affords 0.33 g of 2-{4-[3-(2-methylpyrrolidin-1-yl)propoxy]phenyl}-5-(trifluoroacetyl)-4,5,6,7-tetrahydro[1,3]thiazolo[5,4-c]pyridine 9.

Yield: 16%.

LC-MS (MH⁺): 454.

### Example 3

### Synthesis of 5-benzoyl-2-{4-[3-(2-methylpyrrolidin-1-yl)propoxy]phenyl}-4,5,6,7-tetrahydro[1,3]thiazolo[5,4-c]pyridine 11

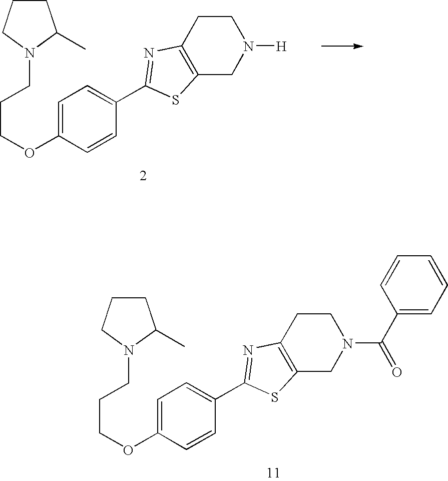

A solution of 2-{4-[3-(2-methylpyrrolidin-1-yl)propoxy]phenyl}-4,5,6,7-tetrahydro[1,3]thiazolo[5,4-c]pyridine 2 (0.16 g, 0.45 mmol, 1 eq) and triethylamine (0.73 ml, 0.9 mmol, 2 eq) in dichloromethane (5 ml) is treated with a solution of benzoyl chloride (70 μl, 0.49 mmol, 1.1 eq) in dichloromethane (1 ml). The mixture is stirred 1 hour at room temperature. Water (5 ml) is then added, the organic layer is collected, dried over magnesium sulfate and concentrated under reduced pressure. The resulting material is purified by LC-MS chromatography (acetonitrile/water/ammonium bicarbonate) to yield 72 mg of pure 5-benzoyl-2-{4-[3-(2-methylpyrrolidin-1-yl)propoxy]phenyl}-4,5,6,7-tetrahydro[1,3]thiazolo[5,4-c]pyridine 11.

Yield: 34%.

LC-MS (MH⁺): 462.

Compounds 12, 13, 14, 15, 16, 20, 21, 26, 36, 37, 79, 84, 99 and 102 may be synthesized according to the same method.

### Example 4

### Synthesis of 5-acetyl-2-{4-[3-(2-methylpyrrolidin-1-yl)propoxy]phenyl}-4,5,6,7-tetrahydro[1,3]thiazolo[5,4-c]pyridine 4

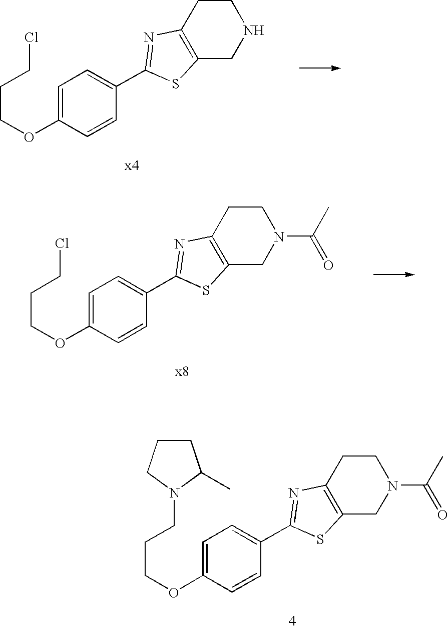

### 4.1 Synthesis of 5-acetyl-2-[4-(3-chloropropoxy)phenyl]-4,5,6,7-tetrahydro[1,3]thiazolo[5,4-c]pyridine x8

A solution of 2-{4-[(3-chloropropyl)oxy]phenyl}-4,5,6,7-tetrahydro[1,3]thiazolo[5,4-c]pyridine x4 (2.5 g, 8.1 mmol, 1 eq) and triethylamine (3.4 ml, 24.3 mmol, 3 eq) in dichloromethane (100 ml) is treated dropwise with a solution of acetyl chloride (0.64 ml, 8.9 mmol, 1.1 eq) in dichloromethane (2 ml). After 2 hours at room temperature, water (50 ml) is added. The layers are separated and the organic layer is washed with saturated ammonium chloride, dried over magnesium sulfate and concentrated under reduced pressure to afford 2.8 g of crude 5-acetyl-2-[4-(3-chloropropoxy)phenyl]-4,5,6,7-tetrahydro[1,3]thiazolo[5,4-c]pyridine x8. This compound is sufficiently pure to be engaged in the following reaction.

Yield: 100%.

LC-MS (MH⁺): 351/353.

2-[4-(3-chloropropoxy)phenyl]-5-(methoxyacetyl)-4,5,6,7-tetrahydro[1,3]thiazolo[5,4-c]pyridine x9 is obtained according to the same method.

Yield: 100%.

LC-MS (MH⁺): 381/383.

### 4.2 Synthesis of 5-acetyl-2-{4-[3-(2-methylpyrrolidin-1-yl)propoxy]phenyl}-4,5,6,7-tetrahydro[1,3]thiazolo[5,4-c]pyridine 4

A mixture of 5-acetyl-2-[4-(3-chloropropoxy)phenyl]-4,5,6,7-tetrahydro[1,3]thiazolo[5,4-c]pyridine x8 (1.6 g, 4.2 mmol, 1 eq), 2-methylpyrrolidine (0.43 g, 5.04 mmol, 1.2 eq), potassium carbonate (2.32 g, 16.8 mmol, 4 eq) and sodium iodide (0.06 g, 0.4 mmol, 0.1 eq) is stirred at 90° C. in acetonitrile (10 ml) for 24 hours. The mixture is concentrated and taken up with dichloromethane. The organic layer is washed with water, dried over magnesium sulfate and concentrated under reduced pressure. Purification over silicagel (dichloromethane/methanol/ammonia 96:4:0.4) affords 5-acetyl-2-{4-[3-(2-methylpyrrolidin-1-yl)propoxy]phenyl}-4,5,6,7-tetrahydro[1,3]thiazolo[5,4-c]pyridine 4.

Yield: 52%.

LC-MS (MH⁺): 400.

Compounds 5, 6, 22, 23 and 24 may be synthesized according to the same method.

### Example 5

### Synthesis of 5-acetyl-2-{4-[3-(2-methylpiperidin-1-yl)propoxy]phenyl}-4,5,6,7-tetrahydro[1,3]thiazolo[5,4-c]pyridine 42

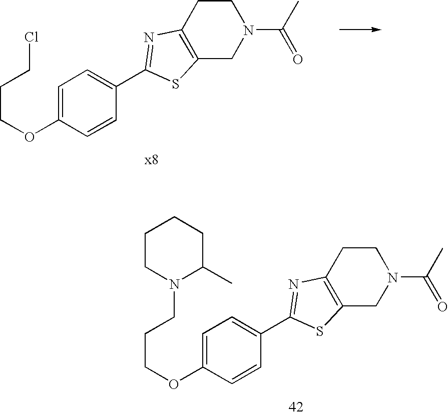

A solution of 5-acetyl-2-[4-(3-chloropropoxy)phenyl]-4,5,6,7-tetrahydro[1,3]thiazolo[5,4-c]pyridine x8 (0.27 g, 0.77 mmole, 1 eq) and 2-methylpiperidine (0.3 ml, 2.94 mmol, 3.4 eq) in acetonitrile (12 ml) is stirred under microwave irradiation for 5 hours at 120° C. The mixture is concentrated under reduced pressure. A 1 M aqueous solution of hydrochloric acid (20 ml) is added and the mixture is extracted with ethyl acetate (2×15 ml). Sodium hydroxide (pellets) is then added to the aqueous layer to reach pH 13. The aqueous layer is then extracted with dichloromethane (3×20 ml). The combined dichloromethane layers are washed with brine, dried over magnesium sulfate and concentrated under reduced pressure. The crude material is purified by chromatography over silicagel (dichloromethane/methanol/ammonia 97:3:0.3) to provide 0.113 g of 5-acetyl-2-{4-[3-(2-methylpiperidin-1-yl)propoxy]phenyl}-4,5,6,7-tetrahydro[1,3]thiazolo[5,4-c]pyridine 42 as a yellow solid.

Yield: 36%.

LC-MS (MH⁺): 414.

Compounds 39, 40, 41, 43, 44, 81 and 83 may be synthesized according to the same method.

### Example 6

### Synthesis of N-ethyl-2-{4-[3-(2-methylpyrrolidin-1-yl)propoxy]phenyl}-6,7-dihydro[1,3]thiazolo[5,4-c]pyridine-5(4H)-carboxamide 17

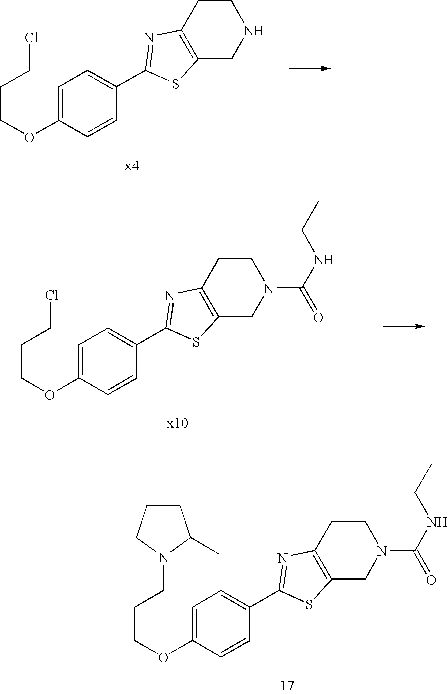

### 6.1 Synthesis of 2-[4-(3-chloropropoxy)phenyl]-N-ethyl-6,7-dihydro[1,3]thiazolo[5,4-c]pyridine-5(4H)-carboxamide x10

A solution of 2-{4-[(3-chloropropyl)oxy]phenyl}-4,5,6,7-tetrahydro[1,3]thiazolo[5,4-c]pyridine x4 (2.0 g, 6.5 mmol, 1 eq) in dichloromethane (100 ml) is treated with a solution of ethylisocyanate (0.6 ml, 7.8 mmol, 1.2 eq) in dichloromethane (50 ml). The mixture is stirred at 20° C. overnight. After concentration under reduced pressure, the solid is triturated in acetonitrile (50 ml) and filtered. This solid is pure enough to be engaged in following reaction.

Yield: 100%.

LC-MS (MH⁺): 380/382.

### 6.2 Synthesis of N-ethyl-2-{4-[3-(2-methylpyrrolidin-1-yl)propoxy]phenyl}-6,7-dihydro[1,3]thiazolo[5,4-c]pyridine-5(4H)-carboxamide 17

A suspension of 2-[4-(3-chloropropoxy)phenyl]-N-ethyl-6,7-dihydro[1,3]thiazolo[5,4-c]pyridine-5(4H)-carboxamide x10 (0.85 g, 2.23 mmol, 1 eq), 2-methylpyrrolidine (0.26 g, 3.12 mmol, 1.4 eq), potassium carbonate (0.68 g, 4.9 mmol, 2.2 eq) and sodium iodide (30 mg, 0.22 mmol, 0.1 eq) is stirred at 90° C. in acetonitrile (10 ml) for 20 hours. The mixture is concentrated and ethyl acetate and water are added to the residue. The organic layer is dried over magnesium sulfate and concentrated under reduced pressure. Purification by chromatography over silicagel (dichloromethane/methanol/ammonia 95:5:0.5) affords 450 mg of N-ethyl-2-{4-[3-(2-methylpyrrolidin-1-yl)propoxy]phenyl}-6,7-dihydro[1,3]thiazolo[5,4-c]pyridine-5(4H)-carboxamide 17.

Yield: 48%.

LC-MS (MH⁺): 429.

Compounds 18 and 19 are prepared according to the same method.

Compound 25 is isolated as a secondary product during the synthesis of compound 19.

### Example 7

### Synthesis of 2-{4-[3-(2-methylpyrrolidin-1-yl)propoxy]phenyl}-N-(2-thien-2-ylethyl)-6,7-dihydro[1,3]thiazolo[5,4-c]pyridine-5(4H)-carboxamide 29

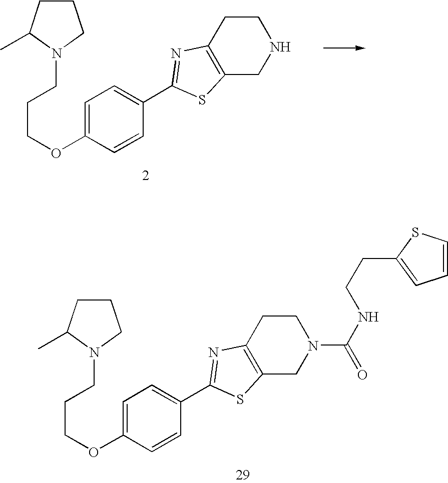

A solution of 2-{4-[3-(2-methylpyrrolidin-1-yl)propoxy]phenyl}-4,5,6,7-tetrahydro[1,3]thiazolo[5,4-c]pyridine 2 (0.3 g, 0.84 mmol, 1 eq) in dichloromethane (15 ml) is treated with 2-(2-thienyl)ethyl isocyanate (0.14 g, 0.92 mmol, 1.1 eq) and the mixture is stirred ten minutes at room temperature. Water (10 ml) is added, the organic layer is separated, dried over magnesium sulfate and concentrated under reduced pressure. Purification by LC-MS chromatography (gradient, dichloromethane/methanol/ammonia 99:0.9:0.1 to 90:9:1) affords 237 mg of pure 2-{4-[3-(2-methylpyrrolidin-1-yl)propoxy]phenyl}-N-(2-thien-2-ylethyl)-6,7-dihydro[1,3]thiazolo[5,4-c]pyridine-5(4H)-carboxamide 29.

Yield: 55%.

LC-MS (MH⁺): 511.

Compounds 27, 28, 30, 31, 32, 33, 34, 35 and 38 may be prepared according to the same method.

### Example 8

### Synthesis of 5-(4-fluorophenyl)-2-{4-[3-(2-methylpyrrolidin-1-yl)propoxy]phenyl}-4,5,6,7-tetrahydro[1,3]thiazolo[5,4-c]pyridine 10

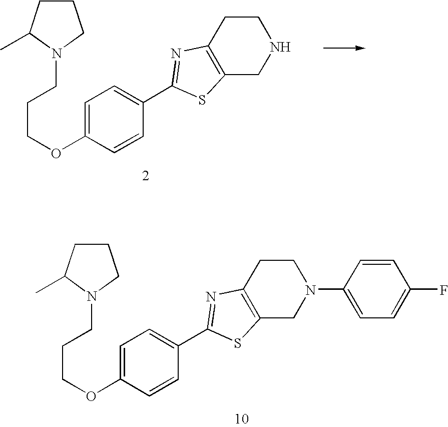

A suspension of 2-{4-[3-(2-methylpyrrolidin-1-yl)propoxy]phenyl}-4,5,6,7-tetrahydro[1,3]thiazolo[5,4-c]pyridine 2 (0.4 g, 1.12 mmol, 1.2 eq), 4-bromofluorobenzene (100 μl, 0.93 mmol, 1 eq), bis(dibenzylideneacetone)palladium (0.4 mg, 0.01 mmol, 0.01 eq), 2-(dicyclohexylphosphino)biphenyl (0.3 mg, 0.01 mmol, 0.01 eq) and sodium tert-butylate (0.13 g, 1.31 mmol, 1.4 eq) in dry toluene (2 ml) is stirred overnight at 120° C. The mixture is diluted with toluene (20 ml) and washed with water (20 ml). The organic phase is dried over magnesium sulfate, concentrated, and the crude material is purified by chromatography over silicagel (dichloromethane/methanol/ammonia 97:3:0.3) to yield 30 mg of pure 5-(4-fluorophenyl)-2-{4-[3-(2-methylpyrrolidin-1-yl)propoxy]phenyl}-4,5,6,7-tetrahydro[1,3]thiazolo[5,4-c]pyridine 10.

Yield: 6%.

LC-MS (MH⁺): 452.

### Example 9

### Synthesis of 5-[(4-methylphenyl)sulfonyl]-2-{4-[3-(2-methylpyrrolidin-1-yl)propoxy]phenyl}-5,6-dihydro-4H-pyrrolo[3,4-d][1,3]thiazole 45

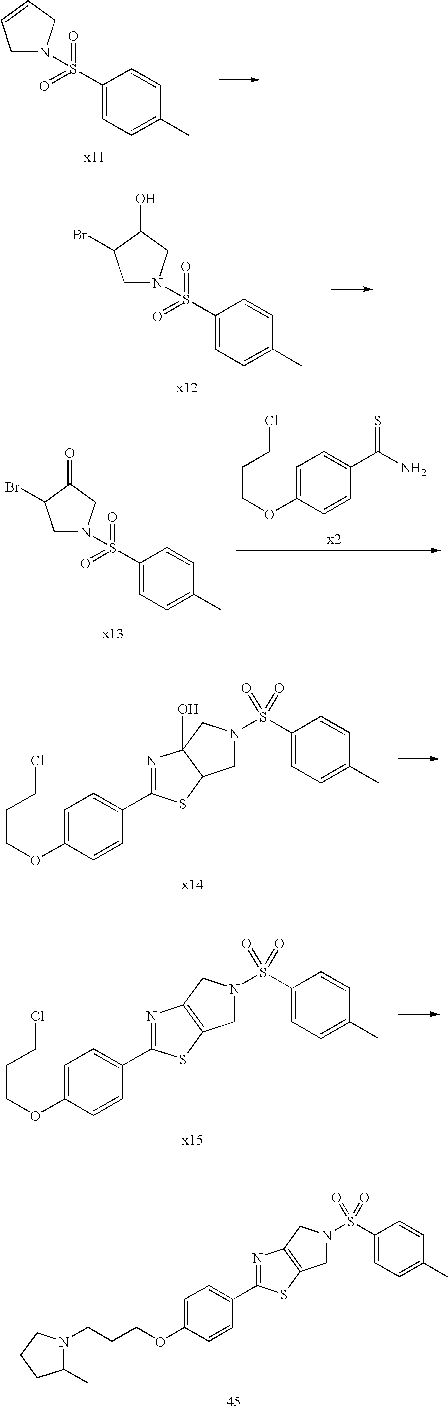

### 9.1 Synthesis of 4-bromo-1-[(4-methylphenyl)sulfonyl]pyrrolidin-3-ol x12

N-bromosuccinimide (22.3 g, 125.6 mmol, 2 eq) is added by portions to a solution of 1-[(4-methylphenyl)sulfonyl]-2,5-dihydro-1H-pyrrole x11 (14 g, 62.8 mmol, 1 eq) in dimethylsulfoxide (150 ml). After 2 hours, the orange mixture is poured into water (500 ml) and extracted with ethyl acetate (2×200 ml). The organic layer is washed with brine, dried over magnesium sulfate and concentrated. After purification over silicagel (dichloromethane/benzine 1:1 then dichloromethane/methanol 9:1), 9.2 g of 4-bromo-1-[(4-methylphenyl)sulfonyl]pyrrolidin-3-ol x12 are obtained together with 6.6 g of 3,4-dibromo-1-[(4-methylphenyl)sulfonyl]-pyrrolidine.

Yield: 45%.

LC-MS (MH⁺): 320/322.

### 9.2 Synthesis of 4-bromo-1-[(4-methylphenyl)sulfonyl]pyrrolidin-3-one x13

A solution of 4-bromo-1-[(4-methylphenyl)sulfonyl]pyrrolidin-3-ol x12 (8 g, 25 mmol, 1 eq) in dichloromethane (400 ml) is treated dropwise with a 15% solution of 1,1,1-triacetoxy-1,1-dihydro-1,2-benziodoxol-3(1H)-one (Dess-Martin periodinane) in dichloromethane (130 ml, 39 mmol, 1.5 eq). The solution is stirred overnight at room temperature. A saturated solution of sodium bisulfite (100 ml) is added and the heterogeneous mixture is stirred for 1 hour. After separation, the organic layer is washed with saturated sodium hydrogenocarbonate and brine, dried over magnesium sulfate and concentrated. The residue is triturated in ethyl acetate and the suspension is filtered. The solid is recovered to yield 5.3 g of 4-bromo-1-[(4-methylphenyl)sulfonyl]pyrrolidin-3-one x13.

Yield: 66%.

LC-MS (MH⁺): 318/320.

### 9.3 Synthesis of 2-[4-(3-chloropropoxy)phenyl]-5-[(4-methylphenyl)sulfonyl]-4,5,6,6a-tetrahydro-3aH-pyrrolo[3,4-d][1,3]thiazol-3a-ol x14

A solution of 4-bromo-1-[(4-methylphenyl)sulfonyl]pyrrolidin-3-one x13 (5.0 g, 15.7 mmol, 1 eq) and 4-(3-chloropropoxy)benzenecarbothioamide x2 (3.24 g, 14.1 mmol, 0.9 eq) in dimethylformamide (100 ml) is stirred at 60° C. during 2 hours. After evaporation, the residue is dissolved in a 9:1 mixture of dichloromethane/methanol (200 ml), washed with water (100 ml) and concentrated. The solid material is triturated in acetonitrile (20 ml), filtered and dried at 40° C. under reduced pressure to afford 5.3 g of 2-[4-(3-chloropropoxy)phenyl]-5-[(4-methylphenyl)sulfonyl]-4,5,6,6a-tetrahydro-3aH-pyrrolo[3,4-d][1,3]thiazol-3a-ol x14.

Yield: 83%.

LC-MS (MH⁺): 467/469.

### 9.4 Synthesis 2-[4-(3-chloropropoxy)phenyl]-5-[(4-methylphenyl)sulfonyl]-5,6-dihydro-4H-pyrrolo[3,4-d][1,3]thiazole x15

A suspension of 2-[4-(3-chloropropoxy)phenyl]-5-[(4-methylphenyl)sulfonyl]-4,5,6,6a-tetrahydro-3aH-pyrrolo[3,4-d][1,3]thiazol-3a-ol x14 (2.5 g, 5.35 mmol, 1 eq) in dichloromethane (250 ml) is treated successively by triethylamine (2.24 ml, 16.0 mmol, 3 eq) and methanesulfonyl chloride (0.83 ml, 10.0 mmol, 2 eq). The mixture is stirred overnight at 20° C. The mixture is washed with water (100 ml) and the organic layer is dried over magnesium sulfate and concentrated to yield 2.3 g of crude 2-[4-(3-chloropropoxy)phenyl]-5-[(4-methylphenyl)sulfonyl]-5,6-dihydro-4H-pyrrolo[3,4-d][1,3]thiazole x15, which is engaged in the following reaction without any further purification.

Yield: 96%.

LC-MS (MH⁺): 449/451.

### 9.5 Synthesis of 5-[(4-methylphenyl)sulfonyl]-2-{4-[3-(2-methylpyrrolidin-1-yl)propoxy]phenyl}-5,6-dihydro-4H-pyrrolo[3,4-d][1,3]thiazole 45

A suspension of 2-[4-(3-chloropropoxy)phenyl]-5-[(4-methylphenyl)sulfonyl]-5,6-dihydro-4H-pyrrolo[3,4-d][1,3]thiazole x15 (0.3 g, 0.67 mmol, 1 eq) and 2-methylpyrrolidine (0.24 g, 2.0 mmol, 3 eq) in acetonitrile (5 ml) is heated at 90° C. overnight in a sealed tube. After concentration under reduced pressure, the crude material is dissolved in ethyl acetate (40 ml), washed with a saturated solution of ammonium chloride, dried over magnesium sulfate and concentrated. Purification over silicagel (dichloromethane/methanol/ammonia 97:2.7:0.3) yields 160 mg of 5-[(4-methylphenyl)sulfonyl]-2-{4-[3-(2-methylpyrrolidin-1-yl)propoxy]phenyl}-5,6-dihydro-4H-pyrrolo[3,4-d][1,3]thiazole 45.

Yield: 48%.

LC-MS (MH⁺): 498.

### Example 10

### Synthesis of 2-{4-[3-(2-methylpyrrolidin-1-yl)propoxy]phenyl}-5,6,7,8-tetrahydro-4H-[1,3]thiazolo[4,5-d]azepine 46

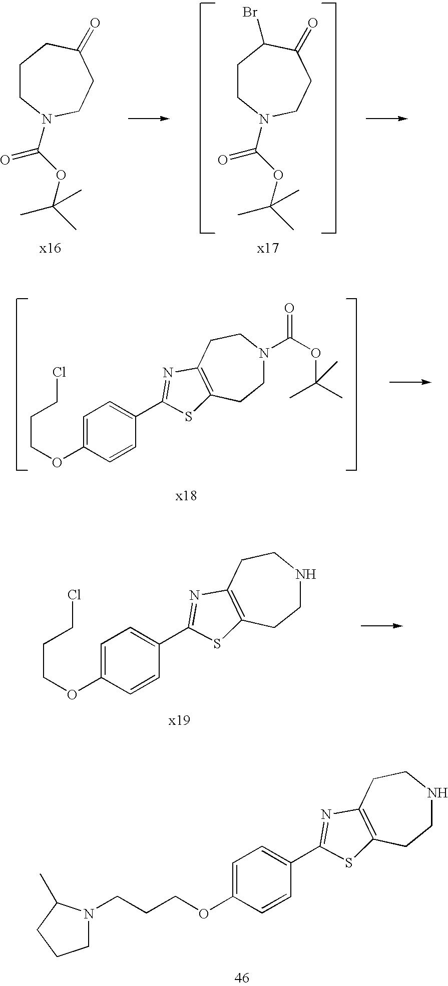

### 10.1 Synthesis of 2-[4-(3-chloropropoxy)phenyl]-5,6,7,8-tetrahydro-4H-[1,3]thiazolo[4,5-d]azepine x19

A solution of tert-butyl 4-oxoazepane-1-carboxylate (0.4 g, 1.8 mmol, 1.06 eq) in tetrahydrofuran (10 ml) is treated with polymer-supported pyridinium tribromide (1 mmol/g) (2 g, 2 mmol, 1.1 eq) and the mixture is stirred overnight. After filtration and concentration of the liquid phase, the residue containing tert-butyl 4-bromo-5-oxoazepane-1-carboxylate x17 is dissolved in isopropanol (5 ml) and 4-(3-chloropropoxy)benzenecarbothioamide x2 (0.4 g, 1.7 mmol, 1 eq) is added. The mixture is stirred overnight at 90° C. After cooling to 20° C., the mixture is taken up with dichloromethane (20 ml) and washed with water (10 ml). The organic layer is dried over magnesium sulfate and concentrated under reduced pressure to afford crude tert-butyl 2-[4-(3-chloropropoxy)phenyl]-4,5,7,8-tetrahydro-6H-[1,3]thiazolo[4,5-d]azepine-6-carboxylate x18. This residue is dissolved in a 1 M HCl aqueous solution (5 ml) and extracted with dichloromethane (10 ml). The aqueous layer is brought to pH 8 by adding sodium bicarbonate. The aqueous phase is extracted with dichloromethane (20 ml). The organic phase is dried over magnesium sulfate and concentrated under reduced pressure to afford 70 mg of 2-[4-(3-chloropropoxy)phenyl]-5,6,7,8-tetrahydro-4H-[1,3]thiazolo[4,5-d]azepine x19.

Yield: 13%.

LC-MS (MH⁺): 323/325.

### 10.2 Synthesis of 2-{4-[3-(2-methylpyrrolidin-1-yl)propoxy]phenyl}-5,6,7,8-tetrahydro-4H-[1,3]thiazolo[4,5-d]azepine 46

A solution of 2-[4-(3-chloropropoxy)phenyl]-5,6,7,8-tetrahydro-4H-[1,3]thiazolo[4,5-d]azepine x19 (70 mg, 0.22 mmol, 1 eq) and 2-methylpyrrolidine (60 μl, 0.65 mmol, 3 eq) in acetonitrile (4 ml) is stirred at 95° C. overnight. After cooling, ethyl acetate (20 ml) is added and the organic layer is washed with water (10 ml), dried over magnesium sulfate and concentrated under reduced pressure to afford 13 mg of 2-{4-[3-(2-methylpyrrolidin-1-yl)propoxy]phenyl}-5,6,7,8-tetrahydro-4H-[1,3]thiazolo[4,5-d]azepine 46.

Yield: 16%.

LC-MS (MH⁺): 372.

### Example 11

### Synthesis of ethyl 2-{4-[3-(2-methylpyrrolidin-1-yl)propoxy]phenyl}-5,6-dihydro-4H-cyclopenta[d][1,3]thiazole-4-carboxylate 56

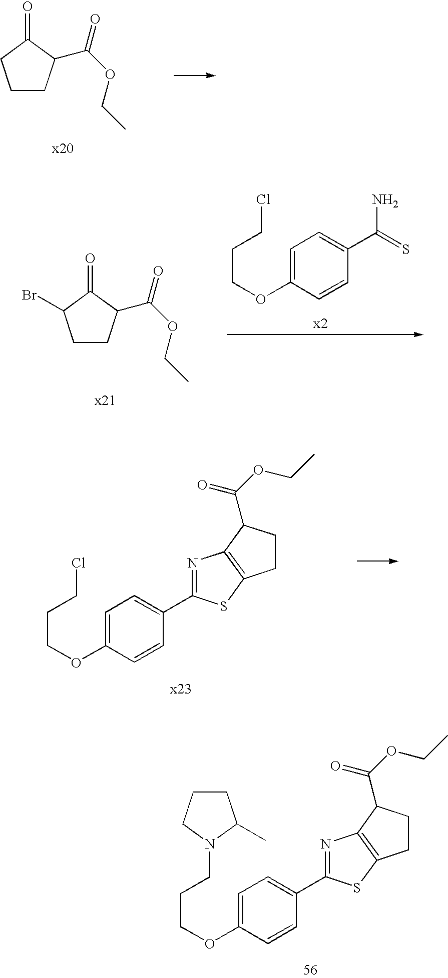

### 11.1 Synthesis of ethyl 3-bromo-2-oxocyclopentanecarboxylate x21

A solution of ethyl 2-oxocyclopentanecarboxylate x20 (5 g, 32.01 mmol) in chloroform (35 ml) is treated dropwise with a solution of bromine (1.81 ml, 35.22 mmol, 1.1 eq) in chloroform (10 ml). The mixture is washed with water, dried over magnesium sulfate and concentrated in vacuo. The residual oil is purified by chromatography over silicagel (dichloromethane) to afford 6.48 g of ethyl 3-bromo-2-oxocyclopentanecarboxylate x21 as a purple oil.

Yield: 86%.

LC-MS (M⁺): 234/236.

Ethyl 3-bromo-2-oxocyclohexanecarboxylate x22 may be prepared according to the same method.

### 11.2 Synthesis of ethyl 2-[4-(3-chloropropoxy)phenyl]-5,6-dihydro-4H-cyclopenta[d][1,3]thiazole-4-carboxylate x23

A solution of 4-(3-chloropropoxy)benzenecarbothioamide x2 (1 g, 4.35 mmol, 1 eq) and 3-bromo-2-oxocyclopentanecarboxylate x21 (1.23 g, 5.22 mmol, 1.2 eq) in ethanol (15 ml) is stirred at 85° C. for 2 hours. The mixture is concentrated and the residue is taken up with ethyl acetate and washed with a 10% aqueous solution of sodium hydroxide. The organic phase is dried over magnesium sulfate and the solvent is removed under high-vacuum to afford a brown oil. The crude material is purified by chromatography over silicagel (dichloromethane/hexane 95:5) to afford 4.29 g of ethyl 2-[4-(3-chloropropoxy)phenyl]-5,6-dihydro-4H-cyclopenta[d][1,3]thiazole-4-carboxylate x23.

Yield: 58%.

LC-MS (MH⁺): 366/388.

Ethyl 2-[4-(3-chloropropoxy)phenyl]-4,5,6,7-tetrahydro-1,3-benzothiazole-4-carboxylate x24 may be prepared according to the same method.

### 11.3 Synthesis of ethyl 2-{4-[3-(2-methylpyrrolidin-1-yl)propoxy]phenyl}-5,6-dihydro-4H-cyclopenta[d][1,3]thiazole-4-carboxylate 56

To a solution of ethyl 2-[4-(3-chloropropoxy)phenyl]-5,6-dihydro-4H-cyclopenta[d][1,3]thiazole-4-carboxylate x23 (150 mg, 0.41 mmol, 1 eq) in acetonitrile (10 ml) is added 2-methylpyrrolidine (79 μl, 0.82 mmol, 2 eq) and sodium iodide (6 mg, 0.04 mmol, 0.1 eq). The mixture is stirred at 85° C. for 48 hours and then taken up with dichloromethane. The organic phase is washed with water, dried over magnesium sulfate, and concentrated to afford a brown oil which is purified by chromatography over silicagel (dichloromethane/methanol-ammonia 97:2.7:0.3) to give 87 mg of ethyl 2-{4-[3-(2-methylpyrrolidin-1-yl)propoxy]phenyl}-5,6-dihydro-4H-cyclopenta[d][1,3]thiazole-4-carboxylate 56.

Yield: 51%.

LC-MS (MH⁺): 415.

Compound 51 has been prepared according to the same method.

### Example 12

### Synthesis of benzyl 2-{4-[3-(2-methylpyrrolidin-1-yl)propoxy]phenyl}-5,6-dihydro-4H-cyclopenta[d][1,3]thiazol-4-ylcarbamate 58, 2-{4-[3-(2-methylpyrrolidin-1-yl)propoxy]phenyl}-5,6-dihydro-4H-cyclopenta[d][1,3]thiazol-4-amine 60 and N-(2-{4-[3-(2-methylpyrrolidin-1-yl)propoxy]phenyl}-5,6-dihydro-4H-cyclopenta[d][1,3]thiazol-4-yl)acetamide 61

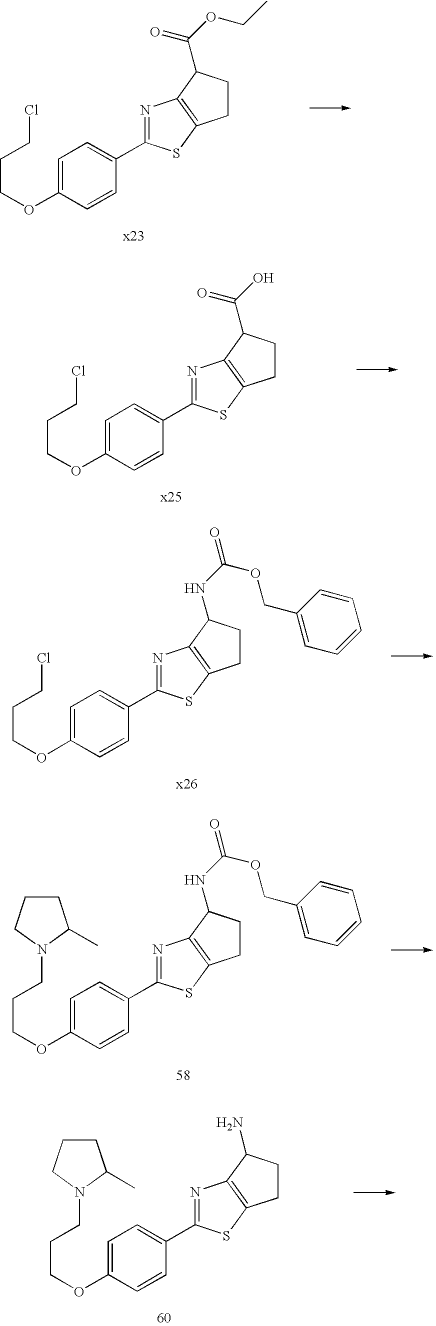

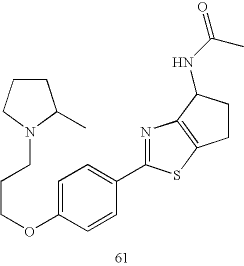

### 12.1 Synthesis of 2-[4-(3-chloropropoxy)phenyl]-5,6-dihydro-4H-cyclopenta[d][1,3]thiazole-4-carboxylic acid x25

A solution of ethyl 2-[4-(3-chloropropoxy)phenyl]-5,6-dihydro-4H-cyclopenta[d][1,3]thiazole-4-carboxylate x23 (4.29 g, 11.73 mmol, 1 eq) in methanol (70 ml) is treated with 1 N aqueous sodium hydroxide (11.73 ml, 11.73 mmol, 1 eq) and the mixture is stirred at room temperature for 48 hours. The mixture is then concentrated to dryness, taken up with diethyl ether, and washed with water. The aqueous phase is acidified with 1 N hydrochloric acid (12 ml). The precipitate is filtered and dried under high-vacuum to give 2.8 g of 2-[4-(3-chloropropoxy)phenyl]-5,6-dihydro-4H-cyclopenta[d][1,3]thiazole-4-carboxylic acid x25 as a yellow solid.

Yield: 71%.

LC-MS (MH⁺): 338/340.

### 12.2 Synthesis of benzyl {2-[4-(3-chloropropoxy)phenyl]-5,6-dihydro-4H-cyclopenta[d][1,3]thiazol-4-yl}carbamate x26

A solution of 2-[4-(3-chloropropoxy)phenyl]-5,6-dihydro-4H-cyclopenta[d][1,3]thiazole-4-carboxylic acid x25 (3.04 g, 9 mmol, 1 eq) in toluene (90 ml) is treated with triethylamine (3.16 ml, 22.5 mmol, 2.5 eq) and diphenylphosphorylazide (2.9 ml, 13.5 mmol, 1.5 eq). The mixture is stirred at 40° C. overnight. Benzyl alcohol (1.87 ml, 18 mmol, 2 eq) is then added and the mixture is stirred at 90° C. for 24 hours. The mixture is then concentrated to dryness. The residue is dissolved in ethyl acetate and washed with a saturated solution of ammonium chloride and a saturated solution of sodium hydrogenocarbonate. The organic phase is dried over magnesium sulfate and concentrated in vacuo to give 5.26 g of a brown oil that is purified by chromatography over silicagel (dichloromethane) to yield 2.64 g of a yellow solid which is still containing benzyl alcohol. The solid is triturated in diethyl ether and filtered to afford 2.18 g of benzyl {2-[4-(3-chloropropoxy)phenyl]-5,6-dihydro-4H-cyclopenta[d][1,3]thiazol-4-yl}carbamate x26 as a white solid.

Yield: 55%.

LC-MS (MH⁺): 443/445.

### 12.3 Synthesis of benzyl 2-{4-[3-(2-methylpyrrolidin-1-yl)propoxy]phenyl}-5,6-dihydro-4H-cyclopenta[d][1,3]thiazol-4-ylcarbamate 58

A solution of benzyl {2-[4-(3-chloropropoxy)phenyl]-5,6-dihydro-4H-cyclopenta[d][1,3]thiazol-4-yl}carbamate x26 (2.17 g, 4.9 mmol, 1 eq) in acetonitrile (50 ml) is treated with potassium carbonate (1.35 g, 9.8 mmol, 2 eq) and sodium iodide (73 mg, 0.49 mmol, 0.1 eq) and the mixture, is stirred at 90° C. for 1 hour. The mixture is then cooled at room temperature and 2-methylpyrrolidine (626 mg, 7.35 mmol, 1.5 eq) is added. The mixture is then stirred at 90° C. for 60 hours, 2-methylpyrrolidine (209 mg, 2.45 mmol, 0.5 eq) is added twice over 4 days while maintaining the temperature at 90° C. The mixture is then concentrated to dryness, the residue is taken up with ethyl acetate and washed twice with water. The combined aqueous phases are extracted with ethyl acetate, the organic phase is dried over magnesium sulfate and concentrated in vacuo to give a brown oil, which is purified by chromatography over silicagel (dichloromethane/methanol/ammonia 96:4:0.4) to afford 1.55 g of benzyl 2-{4-[3-(2-methylpyrrolidin-1-yl)propoxy]phenyl}-5,6-dihydro-4H-cyclopenta[d][1,3]thiazol-4-ylcarbamate 58 as a yellow solid.

Yield: 64%.

LC-MS (MH⁺): 492.

### 12.4 Synthesis of (2-{4-[3-(2-methyl-pyrrolidin-1-yl)-propoxy]-phenyl}-5,6-dihydro-4H-cyclopentathiazol-4-ylamine 60

A solution of benzyl 2-{4-[3-(2-methylpyrrolidin-1-yl)propoxy]phenyl}-5,6-dihydro-4H-cyclopenta[d][1,3]thiazol-4-ylcarbamate 58 (758 mg, 1.54 mmol, 1 eq) in 5N aqueous hydrochloric acid (15 ml) is stirred under reflux for 4 hours. The mixture is then washed with diethyl ether, basified with a solution of 1 N sodium hydroxide and extracted three times with ethyl acetate. The organic phases are washed with a saturated solution of sodium chloride, dried over magnesium sulfate and concentrated in vacuo to give an orange oil, which is purified by chromatography over silicagel (ethyl acetate/ethanol/ammonia 85:15:1.5) to afford 305 mg of (2-{4-[3-(2-methyl-pyrrolidin-1-yl)-propoxy]-phenyl}-5,6-dihydro-4H-cyclopentathiazol-4-ylamine 60 as a yellow oil.

Yield: 55%.

LC-MS (MH⁺): 358.

### 12.5 Synthesis of N-(2-{4-[3-(2-methylpyrrolidin-1-yl)propoxy]phenyl}-5,6-dihydro-4H-cyclopenta[d][1,3]thiazol-4-yl)acetamide 61

To a solution of (2-{4-[3-(2-methyl-pyrrolidin-1-yl)-propoxy]-phenyl}-5,6-dihydro-4H-cyclopentathiazol-4-ylamine 60 (280 mg, 0.78 mmol, 1 eq) in dichloromethane (15 ml) cooled to 0° C. is added triethylamine (165 μl, 1.18 mmol, 1.52 eq) and acetyl chloride (74 mg, 0.94 mmol, 1.2 eq). The mixture is stirred at room temperature for 2 h 30 then dichloromethane (15 ml) is added. The mixture is washed with a saturated solution of sodium hydrogenocarbonate and a saturated solution of sodium chloride. The organic phase is dried over magnesium sulfate and concentrated in vacuo to give of an orange oil, which is purified by chromatography over silicagel (dichloromethane/methanol/ammonia 96:4:0.4) to afford 146 mg of N-(2-{4-[3-(2-methyl-pyrrolidin-1-yl)-propoxy]-phenyl}-5,6-dihydro-4H-cyclopentathiazol-4-yl)-acetamide 61 as an orange solid.

Yield: 47%.

LC-MS (MH⁺): 400.

### Example 13

### Synthesis of 2-{4-[3-(2-methylpyrrolidin-1-yl)propoxy]phenyl}-4-(piperidin-1-ylcarbonyl)-5,6-dihydro-4H-cyclopenta[d][1,3]thiazole 57

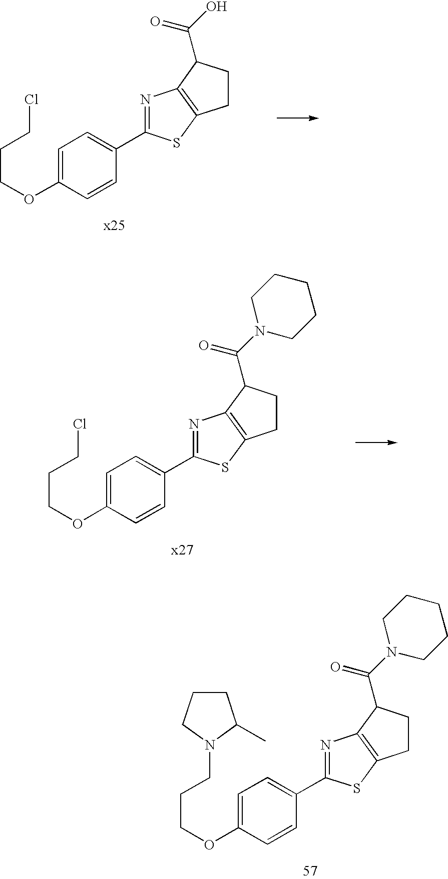

### 13.1 Synthesis of 2-[4-(3-chloropropoxy)phenyl]-4-(piperidin-1-ylcarbonyl)-5,6-dihydro-4H-cyclopenta[d][1,3]thiazole x27

A solution of 2-[4-(3-chloropropoxy)phenyl]-5,6-dihydro-4H-cyclopenta[d][1,3]thiazole-4-carboxylic acid x25 (0.5 g, 1.48 mmol, 1 eq), 1-(3-dimethylaminopropyl)-3-ethylcarbodiimide hydrochloride (0.31 g, 1.63 mmol, 1.1 eq) and 1-hydroxybenzotriazole (0.02 g, 0.16 mmol, 0.11 eq) in dichloromethane (40 ml) is treated with piperidine (0.29 ml, 2.96 mmol, 2 eq) and the mixture is stirred at room temperature for 12 h. The mixture is washed with water, the organic phase is dried over magnesium sulfate and concentrated to afford an orange oil, which is purified by chromatography over silicagel (dichloromethane/methanol-ammonia 99.5:0.5:0.05) to afford 0.42 g of 2-[4-(3-chloropropoxy)phenyl]-4-(piperidin-1-ylcarbonyl)-5,6-dihydro-4H-cyclopenta[d][1,3]thiazole x27 as a yellow oil.

Yield: 70%.

LC-MS (MH⁺): 405/407.

### 13.2 Synthesis of 2-{4-[3-(2-methylpyrrolidin-1-yl)propoxy]phenyl}-4-(piperidin-1-ylcarbonyl)-5,6-dihydro-4H-cyclopenta[d][1,3]thiazole 57

A solution of 2-[4-(3-chloropropoxy)phenyl]-4-(piperidin-1-ylcarbonyl)-5,6-dihydro-4H-cyclopenta[d][1,3]thiazole x27 (0.42 g, 1.03 mmol, 1 eq) and 2-methylpyrrolidine (0.2 ml, 2.06 ml, 2 eq) in acetonitrile (5 ml) is heated at 90° C. under stirring overnight. The mixture is concentrated and then taken up with dichloromethane, washed with water, dried over magnesium sulfate and concentrated. The oil obtained is purified by chromatography over silicagel (dichloromethane/methanol/ammonia 95:4.5:0.5) to afford 0.25 g of 2-{4-[3-(2-methylpyrrolidin-1-yl)propoxy]phenyl}-4-(piperidin-1-ylcarbonyl)-5,6-dihydro-4H-cyclopenta[d][1,3]thiazole 57 as an orange oil.

Yield: 54%.

LC-MS (MH⁺): 454.

### Example 14

### Synthesis of ethyl 4-methyl-2-{4-[3-(2-methylpyrrolidin-1-yl)propoxy]phenyl}-5,6-dihydro-4H-cyclopenta[d][1,3]thiazole-4-carboxylate 59

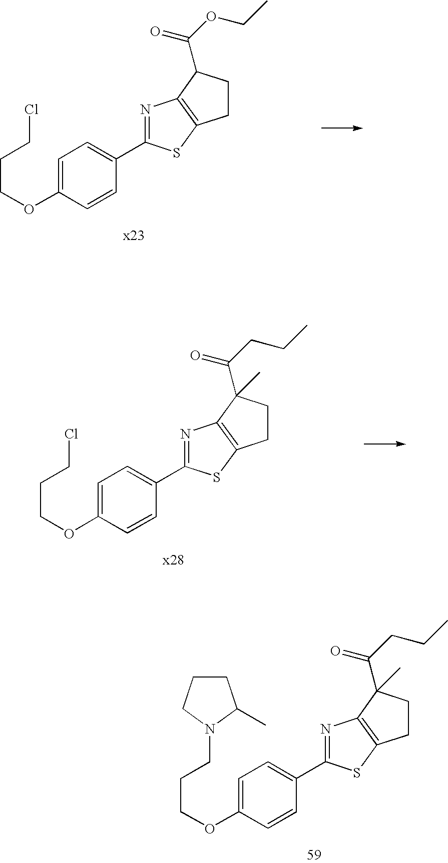

### 14.1 Synthesis of ethyl 2-[4-(3-chloropropoxy)phenyl]-4-methyl-5,6-dihydro-4H-cyclopenta[d][1,3]thiazole-4-carboxylate x28

To a solution of diisopropylamine (0.23 ml, 1.64 mmol, 1.2 eq) in tetrahydrofuran (25 ml) at 0° C. is added dropwise butyl lithium (2 M in hexane, 0.82 ml, 1.2 eq). The mixture is stirred at 0° C. for 15 minutes and then cooled to −70° C. A solution of ethyl 2-[4-(3-chloropropoxy)phenyl]-5,6-dihydro-4H-cyclopenta[d][1,3]thiazole-4-carboxylate x23 (0.5 g, 1.37 mmol, 1 eq) in tetrahydrofuran (10 ml) is added dropwise and the mixture is stirred at −70° C. for 30 minutes. Methyl iodide (0.23 g, 1.64 mmol, 1.2 eq) is added, the mixture is allowed to warm to room temperature and stirred overnight. The mixture is poured into a saturated aqueous solution of ammonium chloride (50 ml), extracted with ethyl acetate, dried over magnesium sulfate and concentrated to give an oil, which is purified by chromatography over silicagel (dichloromethane) to afford 163 mg of ethyl 2-[4-(3-chloropropoxy)phenyl]-4-methyl-5,6-dihydro-4H-cyclopenta[d][1,3]thiazole-4-carboxylate x28.

Yield: 31%.

LC-MS (MH⁺): 380/382.

### 14.2 Synthesis of ethyl 4-methyl-2-{4-[3-(2-methylpyrrolidin-1-yl)propoxy]phenyl}-5, 6 dihydro-4H-cyclopenta[d][1,3]thiazole-4-carboxylate 59

To a solution of ethyl 2-[4-(3-chloropropoxy)phenyl]-4-methyl-5,6-dihydro-4H-cyclopenta[d][1,3]thiazole-4-carboxylate x28 (0.163 g, 0.43 mmol, 1 eq) in acetonitrile (2 ml) is added 2-methylpyrrolidine (83 μl, 0.86 mmol, 2 eq) and sodium iodide (6 mg, 0.04 mmol, 0.1 eq). The mixture is stirred at 80° C. for 24 hours, then poured into dichloromethane and washed with water. The organic phase is dried over magnesium sulfate and concentrated to afford a brown oil. This oil is purified by chromatography over silicagel (eluent: dichloromethane/methanol/ammonia 98:2:0.2) to give 79 mg of ethyl 4-methyl-2-{4-[3-(2-methylpyrrolidin-1-yl)propoxy]phenyl}-5,6-dihydro-4H-cyclopenta[d][1,3]thiazole-4-carboxylate 59 as a yellow oil.

Yield: 43%

LC-MS (MH⁺): 429

### Example 15

### Synthesis of 2-{4-[3-(2-methylpyrrolidin-1-yl)propoxy]phenyl}-4,5,6,7-tetrahydro-1,3-benzothiazol-4-ol 47 and 2-{4-[3-(2-methylpyrrolidin-1-yl)propoxy]phenyl}-4-piperidin-1-yl-4,5,6,7-tetrahydro-1,3-benzothiazole 48

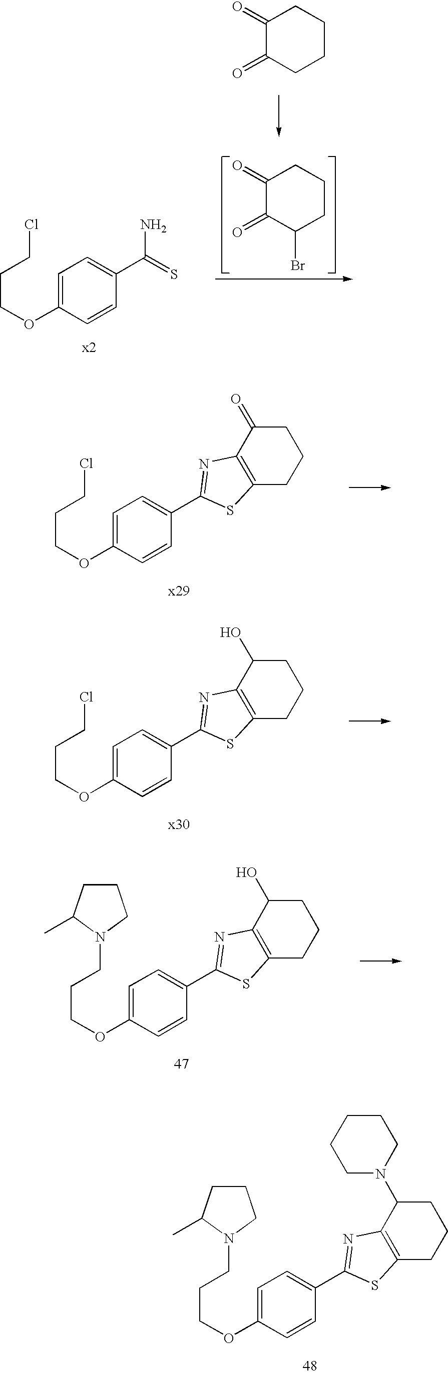

### 15.1 Synthesis of 2-[4-(3-chloropropoxy)phenyl]-6,7-dihydro-1,3-benzothiazol-4(5H)-one x29

A solution of cyclohexane-1,2-dione (2.18 g, 19.44 mmol, 1 eq) in tetrahydrofuran (100 ml) at 0° C. is treated with bromine (3.107 g, 19.44 mmol, 1 eq). After 30 minutes at 30° C., 4-(3-chloropropoxy)benzenecarbothioamide x2 (4.68 g, 20.4 mmol, 1.05 eq) is added. The mixture is stirred at 70° C. overnight. After concentration, ethyl acetate (150 ml) is added. The organic layer is washed with water (2×100 ml) and brine (100 ml), dried over magnesium sulfate and concentrated to give 7.5 g of a brown oil that is purified by chromatography over silicagel (dichloromethane/ethanol/ammonia 99:0.9:0.1) to afford 500 mg of 2-[4-(3-chloropropoxy)phenyl]-6,7-dihydro-1,3-benzothiazol-4(5H)-one x29.

Yield: 23%.

LC-MS (MH⁺): 322.

### 15.2 Synthesis of 2-[4-(3-chloropropoxy)phenyl]-4,5,6,7-tetrahydro-1,3-benzothiazol-4-ol x30

A solution of 2-[4-(3-chloropropoxy)phenyl]-6,7-dihydro-1,3-benzothiazol-4(5H)-one x29 (0.5 g, 1.55 mmol, 1 eq) in tetrahydrofuran (15 ml) at 0° C. is treated with sodium borohydride (0.176 g, 4.66 mmol, 3 eq) and iodine (0.248 g, 1.05 mmol, 0.68 eq) and stirred at 60° C. overnight. The mixture is then poured into cold water (10 ml) before being concentrated. The aqueous layer is extracted with ethyl acetate (3×50 ml and 1×100 ml). The combined organic layers are washed with brine, dried over magnesium sulfate and concentrated in vacuo to afford 0.5 g of an oily solid. The crude 2-[4-(3-chloropropoxy)phenyl]-4,5,6,7-tetrahydro-1,3-benzothiazol-4-ol x30 is used as such in the next step.

Yield: 99%.

LC-MS (MH⁺): 324.

### 15.3 Synthesis of 2-{4-[3-(2-methylpyrrolidin-1-yl)propoxy]phenyl}-4,5,6,7-tetrahydro-1,3-benzothiazol-4-ol 47

A solution of 2-[4-(3-chloropropoxy)phenyl]-4,5,6,7-tetrahydro-1,3-benzothiazol-4-ol x30 (0.337 g, 1.04 mmol, 1 eq), potassium carbonate (0.287 g, 2.09 mmol, 2 eq), and sodium iodide (3 mg, 0.223 mmol, 0.02 eq) in acetonitrile (30 ml) is treated with 2-methylpyrrolidine (0.3 ml, 2.9 mmol, 2.8 eq) and stirred at 80° C. overnight. The mixture is then poured into 20 ml of water and extracted with ethyl acetate (3×50 ml). The organic layers are then washed with brine, dried over magnesium sulfate and concentrated in vacuo to afford 260 mg of an oily solid. The mixture is purified by chromatography over silicagel (dichloromethane/ethanol/ammonia 95:4.5:0.5) to afford 75 mg of 2-{4-[3-(2-methylpyrrolidin-1-yl)propoxy]phenyl}-4,5,6,7-tetrahydro-1,3-benzothiazol-4-ol 47.

Yield: 13%.

LC-MS (MH⁺): 373.

### 15.4 Synthesis of 2-{4-[3-(2-methylpyrrolidin-1-yl)propoxy]phenyl}-4-piperidin-1-yl-4,5,6,7-tetrahydro-1,3-benzothiazole 48

To a solution of 2-{4-[3-(2-methylpyrrolidin-1-yl)propoxy]phenyl}-4,5,6,7-tetrahydro-1,3-benzothiazol-4-ol 47 (0.55 g, 1.48 mmol, 1 eq) in dichloromethane (30 ml) at 0° C. is added triethylamine (0.41 ml, 2.95 mmol, 2 eq) and methanesulfonyl chloride (0.35 ml, 4.43 mmol, 3 eq). The mixture is stirred at room temperature for 1 hour. Piperidine (0.73 ml, 7.38 mmol, 5 eq) is then added and the mixture is stirred overnight. After concentration, the residue is poured into ethyl acetate (150 ml). The organic phase is washed with distilled water (1×50 ml) and brine, dried over magnesium sulfate and concentrated in vacuo. The mixture is successively purified by chromatography over silicagel (dichloromethane/methanol/ammonia 95:4.5:0.5) and by preparative LC-MS chromatography (Sunfire Prep MS C18 ODB column) eluting with a gradient of water, acetonitrile and trifluoroacetic acid to afford 186 mg of 2-{4-[3-(2-methylpyrrolidin-1-yl)propoxy]phenyl}-4-piperidin-1-yl-4,5,6,7-tetrahydro-1,3-benzothiazole 48 as a salt (1.7 CF₃COOH, 2.7H₂O).

Yield: 54%.

LC-MS (MH⁺): 440.

### Example 16

### Synthesis of 2-{4-[3-(2-methylpyrrolidin-1-yl)propoxy]phenyl}-7-piperidin-1-yl-4,5,6,7-tetrahydro-1,3-benzothiazole 49

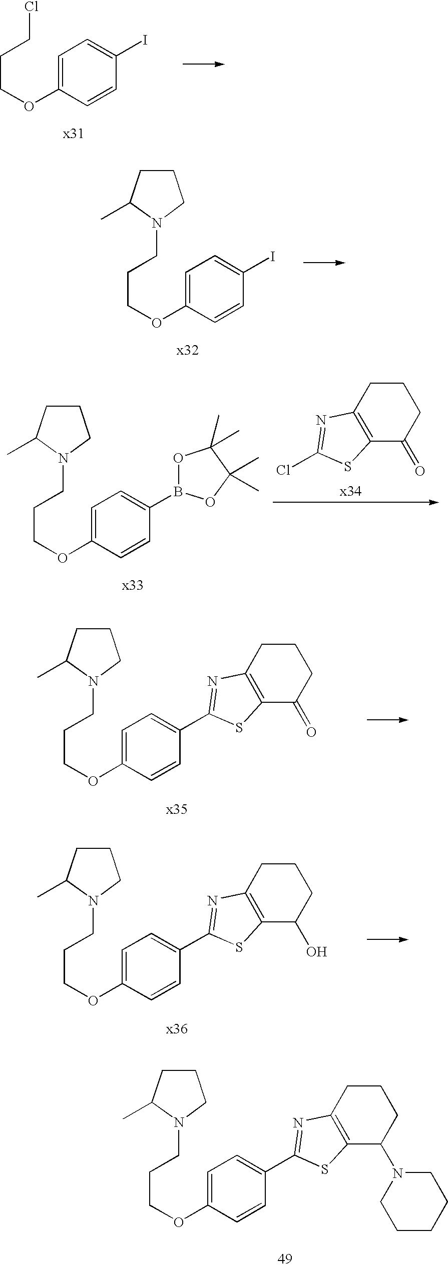

### 16.1 Synthesis of 1-[3-(4-iodophenoxy)propyl]-2-methylpyrrolidine x32

A solution of 1-(3-chloropropoxy)-4-iodobenzene x31 (2.07 g, 6.75 mmol, 1 eq), potassium carbonate (1.95 g, 13.5 mmol, 2 eq) and sodium iodide (0.1 g, 0.65 mmol, 0.1 eq) are stirred at 80° C. in acetonitrile (50 ml) for 1 hour. 2-Methylpyrrolidine (1.5 ml, 14.55 mmol, 2.2 eq) is added, and the mixture is stirred overnight at 80° C. The mixture is then poured into ethyl acetate (150 ml) and washed with water (2×100 ml) and brine. The organic phase is dried over magnesium sulfate and concentrated to yield 2.2 g of 1-[3-(4-iodophenoxy)propyl]-2-methylpyrrolidine x32.

Yield: 95%.

LC-MS (MH⁺): 346.

### 16.2 Synthesis of 2-methyl-1-{3-[4-(4,4,5,5-tetramethyl-1,3,2-dioxaborolan-2-yl)phenoxy]propyl}pyrrolidine x33

Argon is bubbled through a solution of 1-[3-(4-iodo-phenoxy)-propyl]-2-methyl-pyrrolidine (7.2 g, 20.8 mmol, 1 eq), bis(pinacolato)diboron (5.88 g, 23 mmol, 1.1 eq) and potassium acetate (6.25 g, 63.6 mmol, 3.05 eq) in dimethylsulfoxide (60 ml). 1,1′-bis(diphenylphosphino)ferrocene palladium (II) dichloride (0.525 g, 0.7 mmol, 0.03 eq) is then added and the mixture is stirred at 80° C. overnight. The mixture is then poured into toluene (150 ml). After filtration, the mixture is washed with water (3×100 ml) and brine. The organic phase is dried over magnesium sulfate and concentrated to yield 4.5 g of a black solid containing 2-methyl-1-{3-[4-(4,4,5,5-tetramethyl-1,3,2-dioxaborolan-2-yl)phenoxy]propyl}pyrrolidine x33. This crude reaction product is used as such in the next step.

Yield: 63%.

LC-MS (MH⁺): 346.

### 16.3 Synthesis of 2-{4-[3-(2-methylpyrrolidin-1-yl)propoxy]phenyl}-5,6-dihydro-1,3-benzothiazol-7(4H)-one x35

Argon is bubbled through a solution of 2-methyl-1-{3-[4-(4,4,5,5-tetramethyl-1,3,2-dioxaborolan-2-yl)phenoxy]propyl}pyrrolidine x33 (4.5 g, 13.03 mmol, 1 eq), 2-chloro-5,6-dihydro-4H-benzothiazol-7-one (2.45 g, 13.03 mmol, 1 eq) and potassium acetate (6.25 g, 63.6 mmol, 3.05 eq) in dimethylformamide (80 ml). Tetrakis(triphenylphosphine) palladium (0.45 g, 0.55 mmol, 0.04 eq) is then added and the mixture is stirred at 80° C. for 2 hours. The mixture is then poured into water (100 ml) and extracted with ethyl acetate (4×100 ml). The combined organic phases are dried over magnesium sulfate and concentrated in vacuo. The residue is taken up with 0.2 N hydrochloric acid (150 ml), washed with ethyl acetate (2×100 ml) and brought to pH 12 with sodium hydroxide pellets. The aqueous phase is extracted with ethyl acetate (3×100 ml). Combined organic phases are washed with brine, dried over magnesium sulfate and concentrated in vacuo to afford 1.8 g of 2-{4-[3-(2-methylpyrrolidin-1-yl)propoxy]phenyl}-5,6-dihydro-1,3-benzothiazol-7(4H)-one x35 which is used in the next step without any further purification.

Yield: 37%.

LC-MS (MH⁺): 371.

### 16.4 Synthesis of 2-{4-[3-(2-methylpyrrolidin-1-yl)propoxy]phenyl}-4,5,6,7-tetrahydro-1,3-benzothiazol-7-ol x36

2-{4-[3-(2-methylpyrrolidin-1-yl)propoxy]phenyl}-4,5,6,7-tetrahydro-1,3-benzothiazol-7-ol x36 is synthesized as described in example 15.2, starting from 2-{4-[3-(2-methylpyrrolidin-1-yl)propoxy]phenyl}-5,6-dihydro-1,3-benzothiazol-7(4H)-one x35, and is used in the next step without any further purification.

Yield: 56%.

LC-MS (MH⁺): 373.

### 16.5 Synthesis of 2-{4-[3-(2-methylpyrrolidin-1-yl)propoxy]phenyl}-7-piperidin-1-yl-4,5,6,7-tetrahydro-1,3-benzothiazole 49

A cooled (ice bath) solution of 2-{4-[3-(2-methylpyrrolidin-1-yl)propoxy]phenyl}-4,5,6,7-tetrahydro-1,3-benzothiazol-7-ol x36 (1.01 g, 2.72 mmol, 1 eq) in dry dichloromethane (20 ml) is treated with triethylamine (0.75 g, 5.44 mmol, 2 eq) and methanesulfonyl chloride (0.65 ml, 8.4 mmol, 3 eq). The mixture is stirred at 0° C. for 2.5 hours. Piperidine (1.3 ml, 13 mmol, 4.8 eq) is added and the mixture is stirred at room temperature overnight. The reaction mixture is poured into dichloromethane (100 ml), washed with brine, dried over magnesium sulfate and concentrated in vacuo to afford 2 g of crude material. It is treated with a 20% solution of potassium hydroxide in methanol (20 ml) at 50° C. overnight. The mixture is then concentrated, poured into water and extracted with ethyl acetate (2×100 ml). The organic layers are washed with brine, dried over magnesium sulfate and concentrated. The residue is purified by chromatography over silicagel (dichloromethane/benzine gradient from 1:9 to 9:1) to afford 300 mg of 2-{4-[3-(2-methylpyrrolidin-1-yl)propoxy]phenyl}-7-piperidin-1-yl-4,5,6,7-tetrahydro-1,3-benzothiazole 49 as a yellow oil.

Yield: 29%.

LC-MS (MH⁺): 440.

### Example 17

### Synthesis of N-(2-{4-[3-(2-methylpyrrolidin-1-yl)propoxy]phenyl}-4,5,6,7-tetrahydro-1,3-benzothiazol-6-yl)acetamide 50

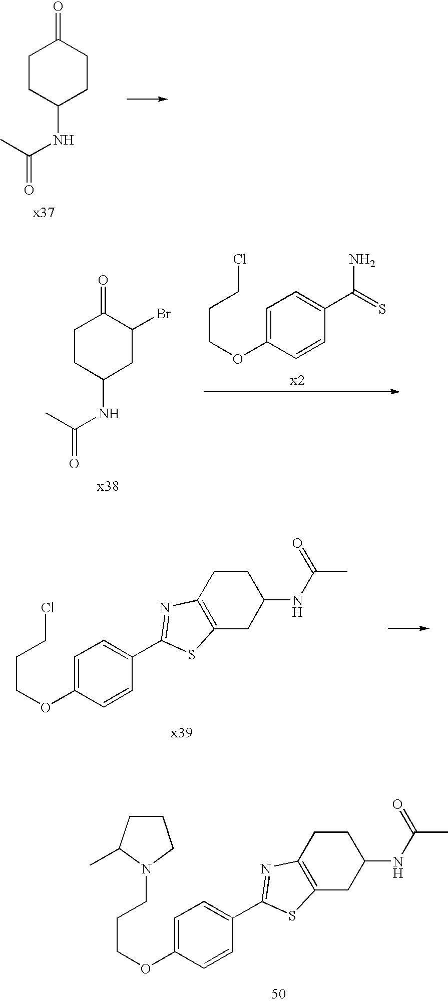

### 17.1 Synthesis of N-(3-bromo-4-oxocyclohexyl)acetamide x38

A stirred solution of N-(4-oxocyclohexyl)acetamide x37 (1.5 g, 9.7 mmol, 1 eq) in chloroform (15 ml) is treated with a 0.5 M solution of bromine in chloroform (20 ml, 9.7 mmol, 1 eq) at 22° C. The reaction mixture is stirred for 20 minutes and concentrated in vacuo to afford 3.15 g of crude N-(3-bromo-4-oxocyclohexyl)acetamide x38 which is used directly in the next step.

Yield: 100%.

LC-MS (MH⁺): 234/236.

### 17.2 Synthesis of N-{2-[4-(3-chloropropoxy)phenyl]-4,5,6,7-tetrahydro-1,3-benzothiazol-6-yl}acetamide x39

A solution of N-(3-bromo-4-oxocyclohexyl)acetamide x38 (2.65 g, 8.4 mmol, 1 eq) and 4-(3-chloropropoxy)benzenecarbothioamide x2 (1.93 g, 8.4 mmol, 1 eq) in dimethylformamide (30 ml) is stirred at reflux overnight. The mixture is concentrated in vacuo. Ethyl acetate (200 ml) is added to the residue and the resulting solution is washed with distilled water (2×50 ml). The combined aqueous layers are extracted with dichloromethane (3×30 ml). The combined organic layers are washed with brine and concentrated in vacuo to afford 1.67 g of crude N-{2-[4-(3-chloropropoxy)phenyl]-4,5,6,7-tetrahydro-1,3-benzothiazol-6-yl}acetamide x39 as an orange solid.

Yield: 54%.

LC-MS (MH⁺): 365/366.

### 17.3 Synthesis of N-{2-[4-(3-chloropropoxy)phenyl]-4,5,6,7-tetrahydro-1,3-benzothiazol-6-yl}acetamide 50

A solution of N-{2-[4-(3-chloropropoxy)phenyl]-4,5,6,7-tetrahydro-1,3-benzothiazol-6-yl}acetamide x39 (1.5 g, 4.1 mmol, 1 eq) and 2-methylpyrrolidine (0.86 g, 10.09 mmol, 2.5 eq) in acetonitrile (8 ml) is stirred under microwave irradiation for 2 hours at 100° C. The mixture is concentrated in vacuo. Dichloromethane (20 ml) is added to the residue and the organic phase is washed with 1 N hydrochloric acid (3×25 ml). The combined aqueous layers are brought to pH 12 with sodium hydroxide (pellets), then extracted with dichloromethane (3×40 ml). The dichloromethane layers are washed with brine, dried over magnesium sulfate and concentrated in vacuo. The residue is purified by chromatography over silicagel (dichloromethane/methanol/ammonia 95:4.5:0.5) to afford 155 mg of N-{2-[4-(3-chloropropoxy)phenyl]-4,5,6,7-tetrahydro-1,3-benzothiazol-6-yl}acetamide 50 as a yellow solid.

Yield: 10%.

LC-MS (MH⁺): 414.

### Example 18

### Synthesis of benzyl 2-{4-[3-(2-methylpyrrolidin-1-yl)propoxy]phenyl}-4,5,6,7-tetrahydro-1,3-benzothiazol-4-ylcarbamate 52 and N-(2-{4-[3-(2-methylpyrrolidin-1-yl)propoxy]phenyl}-4,5,6,7-tetrahydro-1,3-benzothiazol-4-yl)acetamide 53

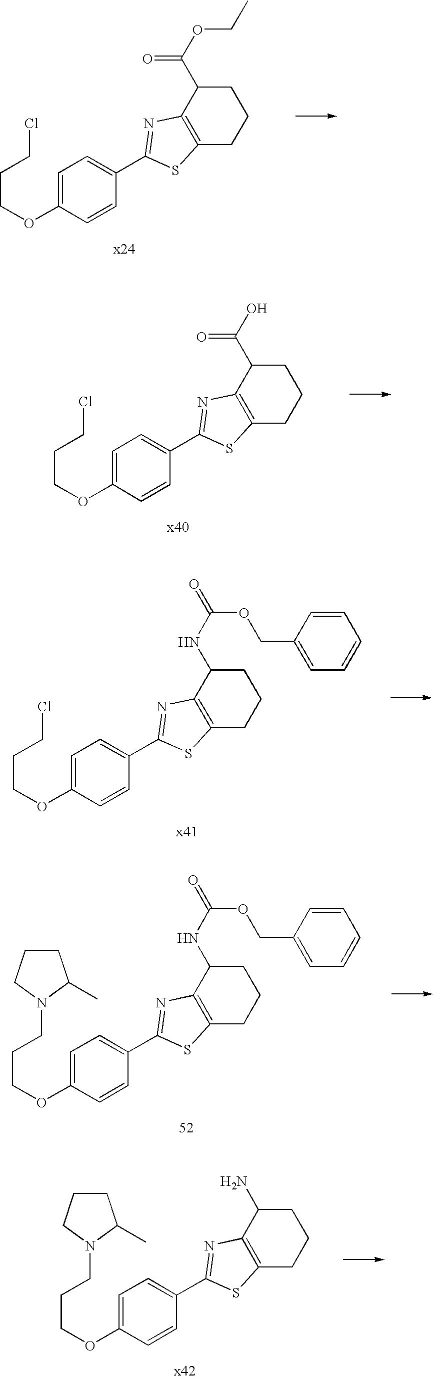

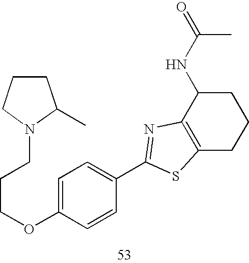

### 18.1 Synthesis of 2-[4-(3-chloropropoxy)phenyl]-4,5,6,7-tetrahydro-1,3-benzothiazole-4-carboxylic acid x40

A solution of ethyl 2-[4-(3-chloropropoxy)phenyl]-4,5,6,7-tetrahydro-1,3-benzothiazole-4-carboxylate x24 (3.2 g, 8.4 mmol, 1 eq) in tetrahydrofuran (180 ml) is treated with a solution of lithium hydroxide monohydrate (0.719 mg, 17.1 mmole, 2 eq) in water (20 ml).

The reaction is stirred at room temperature overnight and at reflux for 1.5 hours. The organic solvent is removed in vacuo. The aqueous mixture is washed with diethyl ether, then acidified to pH 3 and extracted with dichloromethane (3×40 ml). The combined dichloromethane layers are washed with brine, dried over magnesium sulfate and concentrated in vacuo to afford 1.4 g of 2-[4-(3-chloropropoxy)phenyl]-4,5,6,7-tetrahydro-1,3-benzothiazole-4-carboxylic acid x40.

Yield: 47%.

LC-MS (MH⁺): 352/354.

### 18.2 Synthesis of benzyl {2-[4-(3-chloropropoxy)phenyl]-4,5,6,7-tetrahydro-1,3-benzothiazol-4-yl}carbamate x41

A solution of 2-[4-(3-chloropropoxy)phenyl]-4,5,6,7-tetrahydro-1,3-benzothiazole-4-carboxylic acid x40 (1.4 g, 3.98 mmol, 1 eq), triethylamine (1.4 ml, 9.95 mmol, 2.5 eq) in toluene (40 ml) is treated with diphenylphosphoryl azide (1.3 ml, 5.97 mmol, 1.5 eq). The mixture is then stirred at 40° C. overnight. Benzyl alcohol (0.83 ml, 8 mmol, 2 eq) is added and the mixture is stirred at 90° C. overnight. The organic layer is washed with a saturated solution of ammonium chloride (2×30 ml) and a saturated solution of sodium hydrogenocarbonate (2×30 ml), dried over magnesium sulfate and concentrated in vacuo. The residue is purified by chromatography over silicagel (dichloromethane/ethyl acetate gradient from 10:0 to 8:2) to afford 1.2 g of benzyl {2-[4-(3-chloropropoxy)phenyl]-4,5,6,7-tetrahydro-1,3-benzothiazol-4-yl}carbamate x41 as a yellow oil.

Yield: 66%.

LC-MS (MH⁺): 457/459.

### 18.3 Synthesis of benzyl 2-{4-[3-(2-methylpyrrolidin-1-yl)propoxy]phenyl}-4,5,6,7-tetrahydro-1,3-benzothiazol-4-ylcarbamate 52

A solution of benzyl {2-[4-(3-chloropropoxy)phenyl]-4,5,6,7-tetrahydro-1,3-benzothiazol-4-yl}carbamate x41 (0.177 g, 0.35 mmol, 1 eq) and 2-methylpyrrolidine (0.23 g, 2.5 mmol, 2.5 eq) in acetonitrile (10 ml) is stirred at 100° C. for 4 hours. The mixture is concentrated in vacuo and ethyl acetate (50 ml) is added. The organic layer is washed with water (2×20 ml) and brine, dried over magnesium sulfate and concentrated in vacuo. The residue is purified by chromatography over silicagel (dichloromethane/ethyl acetate 10:0 to 9:1) to afford 0.22 g of benzyl 2-{4-[3-(2-methylpyrrolidin-1-yl)propoxy]phenyl}-4,5,6,7-tetrahydro-1,3-benzothiazol-4-ylcarbamate 52 as a yellow solid.

Yield: 44%.

LC-MS (MH⁺): 506.

### 18.4 Synthesis of 2-{4 [3-(2-methylpyrrolidin-1-yl)propoxy]phenyl}-4,5,6,7-tetrahydro-1,3-benzothiazol-4-amine x42

A solution of benzyl 2-{4-[3-(2-methylpyrrolidin-1-yl)propoxy]phenyl}-4,5,6,7-tetrahydro-1,3-benzothiazol-4-ylcarbamate 52 (0.177 g, 0.35 mmol, 1 eq) in 6 N hydrochloric acid (10 ml) is stirred at reflux for 6 hours. The mixture is cooled down to 20° C. and water (15 ml) is added. The reaction mixture is washed with ethyl acetate (2×15 ml). The aqueous layer is brought to pH 12 with sodium hydroxide pellets and extracted with ethyl acetate (3×25 ml). The combined layers are washed with brine, dried over magnesium sulfate and concentrated in vacuo. The residue is purified by chromatography over silicagel (dichloromethane/methanol/ammonia 95:4.5:0.5) to afford 2-{4-[3-(2-methylpyrrolidin-1-yl)propoxy]phenyl}-4,5,6,7-tetrahydro-1,3-benzothiazol-4-amine x42.

Yield: 61%.

LC-MS (MH⁺): 372.

### 18.5 Synthesis of N-(2-{4-[3-(2-methylpyrrolidin-1-yl)propoxy]phenyl}-4,5,6,7-tetrahydro-1,3-benzothiazol-4-yl)acetamide 53

To a cooled (ice bath) solution of 2-{4-[3-(2-methylpyrrolidin-1-yl)propoxy]phenyl}-4,5,6,7-tetrahydro-1,3-benzothiazol-4-amine x42 (0.195 g, 0.52 mmol, 1 eq) and triethylamine (0.08 g, 0.79 mmoles, 1.52 eq) in dichloromethane (10 ml) is added acetyl chloride (0.05 g, 0.64 mmol, 1.2 eq). The mixture is stirred for 3 hours at room temperature. Dichloromethane is added (25 ml) and the organic layer is washed with a saturated solution of sodium hydrogenocarbonate (2×10 ml), dried over magnesium sulfate and concentrated in vacuo. The residue is purified by chromatography over silicagel (dichloromethane/methanol/ammonia 95:4.5:0.5) to afford N-(2-{4-[3-(2-methylpyrrolidin-1-yl)propoxy]phenyl}-4,5,6,7-tetrahydro-1,3-benzothiazol-4-yl)acetamide 53.

Yield: 46%.

LC-MS (MH⁺): 414.

### Example 19

### Synthesis of N-(2-{4-[3-(2-methylpyrrolidin-1-yl)propoxy]phenyl}-4,5,6,7-tetrahydro-1,3-benzothiazol-5-yl)acetamide 55

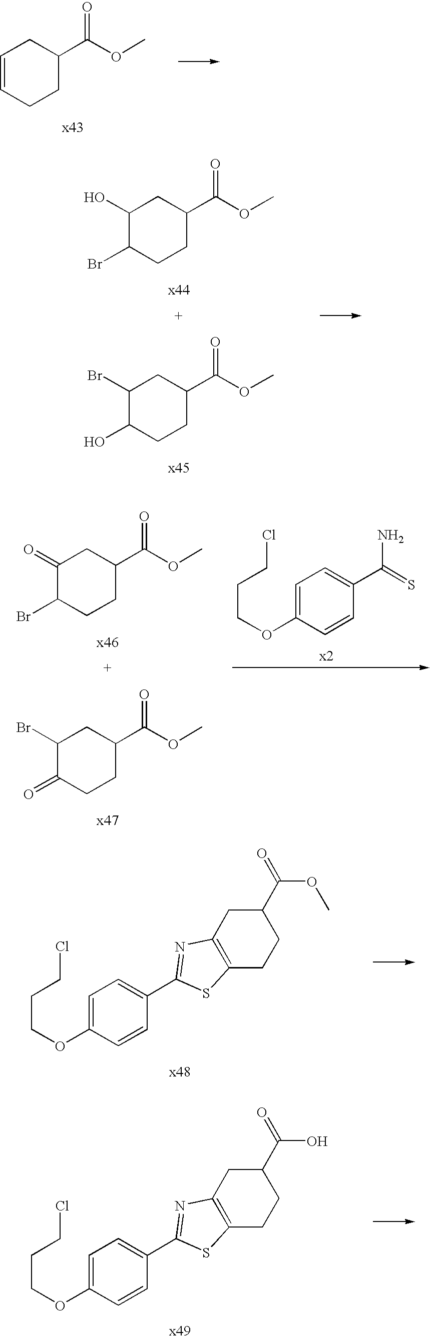

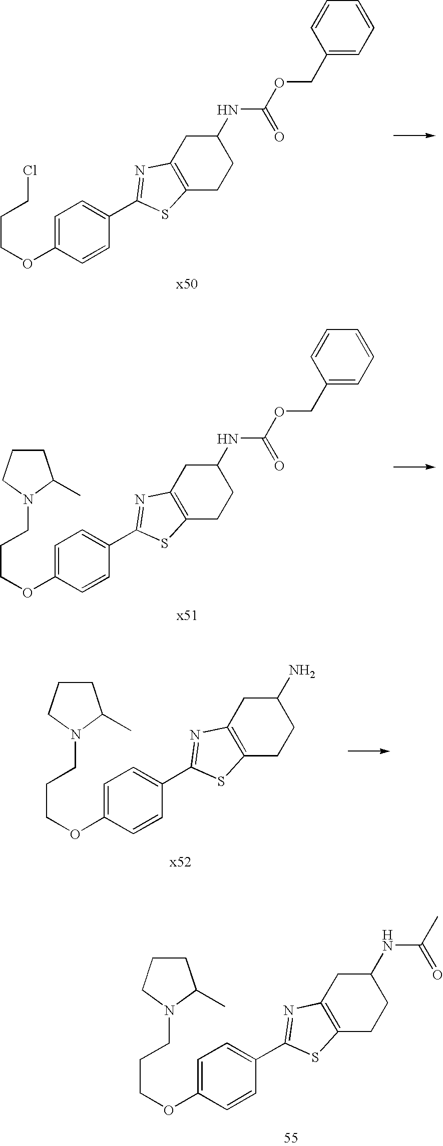

### 19.1 Synthesis of a mixture of methyl 4-bromo-3-hydroxycyclohexanecarboxylate x44 and methyl 3-bromo-4-hydroxycyclohexanecarboxylate x45

A solution of methyl cyclohex-3-ene-1-carboxylate x43 (1.006 g, 7.13 mmol, 1 eq) in acetonitrile (10 ml) is treated with calcium carbonate (0.75 g, 7.5 mmol, 1.06 eq) in water (2.5 ml). The mixture is cooled at 0° C. and a solution of N-bromosuccinimide (1.27 g, 7.13 mmol, 1 eq) in acetonitrile (10 ml) is added slowly. The mixture is stirred at 30° C. for 4 hours. Water (20 ml) is then added and the reaction mixture is extracted with ethyl acetate (3×30 ml). The combined organic layers are washed with water (30 ml), brine, dried over magnesium sulfate and concentrated under vacuum. The residual yellow oil is purified by chromatography over silicagel (benzine/ethyl acetate 90:10) to afford 1.1 g of a mixture of methyl 4-bromo-3-hydroxycyclohexanecarboxylate x44 and methyl 3-bromo-4-hydroxycyclohexanecarboxylate x45 as a colorless oil.

Yield: 65%.

GC-MS (M⁺.): 236/238.

### 19.2 Synthesis of a mixture of methyl 4-bromo-3-oxocyclohexanecarboxylate x46 and methyl 3-bromo-4-oxocyclohexanecarboxylate x47

The above mixture of methyl 4-bromo-3-hydroxycyclohexanecarboxylate x44 and methyl 3-bromo-4-hydroxycyclohexanecarboxylate x45 (4.35 g, 18.35 mmol, 1 eq) in dichloromethane (50 ml) is treated with a 15% solution of Dess-Martin periodinane in dichloromethane (54 ml, 19.1 mmol, 1.04 eq). The mixture is stirred at 35° C. for 2.5 hours, then filtered on a mixture of silicagel and celite, and washed with cyclohexane (3×20 ml). To the residue is then added a 10% aqueous solution of sodium thiosulfate. The mixture is stirred for 1 hour, the two layers are separated and the organic layer is partially concentrated. The white solid that precipitates is removed by filtration. The filtrate is concentrated and the residual yellow oil is purified by chromatography over silicagel (gradient: cyclohexane/ethyl acetate 99:1 to 80:20) to afford a mixture of methyl 4-bromo-3-oxocyclohexanecarboxylate x46 and methyl 3-bromo-4-oxocyclohexanecarboxylate x47 as a colorless oil.

Yield: 42%

GC-MS (M⁺.): 234/236.

### 19.3 Synthesis of methyl 2-[4-(3-chloropropoxy)phenyl]-4,5,6,7-tetrahydro-1,3-benzothiazole-5-carboxylate, enantiomer 1, x48

The above mixture of methyl 4-bromo-3-oxocyclohexanecarboxylate x46 and methyl 3-bromo-4-oxocyclohexanecarboxylate x47 (1.84 g, 7.85 mmol, 1 eq) in N,N-dimethylformamide (8 ml) is treated with 4-(3-chloropropoxy)benzenecarbothioamide x2 (1.32 g, 5.75 mmol, 0.73 eq). The reaction is stirred at 40° C. for 2.5 hours and at room temperature overnight. The mixture is concentrated and the residue taken up with dichloromethane (160 ml). The organic layer is washed with water (1×100 ml, 2×75 ml), brine, dried over magnesium sulfate and concentrated under vacuum. The solid is purified by chromatography over silicagel (cyclohexane/ethyl acetate 80:20) to afford 2.16 g of a yellow solid as a mixture of two regioisomers and their enantiomers.

Yield: 77%.

LC-MS (MH⁺): 366/368.

The two regioisomers and their enantiomers are separated by chiral chromatography (column: Chiralpak AD-H, eluent: ethanol/iso-hexane/diethylamine 50:50:0.1) to afford 0.52 g of methyl 2-[4-(3-chloropropoxy)phenyl]-4,5,6,7-tetrahydro-1,3-benzothiazole-5-carboxylate, enantiomer 1, x48.

LC-MS (MH⁺): 366/368.

Rₜ: 14′4 (Chiralpak AD-H, ethanol/iso-hexane/diethylamine 50:50:0.1).

### 19.4 Synthesis of 2-[4-(3-chloropropoxy)phenyl]-4,5,6,7-tetrahydro-1,3-benzothiazole-5-carboxylic acid, enantiomer 1, x49

A solution of methyl 2-[4-(3-chloropropoxy)phenyl]-4,5,6,7-tetrahydro-1,3-benzothiazole-5-carboxylate, enantiomer 1, x48 (0.52 g, 1.42 mmol, 1 eq) in tetrahydrofuran (55 ml) is treated with a solution of lithium hydroxide monohydrate (2.01 g, 2.86 mmol, 2 eq) in water (10 ml) and the mixture is stirred at reflux overnight. The mixture is concentrated. The resulting white solid is taken up with a 1 N aqueous solution of hydrochloric acid and the pH adjusted to 4. The solid is then filtered, rinsed with distilled water (2×10 ml) and dried under vacuum, at 40° C., overnight, to afford 0.42 g of 2-[4-(3-chloropropoxy)phenyl]-4,5,6,7-tetrahydro-1,3-benzothiazole-5-carboxylic acid, enantiomer 1, x49.

Yield: 87%.

LC-MS (MH⁺): 352/354.

### 19.5 Synthesis of benzyl {2-[4-(3-chloropropoxy)phenyl]-4,5,6,7-tetrahydro-1,3-benzothiazol-5-yl}carbamate, enantiomer 1, x50

A solution of 2-[4-(3-chloropropoxy)phenyl]-4,5,6,7-tetrahydro-1,3-benzothiazole-5-carboxylic acid, enantiomer 1, x49 (0.42 g, 1.19 mmol, 1 eq), triethylamine (0.41 ml, 2.99 mmol, 2.5 eq) in toluene (10 ml) is treated with diphenylphosphoryl azide (0.38 ml, 1.79 mmol, 1.5 eq). The mixture is then stirred at 40° C. overnight. Benzyl alcohol (0.23 ml, 2.2 mmol, 1.9 eq) is then added and the mixture is stirred at 90° C. overnight. The organic layer is washed successively with a saturated solution of ammonium chloride (2×30 ml) and a saturated solution of sodium hydrogenocarbonate (2×30 ml), dried over magnesium sulfate and concentrated in vacuo. The residue is purified by chromatography over silicagel (gradient: dichloromethane/ethyl acetate from 10:0 to 8:2) to afford 0.47 g of benzyl {2-[4-(3-chloropropoxy)phenyl]-4,5,6,7-tetrahydro-1,3-benzothiazol-5-yl}carbamate, enantiomer 1, x50 as a yellow oil.

Yield: 86%.

LC-MS (MH⁺): 457.

### 19.6 Synthesis of benzyl (2-{4-[3-(2-methylpyrrolidin-1-yl)propoxy]phenyl}-4,5,6,7-tetrahydro-1,3-benzothiazol-5-yl)carbamate x51

A solution of benzyl {2-[4-(3-chloropropoxy)phenyl]-4,5,6,7-tetrahydro-1,3-benzothiazol-5-yl}carbamate, enantiomer 1, x50 (0.47 g, 1.03 mmol, 1 eq) and 2-methylpyrrolidine (0.26 ml, 2.5 mmol, 2.5 eq) in acetonitrile (10 ml) is stirred at 100° C. for 24 hours. The mixture is concentrated and the residue is purified by chromatography over silicagel (gradient dichloromethane/ethyl acetate from 9:1 to 10:0) to afford 0.22 g of benzyl (2-{4-[3-(2-methylpyrrolidin-1-yl)propoxy]phenyl}-4,5,6,7-tetrahydro-1,3-benzothiazol-5-yl)carbamate x51 as a yellow solid and as a mixture of diastereoisomers.

Yield: 48%.

LC-MS (MH⁺): 506.

### 19.7 Synthesis of 2-{4-[3-(2-methylpyrrolidin-1-yl)propoxy]phenyl}-4,5,6,7-tetrahydro-1,3-benzothiazol-5-amine x52

A solution of benzyl (2-{4-[3-(2-methylpyrrolidin-1-yl)propoxy]phenyl}-4,5,6,7-tetrahydro-1,3-benzothiazol-5-yl)carbamate x51 (0.25 g, 0.49 mmol, 1 eq) in 6 N hydrochloric acid (10 ml) is stirred at reflux overnight. The mixture is cooled down to 20° C. and water (15 ml) is added. The reaction mixture is washed with ethyl acetate (2×15 ml), brought to pH 12 with sodium hydroxide pellets and extracted with ethyl acetate (3×25 ml). The combined three last organic layers are washed with brine, dried over magnesium sulfate and concentrated in vacuo to afford 0.18 g of 2-{4-[3-(2-methylpyrrolidin-1-yl)propoxy]phenyl}-4,5,6,7-tetrahydro-1,3-benzothiazol-5-amine x52 as a mixture of diastereoisomers.

Yield: 97%.

LC-MS (MH⁺): 372.

### 19.8 Synthesis of N-(2-{4-[3-(2-methylpyrrolidin-1-yl)propoxy]phenyl}-4,5,6,7-tetrahydro-1,3-benzothiazol-5-yl)acetamide 55

To a solution of 2-{4-[3-(2-methylpyrrolidin-1-yl)propoxy]phenyl}-4,5,6,7-tetrahydro-1,3-benzothiazol-5-amine x52 (0.18 g, 0.52 mmol, 1 eq) and triethylamine (0.07 g, 0.72 mmol, 1.5 eq) in dichloromethane (10 ml) at 0° C. is added acetyl chloride (0.1 ml, 1.4 mmol, 2.9 eq). The mixture is stirred for 3 hours at room temperature. Dichloromethane is added (20 ml) and the organic layer is washed with water (15 ml) and with a saturated solution of ammonium chloride (15 ml), dried over magnesium sulfate and concentrated in vacuo. The residue is purified by chromatography over silicagel (gradient: dichloromethane/methanol/ammonia from 99:0.9:0.1 to 95:4.6:0.4) to afford 0.17 g of N-(2-{4-[3-(2-methylpyrrolidin-1-yl)propoxy]phenyl}-4,5,6,7-tetrahydro-1,3-benzothiazol-5-yl)acetamide 55 as a mixture of diastereoisomers.

Yield: 65%.

LC-MS (MH⁺): 414.

### Example 20

### Synthesis of 2-methyl-N-(2-{4-[3-(2-methylpyrrolidin-1-yl)propoxy]phenyl}-4,5,6,7-tetrahydro-1,3-benzothiazol-4-yl)pyrrolidine-1-carboxamide 54

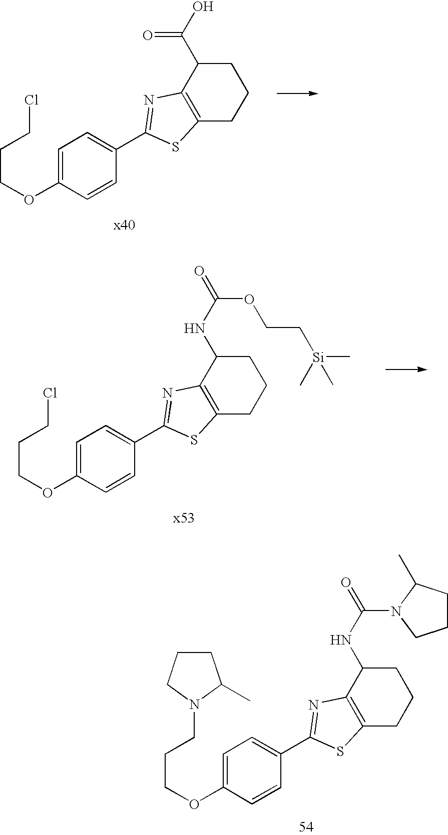

### 20.1 Synthesis of 2-(trimethylsilyl)ethyl {2-[4-(3-chloropropoxy)phenyl]-4,5,6,7-tetrahydro-1,3-benzothiazol-4-yl}carbamate x53

A solution of 2-[4-(3-chloropropoxy)phenyl]-4,5,6,7-tetrahydro-1,3-benzothiazole-4-carboxylic acid x40 (1.53 g, 4.35 mmol, 1 eq) and triethylamine (1.5 ml, 10.79 mmol, 2.5 eq) in toluene (40 ml) is treated with diphenylphosphoryl azide (1.6 ml, 6.95 mmol, 1.6 eq) and stirred at 40° C. overnight. 2-Trimethylsilanyl-ethanol (1.4 ml, 8.73 mmol, 2 eq) is then added and the mixture is stirred at 90° C. overnight. The organic layer is washed with a saturated solution of ammonium chloride (1×30 ml) and a saturated solution of sodium hydrogenocarbonate (1×30 ml), dried over magnesium sulfate and concentrated in vacuo. The residue is purified by chromatography over silicagel (gradient: dichloromethane/cyclohexane from 1:1 to 1:0) to afford 1.7 g of 2-(trimethylsilyl)ethyl {2-[4-(3-chloropropoxy)phenyl]-4,5,6,7-tetrahydro-1,3-benzothiazol-4-yl}carbamate x53.

Yield: 84%.

LC-MS (MH⁺): 467/469.

### 20.2 Synthesis of 2-methyl-N-(2-{4-[3-(2-methylpyrrolidin-1-yl)propoxy]phenyl}-4,5,6,7-tetrahydro-1,3-benzothiazol-4-yl)pyrrolidine-1-carboxamide 54

A solution of 1.7 g of 2-(trimethylsilyl)ethyl {2-[4-(3-chloropropoxy)phenyl]-4,5,6,7-tetrahydro-1,3-benzothiazol-4-yl}carbamate x53 (1.7 g, 3.64 mmol, 1 eq) and 2-methylpyrrolidine (0.74 g, 8.73 mmol, 2.4 eq) in acetonitrile (13 ml) is stirred at 120° C. for 2.5 hours. Ethyl acetate (150 ml) is added to the reaction mixture. The organic layer is washed with water (2×50 ml) and brine, dried over magnesium sulfate and concentrated in vacuo. A 1 M tetrahydrofuran solution of tetrabutylammonium fluoride (2.5 ml, 2.5 mmol, 2.8 eq) is added to the residue in tetrahydrofuran (10 ml). The reaction mixture is stirred at room temperature for 2 hours and concentrated in vacuo. A 1 M aqueous solution of hydrochloric acid (30 ml) is added. The aqueous layer is washed with ethyl acetate (2×15 ml), basified to pH 12 with hydroxide sodium (pellets) and extracted with ethyl acetate (3×20 ml). Combined organic layers are washed with brine, dried over magnesium sulfate and concentrated in vacuo. The residue is purified by chromatography over silicagel (dichloromethane/ethanol/ammonia 95:4.6:0.4) to afford 65 mg of 2-methyl-N-(2-{4-[3-(2-methylpyrrolidin-1-yl)propoxy]phenyl}-4,5,6,7-tetrahydro-1,3-benzothiazol-4-yl)pyrrolidine-1-carboxamide 54 as a yellow solid.

Yield: 3.1%.

LC-MS (MH⁺): 483.

### Example 21

### Synthesis of 4-acetyl-2-{4-[3-(2-methylpyrrolidin-1-yl)propoxy]phenyl}-4,5,6,7-tetrahydro[1,3]thiazolo[4,5-b]pyridine ethanedioate (2:1) 64.

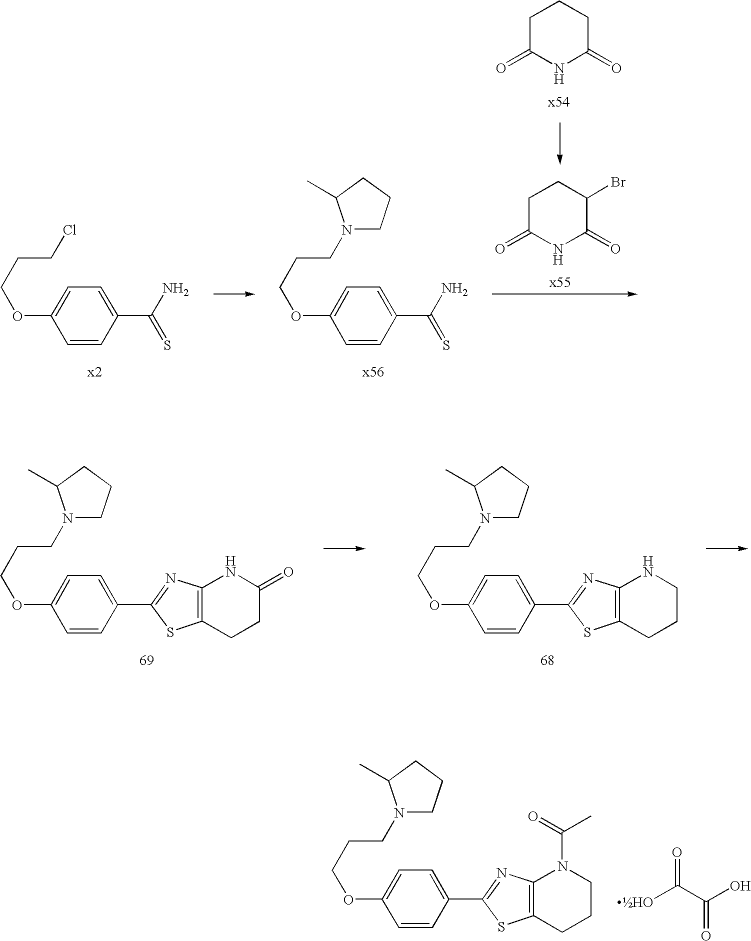

### 21.1 Synthesis of 3-bromopiperidine-2,6-dione x55

Bromine (9.1 ml, 177 mmol, 1 eq) is added to a solution of glutarimide x54 (20 g, 177 mmol, 1 eq) in 1,1,2-trichloroethane (60 ml) at room temperature. The mixture is stirred for 2 h at 110° C., then 1 h at room temperature. The mixture is evaporated to dryness and the residue is purified by chromatography over silicagel (dichloromethane) to afford 14.6 g of 3-bromopiperidine-2,6-dione x55 as white crystals.

Yield: 44%.

LC-MS (MH⁺): 193.

### 21.2 Synthesis of 4-[3-(2-methylpyrrolidin-1-yl)propoxy]benzenecarbothioamide x56

A mixture of 4-(3-chloropropoxy)benzenecarbothioamide x2 (0.5 g, 2.18 mmol, 1 eq), potassium carbonate (1.21 g, 8.73 mmol, 4 eq) and sodium iodide (0.1 g) in acetonitrile (10 ml) is heated at 90° C. for 1 h. The mixture is cooled to 20° C., 2-methylpyrrolidine (0.22 g, 2.62 mmol, 1.2 eq) is added and the mixture is heated at 90° C. for 20 h. The mixture is concentrated under reduced pressure and the residue is dissolved in dichloromethane. The resulting organic layer is washed with water, dried over magnesium sulfate and concentrated. The residue is purified by chromatography over silicagel (dichloromethane/methanol/ammonia 95:5:0.5) to afford 300 mg of 4-[3-(2-methylpyrrolidin-1-yl)propoxy]benzenecarbothioamide x56.

Yield: 50%.

LC-MS (MH⁺): 279.

### 21.3 Synthesis of 2-{4-[3-(2-methylpyrrolidin-1-yl)propoxy]phenyl}-6,7-dihydro[1,3]thiazolo[4,5-b]pyridin-5(4H)-one 69

Trifluoroacetic acid (0.8 ml, 10.4 mmol, 2 eq) is added to a mixture of 3-bromopiperidine-2,6-dione x55 (1.0 g, 5.2 mmol, 1 eq) and 4-[3-(2-methylpyrrolidin-1-yl)propoxy]benzenecarbothioamide x56 (1.45 g, 5.2 mmol, 1 eq) in dioxane (10 ml) and the reaction mixture is stirred overnight at 65° C. An additional amount of 3-bromopiperidine-2,6-dione x55 (0.3 g, 1.56 mmol) is added. The reaction mixture is stirred for an additional 24 h at 65° C., cooled to room temperature and evaporated to dryness. The residue is dissolved in dichloromethane (10 ml), water and an aqueous saturated solution of potassium carbonate are added. The organic layer is washed with brine, dried with sodium sulfate and evaporated in vacuo. The residue is purified by chromatography over silicagel (gradient: dichloromethane/methanol from 1:0 to 9:1) to afford 1.08 g of 2-{4-[3-(2-methylpyrrolidin-1-yl)propoxy]phenyl}-6,7-dihydro[1,3]thiazolo[4,5-b]pyridin-5(4H)-one 69 as a yellow solid.

Yield: 56%.

LC-MS (MH⁺): 372.

### 21.4 Synthesis of 2-{4-[3-(2-methylpyrrolidin-1-yl)propoxy]phenyl}-4,5,6,7-tetrahydro[1,3]thiazolo[4,5-b]pyridine 68

Boran-dimethyl sulfide complex (2 M in diethyl ether) (0.27 ml, 0.54 mmol, 2 eq) is added dropwise to a solution of 2-{4-[3-(2-methylpyrrolidin-1-yl)propoxy]phenyl}-6,7-dihydro[1,3]thiazolo[4,5-b]pyridin-5(4H)-one 69 (0.1 g, 0.27 mmol, 1 eq) in tetrahydrofuran (3 ml) in a stream of argon. The reaction mixture is stirred overnight at 20° C. Water (0.5 ml) and 5 M aqueous solution of sulfuric acid (0.1 ml) are added, the tetrahydrofuran is removed under reduced pressure, the remaining water solution is kept at 80° C. for 10 min and cooled at room temperature. The precipitate is filtered off and washed with water. Aqueous sodium hydroxide (10 N) is added to the filtrate until pH 14 and this aqueous layer is extracted with dichloromethane. The organic layer is dried over sodium sulfate and evaporated in vacuo. The residue is purified by chromatography over silicagel (gradient: dichloromethane/methanol from 100:0 to 89:11) to afford 0.05 g of 2-{4-[3-(2-methylpyrrolidin-1-yl)propoxy]phenyl}-4,5,6,7-tetrahydro[1,3]thiazolo[4,5-b]pyridine 68 as a yellow solid.

Yield: 52%.

LC-MS (MH⁺): 358.

### 21.5 Synthesis of 4-acetyl-2-{4-[3-(2-methylpyrrolidin-1-yl)propoxy]phenyl}-4,5,6,7-tetrahydro[1,3]thiazolo[4,5-b]pyridine ethanedioate (2:1) 64.

Triethylamine (0.35 ml, 2.52 mmol, 3 eq) and acetyl chloride (0.066 ml, 0.93 mmol, 1.25 eq) are added to a solution of 2-{4-[3-(2-methylpyrrolidin-1-yl)propoxy]phenyl}-4,5,6,7-tetrahydro[1,3]thiazolo[4,5-b]pyridine 68 (0.3 g, 0.84 mmol, 1 eq) in dichloromethane (10 ml) at room temperature under stirring and in a stream of argon. After 3 hours stirring, water (3 ml) and an aqueous saturated solution of potassium carbonate are added, and the aqueous layer is extracted with dichloromethane. The organic layer is washed with brine, dried over sodium sulfate and evaporated in vacuo. The residue is purified by chromatography over silicagel (gradient: dichloromethane/methanol from 10:0 to 9:1) to yield 0.25 g of 4-acetyl-2-{4-[3-(2-methylpyrrolidin-1-yl)propoxy]phenyl}-4,5,6,7-tetrahydro[1,3]thiazolo[4,5-b]pyridine as a yellow oil.

Yield: 74%.

LC-MS (MH⁺): 400.

This oil is dissolved in methanol (0.5 ml) and oxalic acid (1 M in water) (0.29 ml, 0.29 mmol, 1 eq) is added. The reaction mixture is evaporated to dryness and the residue is crystallized with diethyl ether to afford 4-acetyl-2-{4-[3-(2-methylpyrrolidin-1-yl)propoxy]phenyl}-4,5,6,7-tetrahydro[1,3]thiazolo[4,5-b]pyridine ethanedioate (2:1) 64 (0.245 g) as pale crystals.

Yield: 85%.

LC-MS (MH⁺): 400.

Compounds 62, 63 and 66 may be synthesized according to the same method.

### Example 22

### Synthesis of N-ethyl-2-{4-[3-(2-methylpyrrolidin-1-yl)propoxy]phenyl}-6,7-dihydro[1,3]thiazolo[4,5-b]pyridine-4(5H)-carboxamide L-(+)-tartrate (2:1) 65.

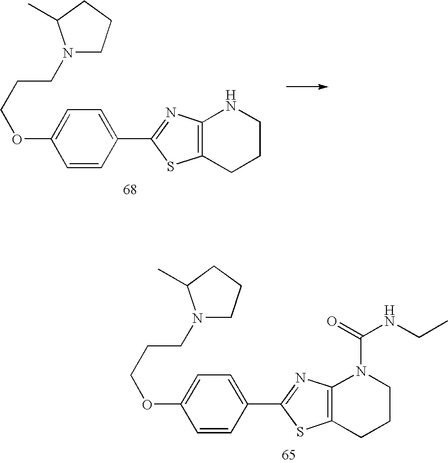

Ethyl isocyanate (0.13 ml, 1.68 mmol, 2 eq) is added to a solution of 2-{4-[3-(2-methylpyrrolidin-1-yl)propoxy]phenyl}-4,5,6,7-tetrahydro[1,3]thiazolo[4,5-b]pyridine 68 (0.3 g, 0.84 mmol, 1 eq) in dichloromethane (3 ml) at room temperature under stirring in a stream of argon. The reaction mixture is stirred at room temperature overnight. Water and an aqueous saturated solution of potassium carbonate are added and the layers are separated. The organic layer is washed with brine, dried with sodium sulfate and evaporated in vacuo. The residue is purified by chromatography over silicagel (gradient: dichloromethane/methanol from 10:0 to 9:1) to afford N-ethyl-2-{4-[3-(2-methylpyrrolidin-1-yl)propoxy]phenyl}-6,7-dihydro[1,3]thiazolo[4,5-b]pyridine-4(5H)-carboxamide as a yellow oil.

Yield: 78%.

LC-MS (MH⁺): 429.

N-ethyl-2-{4-[3-(2-methylpyrrolidin-1-yl)propoxy]phenyl}-6,7-dihydro[1,3]thiazolo[4,5-b]pyridine-4(5H)-carboxamide (0.248 g, 0.58 mmol) is dissolved in methanol (0.5 ml). Tartaric acid (1 M in water, 0.29 ml, 0.29 mmol) is added and the reaction mixture is evaporated to dryness. The residue is crystallized with diethyl ether to afford 0.215 g of N-ethyl-2-{4-[3-(2-methylpyrrolidin-1-yl)propoxy]phenyl}-6,7-dihydro[1,3]thiazolo[4,5-b]pyridine-4(5H)-carboxamide L-(+)-tartrate (2:1) 65 as pale crystals.

Yield: 74%.

LC-MS (MH⁺): 429.

### Example 23

### Synthesis of 2-{4-[3-(2-methylpyrrolidin-1-yl)propoxy]phenyl}-4-(morpholin-4-ylsulfonyl)-4,5,6,7-tetrahydro[1,3]thiazolo[4,5-b]pyridine ethanedioate (2:1) 67.

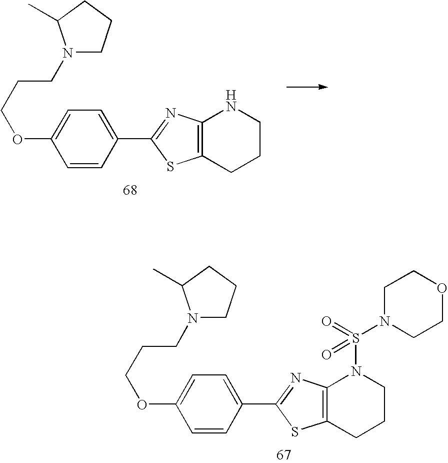

Triethylamine (0.35 ml, 2.52 mmol, 3 eq) and 4-morpholinesulfonyl chloride (0.3 g, 1.68 mmol, 2 eq) are added to a solution of 2-{4-[3-(2-methylpyrrolidin-1-yl)propoxy]phenyl}-4,5,6,7-tetrahydro[1,3]thiazolo[4,5-b]pyridine 68 (0.3 g, 0.84 mmol) in dichloromethane (3 ml) at room temperature under stirring in a stream of argon. The reaction mixture is stirred at room temperature overnight, then 4-morpholinesulfonyl chloride (0.3 g, 1.68 mmol, 2 eq) is added and the reaction mixture is stirred at room temperature for an additional 6 h. Water (3 ml) and an aqueous saturated solution of potassium carbonate are added. The layers are separated and the aqueous layer is extracted with dichloromethane. The combined organic layers are washed with brine, dried over sodium sulfate and evaporated in vacuo. The residue is purified by chromatography over silicagel (gradient: dichloromethane/methanol from 10:0 to 9:1) to afford 0.085 g of 2-{4-[3-(2-methylpyrrolidin-1-yl)propoxy]phenyl}-4-(morpholin-4-ylsulfonyl)-4,5,6,7-tetrahydro[1,3]thiazolo[4,5-b]pyridine as a yellow oil.

Yield: 20%.

LC-MS (MH⁺): 507.

2-{4-[3-(2-methylpyrrolidin-1-yl)propoxy]phenyl}-4-(morpholin-4-ylsulfonyl)-4,5,6,7-tetrahydro[1,3]thiazolo[4,5-b]pyridine (0.08 g, 0.158 mmol) is dissolved in methanol (0.5 ml), oxalic acid (1 M in water) (0.079 ml, 0.079 mmol) is added, and the reaction mixture is evaporated to dryness. The residue is crystallized with diethyl ether to yield 0.057 g of 2-{4-[3-(2-methylpyrrolidin-1-yl)propoxy]phenyl}-4-(morpholin-4-ylsulfonyl)-4,5,6,7-tetrahydro[1,3]thiazolo[4,5-b]pyridine ethanedioate (2:1) 67 as pale crystals.

### Example 24

### Synthesis of 5-methyl-2-{4-[3-(2-methylpyrrolidin-1-yl)propoxy]phenyl}-4,5,6,7-tetrahydro[1,3]thiazolo[4,5-c]pyridine 70

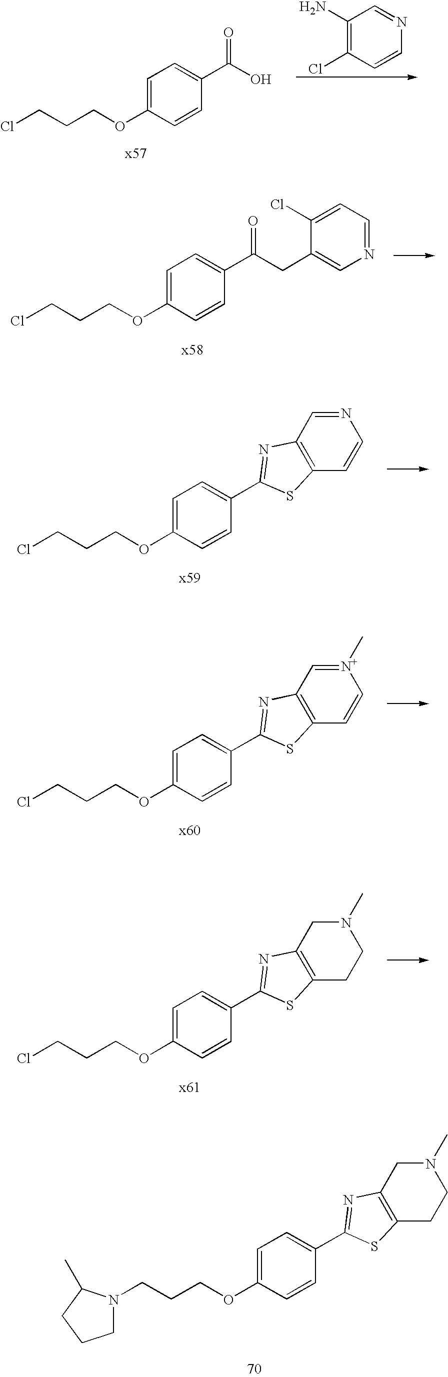

### 24.1 Synthesis of 4-(3-chloropropoxy)-N-(4-chloropyridin-3-yl)benzamide x58

A cold solution (0° C.) of 4-(3-chloropropoxy)benzoic acid x57 (9.18 g, 42.78 mmol, 1 eq) in dichloromethane (200 ml) is treated with oxalyl chloride (5.0 ml, 47.06 mmol, 1.1 eq) and N,N-dimethylformamide (0.5 ml). The mixture is left to warm up to 20° C. and stirred until gas evolution has ceased. The mixture is then concentrated under reduced pressure and the residue is dissolved in tetrahydrofuran (150 ml). A suspension of sodium hydride (60% dispersion in mineral oil, 2.26 g, 94.12 mmol, 2.2 eq) in tetrahydrofuran (200 ml) is treated with a solution of 3-amino-4-chloropyridine (5.5 g, 42.78 mmol, 1 eq) in tetrahydrofuran (100 ml). After 30 minutes stirring at 20° C., the resulting solution is treated dropwise with the previously obtained acyl chloride solution. The mixture is stirred for 24 h at 20° C. and 8 h at reflux. After cooling to room temperature, water (1 ml) is added to the mixture, which is left under stirring for 1 h. The mixture is finally concentrated under reduced pressure. The residue is dissolved in dichloromethane and the organic layer is washed with water, dried over magnesium sulfate and concentrated under reduced pressure. The crude product is purified by chromatography over silicagel (dichloromethane/methanol/ammonia 99:1:0.1) to afford 5.3 g of 4-(3-chloropropoxy)-N-(4-chloropyridin-3-yl)benzamide x58.

Yield: 38%

LC-MS (MH⁺): 325/327

### 24.2 Synthesis of 2-[4-(3-chloropropoxy)phenyl][1,3]thiazolo[4,5-c]pyridine x59

A solution of 4-(3-chloropropoxy)-N-(4-chloropyridin-3-yl)benzamide x58 (9.6 g, 29.52 mmol, 1 eq) in toluene (500 ml) is treated with 2,4-bis(4-methoxyphenyl)-2,4-dithioxo-1,3,2,4-dithiadiphosphetane (Lawesson's reagent, 8.36 g, 20.66 mmol, 0.7 eq) and the mixture is stirred at reflux for 4 h. After cooling down to room temperature, water (200 ml) is added. The aqueous phase is extracted with dichloromethane (2×300 ml). The combined organic layers are washed with an aqueous saturated solution of sodium hydrogenocarbonate, dried over magnesium sulfate and concentrated under reduced pressure to afford 1.2 g of 2-[4-(3-chloropropoxy)phenyl][1,3]thiazolo[4,5-c]pyridine x59 which is used in the next step without any further purification.

Yield: 51%.

LC-MS (MH⁺): 305/307.

### 24.3 Synthesis of 2-[4-(3-chloropropoxy)phenyl]-5-methyl[1,3]thiazolo[4,5-c]pyridin-5-ium x60

A solution of 2-[4-(3-chloropropoxy)phenyl][1,3]thiazolo[4,5-c]pyridine x59 (1.2 g, 3.9 mmol, 1 eq) in N,N-dimethylformamide (10 ml) is treated with methyl idodide (3 ml, 48 mmol, 12 eq). The mixture is stirred at 60° C. for 4 h. The solvent is then removed under reduced pressure and the residue is dissolved in ethyl acetate (10 ml). The resulting suspension is filtered off and the solid is washed with ethylacetate to yield 2 g of 2-[4-(3-chloropropoxy)phenyl]-5-methyl[1,3]thiazolo[4,5-c]pyridin-5-ium x60.

Yield: 100%.

LC-MS (MH⁺): 319/321.

### 24.4 Synthesis of 2-[4-(3-chloropropoxy)phenyl]-5-methyl-4,5,6,7-tetrahydro[1,3]thiazolo[4,5-c]pyridine x61

A solution of 2-[4-(3-chloropropoxy)phenyl]-5-methyl[1,3]thiazolo[4,5-c]pyridin-5-ium x60 (2 g, 4.48 mmol, 1 eq) in methanol (100 ml) is treated with sodium borohydride (0.68 g, 17.91 mmol, 4 eq) in portions. After 1 h stirring at 20° C., the mixture is concentrated under reduced pressure and the residue is taken up with ethyl acetate (50 ml). The organic layer is washed with water (20 ml), dried over magnesium sulfate and concentrated under reduced pressure. Purification by chromatography over silicagel (dichloromethane/methanol/ammonia 90:10:1) affords 330 mg of 2-[4-(3-chloropropoxy)phenyl]-5-methyl-4,5,6,7-tetrahydro[1,3]thiazolo[4,5-c]pyridine x61.

Yield: 26%.

LC-MS (MH⁺): 323/325.

### 24.5 Synthesis of 5-methyl-2-{4-[3-(2-methylpyrrolidin-1-yl)propoxy]phenyl}-4,5,6,7-tetrahydro[1,3]thiazolo[4,5-c]pyridine 70

A mixture of 2-[4-(3-chloropropoxy)phenyl]-5-methyl-4,5,6,7-tetrahydro[1,3]thiazolo[4,5-c]pyridine x61 (0.11 g, 0.34 mmol, 1 eq) in acetonitrile (10 ml), potassium carbonate (0.14 g, 1.02 mmol, 3 eq) and sodium iodide (10 mg) is heated at 80° C. in a sealed tube for 1 h. After cooling to room temperature, 2-methylpyrrolidine (40 mg, 0.51 mmol, 1.5 eq) is added. The mixture is heated at 80° C. for 24 h, then concentrated under reduced pressure and the residue is taken up with dichloromethane (50 ml). The organic layer is washed with water (20 ml), dried over magnesium sulfate and concentrated under reduced pressure. Purification by chromatography over silicagel (gradient: dichloromethane/methanol/ammonia from 97.5:2.5:0.25 to 95:5:0.5) affords 90 mg of 55-methyl-2-{4-[3-(2-methylpyrrolidin-1-yl)propoxy]phenyl}-4,5,6,7-tetrahydro[1,3]thiazolo[4,5-c]pyridine 70.

Yield: 71%.

LC-MS (MH⁺): 372.

### Example 25

### Synthesis of 5-acetyl-2-(4-{3-[(2R)-2-methylpyrrolidin-1-yl]propoxy}phenyl)-4,5,6,7-tetrahydro[1,3]thiazolo[4,5-c]pyridine 71

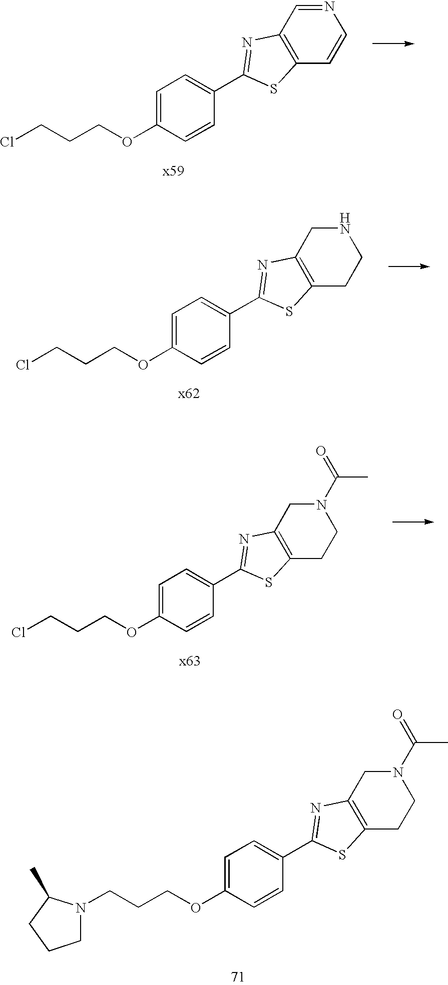

### 25.1 Synthesis of 2-[4-(3-chloro-propoxy)-phenyl]-4,5,6,7-tetrahydro-thiazolo[4,5-c]pyridine x62

A cold (−50° C.) solution of 2-[4-(3-chloropropoxy)phenyl][1,3]thiazolo[4,5-c]pyridine x59 (2.8 g, 9.21 mmol, 1 eq) in tetrahydrofuran (200 ml) is treated with acetyl chloride (0.72 ml, 10.13 mmol, 1.1 eq) and the mixture is stirred 15 minutes at −40° C., before adding sodium borohydride (1.39 g, 36.84 mmol, 4 eq). The mixture is left to warm to room temperature, ethanol (40 ml) is added and the mixture is stirred for 3 h at 20° C. Water (50 ml) is added and the mixture is extracted with ethyl acetate (2×50 ml). The organic layer is dried over magnesium sulfate and concentrated under reduced pressure. Chromatography over silicagel (gradient: dichloromethane/methanol/ammoniac from 98:2:0.2 to 97:2.7:0.3) affords 710 mg of 2-[4-(3-chloro-propoxy)-phenyl]-4,5,6,7-tetrahydro-thiazolo[4,5-c]pyridine x62.

Yield: 24%.

LC-MS (MH⁺): 309/311.

### 25.2 Synthesis of 5-acetyl-2-[4-(3-chloropropoxy)phenyl]-4,5,6,7-tetrahydro[1,3]thiazolo[4,5-c]pyridine x63

This compound is synthesized as described in example 4.1, starting from 2-[4-(3-chloro-propoxy)-phenyl]-4,5,6,7-tetrahydro-thiazolo[4,5-c]pyridine x62.

Yield: 75%.

LC-MS (MH⁺): 351/353.

### 25.3 Synthesis of 5-acetyl-2-{4-(3-[(2R)-2-methylpyrrolidin-1-yl]propoxy}phenyl)-4,5,6,7-tetrahydro[1,3]thiazolo[4,5-c]pyridine 71

This compound is synthesized as described in example 4.2, starting from 5-acetyl-2-[4-(3-chloropropoxy)phenyl]-4,5,6,7-tetrahydro[1,3]thiazolo[4,5-c]pyridine x63.

Yield: 32%.

LC-MS (MH⁺): 400.

### Example 26

### Synthesis of 5-(methoxyacetyl)-2-(4-{3-[(2R)-2-methylpyrrolidin-1-yl]propoxy}phenyl)-4,5,6,7-tetrahydro[1,3]thiazolo[4,5-c]pyridine 73

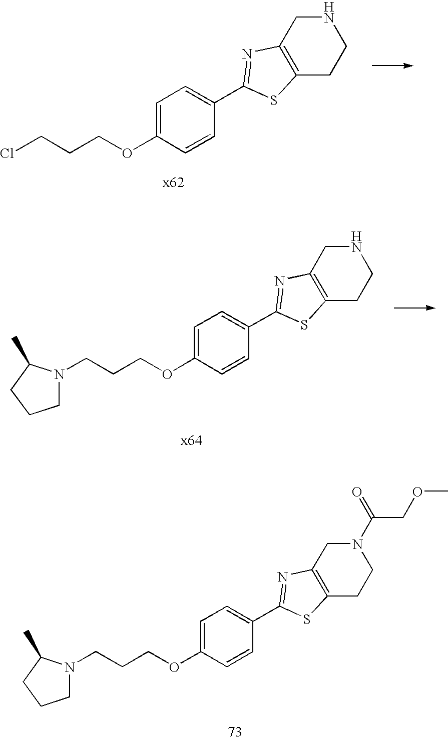

### 26.1 Synthesis of 2-(4-{3-[(2R)-2-methylpyrrolidin-1-yl]propoxy}phenyl)-4,5,6,7-tetrahydro[1,3]thiazolo[4,5-c]pyridine x64

This compound is synthesized as described in example 1.3, starting from 2-[4-(3-chloro-propoxy)-phenyl]-4,5,6,7-tetrahydro-thiazolo[4,5-c]pyridine x62 and (2R)-2-methylpyrrolidine.

Yield: 39%.

LC-MS (MH⁺): 429.

### 26.2 Synthesis of 5-(methoxyacetyl)-2-(4-{3-[(2R)-2-methylpyrrolidin-1-yl]propoxy}phenyl)-4,5,6,7-tetrahydro[1,3]thiazolo[4,5-c]pyridine 73

This compound is synthesized as described in example 3, starting from 2-(4-{3-[(2R)-2-methylpyrrolidin-1-yl]propoxy}phenyl)-4,5,6,7-tetrahydro[1,3]thiazolo[4,5-c]pyridine x64.

Yield: 42%.

LC-MS (MH⁺): 430.

### Example 27

### Synthesis of N-ethyl-2-(4-{3-[(2R)-2-methylpyrrolidin-1-yl]propoxy}phenyl)-6,7-dihydro[1,3]thiazolo[4,5-c]pyridine-5(4H)-carboxamide 72

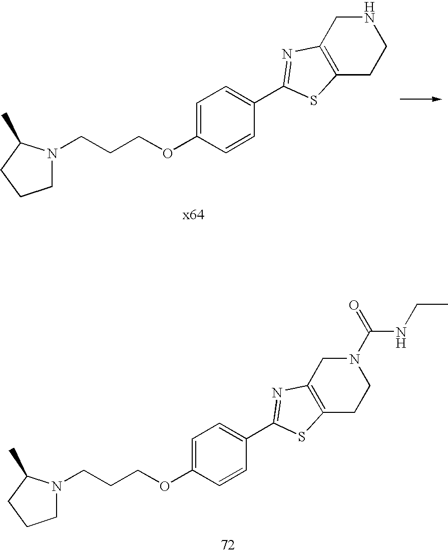

This compound is synthesized as described in example 7, starting from 2-(4-{3-[(2R)-2-methylpyrrolidin-1-yl]propoxy}phenyl)-4,5,6,7-tetrahydro[1,3]thiazolo[4,5-c]pyridine.

Yield: 39%.

LC-MS (MH⁺): 429.

### Example 28

### Synthesis of 5-methyl-2-(4-{3-[(2R)-2-methylpyrrolidin-1-yl]propoxy}phenyl)-4,5,6,7-tetrahydro[1,3]oxazolo[4,5-c]pyridine dihydrochloride 74

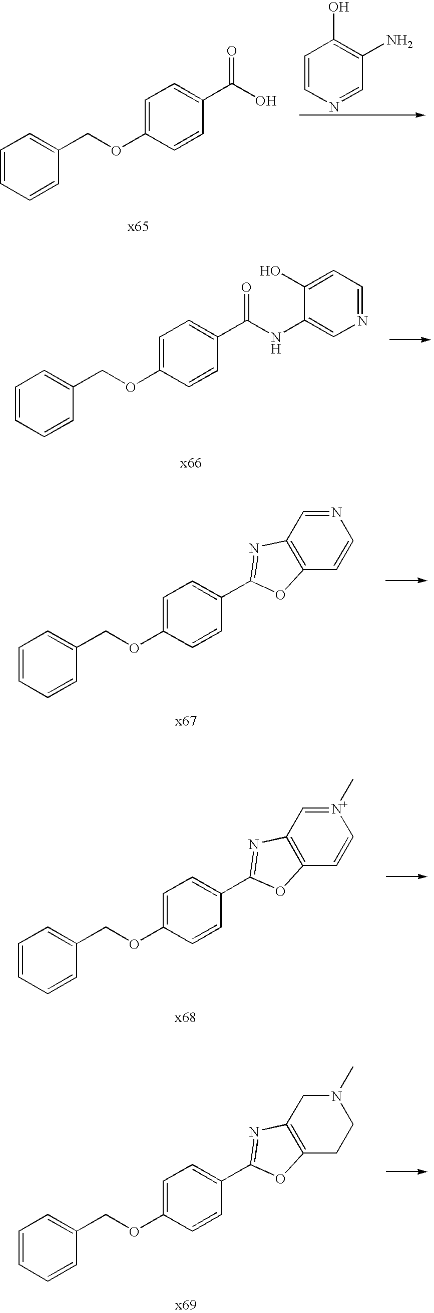

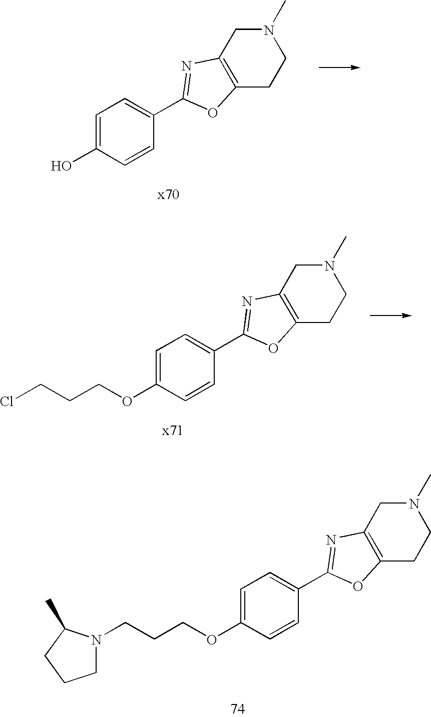

### 28.1 Synthesis of 4-(benzyloxy)-N-(4-hydroxypyridin-3-yl)benzamide x66

A suspension of 4-(benzyloxy)benzoic acid x65 (10.0 g, 43.81 mmol, 1 eq) and N,N-dimethylformamide (0.5 ml) in dichloromethane at 0° C. is treated with oxalyl chloride (5.18 ml, 48.19 mmol, 1.1 eq). The mixture is left to warm up to room temperature. When gas evolution has stopped, half of the solvent is removed under reduced pressure, and the solution is added dropwise to a solution of 3-aminopyridin-4-ol (4.82 g, 43.81 mmol, 1 eq) and triethylamine (12.15 ml, 87.62 mmol, 2 eq) in dichloromethane (300 ml). The mixture is stirred at 20° C. for 24 h and water (200 ml) is added. The aqueous phase is extracted with a 9:1 mixture of dichloromethane and methanol (2×300 ml). The combined organic layers are dried over magnesium sulfate and concentrated under reduced pressure. The residue is triturated with ethyl acetate (50 ml) and the resulting suspension is filtered off. The solid is dried at 40° C. in vacuo to yield 10 g of 4-(benzyloxy)-N-(4-hydroxypyridin-3-yl)benzamide x66.

Yield: 71%.

LC-MS (MH⁺): 321.

### 28.2 Synthesis of 2-[4-(benzyloxy)phenyl][1,3]oxazolo[4,5-c]pyridine x67

A solution of hexachloroethane (16.81 g, 71.02 mmol, 2.5 eq) in dry dichloromethane (300 ml) is treated with triphenylphosphine (22.35 g, 85.22 mmol, 3 eq) and triethylamine (31.68 ml, 227.25 mmol, 8 eq). After 10 minutes stirring at 20° C., 4-(benzyloxy)-N-(4-hydroxypyridin-3-yl)benzamide x66 (9.1 g, 28.41 mmol, 1 eq) is added in several portions. The suspension is stirred overnight at 20° C. and filtered. This solid is triturated with a 1 M aqueous solution of hydrogen chloride (50 ml) and this suspension is filtered. The solid is rinsed with diethyl ether and dried at 40° C. in vacuo to yield 7.9 g of 2-[4-(benzyloxy)phenyl][1,3]oxazolo[4,5-c]pyridine x67.

Yield: 92%.

LC-MS (MH⁺): 303.

### 28.3 Synthesis of 2-[4-(benzyloxy)phenyl]-5-methyl[1,3]oxazolo[4,5-c]pyridin-5-ium x68

A solution of 2-[4-(benzyloxy)phenyl][1,3]oxazolo[4,5-c]pyridine x67 (0.5 g, 1.6 mmol, 1 eq) in N,N-dimethylformamide (10 ml) is treated with methyl iodide (1 ml, 16 mmol, 10 eq) and stirred at 20° C. for 48 h. The mixture is concentrated under reduced pressure and the residue is triturated with ethyl acetate (10 ml). The suspension is filtered and the solid is dried at 40° C. in vacuo to afford 700 mg of 2-[4-(benzyloxy)phenyl]-5-methyl[1,3]oxazolo[4,5-c]pyridin-5-ium x68.

Yield: 100%.

LC-MS (MH⁺): 317.

### 28.4 Synthesis of 2-[4-(benzyloxy)phenyl]-5-methyl-4,5,6,7-tetrahydro[1,3]oxazolo[4,5-c]pyridine x69

A suspension of 2-[4-(benzyloxy)phenyl]-5-methyl[1,3]oxazolo[4,5-c]pyridin-5-ium x68 (0.7 g, 1.5 mmol, 1 eq) in ethanol (20 ml) is treated with sodium borohydride (0.5 g, 13.0 mmol, 8 eq) in portions and the mixture is stirred at 20° C. for 24 h. Water (1 ml) is added and the mixture is concentrated under reduced pressure. The residue is taken up with a 1:1 mixture of dichloromethane and water (40 ml). The organic layer is dried over magnesium sulfate and concentrated under reduced pressure. Chromatography over silicagel (dichloromethane/methanol/ammonia: 97:3:0.3) affords 180 mg of 2-[4-(benzyloxy)phenyl]-5-methyl-4,5,6,7-tetrahydro[1,3]oxazolo[4,5-c]pyridine x69.

Yield: 36%.

LC-MS (MH⁺): 321.

### 28.5 Synthesis of 4-(5-methyl-4,5,6,7-tetrahydro[1,3]oxazolo[4,5-c]pyridin-2-yl)phenol x70

A solution of 2-[4-(benzyloxy)phenyl]-5-methyl-4,5,6,7-tetrahydro[1,3]oxazolo[4,5-c]pyridine x69 (0.16 g, 0.5 mmol, 1 eq) in dichloromethane (4 ml) is treated dropwise with a 1 M solution of phosphorus tribromide in dichloromethane (2 ml, 2.0 mmol, 4 eq). The mixture is stirred 3 h at room temperature and water (50 ml) is added. The aqueous layer is brought to pH 11 with sodium hydrogenocarbonate. The resulting suspension is filtered and dried at 40° C. in vacuo to afford 70 mg of 4-(5-methyl-4,5,6,7-tetrahydro[1,3]oxazolo[4,5-c]pyridin-2-yl)phenol x70.

Yield: 60%.

LC-MS (MH⁺): 231.

### 28.6 Synthesis of 2-[4-(3-chloropropoxy)phenyl]-5-methyl-4,5,6,7-tetrahydro[1,3]oxazolo[4,5-c]pyridine x71

A solution of 4-(5-methyl-4,5,6,7-tetrahydro[1,3]oxazolo[4,5-c]pyridin-2-yl)phenol x70 (0.1 g, 0.43 mmol, 1 eq) in 2-butanone (20 ml) is treated with potassium carbonate (0.18 g, 1.27 mmol, 3 eq) and 3-chloro-1-bromopropane (50 μl, 0.52 ml, 1.2 eq). The mixture is heated at reflux for 4 h. After concentration under reduced pressure, the residue is taken up with a 1:1 mixture of dichloromethane and water (40 ml). The organic layer is dried over magnesium sulfate and concentrated under reduced pressure to afford 130 mg of 2-[4-(3-chloropropoxy)phenyl]-5-methyl-4,5,6,7-tetrahydro[1,3]oxazolo[4,5-c]pyridine x71.

Yield: 100%.

LC-MS (MH⁺): 307/309.

### 28.7 Synthesis of 5-methyl-2-(4-{3-[(2R)-2-methylpyrrolidin-1-yl]propoxy}phenyl)-4,5,6,7-tetrahydro[1,3]oxazolo[4,5-c]pyridine dihydrochloride 74

A suspension of 2-[4-(3-chloropropoxy)phenyl]-5-methyl-4,5,6,7-tetrahydro[1,3]oxazolo[4,5-c]pyridine x71 (0.13 g, 0.42 mmol, 1 eq), potassium carbonate (0.18 g, 1.27 mmol, 3 eq) and sodium iodide (10 mg) in acetonitrile (4 ml) is heated at 80° C. in a sealed tube for 1 h. After cooling to room temperature, (2R)-2-methylpyrrolidine (50 μg, 0.55 mmol, 1.3 eq) is added and the mixture is stirred at 80° C. for a further 2 days. The mixture is concentrated under reduced pressure and the residue is taken up with dichloromethane (20 ml) and water (10 ml). The organic layer is dried over magnesium sulfate and concentrated under reduced pressure. Chromatography over silicagel (dichloromethane/methanol/ammonia 95:5:0.5) affords 30 mg of pure 5-methyl-2-(4-{3-[(2R)-2-methylpyrrolidin-1-yl]propoxy}phenyl)-4,5,6,7-tetrahydro[1,3]oxazolo[4,5-c]pyridine, which is dissolved in diethyl ether (3 ml) and treated with a 2 M aqueous solution of hydrogen chloride solution in diethyl ether (1 ml). The suspension is filtered, the solid is washed with diethyl ether (2 ml) and dried at 40° C. in vacuo to afford 30 mg of 5-methyl-2-(4-{3-[(2R)-2-methylpyrrolidin-1-yl]propoxy}phenyl)-4,5,6,7-tetrahydro[1,3]oxazolo[4,5-c]pyridine dihydrochloride 74.

Yield: 20%.

LC-MS (MH⁺): 356.

### Example 29

### Synthesis of 2-{4-[3-(2-methylpyrrolidin-1-yl)propoxy]phenyl}-5-(morpholin-4-ylcarbonyl)-4,5,6,7-tetrahydro[1,3]thiazolo[5,4-c]pyridine 76

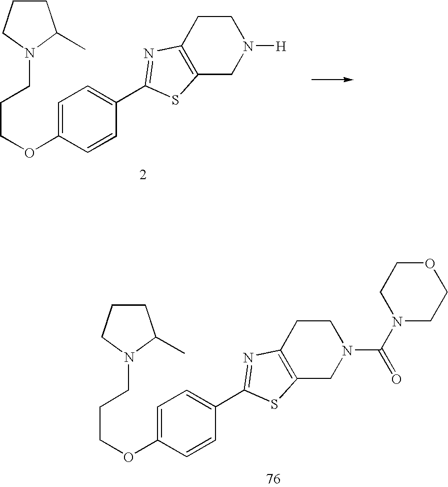

Triphosgene (0.097 g, 0.33 mmol, 0.37 eq) is added to a solution of 2-{4-[3-(2-methylpyrrolidin-1-yl)propoxy]phenyl}-4,5,6,7-tetrahydro[1,3]thiazolo[5,4-c]pyridine 2 (0.317 g, 0.89 mmol, 1 eq) in dichloromethane (10 ml) at 0° C. The mixture is stirred at room temperature for 1 hour. Morpholine (0.078 ml, 0.89 mmol, 1 eq) and triethylamine (0.124 ml, 0.89 ml, 1 eq) are added at 0° C. and the mixture is stirred overnight at room temperature. An aqueous saturated solution of sodium hydrogenocarbonate is added and the aqueous layer is extracted 3 times with dichloromethane. The combined organic layers are dried over magnesium sulfate and concentrated under reduced pressure. Purification over silicagel (dichloromethane/methanol/ammonia 97:3:0.3) yields 204 mg of 2-{4-[3-(2-methylpyrrolidin-1-yl)propoxy]phenyl}-5-(morpholin-4-ylcarbonyl)-4,5,6,7-tetrahydro[1,3]thiazolo[5,4-c]pyridine 76.

Yield: 48%.

LC-MS (MH⁺): 471.

Compounds 75, 77 and 78 may be prepared according to the same method.

### Example 30

### Synthesis of 5-(trans-3-fluorocyclobutyl)-2-{4-[3-(2-methylpyrrolidin-1-yl)propoxy]phenyl}-4,5,6,7-tetrahydro[1,3]thiazolo[5,4-c]pyridine 88

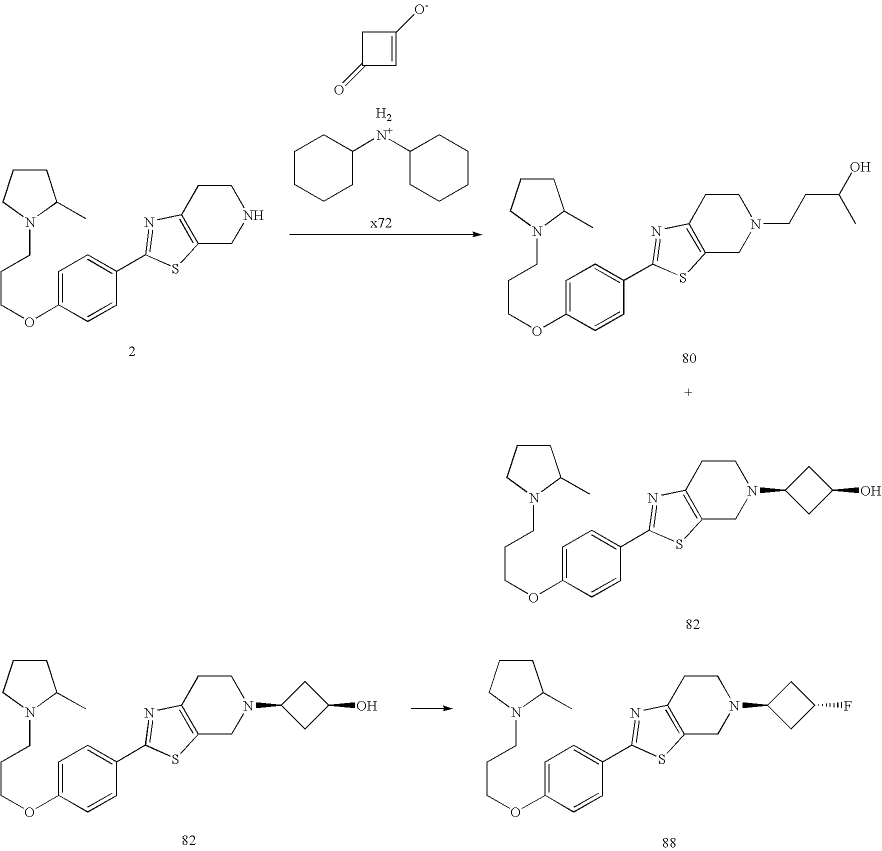

### 30.1 Synthesis of cis-3-[2-{4-[3-(2-methylpyrrolidin-1-yl)propoxy]phenyl}-6,7-dihydro[1,3]thiazolo[5,4-c]pyridin-5(4H)-yl]cyclobutanol 82

A solution of 2-{4-[3-(2-methylpyrrolidin-1-yl)propoxy]phenyl}-4,5,6,7-tetrahydro[1,3]thiazolo[5,4-c]pyridine 2 (0.5 g, 1.4 mmol, 1 eq) in a 98:2 mixture of methanol and acetic acid (50 ml) is treated with N-cyclohexylcyclohexanaminium 3-oxocyclobut-1-en-1-oxalate x72 (0.74 g, 2.8 mmol, 2 eq) and with powdered 4 Å molecular sieves (2 g). The mixture is stirred for 2 hours at room temperature. Sodium cyanoborohydride (0.26 g, 4.2 mmol, 3 eq) is added and the stirring is continued for 1 hour. After filtration on celite and evaporation to dryness, dichloromethane and water are added to the residue. The organic layer is discarded, the aqueous layer is brought to pH 12 with a 3 N aqueous solution of sodium hydroxyde and extracted with dichloromethane. The organic layer is concentrated under reduced pressure. Purification over silicagel (dichloromethane/methanol/ammonia 95:4.5:0.5) affords 15 mg of 4-[2-{4-[3-(2-methylpyrrolidin-1-yl)propoxy]phenyl}-6,7-dihydro[1,3]thiazolo[5,4-c]pyridin-5(4H)-yl]butan-2-ol 80 (yield: 7%, LC-MS (MH⁺): 430) and 270 mg of cis-3-[2-{4-[3-(2-methylpyrrolidin-1-yl)propoxy]phenyl}-6,7-dihydro[1,3]thiazolo[5,4-c]pyridin-5(4H)-yl]cyclobutanol 82 (yield: 47%, LC-MS (MH⁺): 428).

### 30.2 Synthesis of 5-(trans-3-fluorocyclobutyl)-2-{4-[3-(2-methylpyrrolidin-1-yl)propoxy]phenyl}-4,5,6,7-tetrahydro[1,3]thiazolo[5,4-c]pyridine 88

A solution of diethylaminosulfur trifluoride (0.05 ml, 2.3 mmol, 4 eq) in dichloromethane is treated at −50° C. with cis-3-[2-{4-[3-(2-methylpyrrolidin-1-yl)propoxy]phenyl}-6,7-dihydro[1,3]thiazolo[5,4-c]pyridin-5(4H)-yl]cyclobutanol 82 (0.25 g, 0.58 mmol, 1 eq). The mixture is left to warm up to 20° C. and water (20 ml) is added. The aqueous phase is brought to pH 12 with sodium hydrogenocarbonate and extracted with dichloromethane (2×20 ml). The organic layer is dried over magnesium sulfate and concentrated under reduced pressure. The crude product is purified by chromatography over silicagel (dichloromethane/methanol/ammonia 95:5:0.5) to afford 110 mg of 5-(trans-3-fluorocyclobutyl)-2-{4-[3-(2-methylpyrrolidin-1-yl)propoxy]phenyl}-4,5,6,7-tetrahydro[1,3]thiazolo[5,4-c]pyridine 88.

Yield: 36%.

LC-MS (MH⁺): 430.

### Example 31

### Synthesis of 2-[2-{4-[3-(2-methylpyrrolidin-1-yl)propoxy]phenyl}-6,7-dihydro[1,3]thiazolo[5,4-c]pyridin-5(4H)-yl]ethanol 85

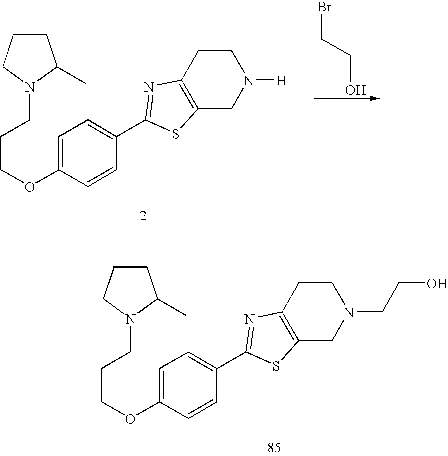

A suspension of sodium hydride (50 mg, 1.34 mmol, 1.2 eq) in tetrahydrofuran (5 ml) is treated with 2-{4-[3-(2-methylpyrrolidin-1-yl)propoxy]phenyl}-4,5,6,7-tetrahydro[1,3]thiazolo[5,4-c]pyridine 2 (0.4 g, 1.12 mmol, 1 eq) and the mixture is stirred 30 minutes at room temperature. 2-Bromoethanol (0.28 g, 2.24 mmol, 2 eq) is added and the mixture is stirred two hours at 80° C. After cooling, dichloromethane and water are added. The organic layer is dried over magnesium sulfate and concentrated to dryness. The crude product is purified by chromatography over silicagel (dichloromethane/methanol/ammonia 95:4.5:0.5) to afford 160 mg of 2-[2-{4-[3-(2-methylpyrrolidin-1-yl)propoxy]phenyl}-6,7-dihydro[1,3]thiazolo[5,4-c]pyridin-5(4H)-yl]ethanol 85.

Yield: 38%.

LC-MS (MH⁺): 402.

### Example 32

### Synthesis of potassium 3-[2-{4-[3-(2-methylpyrrolidin-1-yl)propoxy]phenyl}-6,7-dihydro[1,3]thiazolo[5,4-c]pyridin-5(4H)-yl]-3-oxopropanoate 86

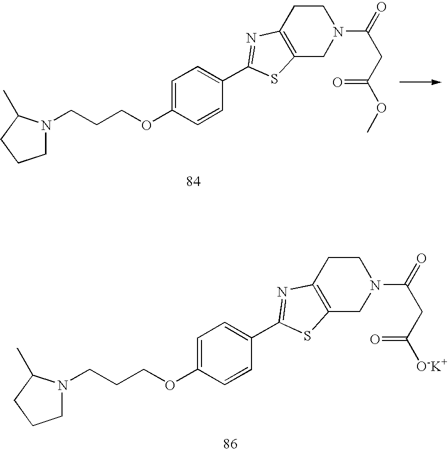

A solution of methyl 3-[2-{4-[3-(2-methylpyrrolidin-1-yl)propoxy]phenyl}-6,7-dihydro[1,3]thiazolo[5,4-c]pyridin-5(4H)-yl]-3-oxopropanoate 84 (0.6 g, 1.31 mmol, 1 eq) in tetrahydrofuran (10 ml) is treated with a 1M aqueous solution of potassium hydroxide (1.32 ml, 1.32 mmol, 1 eq) and the mixture is stirred at room temperature for 2 hours. The tetrahydrofuran is removed under reduced pressure and the resulting aqueous solution is filtered over celite and lyophilized to afford 0.6 g of potassium 3-[2-{4-[3-(2-methylpyrrolidin-1-yl)propoxy]phenyl}-6,7-dihydro[1,3]thiazolo[5,4-c]pyridin-5(4H)-yl]-3-oxopropanoate 86.

Yield: 92%.

LC-MS (MH⁺): 444.

### Example 33

### Synthesis of 3-[2-(4-{3-[(2R)-2-methylpyrrolidin-1-yl]propoxy}phenyl)-6,7-dihydro[1,3]thiazolo[5,4-c]pyridin-5(4H)-yl]cyclobut-2-en-1-one 87

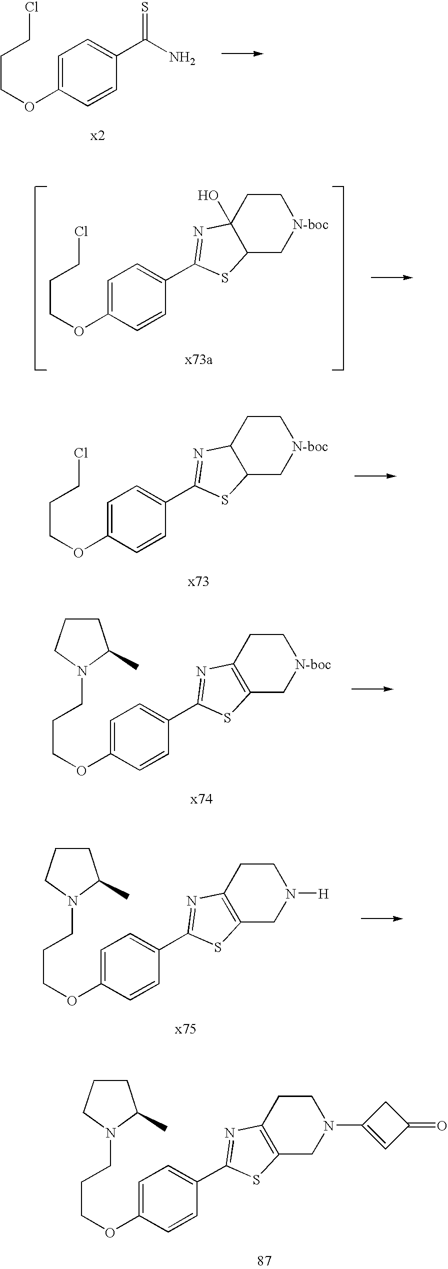

### 33.1 Synthesis of tert-butyl 2-[4-(3-chloropropoxy)phenyl]-6,7-dihydro[1,3]thiazolo[5,4-c]pyridine-5(4H)-carboxylate x73

4-(3-chloropropoxy)benzenecarbothioamide x2 (20 g, 87 mmol, 1 eq), bromoketone (30.0 g, 108 mmol, 1.4 eq) and potassium carbonate (16.2 g, 114 mmol, 1.5 eq) are stirred in iso-propyl alcohol (600 ml) at 55° C. during three hours. The mixture is evaporated to dryness. Ethyl acetate (400 ml) and water are added to the crude. The organic layer is dried over magnesium sulfate and concentrated under reduce pressure to provide 38 g of crude tert-butyl 2-[4-(3-chloropropoxy)phenyl]-7a-hydroxy-3a,6,7,7a-tetrahydro[1,3]thiazolo[5,4-c]pyridine-5(4H)-carboxylate x73a. This compound is dissolved in dichloromethane (300 ml). Triethylamine (39 ml, 270 mmol, 3 eq) is added followed by the addition of a solution of methanesulfonyl chloride (15 ml, 190 mmol, 2.2 eq) in dichloromethane (15 ml). The mixture is stirred one hour at room temperature. After concentration under reduced pressure, ethylacetate (400 ml) and water (200 ml) is added. The organic layer is dried over magnesium sulfate and concentrated to dryness. After purification by chromatography (dichloromethane/methanol/ammonia 98:1.8:0.2), the solid is triturated with benzine to afford 18 g of tert-butyl 2-[4-(3-chloropropoxy)phenyl]-6,7-dihydro[1,3]thiazolo[5,4-c]pyridine-5(4H)-carboxylate x73.

Yield: 57%.

LC-MS (MH⁺): 409/411.

### 33.2 Synthesis of tert-butyl 2-(4-{3-[(2R)-2-methylpyrrolidin-1-yl]propoxy}phenyl)-6,7-dihydro[1,3]thiazolo[5,4-c]pyridine-5(4H)-carboxylate x74

A suspension of tert-butyl 2-[4-(3-chloropropoxy)phenyl]-6,7-dihydro[1,3]thiazolo[5,4-c]pyridine-5(4H)-carboxylate x73 (12.9 g, 31.4 mmol, 1 eq), potassium carbonate (13.0 g, 94.6 mmol, 3 eq) and sodium iodide (0.5 g) in acetonitrile (400 ml) is heated at 80° C. for 1 h. After cooling to room temperature, (R)-2-methylpyrrolidine (4.0, 47.3 mmol, 1.5 eq) is added and the mixture is stirred at 80° C. for 2 days. The mixture is concentrated under reduced pressure and the residue is taken up with dichloromethane (400 ml) and water (200 ml). The organic layer is dried over magnesium sulfate and concentrated under reduced pressure. After purification by chromatography over silicagel (dichloromethane/methanol/ammonia 90:9:1), the solid obtained is triturated with hexane and filtered to afford 12.5 g of tert-butyl 2-(4-{3-[(2R)-2-methylpyrrolidin-1-yl]propoxy}phenyl)-6,7-dihydro[1,3]thiazolo[5,4-c]pyridine-5(4H)-carboxylate x74.

Yield: 86%.

LC-MS (MH⁺): 458.

### 33.3 Synthesis of 2-(4-{3-[(2R)-2-methylpyrrolidin-1-yl]propoxy}phenyl)-4,5,6,7-tetrahydro[1,3]thiazolo[5,4-c]pyridine x75

Tert-butyl 2-(4-{3-[(2R)-2-methylpyrrolidin-1-yl]propoxy}phenyl)-6,7-dihydro[1,3]thiazolo[5,4-c]pyridine-5(4H)-carboxylate x74 (4 g, 8.7 mmol, 1 eq) is dissolved in dichloromethane (50 ml). Trifluoroacetic acid is added and the mixture is stirred overnight at room temperature. Dichloromethane (100 ml) and water (100 ml) are added. The organic layer is removed and the aqueous layer is basified with sodium hydroxide. Dichloromethane (100 ml) is added. The organic layer is dried over magnesium sulfate and concentrated to dryness to provide 3.3 g of 2-(4-{3-[(2R)-2-methylpyrrolidin-1-yl]propoxy}phenyl)-4,5,6,7-tetrahydro[1,3]thiazolo[5,4-c]pyridine x75.

Yield: 100%.

LC-MS (MH⁺): 358

### 33.4 Synthesis of 3-[2-(4-{3-[(2R)-2-methylpyrrolidin-1-yl]propoxy}phenyl)-6,7-dihydro[1,3]thiazolo[5,4-c]pyridin-5(4H)-yl]cyclobut-2-en-1-one 87

A suspension of N-cyclohexylcyclohexanaminium 3-oxocyclobut-1-en-1-oxalate (0.37 g, 1.4 mmol, 1.4 eq) in dioxane (5 ml) is treated with a solution of trifluoroacetic acid (0.11 ml, 1.54 mmol, 1.1 eq) in dioxane (5 ml) and the mixture is stirred four hours at room temperature. The mixture is filtered and the precipitate is washed with dioxane (10 ml). The resulting solution is treated with a solution of 2-(4-{3-[(2R)-2-methylpyrrolidin-1-yl]propoxy}phenyl)-4,5,6,7-tetrahydro[1,3]thiazolo[5,4-c]pyridine x75 (0.5 g, 1.4 mmol, 1 eq) in dioxane (10 ml). After one hour stirring, the solvent is removed and the residue is taken up with dichloromethane (100 ml) and water (50 ml). The organic layer is dried over magnesium sulfate and evaporated to dryness. Purification by chromatography over silicagel (dichloromethane/methanol/ammonia 95:4.5:0.5) affords 300 mg of 3-[2-(4-{3-[(2R)-2-methylpyrrolidin-1-yl]propoxy}phenyl)-6,7-dihydro[1,3]thiazolo[5,4-c]pyridin-5(4H)-yl]cyclobut-2-en-1-one 87.

Yield: 50%.

LC-MS (MH⁺): 424.

### Example 34

### Synthesis of 2-(4-{3-[(2R)-2-methylpyrrolidin-1-yl]propoxy}phenyl)-5-(morpholin-4-ylsulfonyl)-4,5,6,7-tetrahydro[1,3]thiazolo[5,4-c]pyridine 89

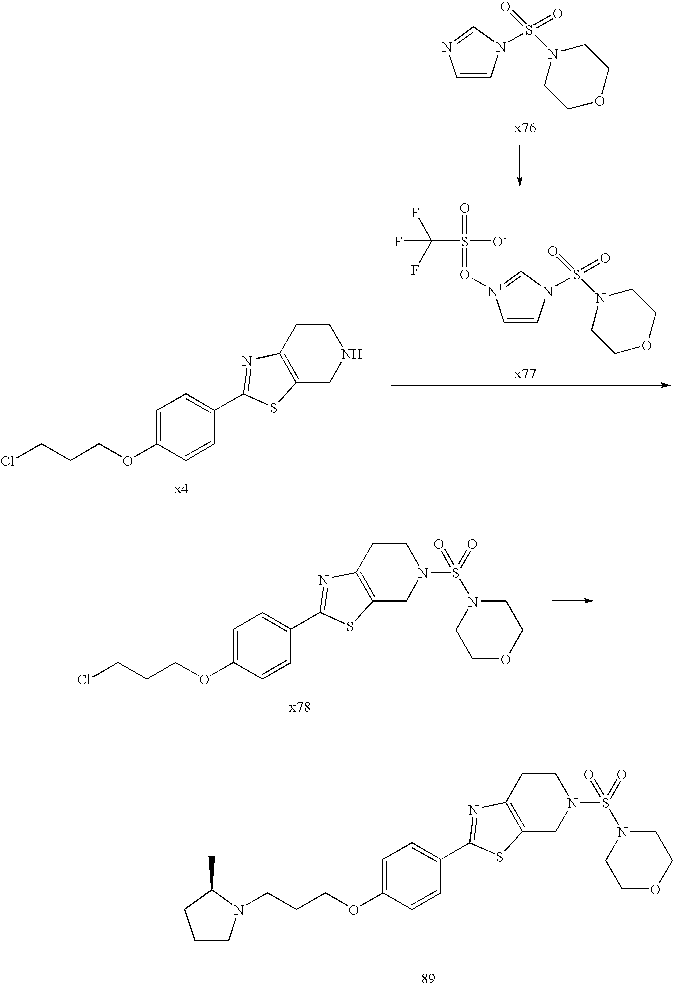

### 34.1 Synthesis of 3-methyl-1-(morpholin-4-ylsulfonyl)-1H-imidazol-3-ium trifluoromethanesulfonate x77

To a solution of 4-(1H-imidazol-1-ylsulfonyl)morpholine (0.19 g, 0.87 mmol, 1 eq) in dichloromethane (10 ml) cooled at 0° C. is added methyl trifluoromethanesulfonate (0.10 ml, 0.87 mmol, 1 eq) and the reaction mixture is stirred for 2 h at 0° C. The solid is filtered and dried under vacuum to give 245 mg of 3-methyl-1-(morpholin-4-ylsulfonyl)-1H-imidazol-3-ium trifluoromethanesulfonate x77, which is used in the next step without any purification.

Yield: 74%.

### 34.2 Synthesis of 2-[4-(3-chloropropoxy)phenyl]-5-(morpholin-4-ylsulfonyl)-4,5,6,7-tetrahydro[1,3]thiazolo[5,4-c]pyridine x78

A mixture of 3-methyl-1-(morpholin-4-ylsulfonyl)-1H-imidazol-3-ium trifluoromethanesulfonate x77 (0.245 g, 0.64 mmol, 1.5 eq), triethylamine (0.06 ml, 0.43 mmol, 1 eq) and 2-{4-[(3-chloropropyl)oxy]phenyl}-4,5,6,7-tetrahydro[1,3]thiazolo[5,4-c]pyridine x4 (0.13 g, 0.43 mmol, 1 eq) in acetonitrile (10 ml) is stirred for 2 hours at 70° C., then the reaction mixture is concentrated under reduced pressure. The residue is dissolved in ethyl acetate and washed twice with an aqueous saturated solution of sodium hydrogenocarbonate. The organic layer is dried over magnesium sulfate, filtered and concentrated under vacuum to give 0.18 g of 2-[4-(3-chloropropoxy)phenyl]-5-(morpholin-4-ylsulfonyl)-4,5,6,7-tetrahydro[1,3]thiazolo[5,4-c]pyridine x78, which is used in the next step without any purification.

Yield: 91%.

LC-MS (MH⁺): 458/460.

### 34.3 Synthesis of 2-(4-{3-[(2R)-2-methylpyrrolidin-1-yl]propoxy}phenyl)-5-(morpholin-4-ylsulfonyl)-4,5,6,7-tetrahydro[1,3]thiazolo[5,4-c]pyridine 89

A suspension of 2-[4-(3-chloropropoxy)phenyl]-5-(morpholin-4-ylsulfonyl)-4,5,6,7-tetrahydro[1,3]thiazolo[5,4-c]pyridine x78 (0.18 g, 0.39 mmol, 1 eq), potassium carbonate (0.054 g, 0.78 mmol, 2 eq) and a catalytic amount of sodium iodide is stirred at reflux for 30 minutes then cooled to room temperature. (2R)-2-methylpyrrolidine (0.05 g, 0.585 mmol, 1.5 eq) is added and the reaction mixture is stirred at reflux for two days. The solution is concentrated under reduce pressure. The residue is dissolved in ethyl acetate and washed twice with an aqueous saturated solution of sodium hydrogenocarbonate. The organic layer is dried over magnesium sulfate, filtered and concentrated under vacuum. Chromatography over silicagel (dichloromethane/methanol/ammonia 97:3:0.3) yields 46 mg of 2-(4-{3-[(2R)-2-methylpyrrolidin-1-yl]propoxy}phenyl)-5-(morpholin-4-ylsulfonyl)-4,5,6,7-tetrahydro[1,3]thiazolo[5,4-c]pyridine 89.

Yield: 23%.

LC-MS (MH⁺): 507.

### Example 35

### Synthesis of 3-[2-(4-{3-[(2R)-2-methylpyrrolidin-1-yl]propoxy}phenyl)-6,7-dihydro[1,3]thiazolo[5,4-c]pyridin-5(4H)-yl]-3-oxopropanamide 90

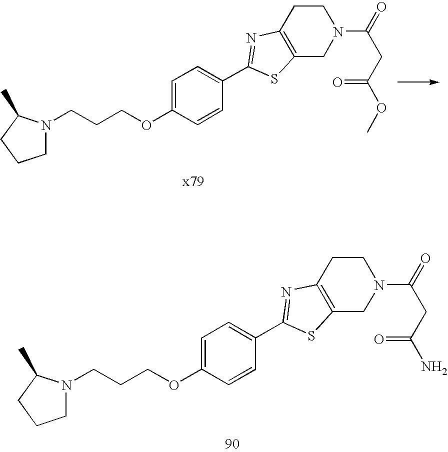

Methyl 3-[2-(4-{3-[(2R)-2-methylpyrrolidin-1-yl]propoxy}phenyl)-6,7-dihydro[1,3]thiazolo[5,4-c]pyridin-5(4H)-yl]-3-oxopropanoate x79 is prepared as described in example 3.

Yield: 100%.

LC-MS (MH⁺): 458.

A solution of methyl 3-[2-(4-{3-[(2R)-2-methylpyrrolidin-1-yl]propoxy}phenyl)-6,7-dihydro[1,3]thiazolo[5,4-c]pyridin-5(4H)-yl]-3-oxopropanoate x79 (0.3 g, 0.66 mmol, 1 eq) in methyl alcohol (5 ml) is treated with a 7 M solution of ammonia in methyl alcohol (5 ml, 35 ml, 55 eq) is added and the mixture is stirred in a sealed tube at 90° C. overnight. After evaporation, the residue is taken up with dichloromethane, washed with water and dried over magnesium sulfate. Purification by chromatography over silicagel (gradient: dichloromethane/methanol/ammonia 95:5:0.5 to 94:5.4:0.6) affords 50 mg of 3-[2-(4-{3-[(2R)-2-methylpyrrolidin-1-yl]propoxy}phenyl)-6,7-dihydro[1,3]thiazolo[5,4-c]pyridin-5(4H)-yl]-3-oxopropanamide 90.

Yield: 14%.

LC-MS (MH⁺): 443.

### Example 36

### Synthesis 2-[2-(4-{3-[(2R)-2-methylpyrrolidin-1-yl]propoxy}phenyl)-6,7-dihydro[1,3]thiazolo[5,4-c]pyridin-5(4H)-yl]acetamide 93

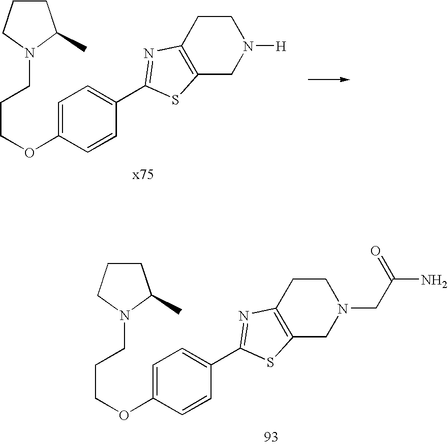

A mixture of 2-(4-{3-[(2R)-2-methylpyrrolidin-1-yl]propoxy}phenyl)-4,5,6,7-tetrahydro[1,3]thiazolo[5,4-c]pyridine x75 (0.3 g, 0.84 mmol, 1 eq), potassium carbonate (0.35 g, 2.52 mmol, 3 eq), sodium iodide (50 mg) and 2-bromoacetamide (10 μl, 1.01 mmol, 1.2 eq) in acetonitrile (20 ml) is stirred 2 hours 20 h at 80° C. The mixture is concentrated to dryness, and the residue is taken up with dichloromethane and washed with water. The organic layer is dried over magnesium sulfate and concentrated. The crude product is purified by chromatography over silicagel (dichloromethane/methanol/ammonia 95:4.5:0.5) to afford 110 mg of 2-[2-(4-{3-[(2R)-2-methylpyrrolidin-1-yl]propoxy}phenyl)-6,7-dihydro[1,3]thiazolo[5,4-c]pyridin-5(4H)-yl]acetamide 93.

Yield: 32%.

LC-MS (MH⁺): 415.

Compounds 91, 94, 95 and 97 may be synthesized according to the same method.

### Example 37

### Synthesis of 2-[2-(4-{3-[(2R)-2-methylpyrrolidin-1-yl]propoxy}phenyl)-6,7-dihydro[1,3]thiazolo[5,4-c]pyridin-5(4H)-yl]-2-oxoethanol 92

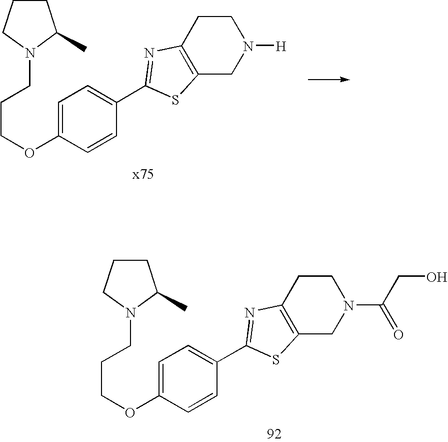

A solution of 2-(4-{3-[(2R)-2-methylpyrrolidin-1-yl]propoxy}phenyl)-4,5,6,7-tetrahydro[1,3]thiazolo[5,4-c]pyridine x75 (0.3 g, 0.84 mmol, 1 eq) in dichloromethane (20 ml) is treated with 1-(3-dimethylaminopropyl)-3-ethylcarbodiimide hydrochloride (0.56 g, 2.94 mmol, 3.5 eq), 1-hydroxybenzotriazole (0.23 g, 1.68 mmol, 2 eq) and dimethylaminopyridine (0.1 g, 0.84 mmol, 1 eq). The mixture is stirred 30 minutes at room temperature and glycolic acid (80 μg, 1.01 mmol, 1.2 eq) is added. After 10 minutes, water is added. The organic layer is washed with brine, dried over magnesium sulfate and evaporated to dryness. The residue is purified by chromatography over silicagel (dichloromethane/methanol/ammonia 95:4.5:0.5), to afford 100 mg of 2-[2-(4-{3-[(2R)-2-methylpyrrolidin-1-yl]propoxy}phenyl)-6,7-dihydro[1,3]thiazolo[5,4-c]pyridin-5(4H)-yl]-2-oxoethanol 92.

Yield: 25%.

LC-MS (MH⁺): 416.

### Example 38

### Synthesis of ((2S)-1-{3-[4-(5-acetyl-4,5,6,7-tetrahydro[1,3]thiazolo[5,4-c]pyridin-2-yl)phenoxy]propyl}pyrrolidin-2-yl)methanol 96

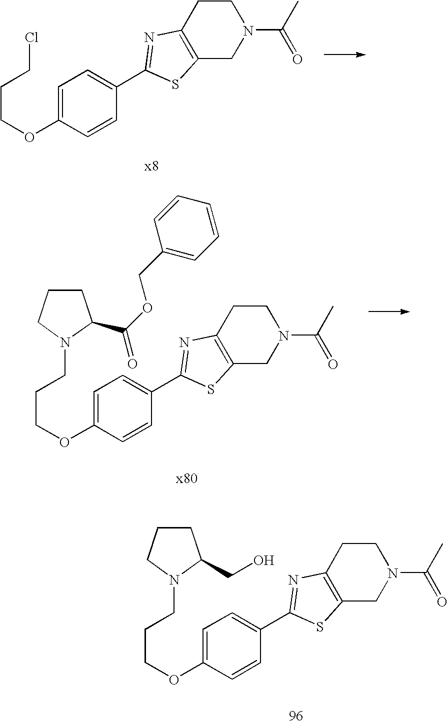

### 38.1 Synthesis of benzyl (2S)-1-{3-[4-(5-acetyl-4,5,6,7-tetrahydro[1,3]thiazolo[5,4-c]pyridin-2-yl)phenoxy]propyl}pyrrolidine-2-carboxylate x80

A mixture of 5-acetyl-2-[4-(3-chloropropoxy)phenyl]-4,5,6,7-tetrahydro[1,3]thiazolo[5,4-c]pyridine x8 (0.74 g, 2.11 mmol, 1 eq), potassium carbonate (0.87 g, 6.33 mmol, 3 eq) and sodium iodide (0.1 g) in acetonitrile (20 ml) is heated at 90° C. for 1 h. The mixture is cooled to 20° C. and benzyl L-prolinate hydrochloride (0.43 g, 2.11 mmol, 1 eq) is added. The mixture is heated at 90° C. for 60 h, concentrated under reduced pressure, and the residue is dissolved in dichloromethane and washed with water. The organic phase is dried over magnesium sulfate and concentrated. The residue is purified by chromatography over silicagel (dichloromethane/methanol/ammonia 98.4:1.44:0.16) to afford 590 mg of benzyl (2S)-1-{3-[4-(5-acetyl-4,5,6,7-tetrahydro[1,3]thiazolo[5,4-c]pyridin-2-yl)phenoxy]propyl}pyrrolidine-2-carboxylate x80.

Yield: 54%.

LC-MS (MH⁺): 520.

### 38.2 Synthesis of ((2S)-1-{3-[4-(5-acetyl-4,5,6,7-tetrahydro[1,3]thiazolo[5,4-c]pyridin-2-yl)phenoxy]propyl}pyrrolidin-2-yl)methanol 96

Lithium borohydride (40 mg, 2.02 mmol, 3 eq) is added to a solution of benzyl (2S)-1-{3-[4-(5-acetyl-4,5,6,7-tetrahydro[1,3]thiazolo[5,4-c]pyridin-2-yl)phenoxy]propyl}pyrrolidine-2-carboxylate x80 (590 mg, 0.65 mmol, 1 eq) in tetrahydrofuran (10 ml). The mixture is stirred overnight at 20° C. Water (2 ml) is added and the mixture is concentrated under reduced pressure. The residue is taken up with dichloromethane (50 ml) and washed with water. The organic layer is dried over magnesium sulfate and concentrated. The residue is purified by chromatography over silicagel (dichloromethane/methanol/ammonia 90:10:1) then by HPLC (gradient: acetonitrile/water from 5:95 to 95:5). Lyophilisation affords 38 mg of ((2S)-1-{3-[4-(5-acetyl-4,5,6,7-tetrahydro[1,3]thiazolo[5,4-c]pyridin-2-yl)phenoxy]propyl}pyrrolidin-2-yl)methanol 96.

Yield: 12%.

LC-MS (MH⁺): 416.

### Example 39

### Synthesis of [2-(4-{3-[(2R)-2-methylpyrrolidin-1-yl]propoxy}phenyl)-6,7-dihydro[1,3]thiazolo[5,4-c]pyridin-5(4H)-yl]acetic acid trifluoroacetate 98

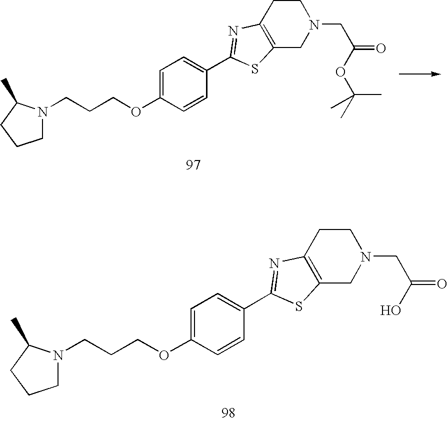

A solution of tert-butyl [2-(4-{3-[(2R)-2-methylpyrrolidin-1-yl]propoxy}phenyl)-6,7-dihydro[1,3]thiazolo[5,4-c]pyridin-5(4H)-yl]acetate 97 (0.32 g, 0.68 mmol, 1 eq) in dichloromethane (5 ml) is treated with trifluoroacetic acid (5 ml). Gas evolution is observed and the mixture is stirred at room temperature overnight. The mixture is concentrated to dryness and dried at 40° C. under reduced pressure overnight to afford 270 mg of [2-(4-{3-[(2R)-2-methylpyrrolidin-1-yl]propoxy}phenyl)-6,7-dihydro[1,3]thiazolo[5,4-c]pyridin-5(4H)-yl]acetic acid trifluoroacetate 98 as a hygroscopic yellow solid.

Yield: 92%.

LC-MS (MH⁺): 416.

### Example 40

### Synthesis of 2-{4-[3-(2-methylpyrrolidin-1-yl)propoxy]phenyl}-6-(morpholin-4-ylcarbonyl)-5,6,7,8-tetrahydro-4H-[1,3]thiazolo[4,5-d]azepine 100

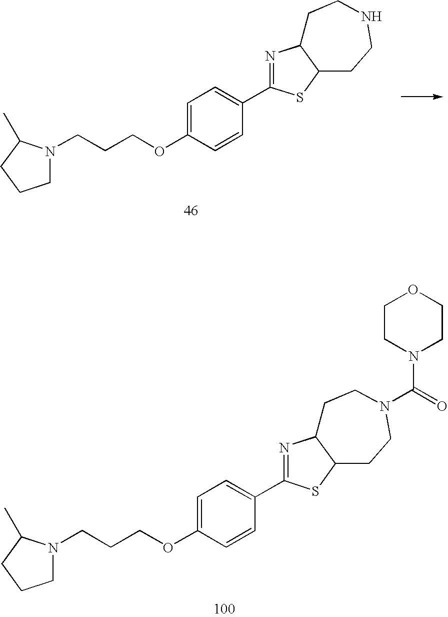

Morpholinecarbonyl chloride (0.06 ml, 0.8 mmol, 1 eq) is added to a solution of 2-{4-[3-(2-methylpyrrolidin-1-yl)propoxy]phenyl}-5,6,7,8-tetrahydro-4H-[1,3]thiazolo[4,5-d]azepine 46 (300 mg, 0.8 mmol, 1 eq) and triethylamine (0.2 ml, 1.4 mmol, 2 eq) in dichloromethane (2.5 ml) at room temperature under stirring in a stream of argon. The reaction mixture is stirred at room temperature for 1 h, then a 5% aqueous solution of potassium carbonate is added, and the organic layer is filtered through potassium carbonate and evaporated. The residue is purified by chromatography over silicagel (12 ml, carbon tetrachloride then chloroform then chloroform/methanol 90:10) to afford 215 mg of 2-{4-[3-(2-methylpyrrolidin-1-yl)propoxy]phenyl}-6-(morpholin-4-ylcarbonyl)-5,6,7,8-tetrahydro-4H-[1,3]thiazolo[4,5-d]azepine 100 as a pale yellow solid.

Yield: 55%.

LC-MS (MH⁺): 485.

### Example 41

### Synthesis of 2-(2-{4-[3-(2-methylpyrrolidin-1-yl)propoxy]phenyl}-4,5,7,8-tetrahydro-6H-[1,3]thiazolo[4,5-d]azepin-6-yl)acetamide 101

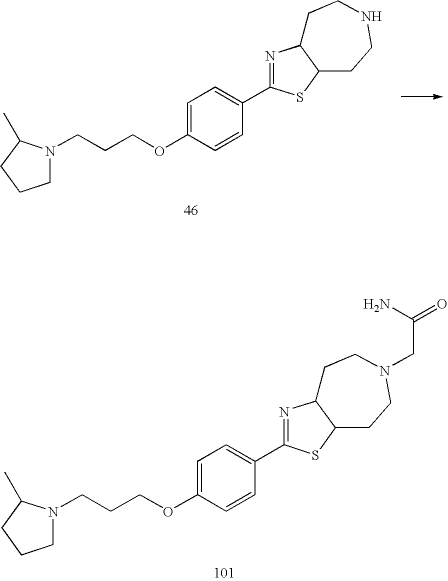

2-Bromoacetamide (0.14 ml, 0.97 mmol, 1.2 eq) is added to a solution 2-{4-[3-(2-methylpyrrolidin-1-yl)propoxy]phenyl}-5,6,7,8-tetrahydro-4H-[1,3]thiazolo[4,5-d]azepine 46 (300 mg, 0.81 mmol, 1 eq) and diisopropyl ethylamine (0.28 ml, 1.61 mmol, 2 eq) in dimethyl sulfoxide (3 ml) at room temperature under stirring in a stream of argon. The reaction mixture is stirred at room temperature overnight. An aqueous saturated solution of potassium carbonate is added, and the organic layer is dried over sodium sulfate and evaporated. The residue is purified by chromatography over silicagel (gradient: dichloromethane/methanol from 10:0 to 9:1) to afford 224 mg of 2-(2-{4-[3-(2-methylpyrrolidin-1-yl)propoxy]phenyl}-4,5,7,8-tetrahydro-6H-[1,3]thiazolo[4,5-d]azepin-6-yl)acetamide 101 as white crystals.

Yield: 65%.

LC-MS (MH⁺): 429.

### Example 42

### Synthesis of 6-isonicotinoyl-2-{4-[3-(2-methylpyrrolidin-1-yl)propoxy]phenyl}-5,6,7,8-tetrahydro-4H-[1,3]thiazolo[4,5-d]azepine L-(+)-tartrate 103

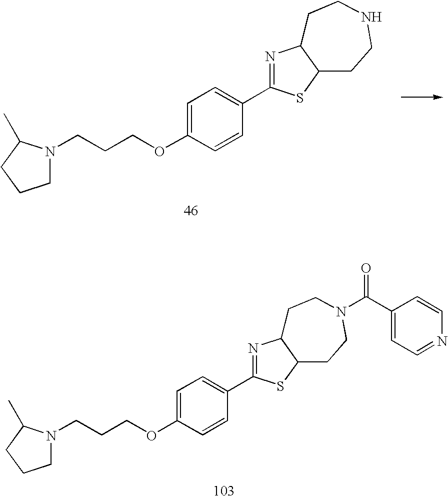

Benzotriazol-1-yloxytris(dimethylamino)-phosphonium hexafluorophosphate (0.54 g, 1.21 mmol) is added by portions to a mixture of 2-{4-[3-(2-methylpyrrolidin-1-yl)propoxy]phenyl}-5,6,7,8-tetrahydro-4H-[1,3]thiazolo[4,5-d]azepine 46 (0.3 g, 0.81 mmol, 1 eq), triethylamine (0.35 ml, 1.62 mmol, 2 eq) and isonicotinic acid (0.12 g, 0.97 mmol, 1.2 eq) in N,N-dimethylformamide (5 ml) at room temperature under stirring in a stream of argon. The reaction mixture is stirred at room temperature overnight. N,N-dimethylformamide is evaporated then water, diethyl ether, dichloromethane and aqueous saturated potassium carbonate are added. The organic layer is washed with 5% aqueous potassium carbonate, dried over potassium carbonate and evaporated in vacuo. The residue is purified by chromatography over silicagel (carbon tetrachloride then dichloromethane then dichloromethane/methanol 80:20) to afford 0.16 g of 6-isonicotinoyl-2-{4-[3-(2-methylpyrrolidin-1-yl)propoxy]phenyl}-5,6,7,8-tetrahydro-4H-[1,3]thiazolo[4,5-d]azepine as a yellow oil.

Yield: 42%.

LC-MS (MH⁺): 477.

6-isonicotinoyl-2-{4-[3-(2-methylpyrrolidin-1-yl)propoxy]phenyl}-5,6,7,8-tetrahydro-4H-[1,3]thiazolo[4,5-d]azepine (0.16 g, 0.34 mmol, 1 eq) is dissolved in methanol (0.5 ml). Tartaric acid (1 M in water, 0.34 ml, 0.34 mmol, 1 eq) is added and the reaction mixture is evaporated to dryness. The residue is crystallized from diethyl ether to afford 0.185 g of 6-isonicotinoyl-2-{4-[3-(2-methylpyrrolidin-1-yl)propoxy]phenyl}-5,6,7,8-tetrahydro-4H-[1,3]thiazolo[4,5-d]azepine L-(+)-tartrate 103 as pale crystals.

### Example 43

### Synthesis of 5,7,7-trimethyl-2-{4-[3-(2-methylpyrrolidin-1-yl)propoxy]phenyl}-5,6,7,8-tetrahydro-4H-[1,3]thiazolo[5,4-c]azepin-4-one 105

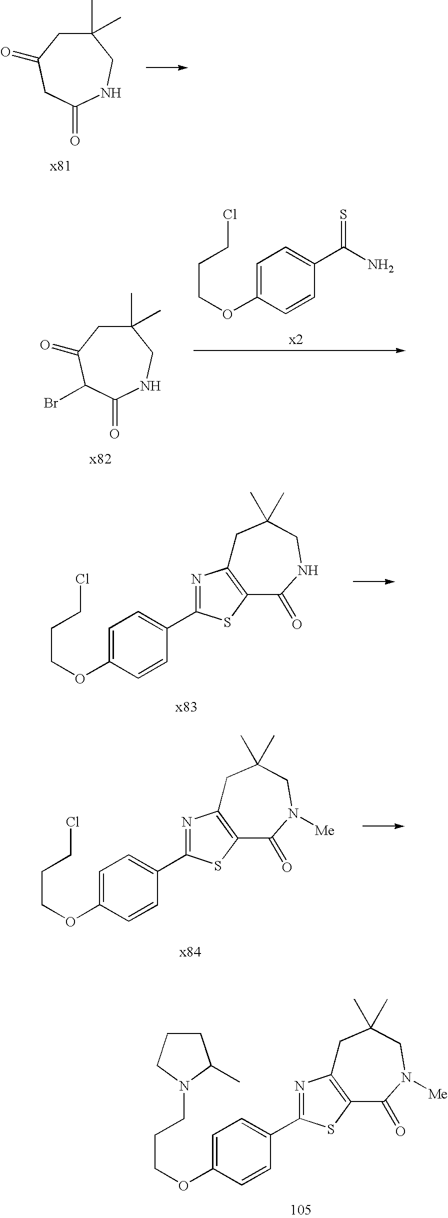

### 43.1 Synthesis of 3-bromo-6,6-dimethylazepane-2,4-dione x82

N-Bromosuccinimide (11.5 g, 64.5 mmol) is added slowly to a solution of 6,6-dimethylazepane-2,4-dione x81 (10.0 g, 64.5 mmol) and NaHSO₄ (1.94 g, 16.1 mmol) in THF (350 ml) at 0° C., and the reaction is warmed to room temperature for 1 h. Aqueous NaHCO₃ (300 ml) is added to the reaction, and the mixture extracted with dichloromethane (3×100 ml). The combined organic extracts are dried over MgSO₄, and concentrated in vacuo to give a white solid. The solid is triturated with isopropyl ether and the resulting precipitate filtered and dried in vacuo to afford 11.18 g of 3-bromo-6,6-dimethylazepane-2,4-dione x82 as a white solid.

Yield: 74%.

LC-MS (MH⁺): 234/236.

### 43.2 Synthesis of 2-[4-(3-chloropropoxy)phenyl]-7,7-dimethyl-5,6,7,8-tetrahydro-4H-[1,3]thiazolo[5,4-c]azepin-4-one x83

A mixture of 3-bromo-6,6-dimethylazepane-2,4-dione x82 (2.24 g, 9.57 mmol, 1.1 eq) and 4-(3-chloropropoxy)benzenecarbothioamide (2 g, 8.7 mmol, 1 eq) in DMF (40 ml) is stirred overnight at 80° C. The reaction mixture is concentrated under reduced pressure. The residue is dissolved in ethyl acetate and washed twice with an aqueous saturated solution of sodium hydrogenocarbonate. The organic layer is dried over magnesium sulfate, filtered and concentrated under vacuum to give 3.15 g of 2-[4-(3-chloropropoxy)phenyl]-7,7-dimethyl-5,6,7,8-tetrahydro-4H-[1,3]thiazolo[5,4-c]azepin-4-one x83, which is used directly in the next step.

Yield: 99%.

LC-MS (MH⁺): 365/367.

### 43.3. Synthesis of 2-[4-(3-chloropropoxy)phenyl]-5,7,7-trimethyl-5,6,7,8-tetrahydro-4H-[1,3]thiazolo[5,4-c]azepin-4-one x84

Sodium hydride (60% dispersion in mineral oil, 0.055 g, 1.39 mmol, 1.7 eq) is added to a cold (0° C.) solution of 2-[4-(3-chloropropoxy)phenyl]-7,7-dimethyl-5,6,7,8-tetrahydro-4H-[1,3]thiazolo[5,4-c]azepin-4-one x83 (0.3 g, 0.82 mmol, 1 eq) in dry DMF (10 ml) at 0° C. The mixture is stirred 15 min. at room temperature, cooled at 0° C., then iodomethane (0.14 g, 0.98 mmol, 1.2 eq) is added and the reaction is stirred at room temperature for two days. The reaction mixture is concentrated under reduce pressure. The residue is dissolved in ethyl acetate and washed twice with an aqueous saturated solution of sodium hydrogenocarbonate. The organic layer is dried over magnesium sulfate, filtered and concentrated under reduced pressure. Purification over silicagel (hexane/ethyl acetate 70:30) yields 80 mg of 2-[4-(3-chloropropoxy)phenyl]-5,7,7-trimethyl-5,6,7,8-tetrahydro-4H-[1,3]thiazolo[5,4-c]azepin-4-one x84.

Yield: 26%.

LC-MS (MH⁺): 379/381.

### 43.4. Synthesis of 5,7,7-trimethyl-2-{4-[3-(2-methylpyrrolidin-1-yl)propoxy]phenyl}-5,6,7,8-tetrahydro-4H-[1,3]thiazolo[5,4-c]azepin-4-one 105

A mixture of 2-[4-(3-chloropropoxy)phenyl]-5,7,7-trimethyl-5,6,7,8-tetrahydro-4H-[1,3]thiazolo[5,4-c]azepin-4-one x84 (0.07 g, 0.185 mmol, 1 eq), potassium carbonate (0.05 g, 0.37 mmol, 2 eq) and a catalytic quantity of sodium iodide is stirred at reflux for 30 minutes then cooled to room temperature. 2-Methylpyrrolidine (0.022 ml, 0.22 mmol, 1.2 eq) is added and the reaction mixture is stirred at reflux overnight. The solution is concentrated under reduced pressure, the residue is dissolved in ethyl acetate and washed twice with an aqueous saturated solution of sodium hydrogenocarbonate. The organic layer is dried over magnesium sulfate, filtered and concentrated under vacuum. Purification over silicagel (gradient: dichloromethane/methanol from 99:1 to 90:10) yields 26 mg of 5,7,7-trimethyl-2-{4-[3-(2-methylpyrrolidin-1-yl)propoxy]phenyl}-5,6,7,8-tetrahydro-4H-[1,3]thiazolo[5,4-c]azepin-4-one 105.

Yield: 33%.

LC-MS (MH⁺): 428.

7,7-dimethyl-2-{4-[3-(2-methylpyrrolidin-1-yl)propoxy]phenyl}-5,6,7,8-tetrahydro-4H-[1,3]thiazolo[5,4-c]azepin-4-one 104 may be synthesized according to the same method, starting from compound x83.

### Example 44

### Synthesis of 4-acetyl-2-(4-{3-[(2R)-2-methylpyrrolidin-1-yl]propoxy}phenyl)-5,6,7,8-tetrahydro-4H-[1,3]thiazolo[5,4-b]azepine 106

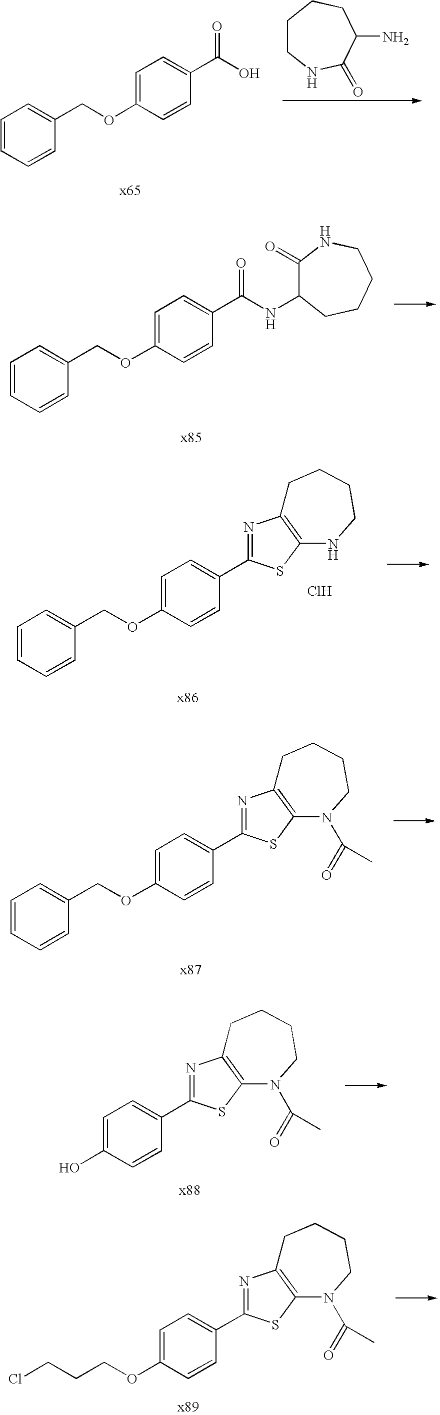

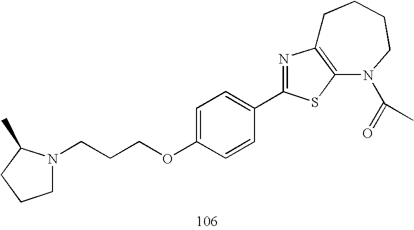

### 44.1 Synthesis of 4-(benzyloxy)-N-(2-oxoazepan-3-yl)benzamide x85

A suspension of 4-(benzyloxy)benzoic acid x65 (5.0 g, 21.93 mmol, 1 eq) and N,N-dimethylformamide (0.5 ml) in dichloromethane (300 ml) at 0° C. is treated with oxalyl chloride (2.83 ml, 26.32 mmol, 1.2 eq). The mixture is left to warm up to room temperature and stirred until gas evolution has ceased. Half of the solvent is removed under reduced pressure and the resulting solution is added to a mixture of DL-alpha-amino-epsilon-caprolactam (3.37 g, 26.32 mmol, 1.2 eq) and triethylamine (6.11 ml, 43.86 mmol, 2 eq) in dichloromethane (300 ml). After 1 h stirring at 20° C., water (200 ml) is added and the organic layer is dried over magnesium sulfate and concentrated. The residue is taken up with ethyl acetate, the resulting suspension is filtered and the solid dried at 40° C. under reduced pressure to afford 5.9 g of 4-benzyloxy-N-(2-oxoazepan-3-yl)benzamide x85.

Yield: 80%.

LC-MS (MH⁺): 339.

### 44.2 Synthesis of 2-[4-(benzyloxy)phenyl]-5,6,7,8-tetrahydro-4H-[1,3]thiazolo[5,4-b]azepine hydrochloride x86 chlorhydrate

A suspension of 4-benzyloxy-N-(2-oxoazepan-3-yl)benzamide x85 (2.0 g, 5.91 mmol, 1 eq) in pyridine (20 ml) is treated with Lawesson's reagent (1.43 g, 3.55 mmol, 0.6 eq) and the mixture is stirred at 100° C. for 20 h. After cooling down to room temperature, the mixture is poured on a saturated aqueous solution of sodium hydrogenocarbonate (150 ml) and the aqueous layer is extracted with dichloromethane (2×100 ml). The combined organic layers are dried over magnesium sulfate and concentrated under reduced pressure. The residue is taken up with a 1:1 mixture of ethyl acetate and dichloromethane (50 ml) and the uncyclised thioamide is filtered out. The organic layer is concentrated and the residue is purified by chromatography over silicagel (heptane/ethyl acetate 3:1). The main fraction from chromatography is concentrated under reduced pressure, the residue is dissolved in a 1:5 mixture of methanol and ether (10 ml) and treated with 2 M hydrogen chloride in ether (2 ml). The obtained solid is dried at 40° C. under vacuum to afford 900 mg of 2-[4-(benzyloxy)phenyl]-5,6,7,8-tetrahydro-4H-[1,3]thiazolo[5,4-b]azepine hydrochloride x86.

Yield: 23%.

LC-MS (MH⁺): 337.

### 44.3 Synthesis of 4-acetyl-2-[4-(benzyloxy)phenyl]-5,6,7,8-tetrahydro-4H-[1,3]thiazolo[5,4-b]azepine x87

A suspension of 2-[4-(benzyloxy)phenyl]-5,6,7,8-tetrahydro-4H-[1,3]thiazolo[5,4-b]azepine hydrochloride x86 (0.9 g, 2.68 mmol, 1 eq) in dichloromethane (40 ml) is treated with triethylamine (1.12 ml, 8.04 mmol, 3 eq) and a solution of acetyl chloride (0.23 ml, 3.21 mmol, 1.2 eq) in dichloromethane (5 ml). After 2 h stirring at 20° C., water (20 ml) is added. The organic layer is dried over magnesium sulfate and concentrated under reduced pressure. Chromatography over silicagel (dichloromethane/methanol/ammonia 95:5:0.5) affords 210 mg of 4-acetyl-2-[4-(benzyloxy)phenyl]-5,6,7,8-tetrahydro-4H-[1,3]thiazolo[5,4-b]azepine x87.

Yield: 64%.

LC-MS (MH⁺): 379.

### 44.4 Synthesis of 4(4-acetyl-5,6,7,8-tetrahydro-4H-[1,3]thiazolo[5,4-b]azepin-2-yl)phenol x88

A solution of 4-acetyl-2-[4-(benzyloxy)phenyl]-5,6,7,8-tetrahydro-4H-[1,3]thiazolo[5,4-b]azepine x87 (0.6 g, 1.5 mmol, 1 eq) in dichloromethane (10 ml) is treated with a 1 M solution of phosphorus tribromide in dichloromethane (8 ml, 8.0 mmol, 5 eq). The mixture is left to stir overnight at 20° C. before addition of water (10 ml). The organic layer is dried over magnesium sulfate and concentrated under reduced pressure. Chromatography over silicagel (dichloromethane/methanol/ammonia 95:5:0.5) affords 360 mg of 4-(4-acetyl-5,6,7,8-tetrahydro-4H-[1,3]thiazolo[5,4-b]azepin-2-yl)phenol x88.

Yield: 79%.

LC-MS (MH⁺): 289.

### 44.5 Synthesis of 4-acetyl-2-[4-(3-chloropropoxy)phenyl]-5,6,7,8-tetrahydro-4H-[1, 3]thiazolo[5,4-b]azepine x89

A solution of 4-(4-acetyl-5,6,7,8-tetrahydro-4H-[1,3]thiazolo[5,4-b]azepin-2-yl)phenol x88 (0.2 g, 0.69 mmol, 1 eq) in 2-butanone (10 ml) is treated with potassium carbonate (0.21 g, 1.53 mmol, 2.2 mmol) and 1-bromo-3-chloropropane (82 μl, 0.83 mmol, 1.2 eq) and the mixture is stirred at reflux for 20 h. The reaction mixture is concentrated under reduced pressure and the residue is dissolved in dichloromethane (20 ml). The organic layer is washed with water (20 ml), dried over magnesium sulfate and concentrated under reduced pressure to afford 300 mg of 4-acetyl-2-[4-(3-chloropropoxy)phenyl]-5,6,7,8-tetrahydro-4H-[1,3]thiazolo[5,4-b]azepine x89 which is used without further purification in the next reaction.

Yield: 100%.

LC-MS (MH⁺): 365/367.

### 44.6 Synthesis of 4-acetyl-2-(4-{3-[(2R)-2-methylpyrrolidin-1-yl]propoxy}phenyl)-5,6,7,8-tetrahydro-4H-[1,3]thiazolo[5,4-b]azepine 106

A suspension of 4-acetyl-2-[4-(3-chloropropoxy)phenyl]-5,6,7,8-tetrahydro-4H-[1,3]thiazolo[5,4-b]azepine x89 (0.3 g, 0.98 mmol, 1 eq), potassium carbonate (0.41 g, 2.93 mmol, 3 eq) and sodium iodide (10 mg) in acetonitrile (10 ml) is stirred at 80° C. over 1 h in a sealed tube. After cooling back to room temperature, the mixture is treated with (2R)-2-methylpyrrolidine (0.11 g, 1.27 mmol, 1.3 eq) and the mixture is heated for a further 24 h at 80° C. The mixture is then concentrated and the residue is taken up with dichloromethane (40 ml). This organic layer is washed with water (20 ml), dried over magnesium sulfate and concentrated under reduced pressure. Chromatography over silicagel (dichloromethane/methanol/ammonia 96:4:0.4) affords 170 mg of 4-acetyl-2-(4-{3-[(2R)-2-methylpyrrolidin-1-yl]propoxy}phenyl)-5,6,7,8-tetrahydro-4H-[1,3]thiazolo[5,4-b]azepine 106.

Yield: 56%.

LC-MS (MH⁺): 414.

### Example 45

### Synthesis of 2-{4-[3-(2-methylpyrrolidin-1-yl)propoxy]phenyl}-5-(morpholin-4-ylcarbonyl)-5,6-dihydro-4H-cyclopenta[d][1,3]thiazole 107

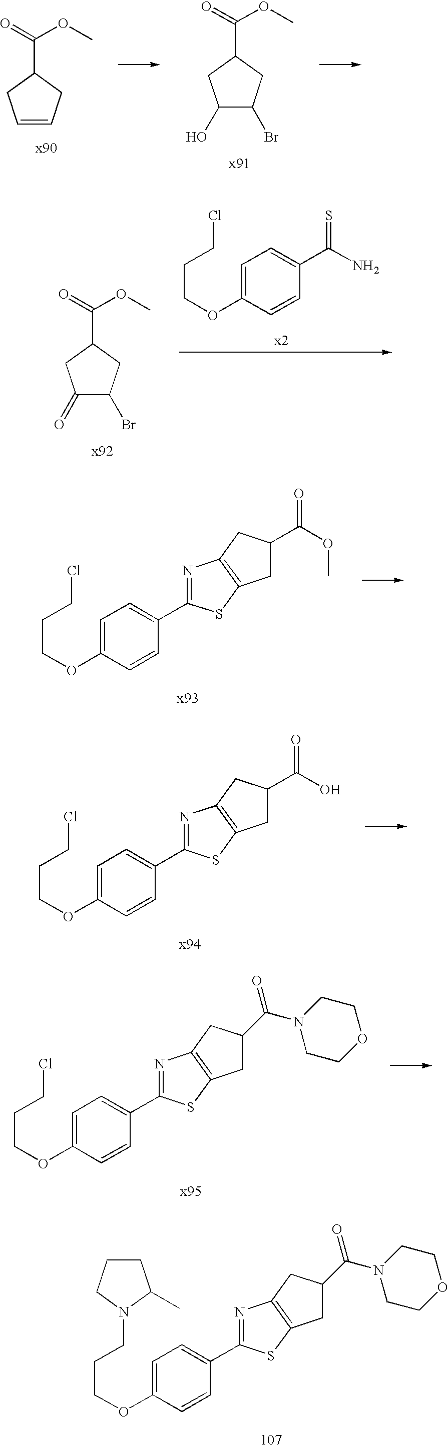

### 45.1. Synthesis of methyl 3-bromo-4-hydroxycyclopentanecarboxylate x91.

A solution of methyl cyclopent-3-ene-1-carboxylate x90 (6.69 g, 53 mmol, 1 eq) in acetonitrile (70 ml) is treated with calcium carbonate (5.3 g, 53 mmol, 1 eq) in water (18 ml). The mixture is cooled at 0° C. and a solution of N-bromosuccinimide (9.44 g, 53 mmol, 1 eq) in acetonitrile (70 ml) is added slowly. The mixture is stirred at room temperature for 4 hours, filtered and concentrated under vacuum. Water is then added and the product is extracted 3 times with ethyl acetate. The combined organic layers are washed with brine, dried over magnesium sulfate and concentrated under reduced pressure. The resulting orange solid is taken up with dichloromethane. The solid is then filtered, rinsed with dichloromethane and the filtrate is concentrated under vacuum to afford 10.71 g of methyl 3-bromo-4-hydroxycyclopentanecarboxylate x91 as an orange oil.

Yield: 90%.

GC-MS (M.⁺): 222/224.

### 45.2. Synthesis of methyl 3-bromo-4-oxocyclopentanecarboxylate x92.

A solution of methyl 3-bromo-4-hydroxycyclopentanecarboxylate x91 (5.58 g, 25 mmol, 1 eq) in dichloromethane (200 ml) is cooled at 0° C. and treated with a 15% solution of 1,1,1-triacetoxy-1,1-dihydro-1,2-benziodoxol-3(1H)-one (Dess-Martin's reagent) in dichloromethane (130 ml). The mixture is stirred at room temperature for 48 hours. A saturated solution of sodium thiosulfate is added and the mixture is stirred for one hour. Water is added, the two layers are separated and the organic layer is washed with a saturated solution of sodium hydrogenocarbonate, then with brine. It is dried over magnesium sulfate and concentrated under reduced pressure. The resulting brown oil is taken up with dichloromethane, heated and the solid is filtered off. The dichloromethane solution is concentrated under vacuum, the residue is taken up with diethyl ether, sonicated, the solid is filtered off and the solution is concentrated under reduced pressure. Methyl 3-bromo-4-oxocyclopentanecarboxylate x92 (4.57 g) is obtained as an orange oil and used in the next step without any further purification.

Yield: 83%.

### 45.3. Synthesis of methyl 2-[4-(3-chloropropoxy)phenyl]-5,6-dihydro-4H-cyclopenta[d][1,3]thiazole-5-carboxylate x93

A solution of methyl 3-bromo-4-oxocyclopentanecarboxylate x92 (2.55 g, 11 mmol, 1 eq) in ethanol (55 ml) is treated with 4-(3-chloropropoxy)benzenecarbothioamide x2 (3.44 g, 15 mmol, 1.4 eq). The reaction is stirred overnight under reflux. The mixture is then concentrated and the residue taken up with ethyl acetate. The organic layer is washed with a 10% solution of sodium hydroxide and water, dried over magnesium sulfate and concentrated under vacuum to afford 4.03 g of methyl 2-[4-(3-chloropropoxy)phenyl]-5,6-dihydro-4H-cyclopenta[1,3]thiazole-5-carboxylate x93 as a brown oil.

Yield: 100%.

LC-MS (MH⁺): 352/354.

### 45.4. Synthesis of 2-[4-(3-chloropropoxy)phenyl]-5,6-dihydro-4H-cyclopenta[d][1,3]thiazole-5-carboxylic acid x94

A solution of methyl 2-[4-(3-chloropropoxy)phenyl]-5,6-dihydro-4H-cyclopenta[1,3]thiazole-5-carboxylate x93 (1.35 g, 3.69 mmol, 1 eq) in tetrahydrofuran (74 ml) is treated with a solution of lithium hydroxide monohydrate (310 mg, 7.38 mmol, 2 eq) in water (15 ml) and the mixture is stirred overnight under reflux. Water is added to the reaction mixture and the aqueous phase is washed with ethyl acetate, acidified to pH 2 with a 2 N aqueous solution of hydrochloric acid and extracted three times with ethyl acetate. The combined organic layers are dried over magnesium sulfate and concentrated under vacuum to afford 1.16 g of 2-[4-(3-chloropropoxy)phenyl]-5,6-dihydro-4H-cyclopenta[1,3]thiazole-5-carboxylic acid x94 as a brown solid.

Yield: 93%.

LC-MS (MH⁺): 338/340.

### 45.5. Synthesis of 2-[4-(3-chloropropoxy)phenyl]-5-(morpholin-4-ylcarbonyl)-5,6-dihydro-4H-cyclopenta[d][1,3]thiazole x95

1-Hydroxybenzotriazole (0.02 g, 0.15 mmol, 0.2 eq) and 1-(3-dimethylaminopropyl)-3-ethylcarbodiimide hydrochloride (0.156 g, 0.81 mmol, 1.1 eq) are added to a solution of 2-[4-(3-chloropropoxy)phenyl]-5,6-dihydro-4H-cyclopenta[1,3]thiazole-5-carboxylic acid x94 (0.25 g, 0.74 mmol, 1 eq), morpholine (0.065 ml, 0.74 mmol, 1 eq) and triethylamine (0.208 ml, 1.48 mmol, 2 eq) in dichloromethane cooled at 0° C. The mixture is stirred at room temperature for 72 h then dichloromethane is added, and the mixture is washed with water and brine. The organic phase is dried over magnesium sulfate, and concentrated under reduced pressure to afford 0.373 g of an orange oil. Purification by chromatography over silicagel (dichloromethane/methanol/ammonia 99.5:0.5:0.05) affords 0.22 g of 2-[4-(3-chloropropoxy)phenyl]-5-(morpholin-4-ylcarbonyl)-5,6-dihydro-4H-cyclopenta[d][1,3]thiazole x95 as a white solid.

Yield: 73%.

LC-MS (MH⁺): 407/409.

The following compounds may be prepared according to the same method;

| x96 | 2-[4-(3-chloropropoxy)phenyl]-5-[(4,4-difluoropiperidin- | LC-MS |
|---|---|---|
| | 1-yl)carbonyl]-5,6-dihydro-4H-cyclopenta[d][1,3]thiazole | (MH⁺): |
| | | 441/443 |
| x97 | 2-[4-(3-chloropropoxy)phenyl]-N-ethyl-5,6-dihydro-4H- | LC-MS |
| | cyclopenta[d][1,3]thiazole-5-carboxamide | (MH⁺): |
| | | 365/367 |
| x98 | 2-[4-(3-chloropropoxy)phenyl]-5-(pyrrolidin-1- | LC-MS |
| | ylcarbonyl)-5,6-dihydro-4H-cyclopenta[d][1,3]thiazole | (MH⁺): |
| | | 391/393 |

### 45.6. Synthesis of 2-{4-[3-(2-methylpyrrolidin-1-yl)propoxy]phenyl}-5-(morpholin-4-ylcarbonyl)-5,6-dihydro-4H-cyclopenta[d][1,3]thiazole 107

A mixture of 2-[4-(3-chloropropoxy)phenyl]-5-(morpholin-4-ylcarbonyl)-5,6-dihydro-4H-cyclopenta[d][1,3]thiazole x95 (0.22 g, 0.54 mmol, 1 eq), potassium carbonate (0.149 g, 1.08 mmol, 2 eq) and sodium iodide (0.008 g, 0.05 mmol, 0.1 eq) in acetonitrile (5 ml) is heated at 90° C. for 1 hour. The reaction mixture is cooled at room temperature and 2-methylpyrrolidine (0.083 ml, 0.81 mmol, 1.5 eq) is added. The reaction mixture is heated at reflux for 56 hours. The mixture is then concentrated and taken up with ethyl acetate. It is washed two times with water, dried over magnesium sulfate and concentrated. The yellow oil obtained is purified twice by chromatography over silicagel (dichloromethane/methanol/ammonia 95:5:0.5) to afford 0.136 g of 2-{4-[3-(2-methylpyrrolidin-1-yl)propoxy]phenyl}-5-(morpholin-4-ylcarbonyl)-5,6-dihydro-4H-cyclopenta[d][1,3]thiazole 107 as a yellow lacquer.

Yield: 59%.

LC-MS (MH⁺): 456.

Compounds 110, 111 and 112 may be synthesized according to the same method.

### Example 46

### Synthesis of N-(2-{4-[3-(2-methylpyrrolidin-1-yl)propoxy]phenyl}-5,6-dihydro-4H-cyclopenta[d][1,3]thiazol-5-yl)acetamide 109

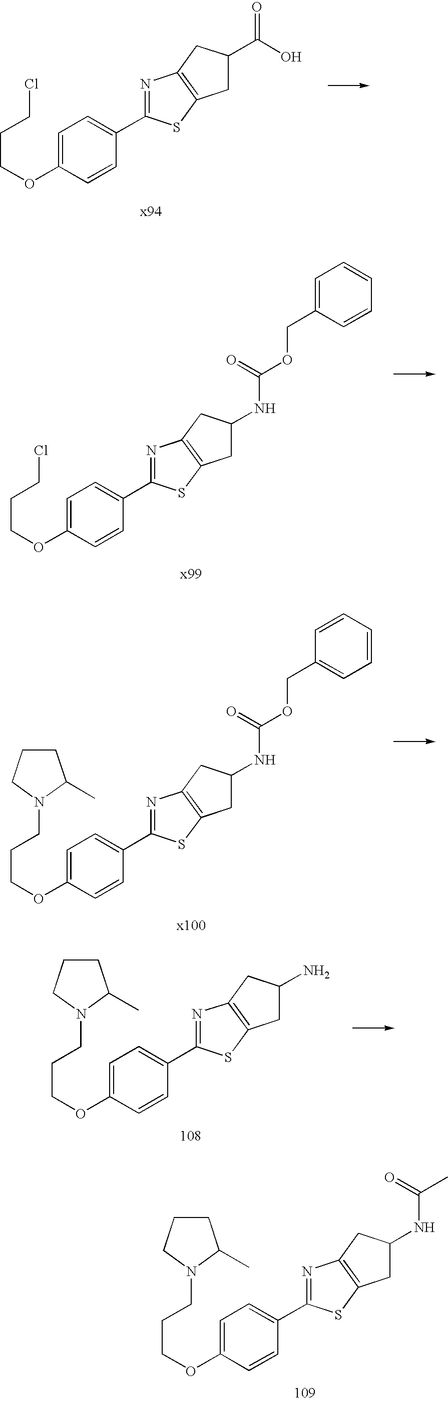

46.1 Synthesis of benzyl {2-[4-(3-chloropropoxy)phenyl]-5,6-dihydro-4H-cyclopenta[d][1,3]thiazol-5-yl}carbamate x99.

A solution of 2-[4-(3-chloropropoxy)phenyl]-5,6-dihydro-4H-cyclopenta[d][1,3]thiazole-5-carboxylic acid x94 (1 g, 2.96 mmol, 1 eq) and triethylamine (1.04 ml, 7.4 mmol, 2.5 eq) in toluene (30 ml) is treated with diphenylphosphoryl azide (0.96 ml, 4.44 mmol, 1.5 eq). The mixture is stirred overnight at 40° C. Benzyl alcohol (0.615 ml, 5.92 mmol, 2 eq) is then added and the mixture is stirred at 90° C. for 24 hours. The reaction mixture is concentrated under vacuum and the residue is taken up with ethyl acetate. The organic layer is washed with a saturated solution of ammonium chloride and a saturated solution of sodium hydrogenocarbonate, dried over magnesium sulfate and concentrated under reduced pressure to afford 1.564 g of benzyl {2-[4-(3-chloropropoxy)phenyl]-5,6-dihydro-4H-cyclopenta[d][1,3]thiazol-5-yl}carbamate x99 as an orange solid.

Yield: 100%.

LC-MS (MH⁺): 443/445.

### 46.2 Synthesis of benzyl (2-{4-[3-(2-methylpyrrolidin-1-yl)propoxy]phenyl}-5,6-dihydro-4H-cyclopenta[d][1,3]thiazol-5-yl)carbamate x100

A mixture of benzyl {2-[4-(3-chloropropoxy)phenyl]-5,6-dihydro-4H-cyclopenta[d][1,3]thiazol-5-yl}carbamate x99 (1.31 g, 2.96 mmol, 1 eq), potassium carbonate (0.818 g, 5.92 mmol, 2 eq) and sodium iodide (0.045 g, 0.30 mmol, 0.1 eq) in acetonitrile (35 ml) is heated at 90° C. for one hour. The reaction mixture is cooled at room temperature, 2-methylpyrrolidine (0.453 ml, 4.44 mmol, 1.5 eq) is added and the reaction mixture is heated at reflux for 4 days. The mixture is concentrated and taken up with ethyl acetate, washed two times with water, dried over magnesium sulfate and concentrated. The orange oil obtained is purified by chromatography over silicagel (dichloromethane/methanol/ammonia 94:6:0.6) to afford 0.534 g of benzyl (2-{4-[3-(2-methylpyrrolidin-1-yl)propoxy]phenyl}-5,6-dihydro-4H-cyclopenta[d][1,3]thiazol-5-yl)carbamate x100 as a brown solid.

Yield: 37%.

LC-MS (MH⁺): 492.

### 46.3 Synthesis of 2-{4-[3-(2-methylpyrrolidin-1-yl)propoxy]phenyl}-5,6-dihydro-4H-cyclopenta[d][1,3]thiazol-5-amine 108

A solution of benzyl (2-{4-[3-(2-methylpyrrolidin-1-yl)propoxy]phenyl}-5,6-dihydro-4H-cyclopenta[d][1,3]thiazol-5-yl)carbamate x100 (0.534 g, 1.08 mmol, 1 eq) in a 5 N aqueous solution of hydrochloric acid (10.9 ml) is stirred at reflux for 1.5 hours. The reaction mixture is cooled to room temperature and washed with diethyl ether. The aqueous phase is brought to pH 12 with a solution of 2 N sodium hydroxide and extracted 3 times with ethyl acetate. The combined organic layers are dried over magnesium sulfate and concentrated in vacuo to afford 0.35 g of 2-{4-[3-(2-methylpyrrolidin-1-yl)propoxy]phenyl}-5,6-dihydro-4H-cyclopenta[d][1,3]thiazol-5-amine 108 as a yellow oil.

Yield: 98%.

LC-MS (MH⁺): 358.

### 46.4 Synthesis of N-(2-{4-[3-(2-methylpyrrolidin-1-yl)propoxy]phenyl}-5,6-dihydro-4H-cyclopenta[d][1,3]thiazol-5-yl)acetamide 109

To a solution of 2-{4-[3-(2-methylpyrrolidin-1-yl)propoxy]phenyl}-5,6-dihydro-4H-cyclopenta[d][1,3]thiazol-5-amine 108 (0.32 g, 0.89 mmol, 1 eq) in dichloromethane (20 ml) at 0° C. is added triethylamine (0.188 ml, 1.35 mmol, 1.52 eq) and acetyl chloride (0.077 ml, 1.07 mmol, 1.2 eq). The mixture is stirred for 2.5 hours at room temperature. Dichloromethane is added (20 ml) and the organic layer is washed with a saturated solution of sodium hydrogenocarbonate and brine, dried over magnesium sulfate and concentrated under reduced pressure. The residue is purified by chromatography over silicagel (gradient: dichloromethane/methanol/ammonia from 95:5:0.5 to 93:7:0.7) to afford 0.174 g of N-(2-{4-[3-(2-methylpyrrolidin-1-yl)propoxy]phenyl}-5,6-dihydro-4H-cyclopenta[d][1,3]thiazol-5-yl)acetamide 109 as a yellow solid.

Yield: 49%.

LC-MS (MH⁺): 400.

### Example 47

### Synthesis of 5-acetyl-2-{4-[3-(2-methylpyrrolidin-1-yl)propoxy]phenyl}-5,6-dihydro-4H-pyrrolo[3,4-d][1,3]thiazole 113

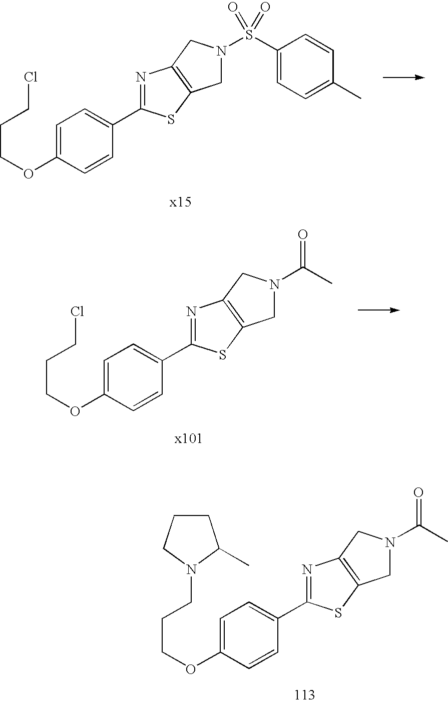

### 47.1 Synthesis of 5-acetyl-2-[4-(3-chloropropoxy)phenyl]-5,6-dihydro-4H-pyrrolo[3,4-d][1,3]thiazole x101

2-[4-(3-chloropropoxy)phenyl]-5-[(4-methylphenyl)sulfonyl]-5,6-dihydro-4H-pyrrolo[3,4-d][1,3]thiazole x15 (2.0 g, 4.45 mmol, 1 eq) is treated with a solution of 4-hydroxybenzoic acid (2.46 g, 17.82 mmol, 4 eq) and hydrogen bromide in acetic acid (40 ml). The mixture is stirred 6 days at 20° C. It is then poured onto ice-cold water (500 ml) and extracted with dichloromethane (2×100 ml). The organic layer is dried over magnesium sulfate and concentrated under reduced pressure. Chromatography over silicagel (dichloromethane/methanol/ammonia 90:9:1) affords 130 mg of 5-acetyl-2-[4-(3-chloropropoxy)phenyl]-5,6-dihydro-4H-pyrrolo[3,4-d][1,3]thiazole x101.

Yield: 8.6%.

LC-MS (MH⁺): 337/339.

### 47.2 Synthesis of 5-acetyl-2-{4-[3-(2-methylpyrrolidin-1-yl)propoxy]phenyl}-5,6-dihydro-4H-pyrrolo[3,4-d][1,3]thiazole 113

A solution of 5-acetyl-2-[4-(3-chloropropoxy)phenyl]-5,6-dihydro-4H-pyrrolo[3,4-d][1,3]thiazole x101 (130 mg, 0.39 mmol, 1 eq) in acetonitrile (5 ml) is treated with 2-methylpyrrolidine (0.2 g, 2.3 mmol, 6 eq). The mixture is stirred at 100° C. for 6 h in a sealed tube. After removal of the volatiles, the residue is purified by chromatography over silicagel (dichloromethane/methanol/ammonia 90:9:1) to obtain 50 mg of 5-acetyl-2-{4-[3-(2-methylpyrrolidin-1-yl)propoxy]phenyl}-5,6-dihydro-4H-pyrrolo[3,4-d][1,3]thiazole 113.

Yield: 33%.

LC-MS (MH⁺): 386.

TABLE 1
Physical Characterisation of Exemplified Compounds.

| n^{o} | IUPAC Name | MH⁺ | ¹H NMR δ (in CDCl₃ unless otherwise specified) |
|---|---|---|---|
| 1 | 2-[4-(3-piperidin-1-ylpropoxy)phenyl]- | 358 | (DMSO): 2.25 (s, 2H), 2.76 (s, 2H), 3.32 (m, 4H), |
| | 4,5,6,7-tetrahydro[1,3]thiazolo[5,4- | | 3.59 (s, 2H), 3.91 (s, 2H), 4.76 (m, 10H), 7.85 (d, |
| | c]pyridine | | 7.53 Hz, 2H), 8.63 (d, 7.53 Hz, 2H) |
| 2 | 2-{4-[3-(2-methylpyrrolidin-1- | 358 | (DMSO): 1.14 (d, 6.04 Hz, 3H), 1.48 (m, 1H), |
| | yl)propoxy]phenyl}-4,5,6,7- | | 1.78 (m, 2H), 1.95 (m, 2H), 2.05 (m, 3H), 2.24 (m, 3H), |
| | tetrahydro[1,3]thiazolo[5,4-c]pyridine | | 2.39 (m, 1H), 2.86 (m, 2H), 3.02 (m, 1H), 3.21 (t, |
| | | | 5.92 Hz, 2H), 4.08 (m, 3H), 6.93 (d, 8.81 Hz, 2H), |
| | | | 7.81 (m, 2H) |
| 3 | 5-ethyl-2-{4-[3-(2-methylpyrrolidin-1- | 386 | 1.09 (d, 6.04 Hz, 3H), 1.20 (t, 7.18 Hz, 3H), |
| | yl)propoxy]phenyl}-4,5,6,7- | | 1.43 (m, 1H), 1.74 (m, 2H), 1.96 (m, 3H), 2.12 (q, 8.81 Hz, |
| | tetrahydro[1,3]thiazolo[5,4-c]pyridine | | 1H), 2.21 (m, 1H), 2.31 (m, 1H), 2.66 (q, 7.11 Hz, |
| | | | 2H), 2.89 (m, 2H), 2.95 (m, 3H), 3.18 (m, 1H), |
| | | | 3.73 (s, 2H), 4.07 (m, 2H), 6.92 (d, 8.81 Hz, 2H), |
| | | | 7.80 (d, 8.81 Hz, 2H) |
| 4 | 5-acetyl-2-{4-[3-(2-methylpyrrolidin-1- | 400 | (DMSO): 1.09 (d, 6.05 Hz, 3H), 1.43 (m, 1H), |
| | yl)propoxy]phenyl}-4,5,6,7- | | 1.74 (m, 2H), 1.96 (m, 3H), 2.12 (q, 8.80 Hz, 1H), |
| | tetrahydro[1,3]thiazolo[5,4-c]pyridine | | 2.21 (m, 4H), 2.29 (m, 1H), 2.97 (m, 3H), 3.18 (m, 1H), |
| | | | 3.80 (t, 5.78 Hz, 1H), 3.96 (t, 5.78 Hz, 1H), 4.07 (m, |
| | | | 2H), 4.69 (s, 1H), 4.82 (s, 1H), 6.94 (d, 8.80 Hz, 2H), |
| | | | 7.80 (m, 2H) |
| 5 | 5-acetyl-2-(4-{3-[(2R)-2- | 400 | (DMSO): 1.09 (d, 6.05 Hz, 3H), 1.43 (m, 1H), |
| | methylpyrrolidin-1-yl]propoxy}phenyl)- | | 1.73 (m, 2H), 1.96 (m, 3H), 2.12 (q, 8.80 Hz, 1H), |
| | 4,5,6,7-tetrahydro[1,3]thiazolo[5,4- | | 2.21 (m, 4H), 2.30 (m, 1H), 2.96 (m, 3H), 3.18 (m, 1H), |
| | c]pyridine | | 3.80 (t, 5.78 Hz, 1H), 3.96 (t, 5.78 Hz, 1H), 4.07 (m, |
| | | | 2H), 4.69 (s, 1H), 4.82 (s, 1H), 6.94 (d, 8.80 Hz, 2H), |
| | | | 7.80 (d, 8.80 Hz, 2H) |
| 6 | 5-acetyl-2-(4-{3-[(2S)-2- | 400 | (DMSO): 1.09 (d, 6.05 Hz, 3H), 1.43 (m, 1H), |
| | methylpyrrolidin-1-yl]propoxy}phenyl)- | | 1.74 (m, 2H), 1.96 (m, 3H), 2.12 (q, 8.80 Hz, 1H), |
| | 4,5,6,7-tetrahydro[1,3]thiazolo[5,4- | | 2.21 (m, 4H), 2.30 (m, 1H), 2.97 (m, 3H), 3.18 (m, 1H), |
| | c]pyridine | | 3.80 (t, 5.87 Hz, 1H), 3.96 (s, 1H), 4.07 (m, 2H), |
| | | | 4.69 (s, 1H), 4.82 (s, 1H), 6.94 (m, 2H), 7.80 (m, 2H) |
| 7 | 5-(cyclohexylmethyl)-2-{4-[3-(2- | 454 | (DMSO): 0.91 (m, 2H), 1.10 (d, 6.02 Hz, 3H), |
| | methylpyrrolidin-1-yl)propoxy]phenyl}- | | 1.22 (m, 4H), 1.43 (m, 1H), 1.57 (m, 1H), 1.76 (m, 5H), |
| | 4,5,6,7-tetrahydro[1,3]thiazolo[5,4- | | 1.96 (m, 4H), 2.13 (q, 8.78 Hz, 1H), 2.21 (m, 1H), |
| | c]pyridine | | 2.31 (d, 6.78 Hz, 1H), 2.37 (d, 7.03 Hz, 2H), |
| | | | 2.84 (m, 2H), 2.96 (m, 3H), 3.18 (m, 1H), 3.68 (s, 2H), |
| | | | 4.07 (m, 2H), 6.92 (d, 9.03 Hz, 2H), 7.80 (d, 8.78 Hz, |
| | | | 2H) |
| 8 | 5-cyclopentyl-2-{4-[3-(2- | 426 | 1.03 (d, 6.02 Hz, 3H), 1.31 (m, 2H), 1.42 (m, 2H), |
| | methylpyrrolidin-1-yl)propoxy]phenyl}- | | 1.53 (dd, 7.53, 4.77 Hz, 2H), 1.66 (m, 4H), 1.88 (m, |
| | 4,5,6,7-tetrahydro[1,3]thiazolo[5,4- | | 5H), 2.18 (m, 2H), 2.38 (m, 1H), 2.79 (m, 4H), |
| | c]pyridine | | 2.95 (m, 1H), 3.12 (s, 1H), 3.70 (s, 2H), 4.07 (t, |
| | | | 6.27 Hz, 2H), 7.01 (d, 8.78 Hz, 2H), 7.78 (d, 8.78 Hz, |
| | | | 2H) |
| 9 | 2-{4-[3-(2-methylpyrrolidin-1- | 454 | (DMSO): 0.99 (d, 6.02 Hz, 3H), 1.27 (m, 1H), |
| | yl)propoxy]phenyl}-5-(trifluoroacetyl)- | | 1.63 (m, 2H), 1.87 (m, 3H), 2.03 (q, 8.72 Hz, 1H), |
| | 4,5,6,7-tetrahydro[1,3]thiazolo[5,4- | | 2.12 (q, 8.78 Hz, 1H), 2.25 (m, 1H), 2.93 (m, 3H), |
| | c]pyridine | | 3.07 (m, 1H), 3.95 (m, 2H), 4.07 (t, 6.27 Hz, 2H), |
| | | | 4.90 (m, 2H), 7.03 (d, 8.78 Hz, 2H), 7.81 (d, 8.78 Hz, 2H) |
| 10 | 5-(4-fluorophenyl)-2-{4-[3-(2- | 452 | (DMSO): 0.99 (d, 6.02 Hz, 3H), 1.27 (m, 1H), |
| | methylpyrrolidin-1-yl)propoxy]phenyl}- | | 1.63 (m, 2H), 1.87 (m, 3H), 2.03 (q, 8.78 Hz, 1H), |
| | 4,5,6,7-tetrahydro[1,3]thiazolo[5,4- | | 2.11 (m, 1H), 2.24 (m, 1H), 2.89 (m, 3H), 3.08 (m, 1H), |
| | c]pyridine | | 3.64 (t, 5.52 Hz, 2H), 4.08 (m, 2H), 4.47 (s, 2H), |
| | | | 7.02 (d, 8.78 Hz, 2H), 7.06 (d, 6.53 Hz, 4H), |
| | | | 7.80 (d, 8.78 Hz, 2H) |
| 11 | 5-benzoyl-2-{4-[3-(2-methylpyrrolidin-1- | 462 | (DMSO): 0.99 (d, 6.02 Hz, 3H), 1.27 (m, 1H), |
| | yl)propoxy]phenyl}-4,5,6,7- | | 1.62 (m, 2H), 1.87 (m, 3H), 2.03 (d, 8.78 Hz, 1H), |
| | tetrahydro[1,3]thiazolo[5,4-c]pyridine | | 2.12 (m, 1H), 2.25 (m, 1H), 2.89 (m, 3H), 3.07 (m, 1H), |
| | | | 3.66 (dd, 1.76, 0.88 Hz, 1H), 4.07 (t, 6.27 Hz, 3H), |
| | | | 4.87 (d, 0.75 Hz, 2H), 7.02 (d, 8.53 Hz, 2H), |
| | | | 7.49 (s, 5H), 7.80 (d, 7.03 Hz, 2H) |
| 12 | 5-(cyclohexylcarbonyl)-2-{4-[3-(2- | 468 | (DMSO): 1.19 (m, 4H), 1.37 (m, 5H), 1.67 (m, 6H), |
| | methylpyrrolidin-1-yl)propoxy]phenyl}- | | 1.79 (d, 5.52 Hz, 2H), 2.02 (s, 3H), 2.75 (s, 2H), |
| | 4,5,6,7-tetrahydro[1,3]thiazolo[5,4- | | 2.88 (s, 2H), 3.23 (m, 3H), 3.83 (d, 5.53 Hz, 2H), |
| | c]pyridine | | 4.11 (t, 6.28 Hz, 2H), 4.71 (s, 1H), 4.83 (s, 1H), |
| | | | 7.04 (d, 8.54 Hz, 2H), 7.81 (d, 8.29 Hz, 2H) |
| 13 | 2-{4-[3-(2-methylpyrrolidin-1- | 468 | (DMSO): 0.99 (d, 6.02 Hz, 3H), 1.27 (m, 1H), |
| | yl)propoxy]phenyl}-5-(thien-2- | | 1.63 (m, 2H), 1.87 (m, 3H), 2.03 (q, 8.78 Hz, 1H), |
| | ylcarbonyl)-4,5,6,7- | | 2.12 (m, 1H), 2.24 (m, 1H), 2.91 (m, 3H), 3.07 (m, 1H), |
| | tetrahydro[1,3]thiazolo[5,4-c]pyridine | | 3.97 (t, 5.77 Hz, 2H), 4.07 (t, 6.40 Hz, 2H), 4.93 (s, |
| | | | 2H), 7.02 (d, 8.78 Hz, 2H), 7.18 (dd, 4.77, 4.02 Hz, |
| | | | 1H), 7.56 (d, 3.26 Hz, 1H), 7.80 (m, 3H) |
| 14 | 5-(2,2-dimethylpropanoyl)-2-{4-[3-(2- | 442 | (DMSO): 0.99 (d, 6.02 Hz, 3H), 1.24 (s, 9H), |
| | methylpyrrolidin-1-yl)propoxy]phenyl}- | | 1.27 (m, 1H), 1.63 (m, 2H), 1.87 (m, 3H), 2.07 (m, 2H), |
| | 4,5,6,7-tetrahydro[1,3]thiazolo[5,4- | | 2.25 (m, 1H), 2.88 (m, 3H), 3.07 (m, 1H), 3.89 (t, |
| | c]pyridine | | 5.65 Hz, 2H), 4.07 (t, 6.40 Hz, 2H), 4.78 (s, 2H), |
| | | | 7.02 (d, 8.78 Hz, 2H), 7.80 (d, 8.78 Hz, 2H) |
| 15 | 5-butyry-2-{4-[3-(2-methylpyrrolidin-1- | 428 | (DMSO): 0.90 (m, 3H), 1.11 (d, 5.27 Hz, 3H), |
| | yl)propoxy]phenyl}-4,5,6,7- | | 1.55 (m, 2H), 1.73 (m, 2H), 1.96 (s, 3H), 2.40 (m, 4H), |
| | tetrahydro[1,3]thiazolo[5,4-c]pyridine | | 2.77 (s, 1H), 2.88 (5, 1H), 3.06 (m, 1H), 3.23 (m, 3H), |
| | | | 3.80 (m, 2H), 4.10 (t, 6.15 Hz, 2H), 4.75 (m, 2H), |
| | | | 7.03 (d, 8.78 Hz, 2H), 7.80 (d, 8.53 Hz, 2H) |
| 16 | tert-butyl 2-{4-[3-(2-methylpyrrolidin-1- | 458 | (DMSO): 1.10 (d, 3.76 Hz, 3H), 1.43 (s, 9H), |
| | yl)propoxy]phenyl}-6,7- | | 1.72 (d, 5.77 Hz, 2H), 1.94 (d, 19.32 Hz, 3H), 2.80 (t, |
| | dihydro[1,3]thiazolo[5,4-c]pyridine- | | 5.52 Hz, 2H), 3.05 (m, 1H), 3.28 (m, 4H), 3.68 (t, |
| | 5(4H)-carboxylate | | 5.77 Hz, 2H), 4.09 (t, 6.15 Hz, 2H), 4.61 (s, 2H), |
| | | | 7.03 (d, 8.78 Hz, 2H), 7.80 (d, 8.78 Hz, 2H) |
| 17 | N-ethyl-2-{4-[3-(2-methylpyrrolidin-1- | 429 | (DMSO, some signals obscured by solvent): 1.03 (t, |
| | yl)propoxy]phenyl}-6,7- | | 7.15 Hz, 3H), 1.13 (d, 5.27 Hz, 2H), 1.41 (m, 1H), |
| | dihydro[1,3]thiazolo[5,4-c]pyridine- | | 1.75 (m, 2H), 1.98 (s, 3H), 2.77 (t, 5.40 Hz, 3H), |
| | 5(4H)-carboxamide | | 3.09 (m, 3H), 3.32 (m, 2H), 3.67 (t, 5.65 Hz, 2H), |
| | | | 4.10 (t, 6.27 Hz, 2H), 4.59 (s, 2H), 6.73 (s, 1H), |
| | | | 7.03 (d, 8.78 Hz, 2H), 7.80 (d, 8.78 Hz, 2H) |
| 18 | N-ethyl-2-(4-{3-[(2R)-2-methylpyrrolidin- | 429 | 1.09 (d, 6.0 Hz, 3H), 1.18 (t, 7.3 Hz, 3H), 1.43 (m, 1H), |
| | 1-yl]propoxy}phenyl)-6,7- | | 1.75 (m, 2H), 1.96 (m, 3H), 2.12 (q, 8.8 Hz, 1H), |
| | dihydro[1,3]thiazolo[5,4-c]pyridine- | | 2.21 (m, 1H), 2.31 (m, 1H), 2.97 (m, 3H), |
| | 5(4H)-carboxamide | | 3.17 (td, 8.8, 2.8 Hz, 1H), 3.32 (m, 2H), 3.71 (t, 5.8 Hz, 2H), |
| | | | 4.08 (td, 6.3, 2.3 Hz, 2H), 4.56 (t, 4.5 Hz, 1H), |
| | | | 4.65 (s, 2H), 6.93 (d, 8.8 Hz, 2H), 7.79 (d, 8.8 Hz, 2H) |
| 19 | N-ethyl-2-(4-{3-[(2S)-2-methylpyrrolidin- | 429 | 1.09 (d, 6.02 Hz, 3H), 1.17 (t, 7.28 Hz, 3H), |
| | 1-yl]propoxy}phenyl)-6,7- | | 1.42 (m, 1H), 1.73 (m, 2H), 1.96 (m, 3H), 2.12 (q, 8.91 Hz, |
| | dihydro[1,3]thiazolo[5,4-c]pyridine- | | 1H), 2.21 (m, 1H), 2.30 (m, 1H), 2.96 (m, 3H), |
| | 5(4H)-carboxamide | | 3.17 (td, 8.53, 2.76 Hz, 1H), 3.32 (dd, 7.28, 5.52 Hz, |
| | | | 2H), 3.72 (t, 5.77 Hz, 2H), 4.07 (m, 2H), 4.62 (m, 3H), |
| | | | 6.93 (d, 8.78 Hz, 2H), 7.80 (d, 8.78 Hz, 2H) |
| 20 | 5-(cyclopropylcarbonyl)-2-{4-[3-(2- | 426 | (DMSO): 0.77 (m, 4H), 0.99 (d, 6.02 Hz, 3H), |
| | methylpyrrolidin-1-yl)propoxy]phenyl}- | | 1.28 (m, 1H), 1.63 (m, 2H), 1.86 (m, 3H), 2.08 (m, 3H), |
| | 4,5,6,7-tetrahydro[1,3]thiazolo[5,4- | | 2.77 (s, 1H), 2.90 (m, 2H), 3.07 (m, 1H), 3.83 (s, 1H), |
| | c]pyridine | | 4.06 (m, 3H), 4.73 (s, 1H), 5.01 (s, 1H), |
| | | | 7.02 (d, 8.78 Hz, 2H), 7.80 (d, 8.53 Hz, 2H) |
| 21 | 5-isonicotinoyl-2-{4-[3-(2- | 463 | (DMSO): 0.99 (d, 6.02 Hz, 3H), 1.27 (m, 1H), |
| | methylpyrrolidin-1-yl)propoxy]phenyl}- | | 1.63 (m, 2H), 1.87 (m, 3H), 2.07 (m, 2H), 2.25 (m, 1H), |
| | 4,5,6,7-tetrahydro[1,3]thiazolo[5,4- | | 2.90 (m, 3H), 3.07 (m, 1H), 3.60 (t, 5.14 Hz, 1H), |
| | c]pyridine | | 4.05 (m, 3H), 4.62 (s, 1H), 4.91 (s, 1H), 7.03 (m, 2H), |
| | | | 7.47 (m, 2H), 7.80 (m, 2H), 8.71 (d, 4.77 Hz, 2H) |
| 22 | 5-(methoxyacetyl)-2-(4-{3-[(2R)-2- | 430 | 1.09 (d, 6.02 Hz, 3H), 1.42 (m, 1H), 1.74 (m, 2H), |
| | methylpyrrolidin-1-yl]propoxy}phenyl)- | | 1.96 (m, 3H), 2.12 (q, 8.78 Hz, 1H), 2.21 (m, 1H), |
| | 4,5,6,7-tetrahydro[1,3]thiazolo[5,4- | | 2.30 (m, 1H), 2.98 (m, 3H), 3.18 (m, 1H), 3.45 (s, 3H), |
| | c]pyridine | | 3.83 (t, 5.52 Hz, 1H), 3.97 (s, 1H), 4.07 (m, 2H), |
| | | | 4.22 (s, 2H), 4.82 (s, 2H), 6.94 (d, 8.78 Hz, 2H), |
| | | | 7.80 (d, 8.53 Hz, 2H) |
| 23 | 5-(methoxyacetyl)-2-{4-[3-(2- | 430 | 1.09 (d, 5.87 Hz, 3H), 1.42 (m, 1H), 1.71 (m, 1H), |
| | methylpyrrolidin-1-yl)propoxy]phenyl}- | | 1.91 (s, 1H), 2.01 (m, 2H), 2.12 (d, 8.80 Hz, 1H), |
| | 4,5,6,7-tetrahydro[1,3]thiazolo[5,4- | | 2.21 (m, 1H), 2.30 (m, 1H), 2.98 (m, 3H), 3.18 (m, |
| | c]pyridine | | 1H), 3.45 (s, 3H), 3.83 (t, 5.59 Hz, 1H), 3.98 (s, 1H), |
| | | | 4.08 (m, 2H), 4.21 (s, 2H), 4.79 (m, 2H), |
| | | | 6.94 (d, 8.80 Hz, 2H), 7.80 (d, 8.62 Hz, 2H) |
| 24 | 5-(methoxyacetyl)-2-(4-{3-[(2S)-2- | 430 | (DMSO, some signals obscured by solvent): |
| | methylpyrrolidin-1-yl]propoxy}phenyl)- | | 0.99 (d, 5.77 Hz, 3H), 1.27 (m, 1H), 1.63 (m, 2H), |
| | 4,5,6,7-tetrahydro[1,3]thiazolo[5,4- | | 1.87 (m, 3H), 2.03 (q, 8.66 Hz, 1H), 2.12 (m, 1H), |
| | c]pyridine | | 2.25 (m, 1H), 2.86 (m, 3H), 3.07 (m, 1H), 3.77 (m, |
| | | | 2H), 4.07 (t, 6.27 Hz, 2H), 4.20 (m, 2H), 4.72 (s, 2H), |
| | | | 7.02 (d, 8.78 Hz, 2H), 7.80 (d, 8.53 Hz, 2H) |
| 25 | N-ethyl-2-(4-{3-[2-(4-{3-[(2S)-2- | 701 | (DMSO): 1.01 (m, 6H), 1.27 (m, 1H), 1.64 (m, 3H), |
| | methylpyrrolidin-1-yl]propoxy}phenyl)- | | 1.98 (m, 7H), 2.25 (m, 1H), 2.70 (t, 6.90 Hz, 2H), |
| | 6,7-dihydro[1,3]thiazolo[5,4-c]pyridin- | | 2.85 (m, 7H), 3.08 (m, 3H), 3.69 (m, 4H), 4.09 (m, |
| | 5(4H)-yl]propoxy}phenyl)-6,7- | | 4H), 4.59 (s, 2H), 6.71 (t, 5.14 Hz, 1H), 7.02 (t, |
| | dihydro[1,3]thiazolo[5,4-c]pyridine- | | 9.29 Hz, 4H), 7.79 (m, 4H) |
| | 5(4H)-carboxamide | | |
| 26 | 2-{4-[3-(2-methylpyrrolidin-1- | 436 | 1.10 (d, 6.02 Hz, 3H), 1.44 (m, 1H), 1.75 (m, 2H), |
| | yl)propoxy]phenyl}-5-(methylsulfonyl)- | | 1.97 (m, 3H), 2.12 (m, 1H), 2.22 (m, 1H), 2.32 (m, |
| | 4,5,6,7-tetrahydro[1,3]thiazolo[5,4- | | 1H), 2.88 (s, 3H), 3.02 (m, 3H), 3.19 (m, 1H), |
| | c]pyridine | | 3.72 (t, 5.90 Hz, 2H), 4.08 (m, 2H), 4.59 (s, 2H), 6.94 (d, |
| | | | 8.78 Hz, 2H), 7.81 (d, 8.53 Hz, 2H) |
| 27 | N-benzyl-2-{4-[3-(2-methylpyrrolidin-1- | 491 | 1.09 (d, 6.0 Hz, 3H), 1.43 (m, 1H), 1.74 (m, 2H), |
| | yl)propoxy]phenyl}-6,7- | | 1.91 (m, 1H), 2.01 (m, 2H), 2.12 (q, 8.8 Hz, 1H), |
| | dihydro[1,3]thiazolo[5,4-c]pyridine- | | 2.21 (m, 1H), 2.30 (m, 1H), 2.97 (m, 3H), 3.18 (td, |
| | 5(4H)-carboxamide | | 8.8, 2.8 Hz, 1H), 3.74 (t, 5.8 Hz, 2H), 4.07 (td, 6.3, |
| | | | 2.3 Hz, 2H), 4.47 (d, 5.3 Hz, 2H), 4.67 (s, 2H), |
| | | | 4.89 (t, 5.0 Hz, 1H), 6.93 (d, 9.0 Hz, 2H), 7.33 (m, 4H), |
| | | | 7.79 (d, 8.8 Hz, 2H) |
| 28 | 2-{4-[3-(2-methylpyrrolidin-1- | 477 | 1.09 (d, 6.0 Hz, 3H), 1.43 (m, 1H), 1.74 (m, 2H), |
| | yl)propoxy]phenyl}-N-phenyl-6,7- | | 1.96 (m, 3H), 2.12 (q, 8.8 Hz, 1H), 2.21 (m, 1H), |
| | dihydro[1,3]thiazolo[5,4-c]pyridine- | | 2.31 (m, 1H), 2.98 (m, 3H), 3.18 (td, 8.5, 2.3 Hz, 1H), |
| | 5(4H)-carboxamide | | 3.83 (t, 5.5 Hz, 2H), 4.07 (td, 6.0, 1.5 Hz, 2H), |
| | | | 4.75 (s, 2H), 6.59 (s, 1H), 6.94 (d, 8.5 Hz, 2H), |
| | | | 7.05 (m, 1H), 7.29 (m, 2H), 7.37 (m, 2H), 7.80 (d, |
| | | | 8.5 Hz, 2H) |
| 29 | 2-{4-[3-(2-methylpyrrolidin-1- | 511 | 1.09 (d, 6.1 Hz, 3H), 1.42 (m, 1H), 1.74 (m, 2H), |
| | yl)propoxy]phenyl}-N-(2-thien-2- | | 1.96 (m, 3H), 2.12 (q, 8.8 Hz, 1H), 2.21 (m, 1H), |
| | ylethyl)-6,7-dihydro[1,3]thiazolo[5,4- | | 2.31 (m, 1H), 2.92 (t, 5.7 Hz, 2H), 2.98 (dt, 11.7, |
| | c]pyridine-5(4H)-carboxamide | | 8.1 Hz, 1H), 3.07 (t, 6.4 Hz, 2H), 3.18 (m, 1H), |
| | | | 3.55 (q, 6.2 Hz, 2H), 3.67 (t, 5.9 Hz, 2H), 4.07 (m, 2H), |
| | | | 4.63 (s, 2H), 4.74 (t, 5.5 Hz, 1H), 6.83 (d, 3.1 Hz, |
| | | | 1H), 6.94 (m, 3H), 7.16 (d, 5.1 Hz, 1H), |
| | | | 7.80 (d, 8.8 Hz, 2H) |
| 30 | 2-{4-[3-(2-methylpyrrolidin-1- | 401 | 1.09 (d, 5.8 Hz, 3H), 1.42 (m, 1H), 1.73 (m, 2H), |
| | yl)propoxy]phenyl}-6,7- | | 1.97 (dd, 8.3, 8.3, 1.0, 0.5 Hz, 3H), 2.12 (q, 8.8 Hz, |
| | dihydro[1,3]thiazolo[5,4-c]pyridine- | | 1H), 2.21 (m, 1H), 2.29 (m, 1H), 2.99 (m, 3H), |
| | 5(4H)-carboxamide | | 3.17 (m, 1H), 3.73 (t, 5.5 Hz, 2H), 4.07 (m, 2H), |
| | | | 4.70 (m, 4H), 6.93 (d, 8.3 Hz, 2H), 7.80 (d, 8.3 Hz, |
| | | | 2H) |
| 31 | N-isopropyl-2-{4-[3-(2-methylpyrrolidin- | 443 | 1.09 (d, 6.0 Hz, 3H), 1.19 (d, 6.5 Hz, 6H), 1.42 (m, |
| | 1-yl)propoxy]phenyl}-6,7- | | 1H), 1.74 (m, 2H), 1.96 (m, 3H), 2.12 (q, 8.8 Hz, 1H), |
| | dihydro[1,3]thiazolo[5,4-c]pyridine- | | 2.21 (m, 1H), 2.30 (m, 1H), 2.97 (m, 3H), |
| | 5(4H)-carboxamide | | 3.18 (td, 8.8, 2.8 Hz, 1H), 3.70 (t, 5.8 Hz, 2H), 4.04 (m, 3H), |
| | | | 4.36 (d, 7.0 Hz, 1H), 4.64 (s, 2H), 6.93 (d, 8.5 Hz, |
| | | | 2H), 7.80 (d, 8.5 Hz, 2H) |
| 32 | N-cyclohexyl-2-{4-[3-(2- | 483 | 1.09 (d, 6.0 Hz, 3H), 1.16 (m, 3H), 1.40 (m, 3H), |
| | methylpyrrolidin-1-yl)propoxy]phenyl}- | | 1.71 (m, 5H), 1.96 (m, 5H), 2.12 (q, 8.8 Hz, 1H), |
| | 6,7-dihydro[1,3]thiazolo[5,4-c]pyridine- | | 2.21 (m, 1H), 2.30 (m, 1H), 2.96 (m, 3H), 3.17 (m, |
| | 5(4H)-carboxamide | | 1H), 3.70 (m, 3H), 4.07 (td, 6.0, 2.3 Hz, 2H), |
| | | | 4.41 (d, 7.5 Hz, 1H), 4.63 (s, 2H), 6.93 (d, 8.8 Hz, 2H), |
| | | | 7.80 (d, 8.8 Hz, 2H) |
| 33 | 2-{4-[3-(2-methylpyrrolidin-1- | 580 | 1.09 (d, 6.0 Hz, 3H), 1.40 (m, 3H), 1.74 (dd, 9.8, |
| | yl)propoxy]phenyl}-N-[1- | | 4.3 Hz, 2H), 1.92 (m, 1H), 2.01 (m, 2H), 2.17 (m, 4H), |
| | (trifluoroacetyl)piperidin-4-yl]-6,7- | | 2.31 (m, 1H), 2.95 (m, 4H), 3.22 (m, 2H), |
| | dihydro[1,3]thiazolo[5,4-c]pyridine- | | 3.72 (t, 5.8 Hz, 2H), 4.04 (m, 4H), 4.55 (m, 2H), 4.65 (s, |
| | 5(4H)-carboxamide | | 2H), 6.93 (d, 8.8 Hz, 2H), 7.80 (d, 8.8 Hz, 2H) |
| 34 | ethyl ({[2-{4-[3-(2-methylpyrrolidin-1- | 487 | 1.09 (d, 6.0 Hz, 3H), 1.29 (t, 7.3 Hz, 3H), 1.42 (m, 1H), |
| | yl)propoxy]phenyl}-6,7- | | 1.74 (m, 2H), 1.91 (m, 1H), 2.00 (m, 2H), |
| | dihydro[1,3]thiazolo[5,4-c]pyridin-5(4H)- | | 2.12 (q, 8.8 Hz, 1H), 2.21 (m, 1H), 2.30 (m, 1H), |
| | yl]carbonyl}amino)acetate | | 2.98 (m, 3H), 3.18 (td, 8.8, 2.8 Hz, 1H), 3.76 (t, 5.8 Hz, 2H), |
| | | | 4.07 (m, 4H), 4.23 (q, 7.0 Hz, 2H), 4.69 (s, 2H), |
| | | | 5.17 (t, 4.8 Hz, 1H), 6.93 (d, 8.8 Hz, 2H), 7.80 (d, |
| | | | 8.8 Hz, 2H) |
| 35 | N-(2,4-difluorophenyl)-2-{4-[3-(2- | 513 | 1.09 (d, 6.3 Hz, 3H), 1.42 (m, 1H), 1.75 (m, 2H), |
| | methylpyrrolidin-1-yl)propoxy]phenyl}- | | 1.92 (m, 1H), 2.01 (m, 2H), 2.12 (q, 8.8 Hz, 1H), |
| | 6,7-dihydro[1,3]thiazolo[5,4-c]pyridine- | | 2.21 (m, 1H), 2.31 (m, 1H), 3.00 (m, 3H), 3.18 (td, |
| | 5(4H)-carboxamide | | 8.8, 2.8 Hz, 1H), 3.86 (t, 5.8 Hz, 2H), 4.08 (td, 6.3, |
| | | | 2.0 Hz, 2H), 4.78 (s, 2H), 6.56 (d, 2.3 Hz, 1H), |
| | | | 6.86 (m, 2H), 6.94 (d, 8.8 Hz, 2H), 7.81 (d, 8.8 Hz, 2H), |
| | | | 7.97 (m, 1H) |
| 36 | 2-{4-[3-(2-methylpyrrolidin-1- | 468 | 1.09 (d, 6.0 Hz, 3H), 1.42 (m, 1H), 1.74 (m, 2H), |
| | yl)propoxy]phenyl}-5-(3,3,3- | | 1.96 (m, 3H), 2.12 (q, 8.8 Hz, 1H), 2.21 (m, 1H), |
| | trifluoropropanoyl)-4,5,6,7- | | 2.30 (m, 1H), 2.99 (m, 3H), 3.18 (td, 8.5, 2.5 Hz, 1H), |
| | tetrahydro[1,3]thiazolo[5,4-c]pyridine | | 3.36 (m, 2H), 3.82 (t, 5.5 Hz, 1H), 4.01 (s, 1H), |
| | | | 4.08 (td, 6.0, 1.8 Hz, 2H), 4.70 (s, 1H), 4.86 (s, 1H), |
| | | | 6.94 (d, 8.8 Hz, 2H), 7.80 (m, 2H) |
| 37 | 5-[(4-methylphenyl)sulfonyl]-2-{4-[3-(2- | 512 | (DMSO): 0.98 (d, 6.0 Hz, 3H), 1.26 (m, 2H), |
| | methylpyrrolidin-1-yl)propoxy]phenyl}- | | 1.62 (m, 2H), 1.85 (m, 3H), 2.02 (q, 8.5 Hz, 1H), |
| | 4,5,6,7-tetrahydro[1,3]thiazolo[5,4- | | 2.11 (m, 1H), 2.24 (m, 1H), 2.37 (s, 3H), 2.80 (t, 5.5 Hz, |
| | c]pyridine | | 2H), 2.88 (m, 1H), 3.06 (d, 2.0 Hz, 1H), 3.45 (t, 5.8 Hz, |
| | | | 2H), 4.06 (t, 6.3 Hz, 2H), 4.39 (s, 2H), 7.00 (d, |
| | | | 9.0 Hz, 2H), 7.42 (d, 8.0 Hz, 2H), 7.74 (dd, 14.1, |
| | | | 8.8 Hz, 4H) |
| 38 | N-butyl-2-{4-[3-(2-methylpyrrolidin-1- | 457 | 0 (DMSO): .87 (t, 7.3 Hz, 3H), 1.00 (d, 6.0 Hz, 3H), |
| | yl)propoxy]phenyl}-6,7- | | 1.28 (m, 3H), 1.40 (m, 2H), 1.64 (m, 2H), 1.87 (m, |
| | dihydro[1,3]thiazolo[5,4-c]pyridine- | | 3H), 2.05 (q, 8.8 Hz, 1H), 2.13 (m, 1H), 2.27 (m, 1H), |
| | 5(4H)-carboxamide | | 2.77 (t, 5.3 Hz, 2H), 2.91 (m, 1H), 3.06 (m, 3H), |
| | | | 3.67 (t, 5.8 Hz, 2H), 4.07 (t, 6.3 Hz, 2H), 4.59 (s, 2H), |
| | | | 6.68 (t, 5.3 Hz, 1H), 7.02 (d, 8.8 Hz, 2H), |
| | | | 7.79 (d, 8.8 Hz, 2H) |
| 39 | 5-acetyl-2-(4-{3-[2-(pyrrolidin-1- | 469 | 1.64 (m, 1H), 1.75 (m, 6H), 2.00 (m, 3H), 2.18 (m, |
| | ylmethyl)pyrrolidin-1- | | 4H), 2.37 (m, 2H), 2.50 (m, 5H), 2.59 (dd, 11.7, 3.7 Hz, |
| | yl]propoxy}phenyl)-4,5,6,7- | | 1H), 2.93 (m, 1H), 2.99 (m, 1H), 3.08 (dt, 11.9, |
| | tetrahydro[1,3]thiazolo[5,4-c]pyridine | | 7.9 Hz, 1H), 3.16 (m, 1H), 3.80 (t, 5.9 Hz, 1H), |
| | | | 3.96 (s, 1H), 4.07 (t, 6.2 Hz, 2H), 4.69 (s, 1H), |
| | | | 4.82 (s, 1H), 6.93 (m, 2H), 7.80 (m, 2H) |
| 40 | 5-acetyl-2-[4-(3-azepan-1- | 414 | 1.63 (m, 8H), 1.98 (m, 2H), 2.21 (m, 3H), 2.68 (t, |
| | ylpropoxy)phenyl]-4,5,6,7- | | 5.3 Hz, 6H), 2.97 (m, 2H), 3.80 (t, 5.9 Hz, 1H), |
| | tetrahydro[1,3]thiazolo[5,4-c]pyridine | | 3.96 (s, 1H), 4.07 (t, 6.4 Hz, 2H), 4.69 (s, 1H), |
| | | | 4.82 (s, 1H), 6.93 (m, 2H), 7.80 (m, 2H) |
| 41 | 5-acetyl-2-{4-[3-(4-isopropylpiperazin-1- | 443 | 1.06 (d, 6.4 Hz, 6H), 1.99 (m, 2H), 2.21 (m, 3H), |
| | yl)propoxy]phenyl}-4,5,6,7- | | 2.58 (m, 10H), 2.96 (m, 2H), 3.80 (m, 1H), 3.96 (m, |
| | tetrahydro[1,3]thiazolo[5,4-c]pyridine | | 2H), 4.06 (t, 6.4 Hz, 2H), 4.69 (s, 1H), 4.82 (s, 1H), |
| | | | 6.93 (m, 2H), 7.80 (m, 2H) |
| 42 | 5-acetyl-2-{4-[3-(2-methylpiperidin-1- | 414 | 1.07 (d, 6.1 Hz, 3H), 1.30 (m, 2H), 1.60 (m, 4H), |
| | yl)propoxy]phenyl}-4,5,6,7- | | 1.96 (m, 2H), 2.19 (m, 4H), 2.31 (m, 1H), 2.50 (m, |
| | tetrahydro[1,3]thiazolo[5,4-c]pyridine | | 1H), 2.92 (m, 4H), 3.80 (t, 5.9 Hz, 1H), 3.96 (s, 1H), |
| | | | 4.04 (m, 2H), 4.69 (s, 1H), 4.82 (s, 1H), |
| | | | 6.93 (d, 8.8 Hz, 2H), 7.80 (m, 2H) |
| 43 | 1-{3-[4-(5-acetyl-4,5,6,7- | 429 | 1.76 (m, 3H), 2.00 (t, 7.0 Hz, 3H), 2.23 (m, 9H), |
| | tetrahydro[1,3]thiazolo[5,4-c]pyridin-2- | | 2.36 (m, 1H), 2.55 (m, 2H), 2.67 (m, 1H), 2.78 (m, |
| | yl)phenoxy]propyl}-N,N- | | 2H), 2.88 (m, 1H), 2.99 (m, 1H), 3.80 (t, 5.8 Hz, 1H), |
| | dimethylpyrrolidin-3-amine | | 3.96 (s, 1H), 4.07 (t, 6.3 Hz, 2H), 4.82 (s, 1H), |
| | | | 6.93 (m, 2H), 7.80 (d, 8.8 Hz, 2H) |
| 44 | 5-acetyl-2-{4-[3-(3,5-dimethylpiperidin- | 428 | 0.53 (d, 12.3 Hz, 1H), 0.86 (d, 6.3 Hz, 6H), 1.46 (t, |
| | 1-yl)propoxy]phenyl}-4,5,6,7- | | 10.8 Hz, 2H), 1.71 (m, 4H), 2.01 (m, 2H), 2.22 (s, 3H), |
| | tetrahydro[1,3]thiazolo[5,4-c]pyridine | | 2.50 (t, 7.3 Hz, 2H), 2.87 (d, 10.5 Hz, 2H), |
| | | | 2.93 (m, 1H), 2.99 (m, 1H), 3.80 (t, 5.8 Hz, 1H), 3.96 (s, |
| | | | 1H), 4.05 (t, 6.5 Hz, 2H), 4.69 (s, 1H), 4.82 (s, 1H), |
| | | | 6.93 (m, 2H), 7.80 (d, 8.8 Hz, 2H) |
| 45 | 5-[(4-methylphenyl)sulfonyl]-2-{4-[3-(2- | 498 | (DMSO): 0.98 (d, 6.0 Hz, 3H), 1.28 (m, 1H), |
| | methylpyrrolidin-1-yl)propoxy]phenyl}- | | 1.63 (m, 2H), 1.85 (m, 3H), 2.03 (q, 8.8 Hz, 1H), |
| | 5,6-dihydro-4H-pyrrolo[3,4- | | 2.11 (m, 1H), 2.25 (m, 1H), 2.37 (s, 3H), 2.89 (m, 1H), |
| | d][1,3]thiazole | | 3.07 (m, 1H), 4.06 (t, 6.3 Hz, 2H), 4.52 (m, 2H), |
| | | | 4.63 (m, 2H), 7.01 (d, 8.8 Hz, 2H), 7.43 (d, 8.0 Hz, |
| | | | 2H), 7.78 (m, 4H) |
| 46 | 2-{4-[3-(2-methylpyrrolidin-1- | 372 | (DMSO, some signals obscured by solvent): 0.99 (d, |
| | yl)propoxy]phenyl}-5,6,7,8-tetrahydro- | | 6.0 Hz, 4H), 1.26 (m, 2H), 1.63 (m, 2H), 1.87 (m, 4H), |
| | 4H-[1,3]thiazolo[4,5-d]azepine | | 2.03 (q, 8.8 Hz, 1H), 2.12 (m, 1H), 2.25 (m, 1H), |
| | | | 2.89 (m, 7H), 3.07 (m, 1H), 4.06 (t, 6.3 Hz, 2H), |
| | | | 6.99 (d, 8.8 Hz, 2H), 7.73 (d, 8.8 Hz, 2H) |
| 47 | 2-{4-[3-(2-methylpyrrolidin-1- | 373 | 1.09 (d, 5.87 Hz, 3H), 1.42 (m, 1H), 1.89 (m, 11H), |
| | yl)propoxy]phenyl}-4,5,6,7-tetrahydro- | | 2.21 (m, 1H), 2.31 (m, 1H), 2.74 (m, 1H), 2.86 (m, |
| | 1,3-benzothiazol-4-ol | | 1H), 2.97 (m, 1H), 3.18 (m, 1H), 4.07 (m, 2H), |
| | | | 4.92 (m, 1H), 6.93 (d, 8.07 Hz, 2H), 7.81 (d, 7.89 Hz, |
| | | | 2H) |
| 48 | 2-{4-[3-(2-methylpyrrolidin-1- | 440 | (DMSO, some signals obscured by solvent): 1.36 (d, |
| | yl)propoxy]phenyl}-4-piperidin-1-yl- | | 6.53 Hz, 4H), 1.92 (m, 15H), 2.89 (m, 3H), 3.13 (m, |
| | 4,5,6,7-tetrahydro-1,3-benzothiazole | | 2H), 3.65 (m, 1H), 4.15 (t, 5.77 Hz, 2H), 4.68 (s, 1H), |
| | | | 7.09 (d, 8.78 Hz, 2H), 7.89 (d, 8.78 Hz, 2H), |
| | | | 9.61 (m, 2H) |
| 49 | 2-{4-[3-(2-methylpyrrolidin-1- | 440 | 1.09 (d, 5.87 Hz, 3H), 1.44 (m, 3H), 1.66 (m, 8H), |
| | yl)propoxy]phenyl}-7-piperidin-1-yl- | | 2.01 (m, 6H), 2.21 (m, 1H), 2.30 (m, 1H), 2.49 (m, |
| | 4,5,6,7-tetrahydro-1,3-benzothiazole | | 2H), 2.62 (m, 2H), 2.77 (m, 2H), 2.98 (m, 1H), |
| | | | 3.18 (m, 1H), 3.95 (s, 1H), 4.07 (m, 2H), 6.92 (d, |
| | | | 8.80 Hz, 2H), 7.83 (d, 8.62 Hz, 2H) |
| 50 | N-(2-{4-[3-(2-methylpyrrolidin-1- | 414 | 1.09 (d, 6.05 Hz, 3H), 1.43 (m, 1H), 1.70 (m, 4H), |
| | yl)propoxy]phenyl}-4,5,6,7-tetrahydro- | | 2.10 (m, 11H), 2.70 (dd, 16.30, 6.23 Hz, 1H), |
| | 1,3-benzothiazol-6-yl)acetamide | | 2.95 (m, 3H), 3.18 (dd, J = 16.1, 5.3 Hz, 2H), 4.07 (m, 2H), |
| | | | 4.45 (m, 1H), 5.59 (d, 7.88 Hz, 1H), 6.93 (d, |
| | | | 8.43 Hz, 2H), 7.80 (d, 8.43 Hz, 2H) |
| 51 | ethyl 2-{4-[3-(2-methylpyrrolidin-1- | 429 | 1.10 (d, 5.77 Hz, 3H), 1.29 (t, 7.15 Hz, 3H), |
| | yl)propoxy]phenyl}-4,5,6,7-tetrahydro- | | 1.43 (m, 1H), 1.98 (m, 12H), 2.89 (m, 3H), 3.18 (m, 1H), |
| | 1,3-benzothiazole-4-carboxylate | | 3.90 (s, 1H), 4.06 (m, 2H), 4.22 (q, 7.0 Hz, 2H), |
| | | | 6.91 (d, 8.53 Hz, 2H), 7.79 (d, 8.53 Hz, 2H) |
| 52 | benzyl 2-{4-[3-(2-methylpyrrolidin-1- | 506 | 1.10 (d, 6.02 Hz, 3H), 1.44 (m, 1H), 1.73 (m, 4H), |
| | yl)propoxy]phenyl}-4,5,6,7-tetrahydro- | | 1.96 (m, 5H), 2.21 (m, 4H), 2.79 (m, 2H), 2.99 (dt, |
| | 1,3-benzothiazol-4-ylcarbamate | | 11.80, 8.03 Hz, 1H), 3.19 (m, 1H), 4.07 (td, 6.02, |
| | | | 2.95 Hz, 2H), 4.87 (d, 5.77 Hz, 1H), 5.16 (s, 2H), |
| | | | 6.92 (d, 8.78 Hz, 2H), 7.36 (m, 5H), 7.79 (d, 8.78 Hz, |
| | | | 2H) |
| 53 | N-(2-{4-[3-(2-methylpyrrolidin-1- | 414 | 1.10 (d, 6.02 Hz, 3H), 1.43 (s, 1H), 1.87 (m, 7H), |
| | yl)propoxy]phenyl}-4,5,6,7-tetrahydro- | | 2.05 (s, 3H), 2.13 (d, 8.78 Hz, 1H), 2.22 (m, 2H), |
| | 1,3-benzothiazol-4-yl)acetamide | | 2.31 (m, 1H), 2.74 (m, 2H), 2.98 (m, 1H), 3.18 (m, |
| | | | 1H), 4.08 (m, 2H), 5.03 (d, 5.77 Hz, 1H), 6.15 (d, |
| | | | 6.02 Hz, 1H), 6.93 (d, 8.78 Hz, 2H), 7.78 (d, 8.78 Hz, |
| | | | 2H) |
| 54 | 2-methyl-N-(2-{4-[3-(2-methylpyrrolidin- | 483 | 1.12 (d, 6.0 Hz, 3H), 1.21 (d, 6.3 Hz, 2H) 1.25 (d, |
| | 1-yl)propoxy]phenyl}-4,5,6,7-tetrahydro- | | 6.0 Hz, 2H), 1.45 (m, 1H), 1.60 (m, 1H), 1.87 (m, |
| | 1,3-benzothiazol-4-yl)pyrrolidine-1- | | 7H), 2.27 (m, 5H), 2.80 (m, 2H), 3.01 (m, 1H), |
| | carboxamide | | 3.21 (m, 1H), 3.31 (s, 1H), 3.38 (m, 1H), 4.02 (m, 3H), |
| | | | 4.86 (m, 2H), 6.92 (m, 2H), 7.80 (m, 2H) |
| 55 | N-(2-{4-[3-(2-methylpyrrolidin-1- | 414 | 1.11 (d, 5.5 Hz, 3H), 1.47 (m, 1H), 1.76 (m, 2H), |
| | yl)propoxy]phenyl}-4,5,6,7-tetrahydro- | | 1.94 (m, 2H), 2.00 (s, 3H), 2.22 (m, 6H), 2.71 (dd, |
| | 1,3-benzothiazol-5-yl)acetamide | | 16.3, 7.0 Hz, 1H), 2.87 (m, 2H), 3.00 (m, 1H), |
| | | | 3.18 (dd, 16.3, 5.3 Hz, 2H), 4.07 (td, 6.3, 3.0 Hz, 2H), |
| | | | 4.44 (m, 1H), 5.60 (d, 7.8 Hz, 1H), 6.93 (d, 8.8 Hz, |
| | | | 2H), 7.79 (d, 8.8 Hz, 2H) |
| 56 | ethyl 2-{4-[3-(2-methylpyrrolidin-1- | 415 | 1.09 (d, 6.02 Hz, 3H), 1.30 (t, 7.15 Hz, 3H), |
| | yl)propoxy]phenyl}-5,6-dihydro-4H- | | 1.42 (m, 1H), 1.74 (m, 3H), 1.96 (m, 3H), 2.12 (q, 8.78 Hz, |
| | cyclopenta[d][1,3]thiazole-4-carboxylate | | 1H), 2.21 (m, 1H), 2.30 (m, 1H), 2.79 (m, 2H), |
| | | | 2.95 (m, 2H), 3.09 (m, 1H), 3.17 (m, 1H), 4.04 (m, |
| | | | 3H), 4.24 (m, 2H), 6.92 (d, 8.78 Hz, 2H), 7.82 (d, |
| | | | 8.78 Hz, 2H) |
| 57 | 2-{4-[3-(2-methylpyrrolidin-1- | 454 | 1.09 (d, 5.86 Hz, 3H), 1.42 (m, 1H), 1.57 (s, 2H), |
| | yl)propoxy]phenyl}-4-(piperidin-1- | | 1.71 (m, 6H), 1.90 (dd, J = 6.97, 6.97, 2.02, 0.46 Hz, |
| | ylcarbonyl)-5,6-dihydro-4H- | | 2H), 1.99 (m, 2H), 2.11 (m, 1H), 2.21 (m, 1H), |
| | cyclopenta[d][1,3]thiazole | | 2.31 (m, 1H), 2.65 (m, 1H), 2.96 (m, 3H), 3.16 (m, |
| | | | 2H), 3.42 (m, 1H), 3.67 (m, 1H), 3.78 (m, 1H), |
| | | | 4.05 (m, 3H), 4.25 (m, 1H), 6.91 (d, 8.62 Hz, 2H), |
| | | | 7.79 (d, 8.62 Hz, 2H) |
| 58 | benzyl 2-{4-[3-(2-methylpyrrolidin-1- | 492 | 1.09 (d, 6.05 Hz, 3H), 1.42 (m, 1H), 1.72 (m, 2H), |
| | yl)propoxy]phenyl}-5,6-dihydro-4H- | | 1.92 (m, 1H), 2.00 (m, 2H), 2.12 (q, 8.85 Hz, 1H), |
| | cyclopenta[d][1,3]thiazol-4-ylcarbamate | | 2.21 (m, 1H), 2.32 (m, 2H), 2.88 (m, 1H), 2.99 (m, |
| | | | 3H), 3.18 (m, 1H), 4.07 (m, 2H), 5.17 (m, 4H), |
| | | | 6.93 (d, 8.80 Hz, 2H), 7.36 (d, 6.24 Hz, 5H), |
| | | | 7.81 (d, 8.80 Hz, 2H) |
| 59 | ethyl 4-methyl-2-{4-[3-(2- | 429 | 1.09 (d, 6.02 Hz, 3H), 1.23 (t, 7.15 Hz, 3H), |
| | methylpyrrolidin-1-yl)propoxy]phenyl}- | | 1.43 (m, 1H), 1.62 (s, 3H), 1.73 (m, 2H), 1.96 (m, 3H), |
| | 5,6-dihydro-4H- | | 2.12 (q, 8.85 Hz, 1H), 2.21 (m, 1H), 2.34 (m, 2H), |
| | cyclopenta[d][1,3]thiazole-4-carboxylate | | 2.97 (m, 4H), 3.18 (td, 8.53, 2.51 Hz, 1H), 4.07 (m, |
| | | | 2H), 4.16 (m, 2H), 6.91 (d, 8.78 Hz, 2H), 7.82 (d, |
| | | | 8.78 Hz, 2H) |
| 60 | (2-{4-[3-(2-methyl-pyrrolidin-1-yl)- | 358 | 1.09 (d, 6.05 Hz, 3H), 1.42 (m, 1H), 1.75 (m, 2H), |
| | propoxy]-phenyl}-5,6-dihydro-4H- | | 1.96 (m, 5H), 2.16 (m, 3H), 2.30 (m, 1H), 2.93 (m, |
| | cyclopentathiazol-4-ylamine | | 4H), 3.18 (m, 1H), 4.07 (m, 2H), 4.40 (m, 1H), |
| | | | 6.93 (d, 8.80 Hz, 2H), 7.82 (d, 8.62 Hz, 2H) |
| 61 | N-(2-{4-[3-(2-methylpyrrolidin-1- | 400 | 1.09 (d, 6.05 Hz, 3H), 1.42 (m, 1H), 1.74 (m, 2H), |
| | yl)propoxy]phenyl}-5,6-dihydro-4H- | | 1.92 (m, 1H), 2.01 (m, 5H), 2.12 (d, 8.99 Hz, 1H), |
| | cyclopenta[d][1,3]thiazol-4-yl)acetamide | | 2.25 (m, 3H), 2.82 (m, 2H), 3.00 (m, 2H), 3.18 (m, |
| | | | 1H), 4.08 (m, 2H), 5.28 (m, 1H), 6.32 (d, 6.60 Hz, 1H), |
| | | | 6.94 (d, 8.80 Hz, 2H), 7.78 (d, 8.80 Hz, 2H) |
| 62 | 2-{4-[3-(2-methylpyrrolidin-1- | 442 | (DMSO): 1.39 (d, 6.4 Hz, 3H), 1.58-1.69 (m, 1H), |
| | yl)propoxy]phenyl}-4-(methylsulfonyl)- | | 1.89-2.05 (m, 4H), 2.11-2.27 (m, 3H), 2.83 (t, 6.4 Hz, |
| | 4,5,6,7-tetrahydro[1,3]thiazolo[4,5- | | 2H), 3.02-3.18 (m, 2H), 3.37-3.50 (m, 5H), |
| | b]pyridine hydrochloride | | 3.58-3.67 (m, 1H), 3.72-3.80 (m, 2H), 4.11-4.19 (m, |
| | | | 2H), 7.07 (d, 8.8 Hz, 2H), 7.83 (d, 8.8 Hz, 2H), |
| | | | 9.95-10.53 (m, 1H) |
| 63 | 4-(methoxyacetyl)-2-{4-[3-(2- | 430 | (DMSO): 1.02 (br.s, 3H), 1.24-1.37 (m, 1H), |
| | methylpyrrolidin-1-yl)propoxy]phenyl}- | | 1.59-1.74 (m, 2H), 1.83-1.99 (m, 5H), 2.02-2.42 (m, 3H), |
| | 4,5,6,7-tetrahydro[1,3]thiazolo[4,5- | | 2.84 (t, 6.4 Hz, 2H), 2.87-3.00 (m, 1H), |
| | b]pyridine ethanedioate (2:1) | | 3.01-3.15 (m, 1H), 3.33 (s, 3H), 3.82-3.88 (m, 2H), 4.08 (t, |
| | | | 6.2 Hz, 2H), 4.63 (s, 2H), 7.05 (d, 8.8 Hz, 2H), |
| | | | 7.80 (d, 8.8 Hz, 2H) |
| 64 | 4-acetyl-2-{4-[3-(2-methylpyrrolidin-1- | 400 | (DMSO): 1.02 (br.s, 3H), 1.24-1.37 (m, 1H), |
| | yl)propoxy]phenyl}-4,5,6,7- | | 1.59-1.74 (m, 2H), 1.79-1.99 (m, 5H), 2.01-2.41 (m, 3H), |
| | tetrahydro[1,3]thiazolo[4,5-b]pyridine | | 2.84 (t, 6.6 Hz, 2H), 2.88-3.02 (m, 1H), |
| | ethanedioate (2:1) | | 3.03-3.16 (m, 1H), 3.79-3.84 (m, 2H), 4.08 (t, 6.2 Hz, 2H), |
| | | | 7.04 (d, 8.8 Hz, 2H), 7.80 (d, 8.8 Hz, 2H) |
| 65 | N-ethyl-2-{4-[3-(2-methylpyrrolidin-1- | 429 | (DMSO): 1.01 (d, 5.4 Hz, 3H), 1.15 (t, 7.1 Hz, 3H), |
| | yl)propoxy]phenyl}-6,7- | | 1.25-1.35 (m, 2H), 1.59-1.72 (m, 2H), |
| | dihydro[1,3]thiazolo[4,5-b]pyridine- | | 1.81-1.98 (m, 5H), 2.02-2.40 (m, 3H), 2.80 (t, 6.4 Hz, 2H), |
| | 4(5H)-carboxamide L-(+)-tartrate (2:1) | | 2.86-2.99 (m, 1H), 3.04-3.15 (m, 1H), 3.21-3.34 (m, |
| | | | 2H + H2O), 3.78-3.85 (m, 2H), 4.09 (t, 6.4 Hz, 2H), |
| | | | 7.05 (d, 8.8 Hz, 2H), 7.78 (d, 8.8 Hz, 2H), 8.74 (t, |
| | | | 5.1 Hz, 1H) |
| 66 | 4-isonicotinoyl-2-{4-[3-(2- | 463 | (DMSO): 0.97 (d, 6.1 Hz, 3H), 1.23-1.31 (m, 1H), |
| | methylpyrrolidin-1-yl)propoxy]phenyl}- | | 1.58-1.68 (m, 2H), 1.77-1.91 (m, 3H), 1.98-2.39 (m, |
| | 4,5,6,7-tetrahydro[1,3]thiazolo[4,5- | | 5H), 2.90 (t, 6.4 Hz, 2H), 3.01-3.08 (m, 1H), |
| | b]pyridine L-(+)-tartrate (2:1) | | 3.21-3.34 (m, 1H + H2O), 3.91-3.98 (m, 2H), 4.02 (t, 6.1 Hz, |
| | | | 2H), 6.86 (d, 8.8 Hz, 2H), 7.15 (d, 8.8 Hz, 2H), |
| | | | 7.35-7.39 (m, 2H), 8.59-8.63 (m, 2H) |
| 67 | 2-{4-[3-(2-methylpyrrolidin-1- | 507 | (DMSO): 1.16 (br.s, 3H), 1.40-1.50 (m, 1H), |
| | yl)propoxy]phenyl}-4-(morpholin-4- | | 1.73-1.83 (m, 2H), 1.95-2.06 (m, 5H), 2.83 (t, 6.4 Hz, 2H), |
| | ylsulfonyl)-4,5,6,7- | | 3.25-3.36 (m, 5H + H2O), 3.40-3.49 (m, 2H), |
| | tetrahydro[1,3]thiazolo[4,5-b]pyridine | | 3.62-3.67 (m, 4H), 3.75-3.79 (m, 2H), 4.04-4.14 (m, |
| | ethanedioate (2:1) | | 6H), 7.04 (d, 8.8 Hz, 2H), 7.78 (d, 8.8 Hz, 2H) |
| 68 | 2-{4-[3-(2-methylpyrrolidin-1- | 358 | (DMSO): 1.00 (d, 5.9 Hz, 3H), 1.22-1.34 (m, 1H), |
| | yl)propoxy]phenyl}-4,5,6,7- | | 1.58-1.70 (m, 2H), 1.80-1.95 (m, 5H), 1.98-2.20 (m, |
| | tetrahydro[1,3]thiazolo[4,5-b]pyridine | | 2H), 2.22-2.34 (m, 1H), 2.69 (t, 6.1 Hz, 2H), |
| | | | 2.85-2.94 (m, 1H), 3.04-3.12 (m, 1H), 3.15-3.21 (m, 2H), |
| | | | 4.05 (t, 6.4 Hz, 2H), 5.65-5.70 (m, 1H), |
| | | | 6.96-7.01 (m, 2H), 7.67-7.72 (m, 2H) |
| 69 | 2-{4-[3-(2-methylpyrrolidin-1- | 372 | (DMSO): 1.02 (br.s, 3H), 1.24-1.37 (m, 1H), |
| | yl)propoxy]phenyl}-6,7- | | 1.58-1.74 (m, 2H), 1.78-1.98 (m, 3H), 2.00-2.38 (m, 3H), |
| | dihydro[1,3]thiazolo[4,5-b]pyridin-5(4H)- | | 2.57-2.65 (m, 2H), 2.86-2.99 (m, 3H), 3.02-3.19 (m, |
| | one | | 1H), 4.08 (t, 6.4 Hz, 2H), 7.02 (d, 8.8 Hz, 2H), |
| | | | 7.75 (d, 8.8 Hz, 2H), 10.60 (s, 1H) |
| 70 | 5-methyl-2-{4-[3-(2-methylpyrrolidin-1- | 372 | 1.11 (m, 3H), 1.43 (m, 1H), 1.74 (m, 2H), 1.91 (m, |
| | yl)propoxy]phenyl}-4,5,6,7- | | 1H), 2.02 (m, 2H), 2.13 (m, 1H), 2.23 (m, 1H), |
| | tetrahydro[1,3]thiazolo[4,5-c]pyridine | | 2.36 (m, 1H), 2.53 (m, 3H), 2.79 (m, 2H), 2.92 (t, |
| | | | 5.5 Hz, 2H), 3.02 (m, 1H), 3.19 (m, 1H), 3.66 (m, 2H), |
| | | | 4.08 (m, 2H), 6.92 (d, 8.8 Hz, 2H), 7.80 (d, 8.5 Hz, |
| | | | 2H) |
| 71 | 5-acetyl-2-(4-{3-[(2R)-2- | 400 | 1.10 (d, 6.0 Hz, 3H), 1,43 (m, 1H), 1.75 (m, 3H), |
| | methylpyrrolidin-1-yl]propoxy}phenyl)- | | 1.91 (m, 1H), 2.02 (m, 2H), 2.13 (m, 1H), 2.22 (m, 3H), |
| | 4,5,6,7-tetrahydro[1,3]thiazolo[4,5- | | 2.31 (m, 1H), 2.94 (m, 3H), 3.18 (td, 8.8 & 2.5 Hz, |
| | c]pyridine | | 1H), 3.86 (m, 2H), 4.08 (m, 2H), 4.75 (m, 2H), |
| | | | 6.94 (m, 2H), 7.80 (m, 2H) |
| 72 | N-ethyl-2-(4-{3-[(2R)-2-methylpyrrolidin- | 429 | 1.09 (d, 6.0 Hz, 3H), 1.17 (t, 7.3 Hz, 3H), 1.42 (m, 1H), |
| | 1-yl]propoxy}phenyl)-6,7- | | 1.74 (m, 2H), 1.92 (m, 1H), 2.02 (m, 2H), |
| | dihydro[1,3]thiazolo[4,5-c]pyridine- | | 2.13 (q, 8.8 Hz, 1H), 2.21 (m, 1H), 2.31 (m, 1H), 2.89 (t, |
| | 5(4H)-carboxamide | | 5.5 Hz, 2H), 2.98 (dt, 11.8 & 7.8 Hz, 1H), 3.18 (td, |
| | | | 8.5 & 2.5 Hz, 1H), 3.33 (m, 2H), 3.81 (t, 5.8 Hz, 2H), |
| | | | 4.07 (m, 2H), 4.53 (s, 3H), 6.93 (d, 8.8 Hz, 2H), |
| | | | 7.79 (d, 8.8 Hz, 2H) |
| 73 | 5-(methoxyacetyl)-2-(4-{3-[(2R)-2- | 430 | 1.09 (d, 6.0 Hz, 3H), 1.42 (m, 1H), 1.74 (m, 2H), |
| | methylpyrrolidin-1-yl]propoxy}phenyl)- | | 1.90 (m, 1H), 2.01 (m, 2H), 2.12 (q, 8.8 Hz, 1H), |
| | 4,5,6,7-tetrahydro[1,3]thiazolo[4,5- | | 2.21 (m, 1H), 2.31 (m, 1H), 2.95 (m, 3H), 3.18 (td, |
| | c]pyridine | | 8.5 & 2.8 Hz, 1H), 3.45 (s, 3H), 3.82 (t, 5.3 Hz, 1H), |
| | | | 3.96 (t, 5.5 Hz, 1H), 4.08 (td, 6.0, 1.5 Hz, 2H), |
| | | | 4.20 (m, 2H), 4.76 (m, 2H), 6.94 (d, 8.5 Hz, 2H), |
| | | | 7.79 (m, 2H) |
| 74 | 5-methyl-2-(4-{3-[(2R)-2- | 356 | 1.37 (m, 3H), 1.63 (m, 1H), 1.93 (m, 2H), 2.17 (m, |
| | methylpyrrolidin-1-yl]propoxy}phenyl)- | | 3H), 2.94 (m, 3H), 3.11 (m, 4H), 3.44 (m, 3H), |
| | 4,5,6,7-tetrahydro[1,3]oxazolo[4,5- | | 3.63 (m, 1H), 3.73 (m, 1H), 4.18 (m, 3H), 4.40 (m, |
| | c]pyridine dihydrochloride | | 1H), 7.11 (m, 2H), 7.94 (m, 2H) |
| 75 | 2-{4-[3-(2-methylpyrrolidin-1- | 455 | 1.37 (m, 3H), 1.63 (m, 1H), 1.93 (m, 2H), 2.17 (m, |
| | yl)propoxy]phenyl}-5-(pyrrolidin-1- | | 3H), 2.94 (m, 3H), 3.11 (m, 4H), 3.44 (m, 3H), |
| | ylcarbonyl)-4,5,6,7- | | 3.63 (m, 1H), 3.73 (m, 1H), 4.18 (m, 3H), 4.40 (m, |
| | tetrahydro[1,3]thiazolo[5,4-c]pyridine | | 1H), 7.11 (m, 2H), 7.94 (m, 2H) |
| 76 | 2-{4-[3-(2-methylpyrrolidin-1- | 471 | 1.37 (m, 3H), 1.63 (m, 1H), 1.93 (m, 2H), 2.17 (m, |
| | yl)propoxy]phenyl}-5-(morpholin-4- | | 3H), 2.94 (m, 3H), 3.11 (m, 4H), 3.44 (m, 3H), |
| | ylcarbonyl)-4,5,6,7- | | 3.63 (m, 1H), 3.73 (m, 1H), 4.18 (m, 3H), 4.40 (m, |
| | tetrahydro[1,3]thiazolo[5,4-c]pyridine | | 1H), 7.11 (m, 2H), 7.94 (m, 2H) |
| 77 | 5-[(4-methylpiperazin-1-yl)carbonyl]-2- | 484 | 1.10 (d, 6.0 Hz, 3H), 1.43 (m, 1H), 1.76 (m, 2H), |
| | {4-[3-(2-methylpyrrolidin-1- | | 1.92 (m, 1H), 2.02 (m, 2H), 2.13 (q, 8.8 Hz, 1H), |
| | yl)propoxy]phenyl}-4,5,6,7- | | 2.22 (m, 1H), 2.32 (s, 4H), 2.44 (m, 4H), 2.99 (m, 3H), |
| | tetrahydro[1,3]thiazolo[5,4-c]pyridine | | 3.18 (td, 8.8 & 2.5 Hz, 1H), 3.36 (m, 4H), |
| | | | 3.59 (t, 5.8 Hz, 2H), 4.07 (m, 2H), 4.48 (s, 2H), 6.93 (d, |
| | | | 8.8 Hz, 2H), 7.80 (d, 8.8 Hz, 2H) |
| 78 | 5-[(4,4-difluoropiperidin-1-yl)carbonyl]- | 505 | 1.10 (d, 6.0 Hz, 3H), 1.43 (m, 1H), 1.70 (m, 1H), |
| | 2-{4-[3-(2-methylpyrrolidin-1- | | 1.79 (m, 1H), 1.92 (m, 1H), 2.02 (m, 6H), 2.14 (m, |
| | yl)propoxy]phenyl}-4,5,6,7- | | 1H), 2.23 (m, 1H), 2.33 (m, 1H), 3.00 (m, 3H), |
| | tetrahydro[1,3]thiazolo[5,4-c]pyridine | | 3.19 (m, 1H), 3.43 (t, 5.8 Hz, 4H), 3.61 (t, 5.8 Hz, 2H), |
| | | | 4.08 (m, 2H), 4.50 (s, 2H), 6.93 (d, 8.8 Hz, 2H), |
| | | | 7.80 (d, 8.8 Hz, 2H) |
| 79 | 2-{4-[3-(2-methylpyrrolidin-1- | 496 | 1.09 (d, 6.0 Hz, 3H), 1.42 (m, 1H), 1.74 (m, 3H), |
| | yl)propoxy]phenyl}-5-(5,5,5- | | 1.97 (m, 4H), 2.12 (q, 9.0 Hz, 1H), 2.26 (m, 4H), |
| | trifluoropentanoyl)-4,5,6,7- | | 2.52 (m, 2H), 2.97 (m, 3H), 3.18 (td, 8.5 & 2.5 Hz, 1H), |
| | tetrahydro[1,3]thiazolo[5,4-c]pyridine | | 3.88 (m, 2H), 3.79 (t, 5.8 Hz), 4.08 (td, 6.3 & 1.8 Hz, |
| | | | 2H), 4.75 (m, 2H), 6.94 (d, 8.5 Hz, 2H), |
| | | | 7.80 (m, 2H) |
| 80 | 4-[2-{4-[3-(2-methylpyrrolidin-1- | 430 | 1.09 (m, 3H), 1.24 (m, 4H), 1.48 (m, 1H), 1.61 (m, |
| | yl)propoxy]phenyl}-6,7- | | 1H), 1.77 (m, 3H), 2.00 (dd, 4.3, 4.3, 3.3 & 2.0 Hz, |
| | dihydro[1,3]thiazolo[5,4-c]pyridin-5(4H)- | | 3H), 2.22 (m, 2H), 2.39 (m, 1H), 2.84 (m, 3H), |
| | yl]butan-2-ol | | 2.94 (m, 2H), 3.01 (dd, 8.3 & 3.8 Hz, 1H), 3.11 (m, |
| | | | 1H), 3.21 (m, 1H), 3.79 (m, 2H), 4.05 (m, 3H), |
| | | | 6.92 (d, 8.8 Hz, 2H), 7.80 (d, 8.8 Hz, 2H) |
| 81 | (3R)-1-{3-[4-(5-acetyl-4,5,6,7- | 429 | 1.72 (m, 1H), 1.98 (m, 4H), 2.20 (m, 8H), 2.33 (m, 1H), |
| | tetrahydro[1,3]thiazolo[5,4-c]pyridin-2- | | 2.48 (m, 1H), 2.57 (m, 1H), 2.66 (m, 1H), |
| | yl)phenoxy]propyl}-N,N- | | 2.77 (m, 2H), 2.85 (m, 1H), 2.96 (m, 2H), 3.88 (m, 2H), |
| | dimethylpyrrolidin-3-amine | | 4.07 (t, 6.5 Hz, 2H), 4.76 (m, 2H), 6.93 (m, 2H), |
| | | | 7.80 (m, 2H) |
| 82 | cis-3-[2-{4-[3-(2-methylpyrrolidin-1- | 428 | 1.07 (m, 3H), 1.42 (m, 1H), 1.72 (m, 2H), 1.91 (m, |
| | yl)propoxy]phenyl}-6,7- | | 3H), 2.01 (m, 2H), 2.12 (m, 1H), 2.27 (m, 3H), |
| | dihydro[1,3]thiazolo[5,4-c]pyridin-5(4H)- | | 2.59 (m, 3H), 2.75 (m, 2H), 2.90 (m, 2H), 3.00 (m, |
| | yl]cyclobutanol | | 1H), 3.18 (m, 1H), 3.61 (m, 2H), 4.06 (m, 3H), |
| | | | 6.92 (d, 8.8 Hz, 2H), 7.79 (d, 8.8 Hz, 2H) |
| 83 | (3S)-1-{3-[4-(5-acetyl-4,5,6,7- | 429 | 1.72 (m, 1H), 1.98 (m, 4H), 2.19 (m, 8H), 2.33 (t, |
| | tetrahydro[1,3]thiazolo[5,4-c]pyridin-2- | | 8.3 Hz, 1H), 2.47 (m, 1H), 2.58 (m, 1H), 2.67 (m, 1H), |
| | yl)phenoxy]propyl}-N,N- | | 2.76 (m, 2H), 2.85 (m, 1H), 2.96 (m, 2H), |
| | dimethylpyrrolidin-3-amine | | 3.88 (m, 2H), 4.08 (m, 2H), 4.75 (m, 2H), 6.93 (d, 8.5 Hz, |
| | | | 2H), 7.80 (m, 2H) |
| 84 | methyl 3-[2-{4-[3-(2-methylpyrrolidin-1- | 458 | 1.09 (d, 6.2 Hz, 3H), 1.43 (m, 1H), 1.73 (m, 2H), |
| | yl)propoxy]phenyl}-6,7- | | 1.90 (m, 1H), 2.02 (m, 2H), 2.12 (q, 9.0 Hz, 1H), |
| | dihydro[1,3]thiazolo[5,4-c]pyridin-5(4H)- | | 2.21 (m, 1H), 2.32 (m, 1H), 2.98 (m, 3H), 3.17 (td, |
| | yl]-3-oxopropanoate | | 8.6 & 2.8 Hz, 1H), 3.58 (m, 2H), 3.77 (m, 4H), |
| | | | 4.03 (m, 3H), 4.76 (m, 2H), 6.93 (d, 8.6 Hz, 2H), |
| | | | 7.80 (m, 2H) |
| 85 | 2-[2-{4-[3-(2-methylpyrrolidin-1- | 402 | 1.09 (d, 6.0 Hz, 3H), 1.42 (m, 1H), 1.73 (m, 2H), |
| | yl)propoxy]phenyl}-6,7- | | 1.91 (m, 1H), 2.00 (m, 2H), 2.12 (q, 8.8 Hz, 1H), |
| | dihydro[1,3]thiazolo[5,4-c]pyridin-5(4H)- | | 2.21 (m, 1H), 2.30 (m, 1H), 2.77 (m, 2H), 2.97 (m, |
| | yl]ethanol | | 5H), 3.17 (m, 1H), 3.72 (m, 2H), 3.80 (m, 2H), |
| | | | 4.07 (m, 2H), 6.93 (d, 8.8 Hz, 2H), 7.80 (d, 8.8 Hz, |
| | | | 2H) |
| 86 | 3-[2-{4-[3-(2-methylpyrrolidin-1- | 444 | (DMSO, some signals obscured by solvent): 0.97 (d, |
| | yl)propoxy]phenyl}-6,7- | | 6.0 Hz, 3H), 1.25 (m, 1H), 1.60 (m, 2H), 1.85 (m, 3H), |
| | dihydro[1,3]thiazolo[5,4-c]pyridin-5(4H)- | | 2.05 (m, 2H), 2.22 (m, 1H), 2.92 (m, 4H), |
| | yl]-3-oxopropanoic acid potassium | | 3.72 (m, 2H), 4.01 (m, 2H), 4.68 (m, 2H), 7.00 (d, 8.8 Hz, |
| | oxopropanoate | | 2H), 7.77 (d, 8.5 Hz, 2H) |
| 87 | 3-[2-(4-{3-[(2R)-2-methylpyrrolidin-1- | 424 | 1.15 (s, 3H), 1.50 (m, 1H), 1.75 (m, 1H), 1.86 (m, 1H), |
| | yl]propoxy}phenyl)-6,7- | | 1.97 (m, 1H), 2.07 (m, 2H), 2.21 (m, 1H), |
| | dihydro[1,3]thiazolo[5,4-c]pyridin-5(4H)- | | 2.31 (m, 1H), 2.44 (dd, 1.5 & 0.8 Hz, 1H), 3.04 (m, 3H), |
| | yl]cyclobut-2-en-1-one | | 3.25 (m, 3H), 3.77 (s, 2H), 4.08 (m, 2H), 4.61 (m, 1H), |
| | | | 4.70 (s, 1H), 4.80 (m, 1H), 6.95 (d, 7.8 Hz, 2H), |
| | | | 7.82 (d, 7.5 Hz, 2H) |
| 88 | 5-(trans-3-fluorocyclobutyl)-2-{4-[3-(2- | 430 | 1.14 (d, 5.8 Hz, 3H), 1.49 (m, 1H), 1.73 (m, 1H), |
| | methylpyrrolidin-1-yl)propoxy]phenyl}- | | 1.84 (m, 1H), 1.96 (m, 1H), 2.04 (m, 2H), 2.23 (m, |
| | 4,5,6,7-tetrahydro[1,3]thiazolo[5,4- | | 1H), 2.39 (m, 6H), 2.77 (t, 5.8 Hz, 2H), 2.94 (m, 2H), |
| | c]pyridine | | 3.02 (m, 1H), 3.24 (s, 1H), 3.36 (m, 1H), |
| | | | 3.64 (s, 2H), 4.07 (m, 2H), 5.19 (m, 1H), 6.92 (d, 8.0 Hz, |
| | | | 2H), 7.80 (d, 7.8 Hz, 2H) |
| 89 | 2-(4-{3-[(2R)-2-methylpyrrolidin-1- | 507 | 1.03 (d, 6.0 Hz, 3H), 1.36 (m, 1H), 1.74 (m, 4H), |
| | yl]propoxy}phenyl)-5-(morpholin-4- | | 1.94 (m, 2H), 2.06 (q, 8.8 Hz, 1H), 2.15 (m, 1H), |
| | ylsulfonyl)-4,5,6,7- | | 2.25 (m, 1H), 2.94 (m, 2H), 3.11 (m, 1H), 3.16 (m, |
| | tetrahydro[1,3]thiazolo[5,4-c]pyridine | | 4H), 3.61 (t, 5.8 Hz, 2H), 3.66 (m, 4H), 4.01 (td, 6.3 |
| | | | & 2.0 Hz, 2H), 4.48 (s, 2H), 6.87 (d, 8.8 Hz, 2H), |
| | | | 7.73 (d, 8.8 Hz, 2H) |
| 90 | 3-[2-(4-{3-[(2R)-2-methylpyrrolidin-1- | 443 | (DMSO, some signals obscured by solvent): 0.99 (d, |
| | yl]propoxy}phenyl)-6,7- | | 5.8 Hz, 3H), 1.28 (m, 1H), 1.63 (m, 2H), 1.87 (m, 3H), |
| | dihydro[1,3]thiazolo[5,4-c]pyridin-5(4H)- | | 2.02 (m, 1H), 2.11 (m, 1H), 2.25 (m, 1H), |
| | yl]-3-oxopropanamide | | 2.85 (m, 3H), 3.07 (m, 1H), 3.82 (m, 2H), 4.06 (m, 2H), |
| | | | 4.76 (m, 2H), 7.00 (m, 2H), 7.11 (m, 1H), 7.50 (m, |
| | | | 1H), 7.80 (m, 2H) |
| 91 | methyl [2-(4-{3-[(2R)-2-methylpyrrolidin- | 430 | 0.99 (d, 6.0 Hz, 3H), 1.27 (m, 1H), 1.62 (m, 2H), |
| | 1-yl]propoxy}phenyl)-6,7- | | 1.87 (m, 3H), 2.03 (q, 8.8 Hz, 1H), 2.11 (m, 1H), |
| | dihydro[1,3]thiazolo[5,4-c]pyridin-5(4H)- | | 2.24 (m, 1H), 2.79 (t, 5.5 Hz, 2H), 2.89 (m, 1H), |
| | yl]acetate | | 2.95 (t, 6.0 Hz, 2H), 3.07 (m, 1H), 3.50 (m, 2H), |
| | | | 3.64 (m, 3H), 3.83 (s, 2H), 4.05 (m, 2H), 7.01 (d, |
| | | | 9.0 Hz, 2H), 7.78 (m, 2H) |
| 92 | 2-[2-(4-{3-[(2R)-2-methylpyrrolidin-1- | 416 | 1.01 (d, 6.0 Hz, 3H), 1.29 (m, 1H), 1.66 (m, 2H), |
| | yl]propoxy}phenyl)-6,7- | | 1.89 (m, 3H), 2.12 (m, 2H), 2.31 (m, 1H), 2.80 (m, |
| | dihydro[1,3]thiazolo[5,4-c]pyridin-5(4H)- | | 1H), 2.92 (m, 2H), 3.11 (m, 1H), 3.72 (m, 1H), |
| | yl]-2-oxoethanol | | 3.82 (m, 1H), 4.07 (t, 6.3 Hz, 2H), 4.22 (m, 2H), |
| | | | 4.75 (m, 3H), 7.02 (d, 8.8 Hz, 2H), 7.80 (d, 8.5 Hz, |
| | | | 2H) |
| 93 | 2-[2-(4-{3-[(2R)-2-methylpyrrolidin-1- | 415 | 0.99 (d, 6.0 Hz, 3H), 1.28 (m, 1H), 1.62 (m, 2H), |
| | yl]propoxy}phenyl)-6,7- | | 1.88 (m, 3H), 2.02 (m, 1H), 2.13 (m, 1H), 2.26 (m, |
| | dihydro[1,3]thiazolo[5,4-c]pyridin-5(4H)- | | 1H), 2.86 (m, 5H), 3.10 (m, 3H), 3.78 (s, 2H), |
| | yl]acetamide | | 4.08 (m, 2H), 7.01 (d, 8.8 Hz, 2H), 7.14 (s, 1H), 7.28 (m, |
| | | | 1H), 7.79 (d, 8.8 Hz, 2H) |
| 94 | 1-[2-(4-{3-[(2R)-2-methylpyrrolidin-1- | 414 | 1.13 (d, 6.0 Hz, 3H), 1.47 (m, 1H), 1.72 (m, 1H), |
| | yl]propoxy}phenyl)-6,7- | | 1.83 (m, 1H), 1.94 (m, 1H), 2.05 (m, 2H), 2.19 (m, |
| | dihydro[1,3]thiazolo[5,4-c]pyridin-5(4H)- | | 4H), 2.28 (m, 1H), 2.39 (m, 1H), 2.94 (m, 4H), |
| | yl]acetone | | 3.03 (m, 1H), 3.24 (m, 1H), 3.46 (s, 2H), 3.84 (m, 2H), |
| | | | 4.07 (m, 2H), 6.92 (m, 2H), 7.80 (m, 2H) |
| 95 | 3-[2-(4-{3-[(2R)-2-methylpyrrolidin-1- | 432 | 1.10 (d, 6.0 Hz, 3H), 1.43 (m, 1H), 1.75 (m, 2H), |
| | yl]propoxy}phenyl)-6,7- | | 1.92 (m, 1H), 2.02 (m, 2H), 2.13 (q, 8.8 Hz, 1H), |
| | dihydro[1,3]thiazolo[5,4-c]pyridin-5(4H)- | | 2.22 (m, 1H), 2.32 (m, 1H), 2.65 (dd, 12.5, 4.0 Hz, |
| | yl]propane-1,2-diol | | 1H), 2.75 (dd, 12.8, 9.5 Hz, 1H), 2.95 (m, 4H), |
| | | | 3.07 (m, 1H), 3.18 (td, 8.8, 2.8 Hz, 1H), 3.54 (dd, 11.3, |
| | | | 4.3 Hz, 1H), 3.76 (m, 2H), 3.91 (m, 2H), 4.07 (m, 2H), |
| | | | 6.93 (d, 8.8 Hz, 2H), 7.80 (m, 2H) |
| 96 | ((2S)-1-{3-[4-(5-acetyl-4,5,6,7- | 416 | (DMSO, some signals obscured by solvent): 1.75 (m, |
| | tetrahydro[1,3]thiazolo[5,4-c]pyridin-2- | | 1H), 1.89 (m, 1H), 2.00 (m, 1H), 2.11 (m, 4H), |
| | yl)phenoxy]propyl}pyrrolidin-2- | | 2.23 (m, 1H), 2.82 (m, 2H), 3.16 (m, 2H), 3.55 (m, |
| | yl)methanol | | 4H), 3.77 (m, 4H), 4.14 (t, 5.8 Hz, 2H), 4.74 (m, 2H), |
| | | | 7.05 (d, 8.5 Hz, 2H), 7.84 (m, 2H), 10.20 (m, 1H) |
| 97 | tert-butyl [2-(4-{3-[(2R)-2- | 412 | 1.24 (d, 6.0 Hz, 3H), 1.49 (s, 9H), 1.61 (m, 1H), |
| | methylpyrrolidin-1-yl]propoxy}phenyl)- | | 1.79 (m, 1H), 1.90 (m, 1H), 2.03 (m, 1H), 2.14 (m, |
| | 6,7-dihydro[1,3]thiazolo[5,4-c]pyridin- | | 2H), 2.34 (m, 1H), 2.45 (m, 1H), 2.63 (m, 1H), |
| | 5(4H)-yl]acetate | | 2.95 (m, 2H), 3.05 (m, 2H), 3.12 (m, 1H), 3.33 (m, |
| | | | 1H), 3.39 (s, 2H), 3.92 (s, 2H), 4.08 (m, 2H), |
| | | | 6.92 (d, 8.8 Hz, 2H), 7.81 (d, 8.8 Hz, 2H) |
| 98 | [2-(4-{3-[(2R)-2-methylpyrrolidin-1- | 416 | 1.66 (d, 6.5 Hz, 3H), 1.91 (m, 1H), 2.27 (m, 2H), |
| | yl]propoxy}phenyl)-6,7- | | 2.46 (m, 3H), 3.43 (m, 4H), 3.75 (m, 2H), 3.97 (m, |
| | dihydro[1,3]thiazolo[5,4-c]pyridin-5(4H)- | | 2H), 4.44 (m, 4H), 4.80 (s, 2H), 7.37 (d, 8.8 Hz, 2H), |
| | yl]acetic acid trifluoroacetate | | 8.16 (d, 8.8 Hz, 2H), 9.85 (m, 1H) |
| 99 | 2-{4-[3-(2-methylpyrrolidin-1- | 450 | (DMSO): 1.01 (d, J5.8 Hz, 3H), 1.30 (m, 2H), |
| | yl)propoxy]phenyl}-6-(methylsulfonyl)- | | 1.65 (m, 2H), 1.89 (m, 3H), 2.95 (s, 3H), 3.07 (m, 5H), |
| | 5,6,7,8-tetrahydro-4H-[1,3]thiazolo[4,5- | | 3.50 (m, 4H), 4.07 (d, 6.0 Hz, 2H), 7.01 (d, 8.8 Hz, 2H), |
| | d]azepine | | 7.76 (d, 8.8 Hz, 2H) |
| 100 | 2-{4-[3-(2-methylpyrrolidin-1- | 485 | (DMSO, some signals obscured by solvent): 1.03 (d, |
| | yl)propoxy]phenyl}-6-(morpholin-4- | | 5.3 Hz, 3H), 1.31 (m, 1H), 1.66 (m, 2H), 1.88 (m, 3H), |
| | ylcarbonyl)-5,6,7,8-tetrahydro-4H- | | 3.04 (m, 2H), 3.10 (m, 7H), 3.53 (m, 4H), |
| | [1,3]thiazolo[4,5-d]azepine | | 3.59 (m, 4H), 4.07 (t, 6.3 Hz, 2H), 7.00 (d, 9.0 Hz, 2H), |
| | | | 7.75 (d, 8.8 Hz, 2H) |
| 101 | 2-(2-{4-[3-(2-methylpyrrolidin-1- | 429 | (DMSO): 0.99 (d, 6.0 Hz, 3H), 1.26 (m, 1H), |
| | yl)propoxy]phenyl}-4,5,7,8-tetrahydro- | | 1.63 (m, 2H), 1.87 (m, 3H), 2.03 (q, 8.8 Hz, 1H), |
| | 6H-[1,3]thiazolo[4,5-d]azepin-6- | | 2.12 (m, 1H), 2.24 (m, 1H), 2.78 (m, 4H), 2.92 (m, 3H), |
| | yl)acetamide | | 3.01 (m, 2H), 3.07 (m, 1H), 3.12 (s, 2H), 4.06 (t, |
| | | | 6.3 Hz, 2H), 6.99 (d, 8.8 Hz, 2H), 7.15 (s, 1H), |
| | | | 7.31 (s, 1H), 7.74 (d, 8.8 Hz, 2H) |
| 102 | 2-{4-[3-(2-methylpyrrolidin-1- | 521 | — |
| | yl)propoxy]phenyl}-6-(morpholin-4- | | |
| | ylsulfonyl)-5,6,7,8-tetrahydro-4H- | | |
| | [1,3]thiazolo[4,5-d]azepine | | |
| 103 | 6-isonicotinoyl-2-{4-[3-(2- | 477 | (DMSO, some signals obscured by solvent): 1.23 (d, |
| | methylpyrrolidin-1-yl)propoxy]phenyl}- | | 6.0 Hz, 3H), 1.53 (m, 1H), 1.85 (m, 2H), 2.08 (m, 3H), |
| | 5,6,7,8-tetrahydro-4H-[1,3]thiazolo[4,5- | | 2.80 (d, 7.5 Hz, 2H), 2.96 (m, 2H), 3.13 (m, 4H), |
| | d]azepine L-(+)-tartrate | | 3,46 (m, 3H), 3.86 (m, 2H), 4.10 (m, 2H), |
| | | | 7.03 (dd, 8.8 & 2.3 Hz, 2H), 7.45 (m, 2H), 7.78 (m, 2H), |
| | | | 8.69 (m, 2H) |
| 104 | 7,7-dimethyl-2-{4-[3-(2-methylpyrrolidin- | 414 | 1.09 (d, 6.1 Hz, 3H), 1.13 (s, 6H), 1.42 (m, 1H), |
| | 1-yl)propoxy]phenyl}-5,6,7,8-tetrahydro- | | 1.70 (m, 1H), 1.80 (m, 1H), 1.91 (m, 1H), 2.01 (m, |
| | 4H-[1,3]thiazolo[5,4-c]azepin-4-one | | 2H), 2.12 (q, 9.0 Hz, 1H), 2.21 (m, 1H), 2.30 (m, 1H), |
| | | | 2.97 (m, 1H), 3.02 (s, 2H), 3.14 (d, 5.3 Hz, 2H), |
| | | | 3.18 (m, 1H), 4.08 (m, 2H), 6.47 (s, 1H), 6.95 (d, |
| | | | 8.6 Hz, 2H), 7.87 (d, 8.8 Hz, 2H) |
| 105 | 5,7,7-trimethyl-2-{4-[3-(2- | 428 | 1.13 (s, 6H), 1.17 (d, 6.0 Hz, 3H), 1.51 (m, 1H), |
| | methylpyrrolidin-1-yl)propoxy]phenyl}- | | 1.76 (m, 1H), 1.85 (m, 1H), 1.98 (m, 1H), 2.08 (m, |
| | 5,6,7,8-tetrahydro-4H-[1,3]thiazolo[5,4- | | 2H), 2.31 (m, 2H), 2.47 (m, 1H), 2.91 (s, 2H), |
| | c]azepin-4-one | | 3.05 (m, 1H), 3.21 (m, 5H), 3.28 (t, 7.5 Hz, 1H), |
| | | | 4.09 (m, 2H), 6.94 (d, 8.8 Hz, 2H), 7.86 (d, 8.8 Hz, 2H) |
| 106 | 4-acetyl-2-(4-{3-[(2R)-2- | 414 | 0.99 (m, 3H), 1.26 (m, 1H), 1.65 (m, 3H), 1.86 (dd, |
| | methylpyrrolidin-1-yl]propoxy}phenyl)- | | 26.4, 26.4, 16.6 & 5.8 Hz, 6H), 2.07 (m, 4H), |
| | 5,6,7,8-tetrahydro-4H-[1,3]thiazolo[5,4- | | 2.25 (m, 2H), 2.89 (m, 3H), 3.08 (m, 1H), 3.66 (m, 2H), |
| | b]azepine | | 4.06 (m, 2H), 7.01 (m, 2H), 7.77 (dd, 16.1 & 8.8 Hz, |
| | | | 2H) |
| 107 | 2-{4-[3-(2-methylpyrrolidin-1- | 456 | 1.10 (d, 6.0 Hz, 3H), 1.43 (m, 1H), 1.75 (m, 3H), |
| | yl)propoxy]phenyl}-5-(morpholin-4- | | 1.92 (m, 1H), 2.01 (m, 2H), 2.13 (m, 1H), 2.22 (m, |
| | ylcarbonyl)-5,6-dihydro-4H- | | 1H), 2.33 (m, 1H), 2.99 (dt, 12.0 & 7.8 Hz, 1H), |
| | cyclopenta[d][1,3]thiazole | | 3.11 (m, 1H), 3.20 (m, 3H), 3.42 (dd, 15.6 & 6.3 Hz, |
| | | | 1H), 3.58 (s, 2H), 3.71 (m, 5H), 3.94 (m, 1H), |
| | | | 4.07 (m, 2H), 6.92 (d, 8.8 Hz, 2H), 7.80 (d, 8.8 Hz, 2H) |
| 108 | 2-{4-[3-(2-methylpyrrolidin-1- | 358 | 1.09 (d, 6.0 Hz, 3H), 1.42 (m, 1H), 1.74 (m, 4H), |
| | yl)propoxy]phenyl}-5,6-dihydro-4H- | | 1.96 (m, 3H), 2.12 (q, 8.8 Hz, 1H), 2.21 (m, 1H), |
| | cyclopenta[d][1,3]thiazol-5-amine | | 2.30 (m, 1H), 2.66 (m, 2H), 2.98 (dt, 11.8, 8.3 Hz, 1H), |
| | | | 3.18 (td, 8.8, 2.5 Hz, 1H), 3.27 (m, 2H), 4.06 (m, |
| | | | 2H), 4.23 (m, 1H), 6.92 (d, 8.8 Hz, 2H), 7.81 (d, 8.8 Hz, |
| | | | 2H) |
| 109 | N-(2-{4-[3-(2-methylpyrrolidin-1- | 400 | 1.09 (d, 6.0 Hz, 3H), 1.42 (m, 1H), 1.69 (m, 1H), |
| | yl)propoxy]phenyl}-5,6-dihydro-4H- | | 1.80 (m, 1H), 1.91 (m, 1H), 1.99 (s, 3H), 2.04 (m, 2H), |
| | cyclopenta[d][1,3]thiazol-5-yl)acetamide | | 2.12 (q, 8.8 Hz, 1H), 2.21 (m, 1H), 2.31 (m, 1H), |
| | | | 2.74 (m, 2H), 2.98 (dt, 1.8 & 7.8 Hz, 1H), |
| | | | 3.18 (td, 8.8 & 2.8 Hz, 1H), 3.37 (m, 2H), 4.07 (m, 2H), |
| | | | 5.10 (m, 1H), 6.01 (d, 7.8 Hz, 1H), 6.93 (d, 8.8 Hz, |
| | | | 2H), 7.80 (d, 8.8 Hz, 2H) |
| 110 | 5-[(4,4-difluoropiperidin-1-yl)carbonyl]- | 490 | 1.09 (d, 6.0 Hz, 3H), 1.42 (m, 1H), 1.69 (m, 3H), |
| | 2-(4-{3-[(2R)-2-methylpyrrolidin-1- | | 1.79 (m, 1H), 1.93 (m, 1H), 2.01 (m, 5H), 2.12 (m, |
| | yl]propoxy}phenyl)-5,6-dihydro-4H- | | 1H), 2.21 (m, 1H), 2.30 (m, 1H), 2.98 (dt, 11.8 & |
| | cyclopenta[d][1,3]thiazole | | 8.0 Hz, 1H), 3.13 (m, 1H), 3.19 (d, 7.8 Hz, 2H), |
| | | | 3.41 (dd, 15.6 & 6.3 Hz, 1H), 3.68 (d, 3.0 Hz, 2H), |
| | | | 3.79 (m, 2H), 3.98 (m, 1H), 4.07 (m, 2H), 6.93 (d, |
| | | | 8.8 Hz, 2H), 7.80 (d, 8.8 Hz, 2H) |
| 111 | N-ethyl-2-(4-{3-[(2R)-2-methylpyrrolidin- | 414 | 1.09 (d, 6.3 Hz, 3H), 1.18 (t, 7.0 Hz, 3H), 1.42 (m, 1H), |
| | 1-yl]propoxy}phenyl)-5,6-dihydro-4H- | | 1.71 (m, 2H), 1.79 (m, 1H), 1.92 (m, 1H), |
| | cyclopenta[d][1,3]thiazole-5- | | 2.01 (m, 2H), 2.12 (q, 8.8 Hz, 1H), 2.21 (m, 1H), |
| | carboxamide | | 2.30 (m, 1H), 2.98 (dt, 11.8 & 7.8 Hz, 1H), 3.16 (d, 7.0 Hz, |
| | | | 2H), 3.20 (m, 1H), 3.27 (m, 1H), 3.35 (m, 2H), |
| | | | 3.56 (m, 1H), 4.07 (m, 2H), 5.55 (s, 1H), 6.93 (d, |
| | | | 8.8 Hz, 2H), 7.80 (d, 8.8 Hz, 2H) |
| 112 | 2-(4-{3-[(2R)-2-methylpyrrolidin-1- | 440 | 1.09 (d, 6.0 Hz, 3H), 1.42 (m, 1H), 1.69 (t, 9.3 Hz, 3H), |
| | yl]propoxy}phenyl)-5-(pyrrolidin-1- | | 1.78 (m, 1H), 1.91 (m, 3H), 2.01 (d, 6.5 Hz, 2H), |
| | ylcarbonyl)-5,6-dihydro-4H- | | 2.04 (m, 1H), 2.12 (m, 1H), 2.21 (m, 1H), |
| | cyclopenta[d][1,3]thiazole | | 2.30 (m, 1H), 2.98 (m, 1H), 3.12 (m, 1H), 3.18 (m, 2H), |
| | | | 3.38 (m, 1H), 3.54 (m, 4H), 3.86 (m, 1H), 4.07 (m, |
| | | | 2H), 6.92 (d, 8.8 Hz, 2H), 7.80 (d, 8.8 Hz, 2H) |
| 113 | 5-acetyl-2-{4-[3-(2-methylpyrrolidin-1- | 386 | 1.13 (d, 6.3 Hz, 3H), 1.47 (m, 1H), 1.77 (m, 2H), |
| | yl)propoxy]phenyl}-5,6-dihydro-4H- | | 1.94 (m, 1H), 2.04 (m, 2H), 2.17 (m, 4H), 2.27 (m, |
| | pyrrolo[3,4-d][1,3]thiazole | | 1H), 2.42 (m, 1H), 3.02 (dt, 11.8, 8.0 Hz, 1H), |
| | | | 3.23 (m, 1H), 4.09 (m, 2H), 4.72 (m, 2H), 4.79 (t, 2.8 Hz, |
| | | | 1H), 4.83 (t, 2.8 Hz, 1H), 6.95 (m, 2H), |
| | | | 7.83 (m, 2H) |

### Example 48

### Affinity for the Histamine H₃-Receptor; Inverse Agonism, Antagonism and Agonism Activity: [³⁵S]GTPγS-Binding Assay Human Histamine H₃-Receptor

### Material and Methods

### Reagents

Reagents and reference compounds are of analytical grade and may be obtained from various commercial sources. [³⁵H]-N-α-methylhistamine (80-85 Ci/mmol) and [³⁵S]-GTPγS (1250 Ci/mmol) may be purchased from e.g. Perkin Elmer (Belgium). Cell culture reagents may be purchased from e.g. Cambrex (Belgium).

Test and reference compounds are dissolved in 100% DMSO to give a 1 mM stock solution. Final DMSO concentration in the assay does not exceed 1%.

A CHO cell line expressing the unspliced full length (445 AA) human H₃ histamine receptor may be purchased from e.g. Euroscreen S. A. (Belgium).

Cells are grown in HAM-F12 culture media containing 10% fetal bovine serum, 100 IU/ml penicillin, 100 μg/ml streptomycin, 1% sodium pyruvate and 400 μg/ml of gentamycin.

Cells are maintained at 37° C. in a humidified atmosphere composed of 95% air and 5% CO₂.

### Membrane Preparation

Confluent cells are detached by 10 min incubation at 37° C. in PBS/EDTA 0.02%. The cell suspension is centrifuged at 1,500×g for 10 min at 4° C. The pellet is homogenized in a 15 mM Tris-HCl buffer (pH 7.5) containing 2 mM MgCl₂, 0.3 mM EDTA, 1 mM EGTA (buffer A). The crude homogenate is frozen in liquid nitrogen and thawed. DNAse (1 μl/ml) is then added and the homogenate is further incubated for 10 min at 25° C. before being centrifuged at 40,000×g for 25 min at 4° C. The pellet is resuspended in buffer A and washed once more under the same conditions. The final membrane pellet is resuspended, at a protein concentration of 1-3 mg/ml, in a 7.5 mM Tris-HCl buffer (pH 7.5) enriched with 12.5 mM MgCl₂, 0.3 mM EDTA, 1 mM EGTA and 250 mM sucrose and stored in liquid nitrogen until used.

### Binding Assays

### [³H]-N-α-methylhistamine Binding Assay

Affinity of compounds for histamine H₃ receptors may be measured by competition with [³H]-N-α-methylhistamine. This binding assay may be performed on any H₃ sequence, human or non-human. Briefly, membranes (20-40 μg proteins) expressing human H₃ histamine receptors are incubated at 25° C. in 0.5 ml of a 50 mM Tris-HCl buffer (pH 7.4) containing 2 mM MgCl₂, 0.2 nM [³H]-N-α-methyl-histamine and increasing concentrations of drugs. The non specific binding (NSB) is defined as the residual binding observed in the presence of 10 μM thioperamide or histamine. Membrane-bound and free radioligand are separated by rapid filtration through glass fiber filters presoaked in 0.1% PEI. Samples and filters are rinsed by at least 6 ml of ice-cold 50 mM Tris-HCl buffer (pH 7.4). The entire filtration procedure does not exceed 10 seconds per sample. Radioactivity trapped onto the filters is counted by liquid scintillation in a β-counter.

### [³⁵S]-GTPγS Binding Assay

Stimulation (agonist) or inhibition (inverse agonist) of [³⁵S]-GTPγS binding to membrane expressing human or non-human H₃ histamine receptors is measured as described by Lorenzen et al. (Mol. Pharmacol. 1993, 44, 115-123) with a few modifications. Briefly, membranes (10-20 μg proteins) expressing human or non-human H₃ histamine receptors are incubated at 25° C. in 0.2 ml of a 50 mM Tris-HCl buffer (pH 7.4) containing 3 mM MgCl₂, 50 mM NaCl, 1 μM GDP, 2 μg saponin and increasing concentrations of the test compound. After 15 min preincubation, 0.2 nM of [³⁵S]-GTPγS are added to the samples. The non specific binding (NSB) is defined as the residual binding observed in the presence of 100 μM Gpp(NH)p. Membrane-bound and free radioligand are separated by rapid filtration through glass fiber filters. Samples and filters are rinsed by at least 6 ml of ice-cold 50 mM Tris-HCl buffer (pH 7.4). The entire filtration procedure does not exceed 10 seconds per sample. Radioactivity trapped onto the filters is counted by liquid scintillation in a β-counter.

### Data Analysis

Determination of pIC₅₀/pKi/pEC₅₀/pEC₅₀INV

### Analysis

Raw data are analyzed by non-linear regression using XLfit™ (IDBS, United Kingdom) according to the following generic equation

B=MIN+[(MAX−MIN)/(1+(((10^{x})/(10^{-pX50}))^{nH}))]

where:
B is the radioligand bound in the presence of the unlabelled compound (dpm),
MIN is the minimal binding observed (dpm)
MAX is maximal binding observed (dpm),
X is the concentration of unlabelled compound (log M),
pX₅₀ (−log M) is the concentration of unlabelled compound causing 50% of its maximal effect (inhibition or stimulation of radioligand binding). It stands for pIC₅₀ when determining the affinity of a compound for the receptor in binding studies with [³H]-N-α-methylhistamine, for pEC₅₀ for compounds stimulating the binding of [³⁵S]-GTPγS (agonists) and for pEC₅₀INV for compounds inhibiting the binding of [³⁵S]-GTPγS (inverse agonists).
nH is the Hill coefficient.

pKi is obtained by applying the following equation (Cheng and Prusoff, 1973, Biochem. Pharmacol., 22: 3099-3108):

pKi=pIC₅₀+log(1+L/Kd)

where:
pKi is the unlabelled compound equilibrium dissociation constant (−log M),
L is the radioligand concentration (nM),
Kd is the radioligand equilibrium dissociation constant (nM).

Compounds of formula (I) according to the invention showed pIC₅₀ values greater than or equal to 6.2 for the histamine H₃ receptor.

Some compounds of formula (I) according to the present invention show pIC₅₀ values greater than or equal to 7.5.

Some compounds of formula (I) according to the present invention show pIC₅₀ values greater than or equal to 8.3.

Compounds of formula (I) according to the invention showed pEC₅₀INV values typically greater than or equal to 6.5 for the histamine H₃ receptor.

### Example 49

### Antagonism Activity: Paced Isolated Guinea Pig Myenteric Plexus—Electric-Field Stimulation Assay

### Material and Methods

### Reagents

Stock solutions (10⁻² M) of compounds to be tested and further dilutions are freshly prepared in DMSO (WNR, Leuven, Belgium). All other reagents (R(−)-α-methylhistamine, mepyramine, ranitidine, propranolol, yohimbine and components of the Krebs' solution) are of analytical grade and obtained from conventional commercial sources.

### Animals

Four week-old male Dunkin-Hartley guinea pigs (200-300 g) are supplied by Charles River (Sultfeld, Germany). All animals are ordered and used under protocol “orgisol-GP” approved by the UCB Pharma ethical committee. Animals are housed in the UCB animal facility in groups of 12, in stainless steel cages (75×50×30 cm) and allowed to acclimatise for a minimum of one week before inclusion in the study. Room temperature is maintained between 20 and 24° C. with 40 to 70% relative humidity. A light and dark cycle of 12 h is applied. Animals have free access to food and water.

### Organ Preparation

The method is adapted from that described by Menkveld et al. in Eur. J. Pharmacol. 1990, 186, 343-347. Longitudinal myenteric plexus is prepared from the isolated guinea pig ileum. Tissues are mounted in 20-ml organ baths containing modified Krebs' solution with 10⁻⁷ M mepyramine, 10⁻⁵ M ranitidine, 10⁻⁵ M propranolol and 10⁻⁶ M yohimbine. The bathing solution is maintained at 37° C. and gassed with 95% O₂-5% CO₂. Tissues are allowed to equilibrate for a 60-min period under a resting tension of 0.5 g and an electrical field stimulation (pulses of 5-20 V, 1 ms and 0.1 Hz is applied during the whole experiment). Such a stimulation induces stable and reproductive twitch contractions. Isometric contractions are measured by force-displacement transducers coupled to an amplifier connected to a computer system (EMKA Technologies) capable of controlling (i) automatic data acquisition, (ii) bath washout by automatic fluid circulation through electrovalves at predetermined times or signal stability and (iii) automatic dilution/injection of drug in the bath at predetermined times or signal stability.

### Protocol

After a 60 min-stabilisation period, tissues are stimulated twice with 10⁻⁶ M R(−)-α-methylhistamine at 30-min interval. After a 60-min incubation period in the presence of solvent or antagonist test compound, a cumulative concentration-response to R(−)-α-methylhistamine is elicited (10⁻¹⁰à 10⁻⁴ M). Only one concentration of antagonist is tested on each tissue.

### Data Analysis

An appropriate estimate of interactions between agonist and antagonist can be made by studying the family of curves observed in the absence or presence of increasing antagonist concentrations. The value of each relevant parameter of each concentration-response curve (pD₂ and Eₘₐₓ) is calculated by an iterative computer software (XLfit, IDBS, Guildford, UK) fitting the experimental data to the four parameter logistic equation. Antagonistic activity of the test substance is estimated by the calculation of pD′₂ and/or pA₂ values according to the methods described by Van Rossum et al. in Arch. Int. Pharmacodyn. Ther. 1963, 143, 299 and/or by Arunlakshana & Schild in Br. J. Pharmacol 1959, 14, 48

Results are expressed as the mean±SD. The number of observations is indicated as n. Compounds of formula (I) according to the invention showed pA₂ values typically greater than or equal to 6.5 for the histamine H₃ receptor.

1. A compound of formula (I), geometrical isomers, enantiomers, diastereoisomers, pharmaceutically acceptable salts and all possible mixtures thereof, 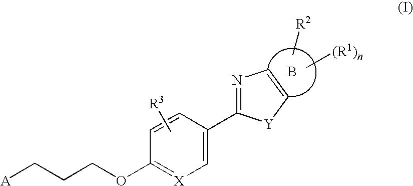
wherein
A is a cyclic amine which is linked to the propylene group via an amino nitrogen;
B is selected from the group consisting of heteroaryl, 5-8-membered heterocycloalkyl, 5-8-membered cycloalkyl;
X is either N or CH;
Y is either O or S;
R¹ is selected from the group comprising or consisting of sulfonyl, amino, C₁-C₆-alkyl, C₂-C₆-alkenyl, C₂-C₆-alkynyl, aryl, heteroaryl, C₃-C₈-cycloalkyl, 3-8-membered heterocycloalkyl, acyl, C₁-C₆-alkyl aryl, C₁-C₆-alkyl heteroaryl, C₂-C₆-alkenyl aryl, C₂-C₆-alkenyl heteroaryl, C₂-C₆-alkynyl aryl, C₂-C₆-alkynyl heteroaryl, C₁-C₆-alkyl cycloalkyl, C₁-C₆-alkyl heterocycloalkyl, C₂-C₆-alkenyl cycloalkyl, C₂-C₆-alkenyl heterocycloalkyl, C₂-C₆-alkynyl cycloalkyl, C₂-C₆-alkynyl heterocycloalkyl, alkoxycarbonyl, aminocarbonyl, C₁-C₆-alkyl carboxy, C₁-C₆-alkyl acyl, aryl acyl, heteroaryl acyl, C₃-C₈-(hetero)cycloalkyl acyl, C₁-C₆-alkyl acyloxy, C₁-C₆-alkyl alkoxy, C₁-C₆-alkyl alkoxycarbonyl, C₁-C₆-alkyl aminocarbonyl, C₁-C₆-alkyl acylamino, acylamino, acylaminocarbonyl, ureido, C₁-C₆-alkyl ureido, C₁-C₆-alkyl carbamate, C₁-C₆-alkyl amino, C₁-C₆-alkyl sulfonyloxy, C₁-C₆-alkyl sulfonyl, C₁-C₆-alkyl sulfinyl, C₁-C₆-alkyl sulfanyl, C₁-C₆-alkyl sulfonylamino, C₁-C₆-alkyl aminosulfonyl, hydroxy, halogen, cyano, carboxy, oxo, thioxo;
n is equal to 0, 1, 2 or 3;
R² is selected from the group comprising or consisting of hydrogen, sulfonyl, amino, C₁-C₆-alkyl, C₂-C₆-alkenyl, C₂-C₆-alkynyl, aryl, heteroaryl, C₃-C₈-cycloalkyl, 3-8-membered heterocycloalkyl, acyl, C₁-C₆-alkyl aryl, C₁-C₆-alkyl heteroaryl, C₂-C₆-alkenyl aryl, C₂-C₆-alkenyl heteroaryl, C₂-C₆-alkynyl aryl, C₂-C₆-alkynyl heteroaryl, C₁-C₆-alkyl cycloalkyl, C₁-C₆-alkyl heterocycloalkyl, C₂-C₆-alkenyl cycloalkyl, C₂-C₆-alkenyl heterocycloalkyl, C₂-C₆-alkynyl cycloalkyl, C₂-C₆-alkynyl heterocycloalkyl, alkoxycarbonyl, aminocarbonyl, C₁-C₆-alkyl carboxy, C₁-C₆-alkyl acyl, aryl acyl, heteroaryl acyl, C₃-C₈-(hetero)cycloalkyl acyl, C₁-C₆-alkyl acyloxy, C₁-C₆-alkyl alkoxy, C₁-C₆-alkyl alkoxycarbonyl, C₁-C₆-alkyl aminocarbonyl, C₁-C₆-alkyl acylamino, acylamino, acylaminocarbonyl, ureido, C₁-C₆-alkyl ureido, C₁-C₆-alkyl carbamate, C₁-C₆-alkyl amino, C₁-C₆-alkyl sulfonyloxy, C₁-C₆-alkyl sulfonyl, C₁-C₆-alkyl sulfinyl, C₁-C₆-alkyl sulfanyl, C₁-C₆-alkyl sulfonylamino, C₁-C₆-alkyl aminosulfonyl, hydroxy, halogen, cyano, carboxy, oxo, thioxo;
R³ is hydrogen or C₁-C₆-alkyl or halogen or C₁-C₆-alkoxy.

2. The compound according to claim 1, wherein Y is S.

3. The compound according to claim 1 wherein X is CH.

4. The compound according to claim 1 wherein A is a pyrrolidinyl, an azepanyl, a piperazinyl or a piperidinyl group.

5. The compound according to claim 1 wherein B is a 5, 6 or 7-membered cycloalkyl or heterocycloalkyl.

6. The compound according to claim 5, wherein B is tetrahydropyridyl, dihydro-1H-pyrrolyl, tetrahydro-1H-azepinyl, cyclohexenyl or cyclopentenyl.

7. The compound according to claim 1 wherein R¹ is selected from the group consisting of C₁-C₆-alkyl, aryl, heteroaryl, C₃-C₈-cycloalkyl, 3-8-membered heterocycloalkyl, acyl, C₁-C₆-alkyl cycloalkyl, C₁-C₆-alkyl heterocycloalkyl, alkoxycarbonyl, aminocarbonyl, C₁-C₆-alkyl alkoxycarbonyl, C₁-C₆-alkyl aminocarbonyl, hydroxy, halogen, cyano, carboxy, oxo, thioxo.

8. The compound according to claim 7, wherein R¹ is selected from the group consisting of C₁-C₆-alkyl, hydroxy, oxo.

9. The compound according to claim 1 wherein n is either 0 or 1.

10. The compound according to claim 1 wherein R² is selected from the group consisting of hydrogen, sulfonyl, C₁-C₆-alkyl, C₂-C₆-alkenyl, C₂-C₆-alkynyl, aryl, heteroaryl, C₃-C₈-cycloalkyl, 3-8-membered heterocycloalkyl, acyl, C₁-C₆-alkyl cycloalkyl, C₁-C₆-alkyl heterocycloalkyl, alkoxycarbonyl, aminocarbonyl, acylamino, ureido, C₁-C₆-alkyl ureido, C₁-C₆-alkyl carbamate, amino, C₁-C₆-alkyl amino, hydroxy, oxo.

11. The compound according to claim 10, wherein R² is selected from the group consisting of hydrogen, sulfonyl, C₁-C₆-alkyl, aryl, C₃-C₈-cycloalkyl, 3-8-membered heterocycloalkyl, acyl, C₁-C₆-alkyl cycloalkyl, alkoxycarbonyl, aminocarbonyl, acylamino, ureido, C₁-C₆-alkyl carbamate, amino, hydroxy, oxo.

12. The compound according to claim 1, wherein R² is selected from the group consisting of hydrogen, methyl, ethyl, acetyl, cyclohexylmethyl, cyclopentyl, trifluoroacetyl, 4-fluorophenyl, benzoyl, cyclohexylcarbonyl, thien-2-ylcarbonyl, 2,2-dimethylpropanoyl, butyryl, tert-butoxycarbonyl, (ethylamino)carbonyl, cyclopropyl-carbonyl, isonicotinoyl, methoxyacetyl, methylsulfonyl, (benzylamino)carbonyl, anilinocarbonyl, {[2-(2-thienyl)ethyl]amino}carbonyl, amino carbonyl, (isopropyl-amino)carbonyl, (cyclohexylamino)carbonyl, {[1-(trifluoroacetyl)piperidin-4-yl]amino}carbonyl, [(2-ethoxy-2-oxoethyl)amino]carbonyl, [(2,4-difluorophenyl)-amino]carbonyl, 3,3,3-trifluoropropanoyl, (benzoylamino)carbonyl, 4-methyl-phenylsulfonyl, (butylamino)carbonyl, hydroxy, piperidin-1-yl, acetylamino, ethoxycarbonyl, [(benzyloxy)carbonyl]amino, [(2-methylpyrrolidin-1-yl)carbonyl]-amino, piperidin-1-ylcarbonyl, [(benzyloxy)carbonyl]amino, amino, oxo, morpholin-4-ylsulfonyl, pyrrolidin-1-carbonyl, morpholin-4-carbonyl, (diethylamino)carbonyl, (4-methylpiperazin-1-yl)carbonyl, (4,4-difluoropiperidin-1-yl)carbonyl, 5,5,5-trifluoropentanoyl, 3-hydroxybutyl, 3-hydroxycyclobutyl, 3-methoxy-3-oxopropanoyl, 2-hydroxyethyl, hydroxyacetyl, 3-oxocyclobut-1-en-1-yl, 3-fluorocyclobutyl, 3-amino-3-oxopropanoyl, 3-methoxy-3-oxopropanoyl, carboxyacetyl, pyrrolidin-1-ylsulfonyl, 2-amino-2-oxoethyl, 2-oxopropyl, 2,3-dihydroxypropyl, 2-tert-butoxy-2-oxoethyl, carboxymethyl, 1,4-dioxa-8-azaspiro[4.5]dec-8-ylcarbonyl, (4-methylpiperazin-1-yl)carbonyl, thiomorpholin-4-ylcarbonyl, (4-isopropylpiperazin-1-yl)carbonyl, octahydroisoquinolin-2(1H)-ylcarbonyl, [(cyclopropylmethyl)amino]carbonyl, [(cyclopropylmethyl)(propyl)amino]carbonyl.

13. The compound according to claim 1 of the formula (IA) 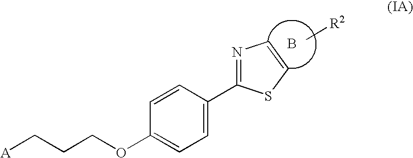
wherein A is either a pyrrolidinyl or a piperidinyl and B and R² are as above defined.

14. The compound according to claim 13, wherein A is a pyrrolidin-1-yl which may be substituted by a C₁-C₆-alkyl or an amino, B is a tetrahydropyridyl or a tetrahydro-1H-azepinyl, and R² is linked to the tetrahydropyridyl or the tetrahydro-1H-azepinyl nitrogen and is selected from hydrogen, C₁-C₆-alkyl, C₃-C₈-cycloalkyl, sulfonyl, acyl, C₁-C₆-alkyl acyl, alkoxycarbonyl, C₁-C₆-alkyl alkoxycarbonyl, aminocarbonyl, C₁-C₆-alkyl aminocarbonyl, C₁-C₆-alkyl carboxy, C₁-C₆-alkyl hydroxy or C₃-C₈-cycloalkyl hydroxy.

15. The compound according to claim 1 of the formula (IA) wherein A is a pyrrolidin-1-yl which may be substituted by a C₁-C₆-alkyl, B is a cyclopentenyl or a cyclohexenyl, and R² is a 3-8 membered heterocycloalkyl, acylamino, carbamate, amino, aminocarbonyl.

16. The compound according to claim 1 of the formula (IB) 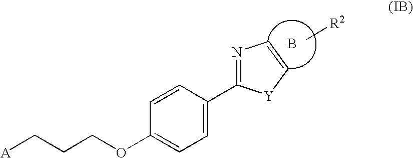
wherein R² is C₃-C₈-cycloalkyl hydroxy, sulfonyl, acyl, aminocarbonyl or C₁-C₆-alkyl aminocarbonyl, A, B and Y are as above defined.

17. The compound according to claim 1 selected from the group consisting of:
2-[4-(3-piperidin-1-ylpropoxy)phenyl]-4,5,6,7-tetrahydro[1,3]thiazolo[5,4-c]pyridine;
2-{4-[3-(2-methylpyrrolidin-1-yl)propoxy]phenyl}-4,5,6,7-tetrahydro[1,3]thiazolo[5,4-c]pyridine;
5-ethyl-2-{4-[3-(2-methylpyrrolidin-1-yl)propoxy]phenyl}-4,5,6,7-tetrahydro[1,3]thiazolo[5,4-c]pyridine;
5-acetyl-2-{4-[3-(2-methylpyrrolidin-1-yl)propoxy]phenyl}-4,5,6,7-tetrahydro[1,3]thiazolo-[5,4-c]pyridine;
5-acetyl-2-(4-{3-[(2R)-2-methylpyrrolidin-1-yl]propoxy}phenyl)-4,5,6,7-tetrahydro[1,3]thiazolo[5,4-c]pyridine;
5-acetyl-2-(4-{3-[(2S)-2-methylpyrrolidin-1-yl]propoxy}phenyl)-4,5,6,7-tetrahydro-[1,3]thiazolo[5,4-c]pyridine;
5-(cyclohexylmethyl)-2-{4-[3-(2-methylpyrrolidin-1-yl)propoxy]phenyl}-4,5,6,7-tetrahydro[1,3]thiazolo[5,4-c]pyridine;
5-cyclopentyl-2-{4-[3-(2-methylpyrrolidin-1-yl)propoxy]phenyl}-4,5,6,7-tetrahydro[1,3]thiazolo[5,4-c]pyridine;
2-{4-[3-(2-methylpyrrolidin-1-yl)propoxy]phenyl}-5-(trifluoroacetyl)-4,5,6,7-tetrahydro[1,3]thiazolo[5,4-c]pyridine;
5-(4-fluorophenyl)-2-{4-[3-(2-methylpyrrolidin-1-yl)propoxy]phenyl}-4,5,6,7-tetrahydro[1,3]thiazolo[5,4-c]pyridine;
5-benzoyl-2-{4-[3-(2-methylpyrrolidin-1-yl)propoxy]phenyl}-4,5,6,7-tetrahydro[1,3]-thiazolo[5,4-c]pyridine;
5-(cyclohexylcarbonyl)-2-{4-[3-(2-methylpyrrolidin-1-yl)propoxy]phenyl}-4,5,6,7-tetrahydro[1,3]thiazolo[5,4-c]pyridine;
2-{4-[3-(2-methylpyrrolidin-1-yl)propoxy]phenyl}-5-(thien-2-ylcarbonyl)-4,5,6,7-tetrahydro[1,3]thiazolo[5,4-c]pyridine;
5-(2,2-dimethylpropanoyl)-2-{4-[3-(2-methylpyrrolidin-1-yl)propoxy]phenyl}-4,5,6,7-tetrahydro[1,3]thiazolo[5,4-c]pyridine;
5-butyryl-2-{4-[3-(2-methylpyrrolidin-1-yl)propoxy]phenyl}-4,5,6,7-tetrahydro[1,3]thiazolo-[5,4-c]pyridine;
tert-butyl 2-{4-[3-(2-methylpyrrolidin-1-yl)propoxy]phenyl}-6,7-dihydro[1,3]thiazolo[5,4-c]pyridine-5(4H)-carboxylate;
N-ethyl-2-{4-[3-(2-methylpyrrolidin-1-yl)propoxy]phenyl}-6,7-dihydro[1,3]thiazolo[5,4-c]pyridine-5(4H)-carboxamide;
N-ethyl-2-(4-{3-[(2R)-2-methylpyrrolidin-1-yl]propoxy}phenyl)-6,7-dihydro[1,3]thiazolo[5,4-c]pyridine-5(4H)-carboxamide;
N-ethyl-2-(4-{3-[(2S)-2-methylpyrrolidin-1-yl]propoxy}phenyl)-6,7-dihydro[1,3]thiazolo[5,4-c]pyridine-5(4H)-carboxamide;
5-(cyclopropylcarbonyl)-2-{4-[3-(2-methylpyrrolidin-1-yl)propoxy]phenyl}-4,5,6,7-tetrahydro[1,3]thiazolo[5,4-c]pyridine;
5-isonicotinoyl-2-{4-[3-(2-methylpyrrolidin-1-yl)propoxy]phenyl}-4,5,6,7-tetrahydro[1,3]-thiazolo[5,4-c]pyridine;
5-(methoxyacetyl)-2-(4-{3-[(2R)-2-methylpyrrolidin-1-yl]propoxy}phenyl)-4,5,6,7-tetrahydro[1,3]thiazolo[5,4-c]pyridine;
5-(methoxyacetyl)-2-{4-[3-(2-methylpyrrolidin-1-yl)propoxy]phenyl}-4,5,6,7-tetrahydro-[1,3]thiazolo[5,4-c]pyridine;
5-(methoxyacetyl)-2-(4-{3-[(2S)-2-methylpyrrolidin-1-yl]propoxy}phenyl)-4,5,6,7-tetrahydro[1,3]thiazolo[5,4-c]pyridine;
N-ethyl-2-(4-{3-[2-(4-{3-[(2S)-2-methylpyrrolidin-1-yl]propoxy}phenyl)-6,7-dihydro[1,3]thiazolo[5,4-c]pyridin-5(4H)-yl]propoxy}phenyl)-6,7-dihydro[1,3]thiazolo[5,4-c]pyridine-5(4H)-carboxamide;
2-{4-[3-(2-methylpyrrolidin-1-yl)propoxy]phenyl}-5-(methylsulfonyl)-4,5,6,7-tetrahydro[1,3]thiazolo[5,4-c]pyridine;
N-benzyl-2-{4-[3-(2-methylpyrrolidin-1-yl)propoxy]phenyl}-6,7-dihydro[1,3]thiazolo[5,4-c]pyridine-5(4H)-carboxamide;
2-{4-[3-(2-methylpyrrolidin-1-yl)propoxy]phenyl}-N-phenyl-6,7-dihydro[1,3]thiazolo[5,4-c]pyridine-5(4H)-carboxamide;
2-{4-[3-(2-methylpyrrolidin-1-yl)propoxy]phenyl}-N-(2-thien-2-ylethyl)-6,7-dihydro[1,3]-thiazolo[5,4-c]pyridine-5(4H)-carboxamide;
2-{4-[3-(2-methylpyrrolidin-1-yl)propoxy]phenyl}-6,7-dihydro[1,3]thiazolo[5,4-c]pyridine-5(4H)-carboxamide;
N-isopropyl-2-{4-[3-(2-methylpyrrolidin-1-yl)propoxy]phenyl}-6,7-dihydro[1,3]thiazolo[5,4-c]pyridine-5(4H)-carboxamide;
N-cyclohexyl-2-{4-[3-(2-methylpyrrolidin-1-yl)propoxy]phenyl}-6,7-dihydro[1,3]thiazolo[5,4-c]pyridine-5(4H)-carboxamide;
2-{4-[3-(2-methylpyrrolidin-1-yl)propoxy]phenyl}-N-[1-(trifluoroacetyl)piperidin-4-yl]-6,7-dihydro[1,3]thiazolo[5,4-c]pyridine-5(4H)-carboxamide;
ethyl ({[2-{4-[3-(2-methylpyrrolidin-1-yl)propoxy]phenyl}-6,7-dihydro[1,3]thiazolo[5,4-c]pyridin-5(4H)-yl]carbonyl}amino)acetate;
N-(2,4-difluorophenyl)-2-{4-[3-(2-methylpyrrolidin-1-yl)propoxy]phenyl}-6,7-dihydro[1,3]thiazolo[5,4-c]pyridine-5(4H)-carboxamide;
2-{4-[3-(2-methylpyrrolidin-1-yl)propoxy]phenyl}-5-(3,3,3-trifluoropropanoyl)-4,5,6,7-tetrahydro[1,3]thiazolo[5,4-c]pyridine;
N-benzoyl-2-{4-[3-(2-methylpyrrolidin-1-yl)propoxy]phenyl}-6,7-dihydro[1,3]thiazolo[5,4-c]pyridine-5(4H)-carboxamide
5-[(4-methylphenyl)sulfonyl]-2-{4-[3-(2-methylpyrrolidin-1-yl)propoxy]phenyl}-4,5,6,7-tetrahydro[1,3]thiazolo[5,4-c]pyridine;
N-butyl-2-{4-[3-(2-methylpyrrolidin-1-yl)propoxy]phenyl}-6,7-dihydro[1,3]thiazolo[5,4-c]pyridine-5(4H)-carboxamide;
5-acetyl-2-(4-{3-[2-(pyrrolidin-1-ylmethyl)pyrrolidin-1-yl]propoxy}phenyl)-4,5,6,7-tetrahydro[1,3]thiazolo[5,4-c]pyridine;
5-acetyl-2-[4-(3-azepan-1-ylpropoxy)phenyl]-4,5,6,7-tetrahydro[1,3]thiazolo[5,4-c]pyridine;
5-acetyl-2-{4-[3-(4-isopropylpiperazin-1-yl)propoxy]phenyl}-4,5,6,7-tetrahydro[1,3]thiazolo[5,4-c]pyridine;
5-acetyl-2-{4-[3-(2-methylpiperidin-1-yl)propoxy]phenyl}-4,5,6,7-tetrahydro[1,3]thiazolo[5,4-c]pyridine;
1-{3-[4-(5-acetyl-4,5,6,7-tetrahydro[1,3]thiazolo[5,4-c]pyridin-2-yl)phenoxy]propyl}-N,N-dimethylpyrrolidin-3-amine;
5-acetyl-2-{4-[3-(3,5-dimethylpiperidin-1-yl)propoxy]phenyl}-4,5,6,7-tetrahydro[1,3]-thiazolo[5,4-c]pyridine;
5-[(4-methylphenyl)sulfonyl]-2-{4-[3-(2-methylpyrrolidin-1-yl)propoxy]phenyl}-5,6-dihydro-4H-pyrrolo[3,4-d][1,3]thiazole;
2-{4-[3-(2-methylpyrrolidin-1-yl)propoxy]phenyl}-5,6,7,8-tetrahydro-4H-[1,3]thiazolo[4,5-d]azepine;
2-{4-[3-(2-methylpyrrolidin-1-yl)propoxy]phenyl}-4,5,6,7-tetrahydro-1,3-benzothiazol-4-ol;
2-{4-[3-(2-methylpyrrolidin-1-yl)propoxy]phenyl}-4-piperidin-1-yl-4,5,6,7-tetrahydro-1,3-benzothiazole;
2-{4-[3-(2-methylpyrrolidin-1-yl)propoxy]phenyl}-7-piperidin-1-yl-4,5,6,7-tetrahydro-1,3-benzothiazole;
N-(2-{4-[3-(2-methylpyrrolidin-1-yl)propoxy]phenyl}-4,5,6,7-tetrahydro-1,3-benzothiazol-6-yl)acetamide;
ethyl 2-{4-[3-(2-methylpyrrolidin-1-yl)propoxy]phenyl}-4,5,6,7-tetrahydro-1,3-benzothiazole-4-carboxylate;
benzyl 2-{4-[3-(2-methylpyrrolidin-1-yl)propoxy]phenyl}-4,5,6,7-tetrahydro-1,3-benzothiazol-4-ylcarbamate;
N-(2-{4-[3-(2-methylpyrrolidin-1-yl)propoxy]phenyl}-4,5,6,7-tetrahydro-1,3-benzothiazol-4-yl)acetamide;
2-methyl-N-(2-{4-[3-(2-methylpyrrolidin-1-yl)propoxy]phenyl}-4,5,6,7-tetrahydro-1,3-benzothiazol-4-yl)pyrrolidine-1-carboxamide;
N-(2-{4-[3-(2-methylpyrrolidin-1-yl)propoxy]phenyl}-4,5,6,7-tetrahydro-1,3-benzothiazol-5-yl)acetamide;
ethyl 2-{4-[3-(2-methylpyrrolidin-1-yl)propoxy]phenyl}-5,6-dihydro-4H-cyclopenta[d][1,3]-thiazole-4-carboxylate;
2-{4-[3-(2-methylpyrrolidin-1-yl)propoxy]phenyl}-4-(piperidin-1-ylcarbonyl)-5,6-dihydro-4H-cyclopenta[d][1,3]thiazole;
benzyl 2-{4-[3-(2-methylpyrrolidin-1-yl)propoxy]phenyl}-5,6-dihydro-4H-cyclopenta[d]-[1,3]thiazol-4-ylcarbamate;
ethyl 4-methyl-2-{4-[3-(2-methylpyrrolidin-1-yl)propoxy]phenyl}-5,6-dihydro-4H-cyclopenta-[d][1,3]thiazole-4-carboxylate;
(2-{4-[3-(2-methyl-pyrrolidin-1-yl)-propoxy]-phenyl}-5,6-dihydro-4H-cyclopentathiazol-4-ylamine;
N-(2-{4-[3-(2-methylpyrrolidin-1-yl)propoxy]phenyl}-5,6-dihydro-4H-cyclopenta[d][1,3]-thiazol-4-yl)acetamide;
2-{4-[3-(2-methylpyrrolidin-1-yl)propoxy]phenyl}-4-(methylsulfonyl)-4,5,6,7-tetrahydro-[1,3]thiazolo[4,5-b]pyridine;
4-(methoxyacetyl)-2-{4-[3-(2-methylpyrrolidin-1-yl)propoxy]phenyl}-4,5,6,7-tetrahydro-[1,3]thiazolo[4,5-b]pyridine;
4-acetyl-2-{4-[3-(2-methylpyrrolidin-1-yl)propoxy]phenyl}-4,5,6,7-tetrahydro[1,3]-thiazolo[4,5-b]pyridine;
N-ethyl-2-{4-[3-(2-methylpyrrolidin-1-yl)propoxy]phenyl}-6,7-dihydro[1,3]thiazolo[4,5-b]pyridine-4(5H)-carboxamide;
4-isonicotinoyl-2-{4-[3-(2-methylpyrrolidin-1-yl)propoxy]phenyl}-4,5,6,7-tetrahydro-[1,3]thiazolo[4,5-b]pyridine;
2-{4-[3-(2-methylpyrrolidin-1-yl)propoxy]phenyl}-4-(morpholin-4-ylsulfonyl)-4,5,6,7-tetrahydro[1,3]thiazolo[4,5-b]pyridine;
2-{4-[3-(2-methylpyrrolidin-1-yl)propoxy]phenyl}-4,5,6,7-tetrahydro[1,3]thiazolo[4,5-b]pyridine;
2-{4-[3-(2-methylpyrrolidin-1-yl)propoxy]phenyl}-4-(morpholin-4-ylcarbonyl)-4,5,6,7-tetrahydro[1,3]thiazolo[4,5-b]pyridine;
2-{4-[3-(2-methylpyrrolidin-1-yl)propoxy]phenyl}-6,7-dihydro[1,3]thiazolo[4,5-b]pyridin-5(4H)-one;
5-methyl-2-{4-[3-(2-methylpyrrolidin-1-yl)propoxy]phenyl}-4,5,6,7-tetrahydro[1,3]-thiazolo[4,5-c]pyridine;
5-acetyl-2-(4-{3-[(2R)-2-methylpyrrolidin-1-yl]propoxy}phenyl)-4,5,6,7-tetrahydro[1,3]-thiazolo[4,5-c]pyridine;
N-ethyl-2-(4-{3-[(2R)-2-methylpyrrolidin-1-yl]propoxy}phenyl)-6,7-dihydro[1,3]thiazolo[4,5-c]pyridine-5(4H)-carboxamide;
5-(methoxyacetyl)-2-(4-{3-[(2R)-2-methylpyrrolidin-1-yl]propoxy}phenyl)-4,5,6,7-tetrahydro[1,3]thiazolo[4,5-c]pyridine;
5-methyl-2-(4-{3-[(2R)-2-methylpyrrolidin-1-yl]propoxy}phenyl)-4,5,6,7-tetrahydro[1,3]-oxazolo[4,5-c]pyridine;
2-{4-[3-(2-methylpyrrolidin-1-yl)propoxy]phenyl}-5-(pyrrolidin-1-ylcarbonyl)-4,5,6,7-tetrahydro[1,3]thiazolo[5,4-c]pyridine;
2-{4-[3-(2-methylpyrrolidin-1-yl)propoxy]phenyl}-5-(morpholin-4-ylcarbonyl)-4,5,6,7-tetrahydro[1,3]thiazolo[5,4-c]pyridine;
N,N-diethyl-2-{4-[3-(2-methylpyrrolidin-1-yl)propoxy]phenyl}-6,7-dihydro[1,3]thiazolo[5,4-c]pyridine-5(4H)-carboxamide;
5-[(4-methylpiperazin-1-yl)carbonyl]-2-{4-[3-(2-methylpyrrolidin-1-yl)propoxy]phenyl}-4,5,6,7-tetrahydro[1,3]thiazolo[5,4-c]pyridine;
5-[(4,4-difluoropiperidin-1-yl)carbonyl]-2-{4-[3-(2-methylpyrrolidin-1-yl)propoxy]phenyl}-4,5,6,7-tetrahydro[1,3]thiazolo[5,4-c]pyridine;
2-{4-[3-(2-methylpyrrolidin-1-yl)propoxy]phenyl}-5-(5,5,5-trifluoropentanoyl)-4,5,6,7-tetrahydro[1,3]thiazolo[5,4-c]pyridine;
4-[2-{4-[3-(2-methylpyrrolidin-1-yl)propoxy]phenyl}-6,7-dihydro[1,3]thiazolo[5,4-c]pyridin-5(4H)-yl]butan-2-ol;
(3R)-1-{3-[4-(5-acetyl-4,5,6,7-tetrahydro[1,3]thiazolo[5,4-c]pyridin-2-yl)phenoxy]propyl}-N,N-dimethylpyrrolidin-3-amine;
cis-3-[2-{4-[3-(2-methylpyrrolidin-1-yl)propoxy]phenyl}-6,7-dihydro[1,3]thiazolo[5,4-c]pyridin-5(4H)-yl]cyclobutanol;
(3S)-1-{3-[4-(5-acetyl-4,5,6,7-tetrahydro[1,3]thiazolo[5,4-c]pyridin-2-yl)phenoxy]propyl}-N,N-dimethylpyrrolidin-3-amine;
methyl 3-[2-{4-[3-(2-methylpyrrolidin-1-yl)propoxy]phenyl}-6,7-dihydro[1,3]thiazolo[5,4-c]pyridin-5(4H)-yl]-3-oxopropanoate;
2-[2-{4-[3-(2-methylpyrrolidin-1-yl)propoxy]phenyl}-6,7-dihydro[1,3]thiazolo[5,4-c]pyridin-5(4H)-yl]ethanol;
3-[2-{4-[3-(2-methylpyrrolidin-1-yl)propoxy]phenyl}-6,7-dihydro[1,3]thiazolo[5,4-c]pyridin-5(4H)-yl]-3-oxopropanoic acid;
3-[2-(4-{3-[(2R)-2-methylpyrrolidin-1-yl]propoxy}phenyl)-6,7-dihydro[1,3]thiazolo[5,4-c]pyridin-5(4H)-yl]cyclobut-2-en-1-one;
5-(trans-3-fluorocyclobutyl)-2-{4-[3-(2-methylpyrrolidin-1-yl)propoxy]phenyl}-4,5,6,7-tetrahydro[1,3]thiazolo[5,4-c]pyridine;
2-(4-{3-[(2R)-2-methylpyrrolidin-1-yl]propoxy}phenyl)-5-(morpholin-4-ylsulfonyl)-4,5,6,7-tetrahydro[1,3]thiazolo[5,4-c]pyridine;
3-[2-(4-{3-[(2R)-2-methylpyrrolidin-1-yl]propoxy}phenyl)-6,7-dihydro[1,3]thiazolo[5,4-c]pyridin-5(4H)-yl]-3-oxopropanamide;
methyl [2-(4-{3-[(2R)-2-methylpyrrolidin-1-yl]propoxy}phenyl)-6,7-dihydro[1,3]thiazolo[5,4-c]pyridin-5(4H)-yl]acetate;
2-[2-(4-{3-[(2R)-2-methylpyrrolidin-1-yl]propoxy}phenyl)-6,7-dihydro[1,3]thiazolo[5,4-c]pyridin-5(4H)-yl]-2-oxoethanol;
2-(4-{3-[(2R)-2-methylpyrrolidin-1-yl]propoxy}phenyl)-5-(pyrrolidin-1-ylsulfonyl)-4,5,6,7-tetrahydro[1,3]thiazolo[5,4-c]pyridine;
2-[2-(4-{3-[(2R)-2-methylpyrrolidin-1-yl]propoxy}phenyl)-6,7-dihydro[1,3]thiazolo[5,4-c]pyridin-5(4H)-yl]acetamide;
1-[2-(4-{3-[(2R)-2-methylpyrrolidin-1-yl]propoxy}phenyl)-6,7-dihydro[1,3]thiazolo[5,4-c]pyridin-5(4H)-yl]acetone;
3-[2-(4-{3-[(2R)-2-methylpyrrolidin-1-yl]propoxy}phenyl)-6,7-dihydro[1,3]thiazolo[5,4-c]pyridin-5(4H)-yl]propane-1,2-diol;
((2S)-1-{3-[4-(5-acetyl-4,5,6,7-tetrahydro[1,3]thiazolo[5,4-c]pyridin-2-yl)phenoxy]-propyl}pyrrolidin-2-yl)methanol;
tert-butyl [2-(4-{3-[(2R)-2-methylpyrrolidin-1-yl]propoxy}phenyl)-6,7-dihydro[1,3]thiazolo-[5,4-c]pyridin-5(4H)-yl]acetate;
[2-(4-{3-[(2R)-2-methylpyrrolidin-1-yl]propoxy}phenyl)-6,7-dihydro[1,3]thiazolo[5,4-c]pyridin-5(4H)-yl]acetic acid;
6-acetyl-2-{4-[3-(2-methylpyrrolidin-1-yl)propoxy]phenyl}-5,6,7,8-tetrahydro-4H-[1,3]thiazolo[4,5-d]azepine;
2-{4-[3-(2-methylpyrrolidin-1-yl)propoxy]phenyl}-6-(methylsulfonyl)-5,6,7,8-tetrahydro-4H-[1,3]thiazolo[4,5-d]azepine;
N-ethyl-2-{4-[3-(2-methylpyrrolidin-1-yl)propoxy]phenyl}-4,5,7,8-tetrahydro-6H-[1,3]thiazolo[4,5-d]azepine-6-carboxamide;
2-{4-[3-(2-methylpyrrolidin-1-yl)propoxy]phenyl}-6-(morpholin-4-ylcarbonyl)-5,6,7,8-tetrahydro-4H-[1,3]thiazolo[4,5-d]azepine;
2-(2-{4-[3-(2-methylpyrrolidin-1-yl)propoxy]phenyl}-4,5,7,8-tetrahydro-6H-[1,3]thiazolo[4,5-d]azepin-6-yl)acetamide;
2-{4-[3-(2-methylpyrrolidin-1-yl)propoxy]phenyl}-6-(morpholin-4-ylsulfonyl)-5,6,7,8-tetrahydro-4H-[1,3]thiazolo[4,5-d]azepine;
6-isonicotinoyl-2-{4-[3-(2-methylpyrrolidin-1-yl)propoxy]phenyl}-5,6,7,8-tetrahydro-4H-[1,3]thiazolo[4,5-d]azepine;
(2-{4-[3-(2-methylpyrrolidin-1-yl)propoxy]phenyl}-4,5,7,8-tetrahydro-6H-[1,3]thiazolo[4,5-d]azepin-6-yl)acetic acid;
6-(1,4-dioxa-8-azaspiro[4.5]dec-8-ylcarbonyl)-2-{4-[3-(2-methylpyrrolidin-1-yl)propoxy]-phenyl}-5,6,7,8-tetrahydro-4H-[1,3]thiazolo[4,5-d]azepine;
6-[(4,4-difluoropiperidin-1-yl)carbonyl]-2-{4-[3-(2-methylpyrrolidin-1-yl)propoxy]phenyl}-5,6,7,8-tetrahydro-4H-[1,3]thiazolo[4,5-d]azepine;
7,7-dimethyl-2-{4-[3-(2-methylpyrrolidin-1-yl)propoxy]phenyl}-5,6,7,8-tetrahydro-4H-[1,3]thiazolo[5,4-c]azepin-4-one;
5,7,7-trimethyl-2-{4-[3-(2-methylpyrrolidin-1-yl)propoxy]phenyl}-5,6,7,8-tetrahydro-4H-[1,3]thiazolo[5,4-c]azepin-4-one;
4-acetyl-2-(4-{3-[(2R)-2-methylpyrrolidin-1-yl]propoxy}phenyl)-5,6,7,8-tetrahydro-4H-[1,3]thiazolo[5,4-b]azepine;
2-{4-[3-(2-methylpyrrolidin-1-yl)propoxy]phenyl}-5-(morpholin-4-ylcarbonyl)-5,6-dihydro-4H-cyclopenta[d][1,3]thiazole;
2-{4-[3-(2-methylpyrrolidin-1-yl)propoxy]phenyl}-5,6-dihydro-4H-cyclopenta[d][1,3]thiazol-5-amine;
N-(2-{4-[3-(2-methylpyrrolidin-1-yl)propoxy]phenyl}-5,6-dihydro-4H-cyclopenta[d][1,3]-thiazol-5-yl)acetamide;
5-[(4,4-difluoropiperidin-1-yl)carbonyl]-2-(4-{3-[(2R)-2-methylpyrrolidin-1-yl]propoxy}-phenyl)-5,6-dihydro-4H-cyclopenta[d][1,3]thiazole;
N-ethyl-2-(4-{3-[(2R)-2-methylpyrrolidin-1-yl]propoxy}phenyl)-5,6-dihydro-4H-cyclopenta-[d][1,3]thiazole-5-carboxamide;
2-(4-{3-[(2R)-2-methylpyrrolidin-1-yl]propoxy}phenyl)-5-(pyrrolidin-1-ylcarbonyl)-5,6-dihydro-4H-cyclopenta[d][1,3]thiazole;
5-[(4-methylpiperazin-1-yl)carbonyl]-2-(4-{3-[(2R)-2-methylpyrrolidin-1-yl]propoxy}phenyl)-5,6-dihydro-4H-cyclopenta[d][1,3]thiazole;
5-[(4,4-difluoropiperidin-1-yl)carbonyl]-2-{4-[3-(4-isopropylpiperazin-1-yl)propoxy]phenyl}-5,6-dihydro-4H-cyclopenta[d][1,3]thiazole;
2-{4-[3-(4-isopropylpiperazin-1-yl)propoxy]phenyl}-5-(thiomorpholin-4-ylcarbonyl)-5,6-dihydro-4H-cyclopenta[d][1,3]thiazole;
2-{4-[3-(4-isopropylpiperazin-1-yl)propoxy]phenyl}-5-(morpholin-4-ylcarbonyl)-5,6-dihydro-4H-cyclopenta[d][1,3]thiazole;
5-[(4-isopropylpiperazin-1-yl)carbonyl]-2-{4-[3-(4-isopropylpiperazin-1-yl)propoxy]phenyl}-5,6-dihydro-4H-cyclopenta[d][1,3]thiazole;
2-[(2-{4-[3-(4-isopropylpiperazin-1-yl)propoxy]phenyl}-5,6-dihydro-4H-cyclopenta[d]-[1,3]thiazol-5-yl)carbonyl]decahydroisoquinoline;
N-(cyclopropylmethyl)-2-{4-[3-(4-isopropylpiperazin-1-yl)propoxy]phenyl}-5,6-dihydro-4H-cyclopenta[d][1,3]thiazole-5-carboxamide;
N-(cyclopropylmethyl)-2-{4-[3-(4-isopropylpiperazin-1-yl)propoxy]phenyl}-N-propyl-5,6-dihydro-4H-cyclopenta[d][1,3]thiazole-5-carboxamide;
2-{4-[3-(4-isopropylpiperazin-1-yl)propoxy]phenyl}-5-(pyrrolidin-1-ylcarbonyl)-5,6-dihydro-4H-cyclopenta[d][1,3]thiazole;
5-acetyl-2-{4-[3-(2-methylpyrrolidin-1-yl)propoxy]phenyl}-5,6-dihydro-4H-pyrrolo[3,4-d][1,3]thiazole; and
N-ethyl-2-{4-[3-(2-methylpyrrolidin-1-yl)propoxy]phenyl}-4,6-dihydro-5H-pyrrolo[3,4-d][1,3]thiazole-5-carboxamide.

18. (canceled)

19. A pharmaceutical composition comprising an effective amount of a compound according to claim 1, or a pharmaceutically acceptable salt thereof as well as a pharmaceutically acceptable diluent or carrier.

20. A method treating mild-cognitive impairment, Alzheimer's disease, learning and memory disorders, attention-deficit hyperactivity disorder, Parkinson's disease, schizophrenia, dementia, depression, epilepsy, seizure disorders, convulsions, sleep/wake disorders, cognitive dysfunctions, narcolepsy, hypersomnia, obesity, upper airway allergic disorders, Down's syndrome, anxiety, stress, cardiovascular disorders, inflammation or pain in a subject, the method comprising administering a therapeutically effective amount of a compound according to claim 1 to a patient in need of such treatment.
